(19) United States
(12) Patent Application Publication
(10) Pub. No.: US 2024/0122950 A1
(43) Pub. Date: Apr. 18, 2024

(54) MULTI-SUBSTITUENT PSILOCYBIN DERIVATIVES AND METHODS OF USING

(71) Applicant: Enveric Biosciences Canada Inc., Calgary (CA)

(72) Inventors: Jillian M. Hagel, Calgary (CA); Peter J. Facchini, Calgary (CA); Chang-Chun Ling, Calgary (CA)

(21) Appl. No.: 18/390,195

(22) Filed: Dec. 20, 2023

Related U.S. Application Data
(63) Continuation of application No. 17/956,139, filed on Sep. 29, 2022, now Pat. No. 11,918,594.
Application No. 17/956,139 is a continuation of application No. PCT/CA2022/050206, filed on Feb. 11, 2022.
(60) Provisional application No. 63/247,881, filed on Sep. 24, 2021.
(60) Provisional application No. 63/149,001, filed on Feb. 12, 2021.

(51) Int. Cl. A61K 31/675; A61P 25/18

(52) U.S. Cl. CPC A61K 31/675; A61P 25/18

(57) ABSTRACT

Disclosed are novel multi-substituent psilocybin derivative compounds and pharmaceutical and recreational drug formulations containing the same. The compounds may be produced by reacting a reactant psilocybin derivative with a substituent containing compound.

### RELATED APPLICATIONS

This application is a continuation of U.S. patent application Ser. No. 17/956,139 filed Sep. 29, 2022, which is a continuation of PCT Application No. PCT/CA2022/050206 filed Feb. 11, 2022, which claims the benefit of U.S. Provisional Application No. 63/149,001 filed Feb. 12, 2021 and U.S. Provisional Application No. 63/247,881 filed Sep. 24, 2021; the entire contents of patent application Ser. No. 17/956,139, PCT/CA2022/050206, 63/149,001 and 63/247,881 are hereby incorporated by reference.

### INCORPORATION OF SEQUENCE LISTING

A computer readable form of the Sequence Listing “29664-P63978US03_SequenceListing” (151,056 bytes), submitted via EFS-WEB and created on Dec. 18, 2023, is herein incorporated by reference.

### FIELD OF THE DISCLOSURE

The compositions and methods disclosed herein relate to a chemical compound known as psilocybin. Furthermore, the compositions and methods disclosed herein relate in particular to derivatives of psilocybin comprising multiple substituent groups.

### BACKGROUND OF THE DISCLOSURE

The following paragraphs are provided by way of background to the present disclosure. They are not however an admission that anything discussed therein is prior art or part of the knowledge of a person of skill in the art.

The biochemical pathways in the cells of living organisms may be classified as being part of primary metabolism, or as being part of secondary metabolism. Pathways that are part of a cell's primary metabolism are involved in catabolism for energy production or in anabolism for building block production for the cell. Secondary metabolites, on the other hand, are produced by the cell without having an obvious anabolic or catabolic function. It has long been recognized that secondary metabolites can be useful in many respects, including as therapeutic compounds.

Psilocybin, for example, is a secondary metabolite that is naturally produced by certain mushrooms which taxonomically can be classified as belonging the Basidiomycota division of the fungi kingdom. Mushroom species which can produce psilocybin include species belonging to the genus Psilocybe, such as Psilocybe azurescens, Psilocybe semilanceata, Psilocybe serbica, Psilocybe mexicana, and Psilocybe cyanescens, for example. The interest of the art in psilocybin is well established. Thus, for example, psilocybin is a psychoactive compound and is therefore used as a recreational drug. Furthermore, psilocybin is used as a research tool in behavioral and neuro-imaging studies in psychotic disorders, and has been evaluated for its clinical potential in the treatment of mental health conditions (Daniel, J. et al., Mental Health Clin/, 2017; 7(1): 24-28), including to treat anxiety in terminal cancer patients (Grob, C. et al., Arch. Gen. Psychiatry, 2011, 68(1) 71-78) and to alleviate symptoms of treatment-resistant depression (Cathart-Harris, R. L. et al., Lancet Psychiatry, 2016, 3: 619-627).

Although the toxicity of psilocybin is low, adverse side effects, including, for example, panic attacks, paranoia, and psychotic states, sometimes together or individually referred to as “a bad trip”, are not infrequently experienced by recreational psilocybin users.

There exists therefore a need in the art for improved psilocybin compounds.

### SUMMARY OF THE DISCLOSURE

The following paragraphs are intended to introduce the reader to the more detailed description, not to define or limit the claimed subject matter of the present disclosure.

In one aspect, the present disclosure relates to psilocybin and derivative compounds thereof.

In another aspect, the present disclosure relates to psilocybin derivative compounds and methods of making and using these compounds.

In another aspect, the present disclosure relates to multiple-substituent psilocybin derivative compounds.

Accordingly, in one aspect, the present disclosure provides, in at least one embodiment, in accordance with the teachings herein, a chemical compound or a salt thereof having a formula (I):

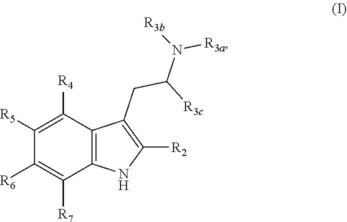

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, and wherein R₃ₐ and R_{3b} are each independently a hydrogen atom, an alkyl group, a cycloalkyl group, an acyl group, or an aryl group, and R_{3c} is a hydrogen atom or a carboxyl group.

In at least one embodiment, in an aspect, at least three of R₂, R₄, R₅, R₆, or R₇ can be substituents selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

In at least one embodiment, in an aspect, at least three of R₂, R₄, R₅, R₆, or R₇ can be substituents selected from at least three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

In at least one embodiment, in an aspect, when R₄ is not substituted with a substituents, R₄ can be a hydrogen atom.

In at least one embodiment, in an aspect, R₄ and R₅ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₆ and R₇ can be hydrogen atoms.

In at least one embodiment, in an aspect, R₄ and R₅ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₄ and R₅ can be a prenyl group or a halogen atom, and R₂, R₆ and R₇ can be hydrogen atoms.

In at least one embodiment, in an aspect, R₄ and R₆ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₄ and R₇ can be hydrogen atoms.

In at least one embodiment, in an aspect, R₄ and R₆ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₄ and R₆ can be a prenyl group or a halogen atom, and R₂, R₄ and R₇ can be hydrogen atoms.

In at least one embodiment, in an aspect, R₄ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₅ and R₆ can be hydrogen atoms.

In at least one embodiment, in an aspect, R₄ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₄ and R₇ can be a prenyl group or a halogen atom, and R₂, R₅ and R₆ can be hydrogen atoms.

In at least one embodiment, in an aspect, R₅ and R₆ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₄ and R₇ can be hydrogen atoms.

In at least one embodiment, in an aspect, R₅ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₅ and R₆ can be a prenyl group or a halogen atom, and R₂, R₄ and R₆ can be hydrogen atoms.

In at least one embodiment, in an aspect, R₅ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₄ and R₆ can be hydrogen atoms.

In at least one embodiment, in an aspect, R₅ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₅ and R₇ can be a prenyl group or a halogen atom, and R₂, R₄ and R₆ can be hydrogen atoms.

In at least one embodiment, in an aspect, R₆ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and R₂, R₄ and R₅ can be hydrogen atoms.

In at least one embodiment, in an aspect, R₆ and R₇ can be selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and at least one of R₆ and R₇ can be a prenyl group or a halogen atom, and R₂, R₄ and R₅ can be hydrogen atoms.

In at least one embodiment, in an aspect, R₂ can be hydrogen, and only two of R₄, R₅, R₆, or R₇, are substituted, wherein the first substituent is selected from (i) a halogen atom, (ii) a prenyl group, and (iii) a nitrile group, and the second substituent is selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein the first and second substituents are from different groups.

In at least one embodiment, in an aspect, R₂ can be hydrogen, and only two of R₄, R₅, R₆, or R₇, are substituted, wherein the first substituent is a halogen atom, and the second substituent is selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a nitrile group.

In at least one embodiment, in an aspect, R₂ can be hydrogen, and only two of R₄, R₅, R₆, or R₇, are substituted, wherein the first substituent is a prenyl group, and the second substituent is selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, and (viii) a nitrile group.

In at least one embodiment, in an aspect, R₂ can be hydrogen, and only two of R₄, R₅, R₆, or R₇, are substituted, wherein the first substituent is a nitrile atom, and the second substituent is selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, and (viii) a prenyl group.

In at least one embodiment, in an aspect, R₂ can be hydrogen, and only two of R₄, R₅, R₆, or R₇, are substituted, wherein the first substituent is selected from (i) a halogen atom, (ii) a prenyl group, and (iii) a nitrile group, and the second substituent is selected from (i) a hydroxy group, (ii) a nitro group, (iii) a glycosyloxy group, (iv) an amino group or an N-substituted amino group, (v) a carboxyl group or a carboxylic acid derivative, and (vi) an aldehyde or a ketone group.

In at least one embodiment, in an aspect, R₂ can be hydrogen, and only two of R₄, R₅, R₆, or R₇, are substituted, wherein the first substituent is a halogen atom, and the second substituent is selected from can be selected from (i) an amino group or N-substituted amino group, (ii) a nitrile group, (iii) a nitro group, (iv) a hydroxy group and (v) a prenyl group.

In at least one embodiment, in an aspect, R₂ can be hydrogen, and only two of R₄, R₅, R₆, or R₇, are substituted, wherein the first substituent is a prenyl group, and the second substituent is selected from (i) a carboxyl group or a carboxylic acid derivative, (ii) a halogen, and (iii) a hydroxy group, wherein R₂ can be a hydrogen atom, and two of R₄, R₅, R₆, or R₇ can be substituents.

In at least one embodiment, in an aspect, R₂ can be hydrogen, and only two of R₄, R₅, R₆, or R₇, are substituted, wherein the first substituent is a halogen atom, and the second substituent is a prenyl group.

In at least one embodiment, in an aspect, R₂ can be hydrogen, and only two of R₄, R₅, R₆, or R₇, are substituted, wherein the first substituent is a nitrile group, and the second substituent is an amino group or N-substituted amino group.

In at least one embodiment, in an aspect, R₅ can be a carboxyl group or an acetyl group, and R₇ can be an amino group, a nitrile group, a hydroxy group, or a halogen.

In at least one embodiment, in an aspect, R₅ can be an acetamidyl group, and R₇ can be an aldehyde group, a carboxyl group, or a carboxyester.

In at least one embodiment, in an aspect, R₅ can be an acetamidyl group, R₆ can be an amino group, a nitro group, or a halogen, and R₇ can be an aldehyde group, a carboxyl group, or a carboxyester.

In at least one embodiment, in an aspect, R₅ can be a carboxy-methyl group or an amide group, and R₇ can be a nitro group, and amino group or a halogen.

In at least one embodiment, in an aspect, R₄ can be a glycosyloxy group, R₅ can be a carboxy-methyl group or an amide group, and R₇ can be a nitro group, and amino group or a halogen.

In at least one embodiment, in an aspect, chemical compound (I) can be selected from a compound having a chemical formula (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XLVIII), (XLIX), (L), (LI), (LII), (LIII), (LIV), (LV) or (LXXVI):

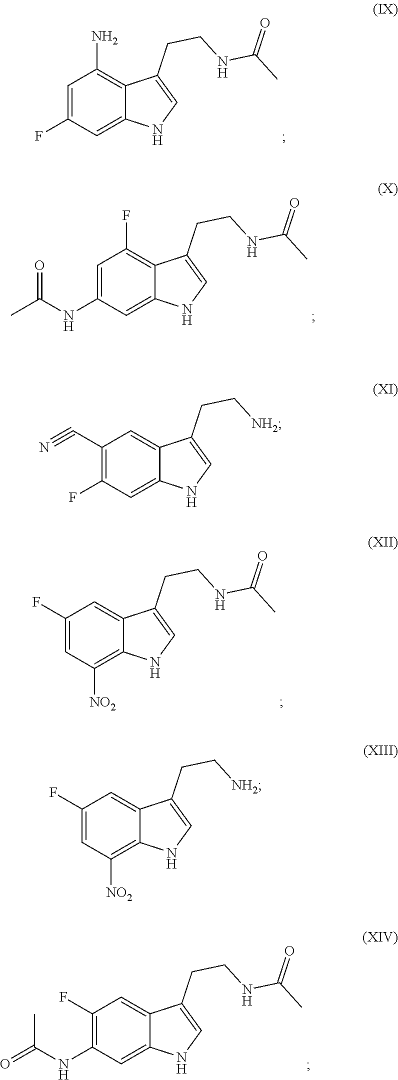

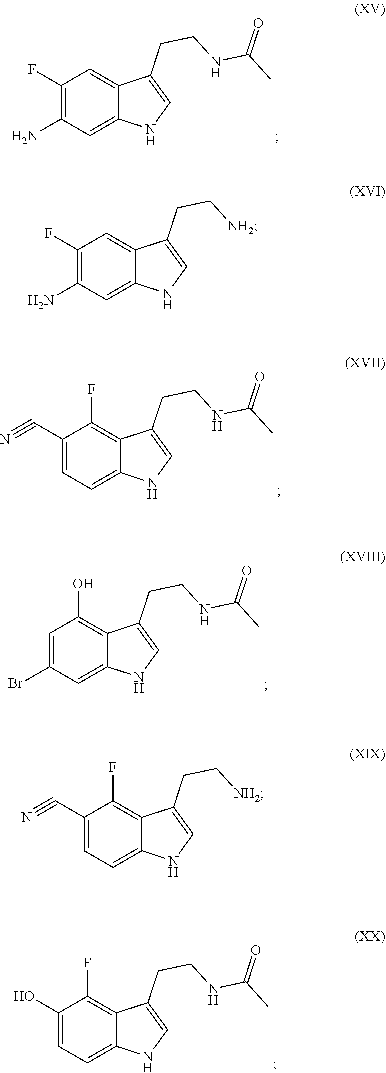 [SMILES: CC(=O)NCCc1c[nH]c2cc(N)c(F)cc12 ; NCCc1c[nH]c2cc(N)c(F)cc12 ; CC(=O)NCCc1c[nH]c2ccc(C#N)c(F)c12 ; CC(=O)NCCc1c[nH]c2cc(Br)cc(O)c12 ; N#Cc1ccc2[nH]cc(CCN)c2c1F ; CC(=O)NCCc1c[nH]c2ccc(O)c(F)c12]

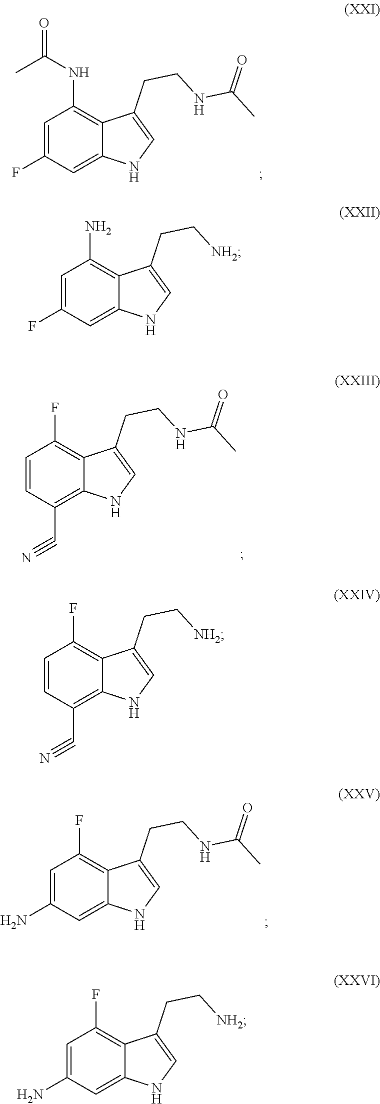 [SMILES: CC(=O)NCCc1c[nH]c2cc(F)cc(NC(C)=O)c12 ; NCCc1c[nH]c2cc(F)cc(N)c12 ; CC(=O)NCCc1c[nH]c2c(C#N)ccc(F)c12 ; N#Cc1ccc(F)c2c(CCN)c[nH]c12 ; CC(=O)NCCc1c[nH]c2cc(N)cc(F)c12 ; NCCc1c[nH]c2cc(N)cc(F)c12]

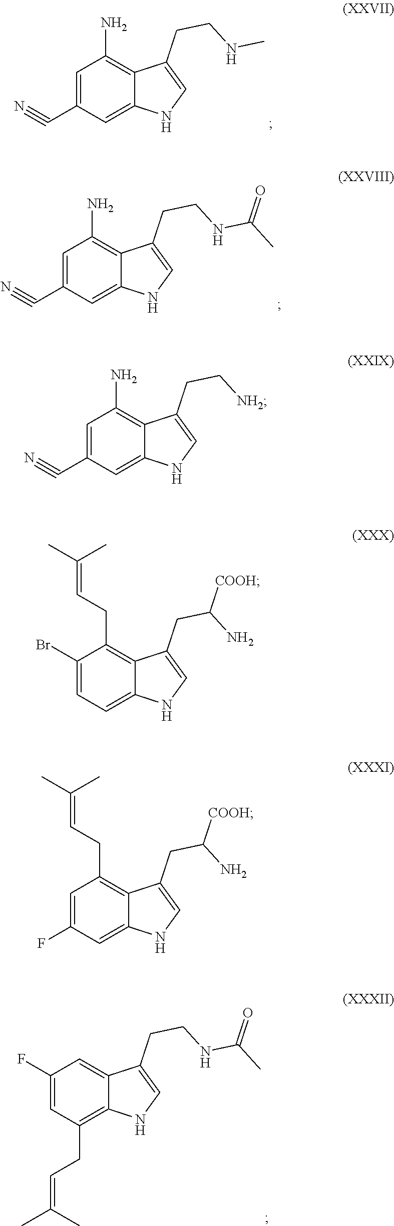

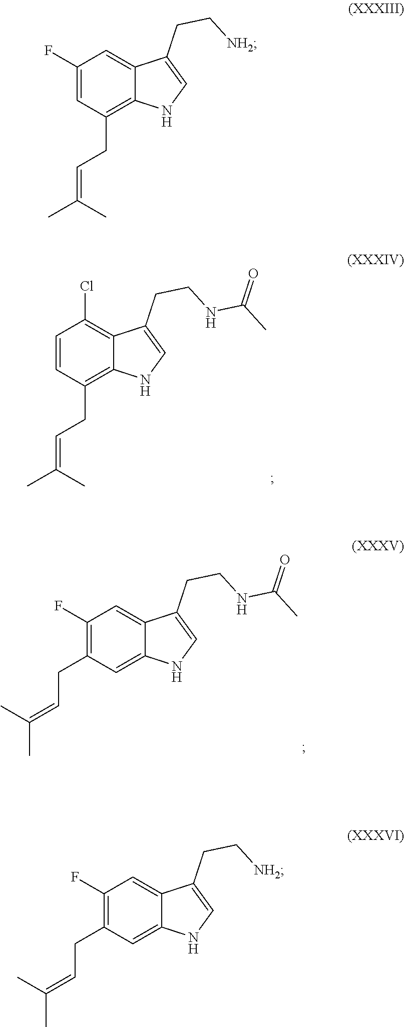

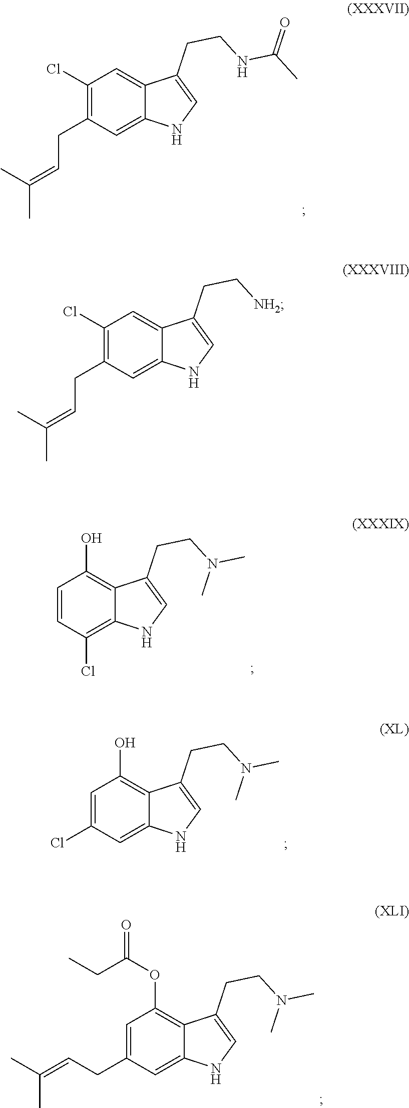

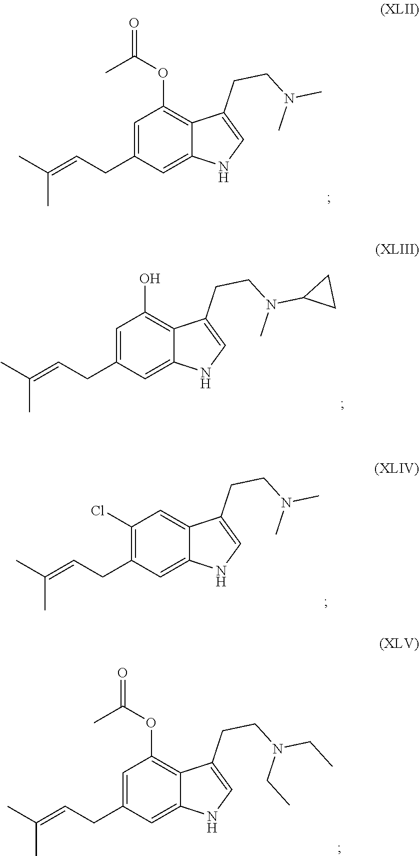

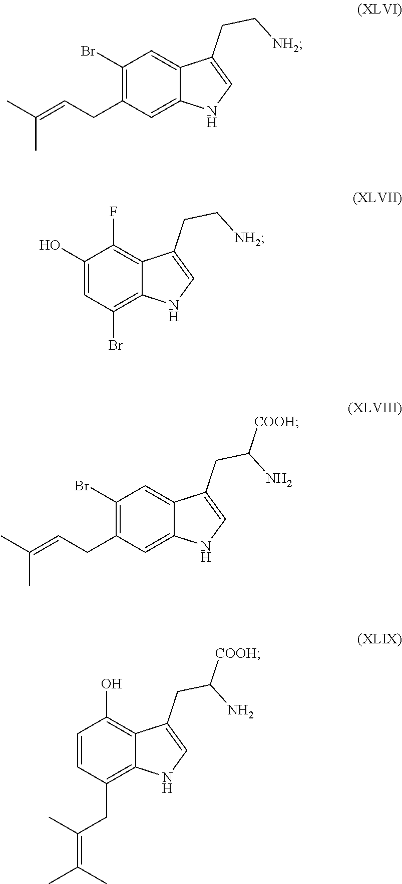 [SMILES: CC(C)=CCc1cc2[nH]cc(CCN)c2cc1Br ; NCCc1c[nH]c2c(Br)cc(O)c(F)c12 ; CC(C)=CCc1cc2[nH]cc(CC(N)C(=O)O)c2cc1Br ; CC(C)=C(C)Cc1ccc(O)c2c(CC(N)C(=O)O)c[nH]c12]

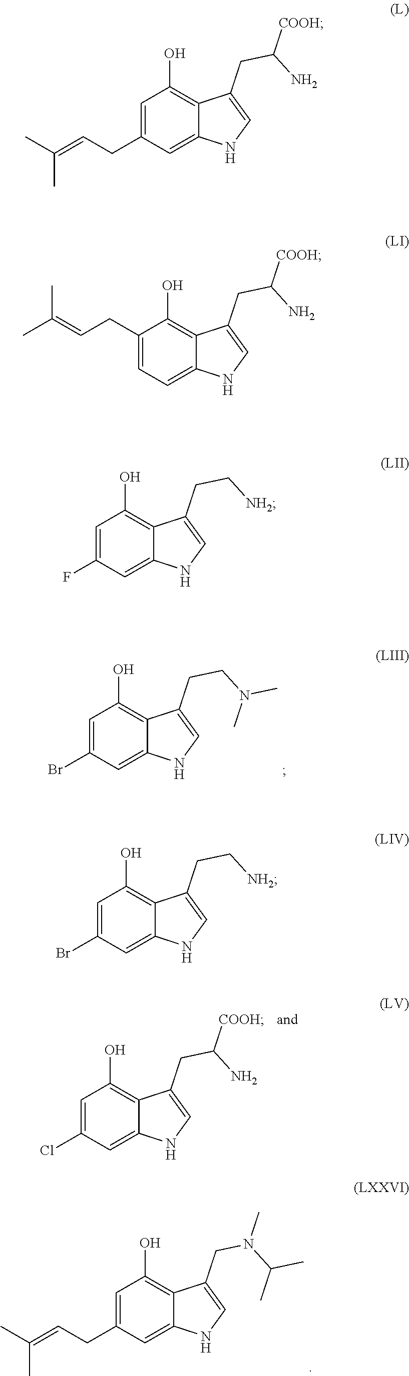

In at least one embodiment, chemical compound (I) can be any one of the compounds shown in FIG. 13A, FIG. 13B and FIG. 13C and labeled therein as 13A-3, 13A-4, 13A-5, 13A-6, 13A-7, 13A-8, 13A-9, 13A-10, 13B-3, 13B-4, 13B-5, 13B-6, 13B-7, 13B-8, 13B-8, 13C-6, 13C-7, 13C-8, 13C-9, 13C-10, or 13C-11.

In another aspect, the present disclosure relates to pharmaceutical and recreational drug formulations comprising psilocybin derivative compounds. Accordingly, in one aspect, the present disclosure provides, in at least one embodiment, a pharmaceutical or recreational drug formulation comprising an effective amount of a chemical compound or a salt thereof having a formula (I):

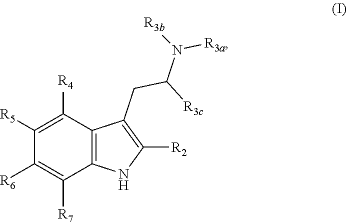

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, together with a pharmaceutically acceptable excipient, diluent or carrier.

In another aspect, the present disclosure relates to methods of treatment of psychiatric disorders. Accordingly, the present disclosure further provides, in one embodiment a method for treating a psychiatric disorder, the method comprising administering to a subject in need thereof a pharmaceutical formulation comprising a chemical compound or a salt thereof having a formula (I):

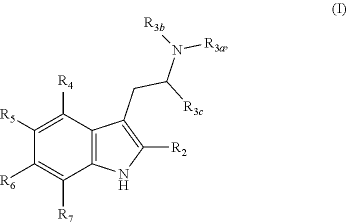

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, wherein the pharmaceutical formulation is administered in an effective amount to treat the psychiatric disorder in the subject.

In at least one embodiment, in an aspect, the disorder can be a 5-HT_{2A} receptor mediated disorder, or a 5-HT_{1A} receptor mediated disorder.

In at least one embodiment, in an aspect, a dose can be administered of about 0.001 mg to about 5,000 mg.

In another aspect, the present disclosure provides, in at least one embodiment, a method for modulating a 5-HT_{2A} receptor or a 5-HT_{1A} receptor, the method comprising contacting a 5-HT_{2A} receptor or a 5-HT_{1A} receptor with a chemical compound or salt thereof having a formula (I):

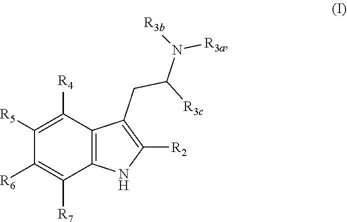

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group.

In at least one embodiment, in an aspect, the reaction conditions can be in vitro reaction conditions.

In at least one embodiment, in an aspect, the reaction conditions can be in vivo reaction conditions.

In another aspect, the present disclosure relates to methods of making multi-substituent psilocybin derivative compounds. Accordingly, in one aspect, the present disclosure provides, in at least one embodiment, a method of making a psilocybin derivative or salt thereof having a chemical formula (I):

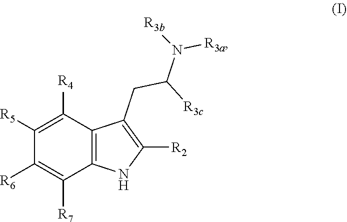

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, the method comprising:
reacting a reactant psilocybin derivative having a chemical formula (II):

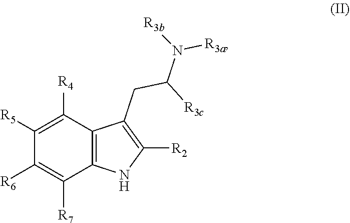

wherein, one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom or an alcohol group and when R₄ is not substituted with any of the foregoing substituents, R₄, is a hydrogen atom, a hydroxy group, an O-alkyl group, O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group
with
   a substituent containing compound, wherein the substituent in the substituent containing compound is selected from (i) a halogen containing compound, (ii) a hydroxy group containing compound, (iii) a nitro group containing compound, (iv) a glycosyloxy group containing compound, (v) an amino group or an N-substituted amino group containing compound, (vi) a carboxyl group or a carboxylic acid derivative containing compound, (vii) an aldehyde or a ketone group containing compound, (viii) a prenyl group containing compound, and (ix) a nitrile group containing compound under reaction conditions sufficient to form the psilocybin substituent or salt thereof having chemical formula (I).

In at least one embodiment, in an aspect, the substituent in the reactant psilocybin derivative having the formula (II) can be a nitro group, the substituent containing compound can be the carboxylic acid derivative acetic anhydride (Ac₂O), and the reactant psilocybin derivative and the substituent containing compound can be reacted in a Friedl-Crafts acylation reaction to form a first psilocybin derivative having formula (I), wherein one of R₂, R₄, R₅, R₆, or R₇ is a nitro group, and at least one of R₂, R₄, R₅, R₆, or R₇ is an acetyl group.

In at least one embodiment, in an aspect, the formed first psilocybin derivative can be the compound shown in FIG. 13A and labeled 13A-3.

In at least one embodiment, in an aspect, the formed first psilocybin derivative having formula (I) can be reacted to oxidize the acetyl group and form a second psilocybin derivative having formula (I), wherein one of R₂, R₄, R₅, R₆, or R₇ can be a nitro group, and at least one of R₂, R₄, R₅, R₆, or R₇ can be a carboxyl group.

In at least one embodiment, in an aspect, the formed second psilocybin derivative can be the compound shown in FIG. 13A and labeled 13A-4.

In at least one embodiment, in an aspect, the formed second psilocybin derivative having formula (I) can be reacted to reduce the nitro group and form an amino group, and a third psilocybin derivative having formula (I), wherein one of R₂, R₄, R₅, R₆, or R₇ can be an amino group, and at least one of R₂, R₄, R₅, R₆, or R₇ can be a carboxyl group.

In at least one embodiment, in an aspect, the formed third psilocybin derivative can be the compound shown in FIG. 13A and labeled 13A-5.

In at least one embodiment, in an aspect, the formed third psilocybin derivative having formula (I) can be reacted with a nitrite to convert the amino group in a diazonium salt and form an intermediate psilocybin derivative having formula (I), wherein one of R₂, R₄, R₅, R₆, or R₇ can be a diazonium group, and at least one of R₂, R₄, R₅, R₆, or R₇ can be a carboxyl group.

In at least one embodiment, in an aspect, the intermediate formed psilocybin derivative can be the compound shown in FIG. 13A and labeled 13A-6.

In at least one embodiment, in an aspect, the intermediate formed psilocybin derivative having formula (I) can be reacted with a nitrile containing compound to convert the diazonium group and form a fourth psilocybin derivative having formula (I), wherein one of R₂, R₄, R₅, R₆, or R₇ can be a nitrile group, and at least one of R₂, R₄, R₅, R₆, or R₇ can be a carboxyl group.

In at least one embodiment, in an aspect, the formed fourth psilocybin derivative can be the compound shown in FIG. 13A and labeled 13A-7.

In at least one embodiment, in an aspect, the intermediate formed psilocybin derivative having formula (I) can be reacted with water to convert the diazonium group and form a fifth psilocybin derivative having formula (I), wherein one of R₂, R₄, R₅, R₆, or R₇ can be a hydroxy group, and at least one of R₂, R₄, R₅, R₆, or R₇ can be a carboxyl group.

In at least one embodiment, in an aspect, the formed fifth psilocybin derivative can be the compound shown in FIG. 13A and labeled 13A-8.

In at least one embodiment, in an aspect, the intermediate formed psilocybin derivative having formula (I) can be reacted with a halogen containing compound to convert the diazonium group and form a sixth psilocybin derivative having formula (I), wherein one of R₂, R₄, R₅, R₆, or R₇ can be a halogen atom, and at least one of R₂, R₄, R₅, R₆, or R₇ can be a carboxyl group.

In at least one embodiment, in an aspect, the formed sixth psilocybin derivative can be the compound shown in FIG. 13A and labeled 13A-9 or 13A-10.

In at least one embodiment, in an aspect, the substituent in the reactant psilocybin derivative having formula (II) can be a methoxycarbonyl group, the substituent containing compound can be the halogen containing compound N-halo-succinimide, and the reactant psilocybin derivative and the substituent containing compound can be reacted to form a first psilocybin derivative having formula (I), wherein one of R₂, R₄, R₅, R₆, or R₇ can be a methoxy carbonyl group, and at least one of R₂, R₄, R₅, R₆, or R₇ can be a halogen atom.

In at least one embodiment, in an aspect, the N-halo-succinimide can be N-chloro-succinimide, and the formed first psilocybin derivative can be the compound shown in FIG. 13B and labeled 13B-3.

In at least one embodiment, in an aspect, the substituent in the reactant psilocybin derivative having formula (II) can be a methoxycarbonyl group, the substituent containing compound can be the nitro containing compound nitronium tetrafluoroborate, and the reactant psilocybin derivative and the substituent containing compound can be reacted to form a second psilocybin derivative having formula (I), wherein one of R₂, R₄, R₅, R₆, or R₇ is a methoxy carbonyl group, and at least one of R₂, R₄, R₅, R₆, or R₇ is a nitro group.

In at least one embodiment, in an aspect, and the formed second psilocybin derivative can be the compound shown in FIG. 13B and labeled 13B-4.

In at least one embodiment, in an aspect, the formed first psilocybin derivative having formula (I) can be reacted with an acetylated glycosyl compound and form a third psilocybin derivative having formula (I), wherein one of R₂, R₄, R₅, R₆, or R₇ is a methoxy carbonyl group, at least one of R₂, R₄, R₅, R₆, or R₇ can be a glycosyloxy group, and at least one of R₂, R₄, R₅, R₆, or R₇ can be a halogen atom.

In at least one embodiment, in an aspect, the formed third psilocybin derivative can be the compound shown in FIG. 13B and labeled 13B-5.

In at least one embodiment, in an aspect, the formed second psilocybin derivative having formula (I) can be reacted with an acetylated glycosyl compound and form a fourth psilocybin derivative having formula (I), wherein one of R₂, R₄, R₅, R₆, or R₇ can be a methoxycarbonyl group, at least one of R₂, R₄, R₅, R₆, or R₇ can be a glycosyloxy group, and at least one of R₂, R₄, R₅, R₆, or R₇ is a nitro group.

In at least one embodiment, in an aspect, the formed fourth psilocybin derivative can be the compound shown in FIG. 13B and labeled 13B-6.

In at least one embodiment, in an aspect, the formed third psilocybin derivative having formula (I) can be reacted with ammonia to convert the methoxycarbonyl group in an amido group and form a fifth psilocybin derivative having formula (I), wherein one of R₂, R₄, R₅, R₆, or R₇ can be an amido group, at least one of R₂, R₄, R₅, R₆, or R₇ is a glycosyloxy group, and at least one of R₂, R₄, R₅, R₆, or R₇ can be a halogen atom.

In at least one embodiment, in an aspect, the formed fifth psilocybin derivative can be the compound shown in FIG. 13B and labeled 13B-7.

In at least one embodiment, in an aspect, the formed fourth psilocybin derivative having formula (I) can be reacted with ammonia to convert the methoxycarbonyl group in an amido group and form a sixth psilocybin derivative having formula (I), wherein one of R₂, R₄, R₅, R₆, or R₇ can be an amido group, at least one of R₂, R₄, R₅, R₆, or R₇ can be a glycosyloxy group, and at least one of R₂, R₄, R₅, R₆, or R₇ is a nitro group.

In at least one embodiment, in an aspect, the formed sixth psilocybin derivative can be the compound shown in FIG. 13B and labeled 13B-8.

In at least one embodiment, in an aspect, the formed sixth psilocybin derivative having formula (I) can be reacted to reduce the nitro group to form an amino group and a seventh psilocybin derivative having formula (I), wherein one of R₂, R₄, R₅, R₆, or R₇ can be an amido group, at least one of R₂, R₄, R₅, R₆, or R₇ is a glycosyloxy group, and at least one of R₂, R₄, R₅, R₆, or R₇ can be a nitro group.

In at least one embodiment, in an aspect, the formed seventh psilocybin derivative can be the compound shown in FIG. 13B and labeled 13B-9.

In at least one embodiment, in an aspect, the substituent in the reactant psilocybin derivative having formula (II) can be an acetamidyl group, the substituent containing compound can be the halogen containing compound N-halo-succinimide, and the reactant psilocybin derivative and the substituent containing compound can be reacted to form a first psilocybin derivative having formula (I), wherein one of R₂, R₄, R₅, R₆, or R₇ can be an acetamidyl group, and at least one of R₂, R₄, R₅, R₆, or R₇ can be a halogen atom.

In at least one embodiment, in an aspect, the N-halo-succinimide can be N-bromo-succinimide (NBS), and the formed first psilocybin derivative can be the compound shown in FIG. 13C and labeled 13C-6.

In at least one embodiment, in an aspect, the substituent in the reactant psilocybin derivative having formula (II) can be an acetamidyl group, the substituent containing compound can be dimethyl formamide, and the reactant psilocybin derivative and the substituent containing compound can be reacted to form an intermediate psilocybin derivative having formula (I), wherein one of R₂, R₄, R₅, R₆, or R₇ is an acetamidyl group, and at least one of R₂, R₄, R₅, R₆, or R₇ can be a methanol group, and wherein the intermediate psilocybin derivative can be reacted to oxidize the methanol group, and form a second psilocybin derivative having formula (I), wherein one of R₂, R₄, R₅, R₆, or R₇ can be an acetamidyl group, and at least one of R₂, R₄, R₅, R₆, or R₇ can be a carboxy group.

In at least one embodiment, in an aspect, the intermediate psilocybin derivative can be the compound shown in FIG. 13C and labeled 13C-5, and the formed second psilocybin derivative can be the compound shown in FIG. 13C and labeled 13C-7.

In at least one embodiment, in an aspect, the formed second psilocybin derivative having formula (I) can be reacted with an alcohol to esterify the carboxy group to form an ester and a third psilocybin derivative having formula (I), wherein one of R₂, R₄, R₅, R₆, or R₇ can be an acetamidyl group, at least one of R₂, R₄, R₅, R₆, or R₇ can be a carboxyl ester.

In at least one embodiment, in an aspect, the formed third psilocybin derivative can be the compound shown in FIG. 13C and labeled 13C-8.

In at least one embodiment, in an aspect, the formed third psilocybin derivative having formula (I) can be reacted with a nitro group containing compound and form a fourth psilocybin derivative having formula (I), wherein one of R₂, R₄, R₅, R₆, or R₇ can be an acetamidyl group, at least one of R₂, R₄, R₅, R₆, or R₇ can be a carboxyl ester, and at least one of R₂, R₄, R₅, R₆, or R₇ is a nitro group.

In at least one embodiment, in an aspect, the formed fourth psilocybin derivative can be the compound shown in FIG. 13C and labeled 13C-9.

In at least one embodiment, in an aspect, the formed fourth psilocybin derivative having formula (I) can be reacted to reduce the nitro group to form an amino group and a fifth psilocybin derivative having formula (I), wherein one of R₂, R₄, R₅, R₆, or R₇ is an acetamidyl group, at least one of R₂, R₄, R₅, R₆, or R₇ is a carboxyl ester, and at least one of R₂, R₄, R₅, R₆, or R₇ is an amino group.

In at least one embodiment, in an aspect, the formed fifth psilocybin derivative can be the compound shown in FIG. 13C and labeled 13C-10.

In at least one embodiment, in an aspect, the formed fifth psilocybin derivative having formula (I) can be reacted with ammonia to form an amido group and a sixth psilocybin derivative having formula (I), wherein one of R₂, R₄, R₅, R₆, or R₇ can be an acetamidyl group, at least one of R₂, R₄, R₅, R₆, or R₇ is a carboxyester, and at least one of R₂, R₄, R₅, R₆, or R₇ can be an amido group.

In at least one embodiment, in an aspect, the formed sixth psilocybin derivative can be the compound shown in FIG. 13C and labeled 13C-11.

In another aspect, the present disclosure relates to further methods of making multi-substituent psilocybin derivatives. Accordingly, in one aspect, the present disclosure provides, in at least one aspect, a method of making a multi-substituent psilocybin derivative, the method comprising contacting a psilocybin derivative precursor compound having a formula (LVII):

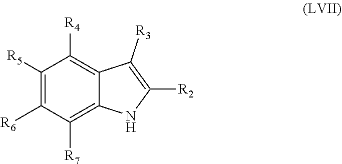

wherein at least one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, and wherein R₃ is a hydrogen atom or —CH₂—CHNH₂COOH or —CH₂—CH₂NH₂,
   with a catalytic quantity of a psilocybin biosynthetic enzyme complement under reaction conditions permitting an enzyme catalyzed conversion of the psilocybin derivative precursor compound to form a multi-substituent psilocybin derivative compound having a formula (I):

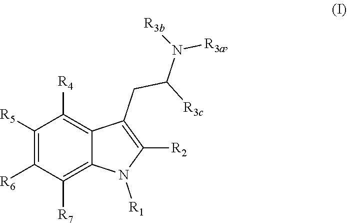

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄, is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

In at least one embodiment, in an aspect, the reaction conditions can be in vitro reaction conditions.

In at least one embodiment, in an aspect, the reaction conditions can be in vivo reaction conditions.

In at least one embodiment, in an aspect, the psilocybin derivative precursor compound and the substituent containing compound can be contacted with the psilocybin biosynthetic enzyme complement in a host cell, wherein the host cell can comprise a chimeric nucleic acid sequence comprising as operably linked components:
(i) a nucleic acid sequence controlling expression in the host cell, and
(ii) a nucleic acid sequence encoding psilocybin biosynthetic enzyme complement,
   and the host cell can be grown to express the psilocybin biosynthetic enzyme complement and to produce the multi-substituent psilocybin derivative compound.

In at least one embodiment, in an aspect, the psilocybin biosynthetic enzyme complement can comprise at least one enzyme encoded by a nucleic acid selected from:
(a) SEQ. ID NO: 1, SEQ. ID NO: 3, SEQ. ID NO: 5, SEQ. ID NO: 7, SEQ. ID NO: 9, SEQ. ID NO 11, SEQ. ID NO: 13, SEQ. ID NO: 15, SEQ. ID NO: 17, SEQ. ID NO: 19, SEQ. ID NO: 21, SEQ. ID NO 23, SEQ. ID NO: 25, and SEQ. ID NO: 48;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 2, SEQ. ID NO: 4, SEQ. ID NO: 6, SEQ. ID NO: 8, SEQ. ID NO: 10, SEQ. ID NO 12, SEQ. ID NO: 14, SEQ. ID NO: 16, SEQ. ID NO: 18, SEQ. ID NO: 20, SEQ. ID NO: 22, SEQ. ID NO 24, SEQ. ID NO: 26, and SEQ. ID NO: 49;
(f) a nucleic acid sequence that encodes a functional variant of any one of the amino acid sequences set forth in SEQ. ID NO: 2, SEQ. ID NO: 4, SEQ. ID NO: 6, SEQ. ID NO: 8, SEQ. ID NO: 10, SEQ. ID NO 12, SEQ. ID NO: 14, SEQ. ID NO: 16, SEQ. ID NO: 18, SEQ. ID NO: 20, SEQ. ID NO: 22, SEQ. ID NO 24, SEQ. ID NO: 26, and SEQ. ID NO: 49; and
(g) a nucleic acid sequence that hybridizes under stringent conditions to any one of the nucleic acid sequences set forth in (a), (b), (c), (d), (e) or (f).

In at least one embodiment, in an aspect, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group.

In at least one embodiment, in an aspect, the psilocybin derivative precursor compound can be a chemical compound having a formula (LVIII):

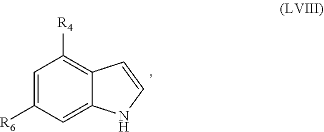

wherein R₄ is a hydroxy group, and wherein R₆ is a chlorine atom, and the first formed multi-substituent psilocybin derivative compound has a formula (LV):

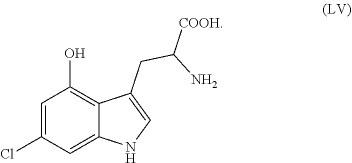 [SMILES: NC(Cc1c[nH]c2cc(Cl)cc(O)c12)C(=O)O]

In at least one embodiment, in an aspect, the psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, and SEQ. ID NO 8;
(f) a nucleic acid sequence that encodes a functional variant of any one of the amino acid sequences set forth in SEQ. ID NO: 4, SEQ. ID NO: 6, and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LVIII):

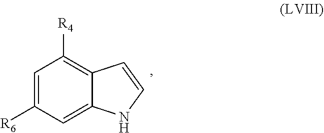

wherein R₄ is a fluorine atom, an amino group, or a hydroxy group, wherein R₆ is a fluorine atom, an amino group, a nitrile, or a bromine atom, and the first formed multi-substituent psilocybin derivative compound has a formula (LIX):

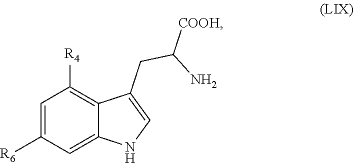

wherein R₄ is a fluorine atom, an amino group, or a hydroxy group, wherein R₆ is a fluorine atom, an amino group, a nitrile, or a bromine atom, and wherein the second multi-substituent psilocybin derivative has a formula (XXII), (XXVI), (XXIX), (LII), or (LIV):

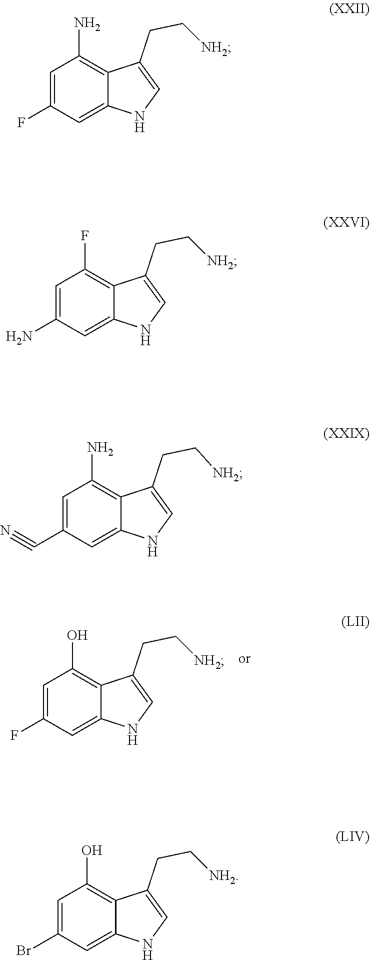 [SMILES: NCCc1c[nH]c2cc(F)cc(N)c12 ; NCCc1c[nH]c2cc(N)cc(F)c12 ; N#Cc1cc(N)c2c(CCN)c[nH]c2c1 ; NCCc1c[nH]c2cc(F)cc(O)c12 ; "or" ; NCCc1c[nH]c2cc(Br)cc(O)c12]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of the amino acid sequence set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LVIII):

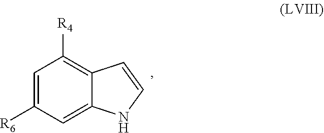

wherein R₄ is an acetamidyl group, a fluorine atom, an amino group, or a hydroxy group, wherein R₆ is a fluorine atom, an amino group, a nitrile, or a bromine atom, and the first formed multi-substituent psilocybin derivative compound has a formula (LIX):

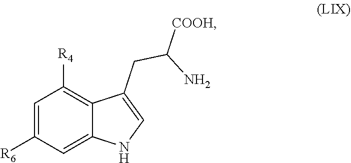

wherein R₄ is an acetamidyl group, a fluorine atom, an amino group, or a hydroxy group, wherein R₆ is a fluorine atom, an amino group, a nitrile, or a bromine atom, and wherein the second multi-substituent psilocybin derivative has a formula (LX):

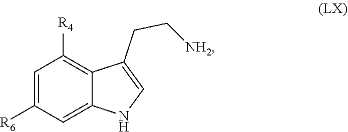

and wherein the third multi-substituent psilocybin derivative has a formula (IX), (X), (XVIII), (XXI), (XXV), or (XXVIII):

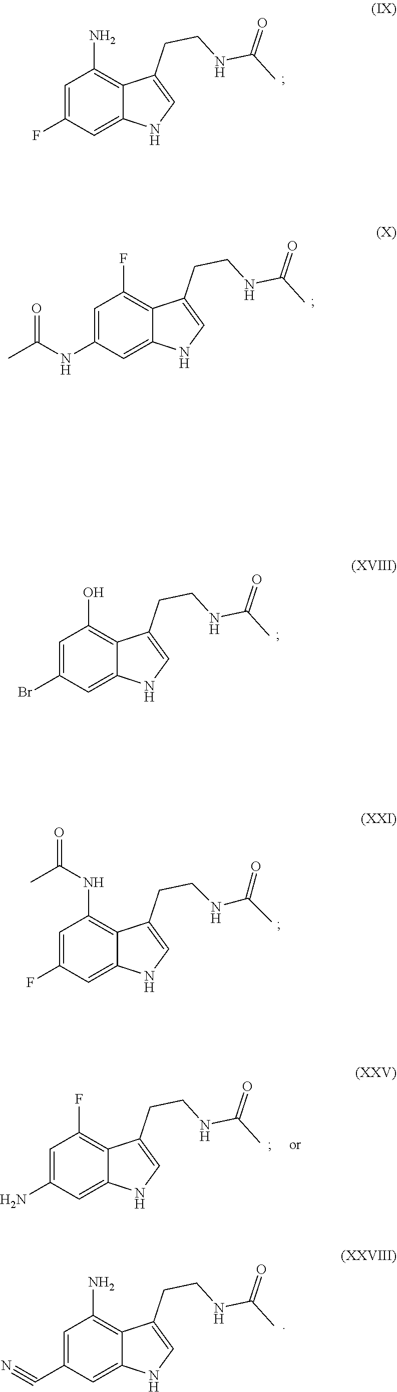

In at least one embodiment, in an aspect, the psilocybin biosynthetic enzyme complement can further comprise an N-methyl transferase to methylate the R₃ amino group at R₃ and form a fourth multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11 and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14;
(f) a nucleic acid sequence that encodes a functional variant of any one of the amino acid sequences set forth in SEQ. ID NO: 12 and SEQ. ID NO 14; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LVIII):

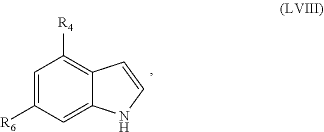

wherein R₄ is an amino group or a hydroxy group, wherein R₆ is a chlorine atom, a nitrile group, or a bromine atom, and the first formed multi-substituent psilocybin derivative compound has a formula (LIX):

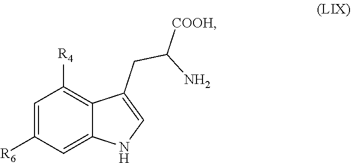

wherein R₄ is an amino group or a hydroxy group, wherein R₆ is a chlorine atom, a nitrile group, or a bromine atom, and wherein the second multi-substituent psilocybin derivative has a formula (LX):

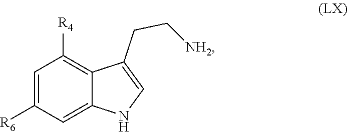

and wherein the fourth multi-substituent psilocybin derivative has a formula (XXVII), (XL), or (LIII):

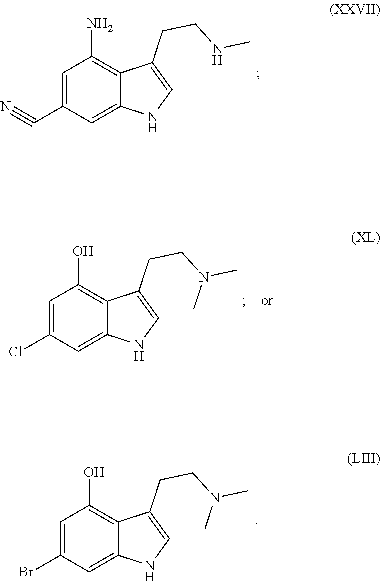

In at least one embodiment, in an aspect, the psilocybin biosynthetic enzyme complement can comprise a prenyl transferase, encoded by a nucleic acid selected from:
(a) SEQ. ID NO: 15, SEQ. ID NO: 17, SEQ. ID NO: 19, and SEQ. ID NO 21;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 16, SEQ. ID NO: 18, SEQ. ID NO: 20, and SEQ. ID NO 22;
(f) a nucleic acid sequence that encodes a functional variant of any one of the amino acid sequences set forth in SEQ. ID NO: 16, SEQ. ID NO: 18, SEQ. ID NO: 20, and SEQ. ID NO 22; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVII), one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is —CH₂—CHNH₂COOH, and wherein a first multi-substituent psilocybin derivative compound having formula (I) is formed wherein at least one of R₂, R₄, R₅, R₆, or R₇, is a prenyl group, and R_{3c} is a hydrogen atom.

In at least one embodiment, in an aspect, the psilocybin derivative precursor compound can be a chemical compound having a formula (LXI):

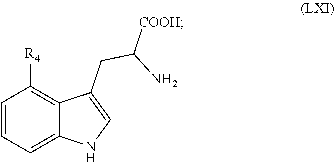

wherein R₄ is a hydroxy group, and the first formed multi-substituent psilocybin derivative compound has a formula (L):

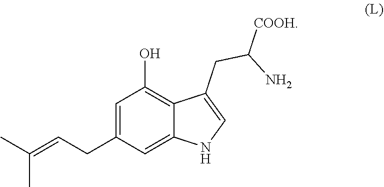 [SMILES: CC(C)=CCc1cc(O)c2c(CC(N)C(=O)O)c[nH]c2c1]

In at least one embodiment, in an aspect, the psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin biosynthetic enzyme complement can further comprise an N-methyl transferase to methylate the R₃ amino group at R₃ and form a fourth multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14; (f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXI):

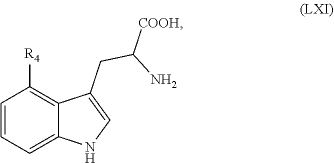

wherein R₄ is a propionyloxy or an acetoxy group, and the first formed multi-substituent psilocybin derivative compound has a formula (LIX):

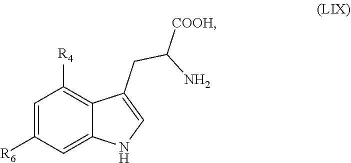

wherein R₄ is a propionyloxy or an acetoxy group, wherein R₆ is a prenyl group, and wherein the second multi-substituent psilocybin derivative has a formula:

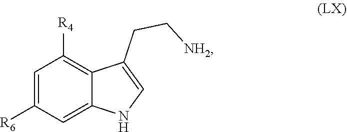

wherein R₄ is a propionyloxy or an acetoxy group, wherein R₆ is a prenyl group, wherein the third multi-substituent psilocybin derivative has a formula (XLI) or (XLII):

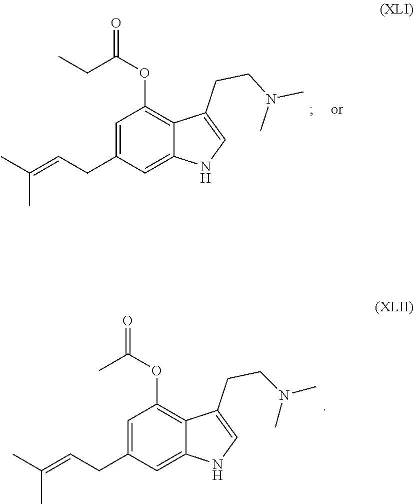

In at least one embodiment, in an aspect, the psilocybin derivative precursor compound can be a chemical compound having a formula (LXII):

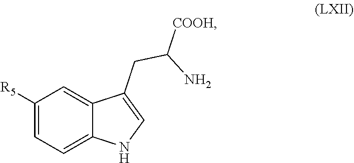

wherein R₅ is a chlorine or a fluorine atom, and the first formed multi-substituent psilocybin derivative compound has a formula (LXIII):

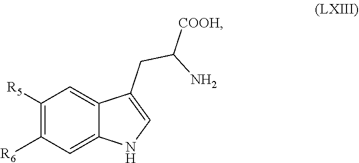

wherein R₅ is a chlorine or a fluorine atom, and wherein R₆ is a prenyl group, and wherein the second formed multi-substituent psilocybin derivative compound has a formula (XXXVI) and (XXXVIII):

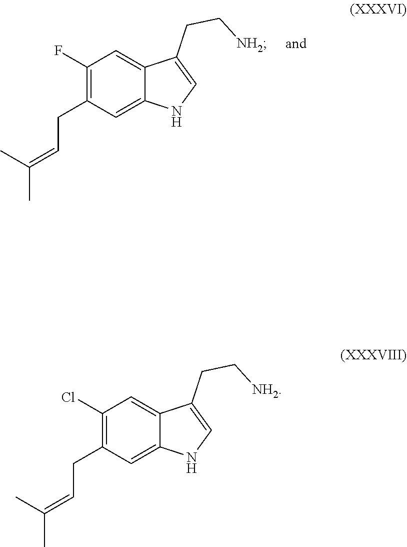 [SMILES: CC(C)=CCc1cc2[nH]cc(CCN)c2cc1F ; "and" ; CC(C)=CCc1cc2[nH]cc(CCN)c2cc1Cl]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXI):

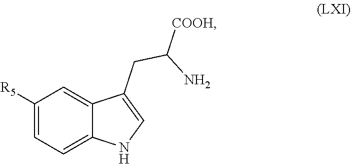

wherein R₅ is a chlorine or a fluorine atom, and the first formed multi-substituent psilocybin derivative compound has a formula (LXIII):

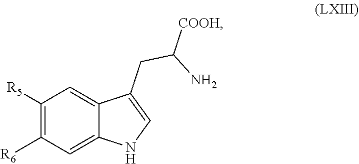

wherein R₅ is a chlorine or a fluorine atom, and wherein R₆ is a prenyl group, and wherein the second formed multi-substituent psilocybin derivative compound has a formula (LXIV):

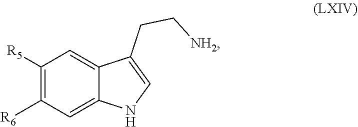

wherein R₅ is a chlorine or a fluorine atom, and wherein R₆ is a prenyl group, and wherein the third multi-substituent psilocybin derivative has a formula (XXXV) or (XXXVII):

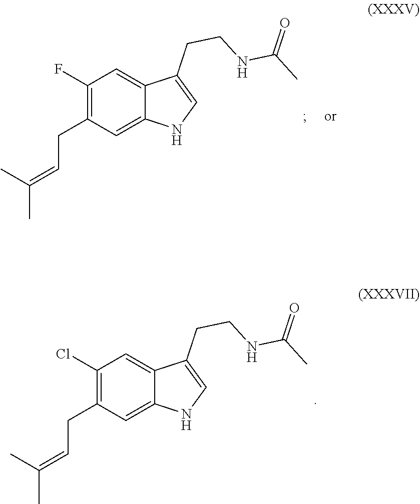

In at least one embodiment, in an aspect, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group.

In at least one embodiment, in an aspect, the psilocybin derivative precursor compound can be a chemical compound having a formula (LXV):

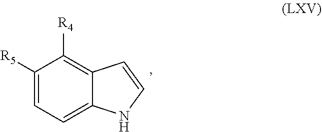

wherein R₄ is a hydroxy group, and wherein R₅ is a prenyl group, and the first formed multi-substituent psilocybin derivative compound has a formula (LI):

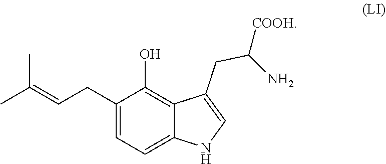 [SMILES: CC(C)=CCc1ccc2[nH]cc(CC(N)C(=O)O)c2c1O]

In at least one embodiment, in an aspect, the psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXV):

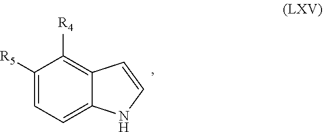

wherein R₄ is a fluorine atom and R₅ is nitrile group, and the first formed multi-substituent psilocybin derivative compound has a formula (LXIX):

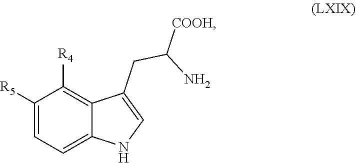

wherein R₄ is a fluorine atom and wherein R₅ is a nitrile group, and wherein the second formed multi-substituent psilocybin derivative compound has a formula (XIX):

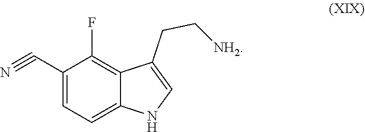 [SMILES: N#Cc1ccc2[nH]cc(CCN)c2c1F]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXV):

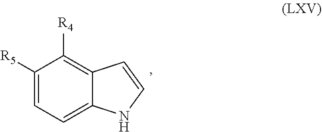

wherein R₄ is a fluorine atom and R₅ is a hydroxy group or a nitrile group, and the first formed multi-substituent psilocybin derivative compound has a formula (LXIX):

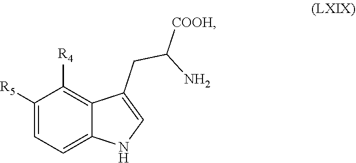

wherein R₄ is a fluorine atom and wherein R₅ is a hydroxy group or a nitrile group, and wherein the second formed multi-substituent psilocybin derivative compound has a formula (LXXII):

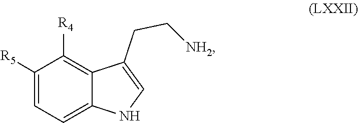

wherein R₄ is a fluorine atom, and wherein R₅ is a hydroxy group or a nitrile group, and wherein the third multi-substituent psilocybin derivative has a formula (XVII) or (XX):

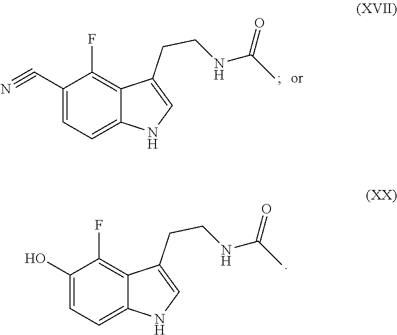 [SMILES: CC(=O)NCCc1c[nH]c2ccc(C#N)c(F)c12 ; CC(=O)NCCc1c[nH]c2ccc(O)c(F)c12]

In at least one embodiment, in an aspect, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group.

In at least one embodiment, in an aspect, the psilocybin derivative precursor compound can be a chemical compound having a formula (LXVI):

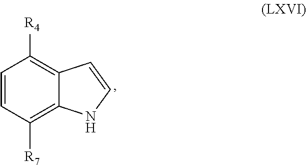

wherein R₄ is a hydroxy group, and wherein R₇ is a prenyl group, and the first formed multi-substituent psilocybin derivative compound has a formula (XLIX):

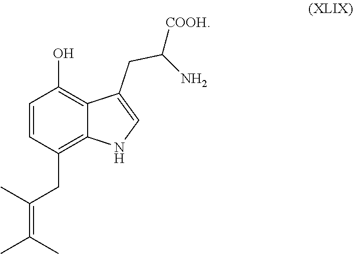 [SMILES: CC(C)=C(C)Cc1ccc(O)c2c(CC(N)C(=O)O)c[nH]c12]

In at least one embodiment, in an aspect, the psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVI):

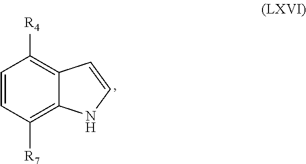

wherein R₄ is a fluorine atom and R₇ is nitrile group, and the first formed multi-substituent psilocybin derivative compound has a formula (LXX):

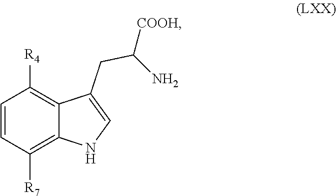

wherein R₄ is a fluorine atom and wherein R₇ is a nitrile group, and wherein the second formed multi-substituent psilocybin derivative compound has a formula (XXIV):

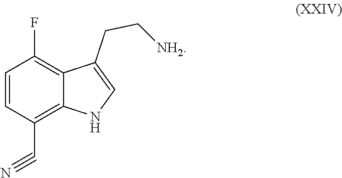 [SMILES: N#Cc1ccc(F)c2c(CCN)c[nH]c12]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVI):

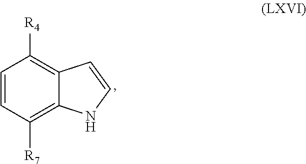

wherein R₄ is a fluorine atom or a chlorine atom and R₇ is a prenyl group or a nitrile group, and the first formed multi-substituent psilocybin derivative compound has a formula (LXX):

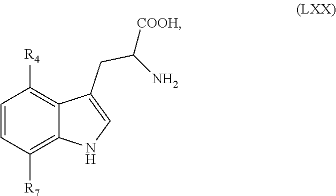

wherein R₄ is a fluorine atom or a chlorine atom and wherein R₇ is a prenyl group or a nitrile group, and wherein the second formed multi-substituent psilocybin derivative compound has a formula (LXXIII):

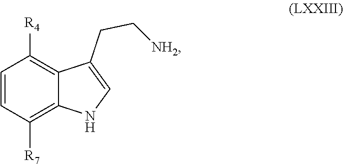

wherein R₄ is a fluorine atom or a chlorine atom, and wherein R₇ is a prenyl group or a nitrile group, and wherein the third multi-substituent psilocybin derivative has a formula (XXIII) or (XX):

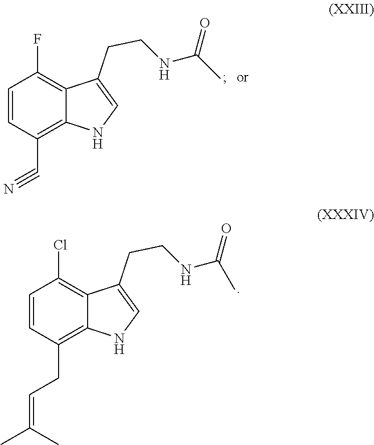

In at least one embodiment, in an aspect, the psilocybin biosynthetic enzyme complement can further comprise an N-methyl transferase to methylate the R₃ amino group at R₃ and form a further multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14;
(f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVI):

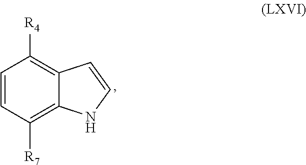

wherein R₄ is a chlorine atom and R₇ is a hydroxy group, and the first formed multi-substituent psilocybin derivative compound has a formula (LXX):

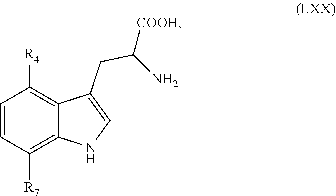

wherein R₄ is a hydroxy group and wherein R₇ is a chlorine atom, and wherein the second formed multi-substituent psilocybin derivative compound has a formula

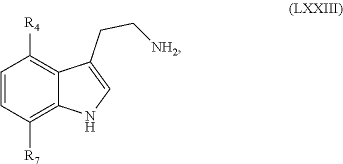

wherein R₄ is a hydroxy group, and wherein R₇ is a chlorine atom, and wherein the third multi-substituent psilocybin derivative has a formula (XXXIX):

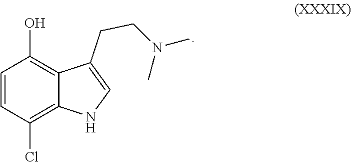 [SMILES: CN(C)CCc1c[nH]c2c(Cl)ccc(O)c12]

In at least one embodiment, in an aspect, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group, and the psilocybin biosynthetic enzyme complement further comprises a tryptophan decarboxylase to decarboxylate a R₃—CH₂—CHNH₂COOH group of the first multi-substituent psilocybin derivative compound, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVII):

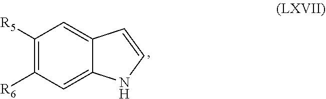

wherein R₅ is a fluorine atom, a chlorine atom, or a nitrile group and R₆ is a fluorine atom, an amino group or a prenyl group, and the first formed multi-substituent psilocybin derivative compound has a formula (LXIII):

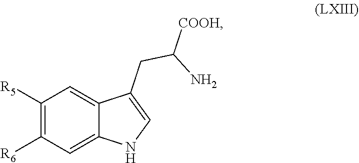

wherein R₅ is a fluorine atom, a chlorine atom, or a nitrile group and wherein R₆ is a is a fluorine atom, an amino group or a prenyl group, and wherein the second formed multi-substituent psilocybin derivative compound has a formula (XI), (XVI), (XXXVI), or (XXXVIII):

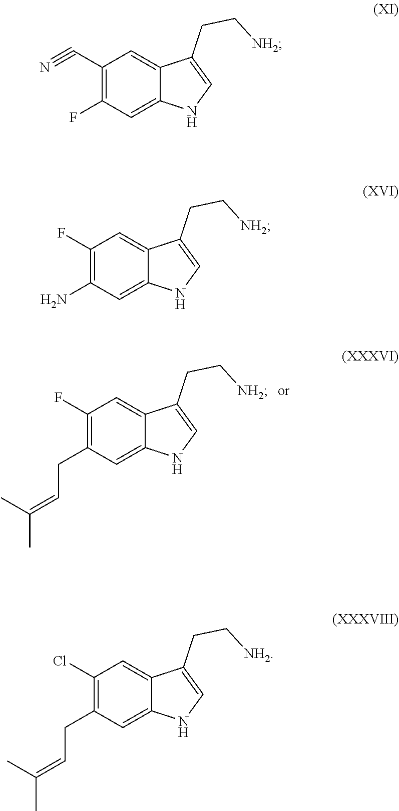 [SMILES: N#Cc1cc2c(CCN)c[nH]c2cc1F ; NCCc1c[nH]c2cc(N)c(F)cc12 ; CC(C)=CCc1cc2[nH]cc(CCN)c2cc1F ; CC(C)=CCc1cc2[nH]cc(CCN)c2cc1Cl]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVII):

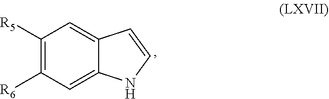

wherein R₅ is a fluorine atom or a chlorine atom and R₆ is an amino group, an acetamidyl group, or a prenyl group, and the first formed multi-substituent psilocybin derivative compound has a formula (LXIII):

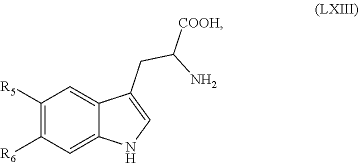

wherein R₅ is a fluorine atom or a chlorine atom and wherein R₆ is an amino group, an acetamidyl group, or a prenyl group, and wherein the second formed multi-substituent psilocybin derivative compound has a formula (LXXIV):

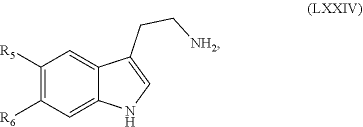

wherein R₅ is a fluorine atom or a chlorine atom, and wherein R₆ is an amino group, an acetamidyl group, or a prenyl group, and wherein the third multi-substituent psilocybin derivative has a formula (XIV), (XV), (XXXV), or (XXXVII):

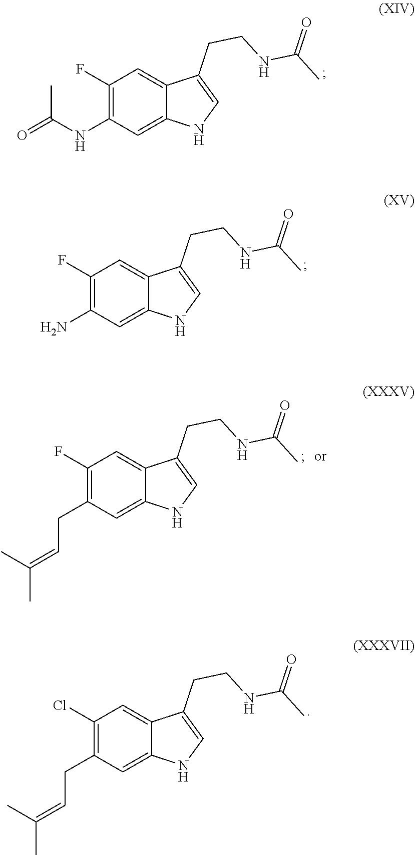

In at least one embodiment, in an aspect, the psilocybin biosynthetic enzyme complement can further comprise an N-methyl transferase to methylate the R₃ amino group at R₃ and form a fourth multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14; (f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVII):

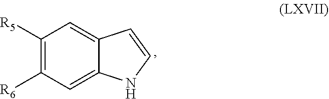

wherein R₅ is a chlorine atom and R₆ is a prenyl group, and the first formed multi-substituent psilocybin derivative compound has a formula (LXIII):

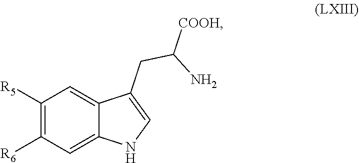

wherein R₅ is a chlorine atom and wherein R₆ is a prenyl group, and wherein the second formed multi-substituent psilocybin derivative compound has a formula (LXXIV):

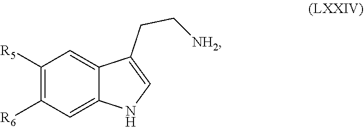

wherein R₅ is a chlorine atom, and wherein R₆ is a prenyl group, and wherein the third multi-substituent psilocybin derivative has a formula (XLIV):

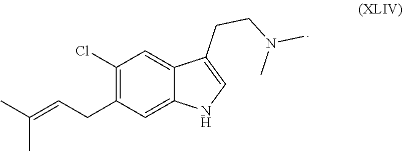 [SMILES: CC(C)=CCc1cc2[nH]cc(CCN(C)C)c2cc1Cl]

In at least one embodiment, in an aspect, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group, and the psilocybin biosynthetic enzyme complement further comprises a tryptophan decarboxylase to decarboxylate a R₃—CH₂—CHNH₂COOH group of the first multi-substituent psilocybin derivative compound, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVIII):

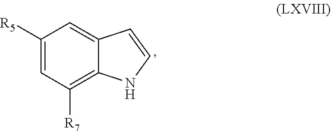

wherein R₅ is a fluorine atom, and R₇ is a nitro group or a prenyl group, and the first formed multi-substituent psilocybin derivative compound has a formula (LXXI):

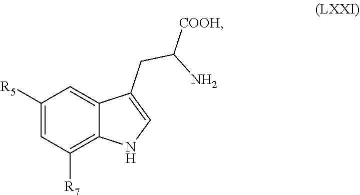

wherein R₅ is a fluorine atom, and wherein R₇ is a nitro group atom or a prenyl group, and wherein the second formed multi-substituent psilocybin derivative compound has a formula (XIII) or (XXXIII):

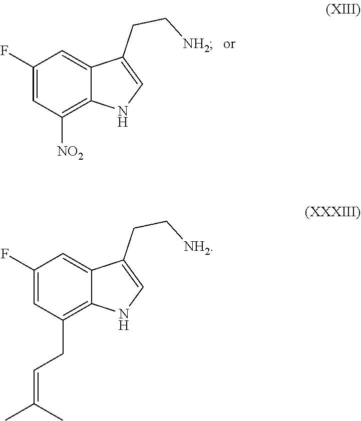 [SMILES: NCCc1c[nH]c2c([N+](=O)[O-])cc(F)cc12 ; CC(C)=CCc1cc(F)cc2c(CCN)c[nH]c12]

In at least one embodiment, in an aspect, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

In at least one embodiment, in an aspect, the psilocybin derivative precursor compound can be a chemical compound having a formula (LXVIII):

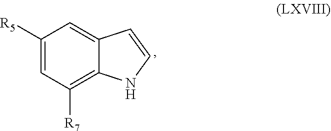

wherein R₅ is a fluorine atom, and R₇ is a nitro group or a prenyl group, and the first formed multi-substituent psilocybin derivative compound has a formula (LXXI):

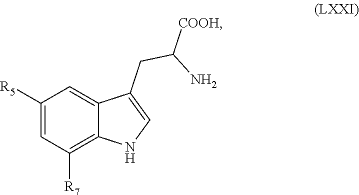

wherein R₅ is a fluorine atom, and wherein R₇ is a nitro group atom or a prenyl group, and wherein the second formed multi-substituent psilocybin derivative compound has a formula (LXXV):

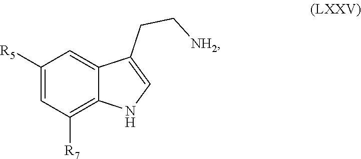

wherein R₅ is a fluorine atom, and wherein R₇ is a nitro group or a prenyl group, and wherein the third multi-substituent psilocybin derivative has a formula (XII) or (XXXII):

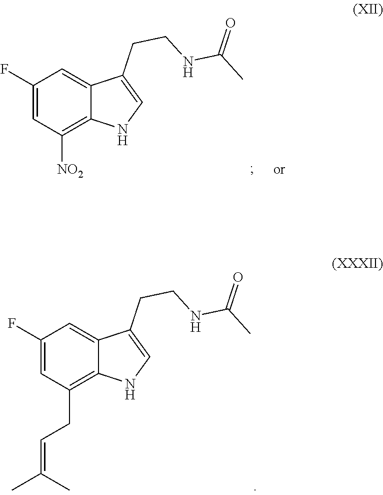 [SMILES: CC(=O)NCCc1c[nH]c2c([N+](=O)[O-])cc(F)cc12 ; CC(=O)NCCc1c[nH]c2c(CC=C(C)C)cc(F)cc12]

In at least one embodiment, in an aspect, the psilocybin biosynthetic enzyme complement can contain a prenyl transferase encoded by a nucleic acid selected from:
(a) SEQ. ID NO: 15, SEQ. ID NO: 17, SEQ. ID NO: 19, and SEQ. ID NO 21;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 16, SEQ. ID NO: 18, SEQ. ID NO: 20, and SEQ. ID NO 22;
(f) a nucleic acid sequence that encodes a functional variant of any one of the amino acid sequences set forth in SEQ. ID NO: 16, SEQ. ID NO: 18, SEQ. ID NO: 20, and SEQ. ID NO 22; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group or a hydrogen atom.

In at least one embodiment, the psilocybin derivative precursor compound having formula (LXXVII):

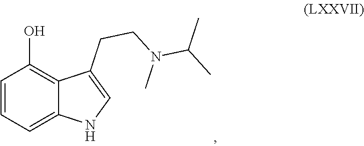 [SMILES: CC(C)N(C)CCc1c[nH]c2cccc(O)c12]

and the first multi-substituent psilocybin derivative compound has the formula (LXXVI):

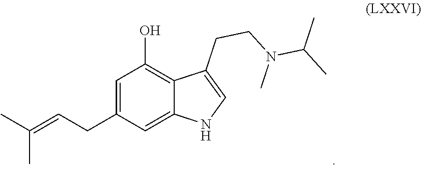 [SMILES: CC(C)=CCc1cc(O)c2c(CCN(C)C(C)C)c[nH]c2c1]

In at least one embodiment, in an aspect, the method can further include a step comprising isolating the multi-substituent psilocybin derivative compound, from the host cell and/or a host cell medium.

In at least one embodiment, in an aspect, the host cell can be a microorganism.

In at least one embodiment, in an aspect, the host cell can be a bacterial cell or a yeast cell.

In at least one embodiment, in an aspect, the host cell can be an Escherichia coli cell or a Saccharomyces cerevisiae cell.

In another aspect the present disclosure provides, in at least one embodiment, a use of a chemical compound or a salt thereof having a formula (I):

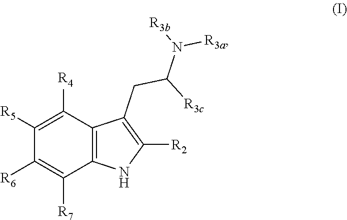

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, in the manufacture of a pharmaceutical or recreational drug formulation.

In at least one embodiment the manufacture can comprise formulating the chemical compound with an excipient, diluent, or carrier.

In another aspect the present disclosure provides, in at least one embodiment, a use of a chemical compound or a salt thereof having a formula (I):

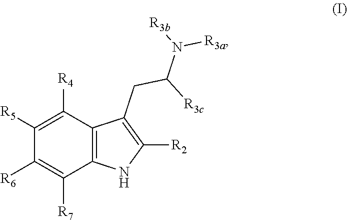

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, together with a diluent, carrier, or excipient as a pharmaceutical or recreational drug formulation.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 depicts the chemical structure of psilocybin.

FIG. 2 depicts a certain prototype structure of psilocybin and psilocybin derivative compounds, namely an indole. Certain carbon and nitrogen atoms may be referred to herein by reference to their position within the indole structure, i.e., N₁, C₂, C₃ etc. The pertinent atom numbering is shown.

FIGS. 3A, 3B, 3C, 3D, 3E, 3F, 3G, 3H, 3I, 3J, 3K and 3L depict the chemical structures of certain example psilocybin derivatives, notably a 2,5-di-S1,S2-psilocybin derivative (FIG. 3A), a 2,5-di-S2,S1-psilocybin derivative (FIG. 3B), a 2,6-d-S1,S2-psilocybin derivative (FIG. 3C), a 2,6-di-S2,S1-psilocybin derivative (FIG. 3D), a 2,7-di-S1,S2-psilocybin derivative (FIG. 3E), a 2,7-di-S2,S1-psilocybin derivative (FIG. 3F), a 5,6-di-S1,S2-psilocybin derivative (FIG. 3G), a 5,6-di-S2,S1-psilocybin derivative (FIG. 3H), a 5,7-di-S1,S2-psilocybin derivative (FIG. 3I), a 5,7-di-S2,S1-psilocybin derivative (FIG. 3J), a 6,7-di-S1,S2-psilocybin derivative (FIG. 3K), and a 6,7-di-S2,S1-psilocybin derivative (FIG. 3L). In each of the foregoing S1 and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone, (viii) a prenyl group, and (ix) a nitrile group. It is noted that in each of FIGS. 3A-3L, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 4A, 4B, 4C, 4D, 4E, and 4F depict the chemical structures of certain example psilocybin derivatives, notably a 2,5,6-tri-S1,S2,S3-psilocybin derivative (FIG. 4A), a 2,5,6-tri-S1,S3,S2-psilocybin derivative (FIG. 48), a 2,5,6-tri-S3,S1,S2-psilocybin derivative (FIG. 4C), a 2,5,6-tri-S2,S1,S3-psilocybin derivative (FIG. 4D), a 2,5,6-tri-S3,S2,S1-psilocybin derivative (FIG. 4E), and a 2,5,6-tri-S2,S3,S1-psilocybin derivative (FIG. 4F). In each of the foregoing S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 4A-4F, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 4G, 4H, and 4I depict the chemical structures of certain example psilocybin derivatives, notably a 2,5,6-tri-S1,S2,S2-psilocybin derivative (FIG. 4G), a 2,5,6-tri-S2,S1,S2-psilocybin derivative (FIG. 4H), and a 2,5,6-tri-S2,S2,S1-psilocybin derivative (FIG. 4I). In each of the foregoing S1 and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 4G-4I, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 5A, 5B, 5C, 5D, 5E, and 5F depict the chemical structures of certain example psilocybin derivatives, notably a 2,5,7-tri-S1,S2,S3-psilocybin derivative (FIG. 5A), a 2,5,7-tri-S1, S3,S2-psilocybin derivative (FIG. 5B), a 2,5,7-tri-S3,S1,S2-psilocybin derivative (FIG. 5C), a 2,5,7-tri-S2,S1,S3-psilocybin derivative (FIG. 5D), a 2,5,7-tri-S3,S2,S1-psilocybin derivative (FIG. 5E), and a 25,7-tri-S2,S3,S1-psilocybin derivative (FIG. 5F). In each of the foregoing S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 5A-5F, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 5G, 5H, and 5I depict the chemical structures of certain example psilocybin derivatives, notably a 2,5,7-tri-S1,S2,S2-psilocybin derivative (FIG. 5G), a 2,5,7-tri-S2,S1,S2-psilocybin derivative (FIG. 5H), and a 2,5,7-tri-S2,S2,S1-psilocybin derivative (FIG. 5I). In each of the foregoing S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) and a nitrile group. It is noted that in each of FIGS. 5A-5I, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 6A, 6B, 6C, 6D, 6E, and 6F depict the chemical structures of certain example psilocybin derivatives, notably a 2,6,7-tri-S1,S2,S3-psilocybin derivative (FIG. 6A), a 2,6,7-tri-S1,S3,S2-psilocybin derivative (FIG. 6I), a 2,6,7-tri-S3,S1,S2-psilocybin derivative (FIG. 6C), a 2,6,7-tri-S2,S1,S3-psilocybin derivative (FIG. 6D), a 2,6,7-tri-S3,S2,S1-psilocybin derivative (FIG. 6E), and a 2,6,7-tri-S2,S3,S1-psilocybin derivative (FIG. 6F). In each of the foregoing S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 6A-6F, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 6G, 6H, and 6I depict the chemical structures of certain example psilocybin derivatives, notably a 2,6,7-tri-S1,S2,S2-psilocybin derivative (FIG. 6G), a 2,6,7-tri-S2,S1,S2-psilocybin derivative (FIG. 6H), and a 2,6,7-tri-S2,S2,S1-psilocybin derivative (FIG. 6I). In each of the foregoing S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 6G-6I, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 7A, 7B, 7C, 7D, 7E, and 7F depict the chemical structures of certain example psilocybin derivatives, notably a 5,6,7-tri-S1,S2,S3-psilocybin derivative (FIG. 7A), a 5,6,7-tri-S1,S3,S2-psilocybin derivative (FIG. 7B), a 5,6,7-tri-S3,S1,S2-psilocybin derivative (FIG. 7C), a 5,6,7-tri-S2,S1,S3-psilocybin derivative (FIG. 7D), a 5,6,7-tri-S3,S2,S1-psilocybin derivative (FIG. 7E), and a 56,7-tri-S2,S3,S1-psilocybin derivative (FIG. 7F). In each of the foregoing S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 7A-7F, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 7G, 7H, and 7I depict the chemical structures of certain example psilocybin derivatives, notably a 5,6,7-tri-S1,S2,S2-psilocybin derivative (FIG. 7G), a 5,6,7-tri-S2,S1,S2-psilocybin derivative (FIG. 7H), and a 5,6,7-tri-S2,S2,S1-psilocybin derivative (FIG. 7I). In each of the foregoing S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. It is noted that in each of FIGS. 7G-7I, R₃ₐ and R_{3b} can each be a hydrogen atom, an alkyl group, a cycloalkyl group, an acyl group, or an aryl group, and R_{3c} can be a hydrogen atom or a carboxyl group.

FIGS. 8A, 8B, 8C, 8D, 8E, 8F, and 8G depict the chemical structures of certain example psilocybin derivatives, notably O-alkylated psilocybin derivatives, notably a 4-O-methyl-5-chloro-psilocybin derivative (FIG. 8A), a 4-O-ethyl-5-chloro-psilocybin derivative (FIG. 8B), a 4-acetoxy-5-chloro-psilocybin derivative (FIG. 8C), a 4-propionyloxy-5-chloro-psilocybin derivative (FIG. 8D), a 4-hydroxy-5-chloro-psilocybin derivative (FIG. 8E), a 4-phospho-5-chloro-psilocybin derivative (FIG. 8F), and a 5-chloro-psilocybin derivative (FIG. 8H). It is noted that in each of FIGS. 8A-8G, R₃ₐ and R_{3b} can each be a hydrogen atom, an alkyl group, a cycloalkyl group, an acyl group, or an aryl group, and R_{3c} can be a hydrogen atom or a carboxyl group.

FIG. 9 depicts an example chemical reaction for synthesizing a nitrated psilocybin derivative, notably a reaction wherein a 4-O-methyl-psilocybin derivative is reacted with nitric acid in the presence of sulfuric acid to form a 4-O-methyl-5-nitro-psilocybin derivative.

FIG. 10 depicts an example chemical reaction for synthesizing a glycosylated psilocybin derivative, notably a reaction wherein a 4-hydroxy-psilocybin derivative is reacted with a glycosyl compound to form a 4-glycosyl-psilocybin derivative.

FIGS. 11A, 11B and 11C depict certain example chemical reactions for synthesizing aminated psilocybin derivatives with subsequent N-substitutions, notably a reaction wherein a 4-O-methyl-5-nitro-psilocybin derivative is reacted with hydrogen under the catalysis of palladium on charcoal to form a 4-O-methyl-5-amino-psilocybin derivative (FIG. 11A). The formed amino group at the 5-position can then be substituted with different group such as an acylation with acetic anhydride. The amino group can also be alkylated via a condensation with an aldehyde (such as acetaldehyde) followed by a reduction of the intermediate imine with borohydride (FIG. 11B). FIG. 11C depicts a possible direction amination method with H₂O₂ and NH₃·H₂O with the help of a catalyst.

FIGS. 12A, 12B, 12C and 12D depicts depict example chemical reactions showing the formation of a 4-hydroxy-5-carboxyl psilocybin derivative using a 4-hydroxy-psilocybin derivative as a reactant (FIG. 12A); a 4-hydroxy-7-carboxyl psilocybin derivative using an arylhalide-psilocybin derivative as a reactant (FIG. 12B); a carboxylated protected psilocybin derivate using a protected psilocybin derivative as a reagent (FIG. 12C), and a sodium salt of a carboxylated psilocybin derivative (a); an OH substituted carboxyl group forming an ester; (b) or an OH substituted carboxyl group forming an amide (c), using a carboxylated psilocybin derivative as a reagent (FIG. 12D)

FIGS. 13A, 13B, 13C and 13D depict example chemical reactions for making psilocybin derivatives. FIG. 13A depicts transformations for an initially synthesized 7-nitrated psilocybin derivative to other psilocybin derivatives containing two types of groups at the C₅ and C₇ atoms, notably the group at the C₅ atom is either a keto group or a carboxylic acid, and the group at the C₇ atom is either a nitro, amine, nitrile, hydroxyl, iodide, or fluoride. FIG. 13B depicts example chemical transformations using an initially synthesized 5-carboxy psilocybin derivative to other O-4-glycosylated psilocybin derivatives containing two types of groups at the C₅ atom and the C₇ atom, notably the group at the C₅ atom is either an ester or an amide, the group at the C₇ atom is either a nitro or amine. FIG. 13C depicts further example chemical transformations of an initially synthesized 5-nitrated psilocybin derivative to form 5,6,7-tri-substituted psilocybin derivatives containing up to three types of groups at the C₅, C₆ and C₇ carbon atoms. The groups attached to the C₅ carbon atom can be either a nitro, amino, or an N-acetamido group, while the group attached to the C₆ carbon atom can be either a halide, a nitro group or an amino group, and the group attached to the C₇ carbon atom can be either a formyl, carboxy or amide group. FIG. 13-D depicts the preparation of a hydroxy-psilocybin derivative (Compound 13-D4) which can then be prenylated, either in vitro or in vivo, using a prenyl transferase to form, for example, a C₆ prenylated derivative of compound 13-D4. It is noted that specific chemical compounds in FIGS. 13A-13D are labeled as 13A-1, 13A-2 etc. (FIG. 13A); 13B-1, 13B-2 etc. (FIG. 13B); 13C-1, 13C-2 etc. (FIG. 13C) and 13D-1, 13D-2 etc. (FIG. 13D).

FIGS. 14A, 14B, 14C, 14D, 14E, 14F and 14G depict certain pathways to biosynthetically make example multi-substituent psilocybin derivative compounds, notably example compounds possessing a S1₄ and a S2₆ substituent (FIG. 14A), example compounds possessing a S1₄ and a S2₆ prenyl group (FIG. 14B), example compounds possessing a S1₅ and a S2₆ prenyl group (FIG. 14C), example compounds possessing a S1₄ and a S2₅ substituent (FIG. 14D), example compounds possessing a S1₄ and a S2₇ substituent (FIG. 14E), example compounds possessing a S1₅ and a S2₆ substituent (FIG. 14F), and example compounds possessing a S1₅ and a S2₇ substituent (FIG. 14G). Roman numerals adjacent to the example compounds in each of FIGS. 14A-14G refer to the example compounds thus numbered in this disclosure.

FIGS. 15A, 15B, 15C, 15D, 15E, 15F, 15G and 15H depict various graphs, obtained in the performance of experimental assays to evaluate the efficacy of an example prenylated psilocybin derivative having the chemical formula (IX) set forth herein, notably a cell viability assay for a multi-substituent psilocybin derivative having the chemical formulae (IX) (FIG. 15A); a saturation binding assay for [³H]ketanserin at the 5-HT_{2A} receptor (FIG. 15B); a competition assay for psilocin as a positive control (binding) (Panel A); and a competition assay for tryptophan as a negative control (no binding) (Panel B) (FIG. 15C); a competition assay for a multi-substituent psilocybin derivative compound with formula (IX), designated “IX” (FIG. 15D, plotted with two different Y-axes, panel A and panel B, for clarity); a cAMP assay in the presence of constant (4 μM) forskolin and with increasing concentration of psilocin in +5HT_{1A} cells and −5HT_{1A} cells (FIG. 15E); a cAMP assay in the presence of constant (4 μM) forskolin and 10 μM serotonin, and with increasing concentrations of serotonin+5HT_{1A} cells and −5HT_{1A} cells (FIG. 15F); a cAMP assay in the presence of constant (4 μM) forskolin and, and with increasing concentrations of tryptophan in +5HT_{1A} cells and −5HT_{1A} cells a cAMP assay (FIG. 14G); and a cAMP assay in the of constant (4 μM) forskolin, and with increasing concentration of multi-substituent psilocybin derivative compound having formula (IX), designated “IX” in +5HT_{1A} cells and −5HT_{1A} cells (FIG. 15H).

FIGS. 16A, 16B, and 16C depict various graphs, obtained in the performance of experimental assays to evaluate the efficacy of an example prenylated psilocybin derivative having the chemical formula (X) set forth herein, notably a cell viability assay for a multi-substituent psilocybin derivative having the chemical formulae (IX) (FIG. 16A); a competition assay for a multi-substituent psilocybin derivative compound with formula (IX), designated “IX” (FIG. 16B); and a cAMP assay in the of constant (4 μM) forskolin, and with increasing concentration of multi-substituent psilocybin derivative compound having formula (X), designated “X” in +5HT_{1A} cells and −5HT_{1A} cells (FIG. 15H).

FIGS. 17 and 17B depict a representation of mass spectrometry data in the form of a chromatogram, notably a chromatogram obtained in the performance of an experiment to synthesize an example multi-substituent psilocybin derivative compound having the chemical formula (XII) set forth herein (FIG. 17A); and a representation of mass spectrometry data in the form of a further mass spectrometry spectrum obtained in the performance of an experiment to identify a multi-substituent psilocybin derivative compound having the chemical formula (XII) set forth herein (FIG. 17B).

FIGS. 18A and 18B depict a representation of further mass spectrometry data in the form of a chromatogram, notably a chromatogram obtained in the performance of an experiment to synthesize an example multi-substituent psilocybin derivative compound having the chemical formula (XIV) set forth herein (FIG. 18A); and a representation of mass spectrometry data in the form of a further mass spectrometry spectrum obtained in the performance of an experiment to identify a multi-substituent psilocybin derivative compound having the chemical formula (XII) set forth herein (FIG. 18B).

FIGS. 19A and 19B depict a representation of further mass spectrometry data in the form of a chromatogram, notably a chromatogram obtained in the performance of an experiment to synthesize an example multi-substituent psilocybin derivative compound having the chemical formula (XV) set forth herein (FIG. 19A); and a representation of mass spectrometry data in the form of a further mass spectrometry spectrum obtained in the performance of an experiment to identify a multi-substituent psilocybin derivative compound having the chemical formula (XV) set forth herein (FIG. 19B).

FIG. 20 depicts a representation of further mass spectrometry data in the form of a chromatogram, notably a chromatogram obtained in the performance of an experiment to synthesize an example multi-substituent psilocybin derivative compound having the chemical formula (XVII) set forth herein.

FIGS. 21A and 21B depict a representation of further mass spectrometry data in the form of a chromatogram, notably a chromatogram obtained in the performance of an experiment to synthesize an example multi-substituent psilocybin derivative compound having the chemical formula (XVIII) set forth herein (FIG. 21A); and a representation of mass spectrometry data in the form of a further mass spectrometry spectrum obtained in the performance of an experiment to identify a multi-substituent psilocybin derivative compound having the chemical formula (XVIII) set forth herein (FIG. 21B).

FIGS. 22A and 22B depict a representation of further mass spectrometry data in the form of a chromatogram, notably a chromatogram obtained in the performance of an experiment to synthesize an example multi-substituent psilocybin derivative compound having the chemical formula (XXI) set forth herein (FIG. 22A); and a representation of mass spectrometry data in the form of a further mass spectrometry spectrum obtained in the performance of an experiment to identify a multi-substituent psilocybin derivative compound having the chemical formula (XXI) set forth herein (FIG. 22B).

FIGS. 23A and 23B depict a representation of further mass spectrometry data in the form of a chromatogram, notably a chromatogram obtained in the performance of an experiment to synthesize an example multi-substituent psilocybin derivative compound having the chemical formula (XXIII) set forth herein (FIG. 23A); and a representation of mass spectrometry data in the form of a further mass spectrometry spectrum obtained in the performance of an experiment to identify a multi-substituent psilocybin derivative compound having the chemical formula (XXIII) set forth herein (FIG. 23B).

FIG. 24 depicts a representation of further mass spectrometry data in the form of a chromatogram, notably a chromatogram obtained in the performance of an experiment to synthesize an example multi-substituent psilocybin derivative compound having the chemical formula (XXV) set forth herein.

FIGS. 25A and 25B depict a representation of further mass spectrometry data in the form of a chromatogram, notably a chromatogram obtained in the performance of an experiment to synthesize an example multi-substituent psilocybin derivative compound having the chemical formula (XXVIII) set forth herein (FIG. 25A); and a representation of mass spectrometry data in the form of a further mass spectrometry spectrum obtained in the performance of an experiment to identify a multi-substituent psilocybin derivative compound having the chemical formula (XXVIII) set forth herein (FIG. 25B).

FIG. 26 depicts a representation of further mass spectrometry data in the form of a chromatogram, notably a chromatogram obtained in the performance of an experiment to synthesize an example multi-substituent psilocybin derivative compound having the chemical formula (XX) set forth herein.

FIG. 27 depicts a representation of further mass spectrometry data in the form of a chromatogram, notably a chromatogram obtained in the performance of an experiment to synthesize an example multi-substituent psilocybin derivative compound having the chemical formula (XIII) set forth herein.

FIG. 28 depicts a representation of further mass spectrometry data in the form of a chromatogram, notably a chromatogram obtained in the performance of an experiment to synthesize an example multi-substituent psilocybin derivative compound having the chemical formula (XVI) set forth herein.

FIG. 29 depicts a representation of further mass spectrometry data in the form of a chromatogram, notably a chromatogram obtained in the performance of an experiment to synthesize an example multi-substituent psilocybin derivative compound having the chemical formula (XIX) set forth herein.

FIG. 30 depicts a representation of further mass spectrometry data in the form of a chromatogram, notably a chromatogram obtained in the performance of an experiment to synthesize an example multi-substituent psilocybin derivative compound having the chemical formula (XXII) set forth herein.

FIG. 31 depicts a representation of further mass spectrometry data in the form of a chromatogram, notably a chromatogram obtained in the performance of an experiment to synthesize an example multi-substituent psilocybin derivative compound having the chemical formula (XXVI) set forth herein.

FIGS. 32A and 32B depict a representation of further mass spectrometry data in the form of a chromatogram, notably a chromatogram obtained in the performance of an experiment to synthesize an example multi-substituent psilocybin derivative compound having the chemical formula (XXIX) set forth herein (FIG. 32A); and a representation of mass spectrometry data in the form of a further mass spectrometry spectrum obtained in the performance of an experiment to identify a multi-substituent psilocybin derivative compound having the chemical formula (XXIX) set forth herein (FIG. 32B).

FIG. 33 depicts a representation of further mass spectrometry data in the form of a chromatogram, notably a chromatogram obtained in the performance of an experiment to synthesize an example multi-substituent psilocybin derivative compound having the chemical formula (XXVII) set forth herein.

FIGS. 34A and 34B depict a representation of further mass spectrometry data in the form of a chromatogram, notably a chromatogram obtained in the performance of an experiment to synthesize an example multi-substituent psilocybin derivative compound having the chemical formula (XXXIV) set forth herein (FIG. 34A); and a representation of mass spectrometry data in the form of a further mass spectrometry spectrum obtained in the performance of an experiment to identify a multi-substituent psilocybin derivative compound having the chemical formula (XXXIV) set forth herein (FIG. 34B).

FIGS. 35A and 35B depict a representation of further mass spectrometry data in the form of a chromatogram, notably a chromatogram obtained in the performance of an experiment to synthesize an example multi-substituent psilocybin derivative compound having the chemical formula (XXXII) set forth herein (FIG. 35A); and a representation of mass spectrometry data in the form of a further mass spectrometry spectrum obtained in the performance of an experiment to identify a multi-substituent psilocybin derivative compound having the chemical formula (XXXII) set forth herein (FIG. 35B).

FIG. 36 depicts a representation of further mass spectrometry data in the form of a chromatogram, notably a chromatogram obtained in the performance of an experiment to synthesize an example multi-substituent psilocybin derivative compound having the chemical formula (XXXVI) set forth herein.

FIG. 37 depicts a representation of further mass spectrometry data in the form of a chromatogram, notably a chromatogram obtained in the performance of an experiment to synthesize an example multi-substituent psilocybin derivative compound having the chemical formula (XLIX) set forth herein.

FIG. 38 depicts a representation of further mass spectrometry data in the form of a chromatogram, notably a chromatogram obtained in the performance of an experiment to synthesize an example multi-substituent psilocybin derivative compound having the chemical formula (L) set forth herein.

FIG. 39 depicts a representation of further mass spectrometry data in the form of a chromatogram, notably a chromatogram obtained in the performance of an experiment to synthesize an example multi-substituent psilocybin derivative compound having the chemical formula (XLVII) set forth herein.

FIG. 40 depicts a representation of further mass spectrometry data in the form of a chromatogram, notably a chromatogram obtained in the performance of an experiment to synthesize an example multi-substituent psilocybin derivative compound having the chemical formula (LI) set forth herein.

FIG. 41 depicts a representation of further mass spectrometry data in the form of a chromatogram, notably a chromatogram obtained in the performance of an experiment to synthesize an example multi-substituent psilocybin derivative compound having the chemical formula (XXXVI) set forth herein.

FIG. 42 depicts a representation of further mass spectrometry data in the form of a chromatogram, notably a chromatogram obtained in the performance of an experiment to synthesize an example multi-substituent psilocybin derivative compound having the chemical formula (XXXV) set forth herein.

FIGS. 43A and 43B depict a representation of further mass spectrometry data in the form of a chromatogram, notably a chromatogram obtained in the performance of an experiment to synthesize an example multi-substituent psilocybin derivative compound having the chemical formula (XXXVI) set forth herein (FIG. 43A); and a representation of mass spectrometry data in the form of a further mass spectrometry spectrum obtained in the performance of an experiment to identify a multi-substituent psilocybin derivative compound having the chemical formula (XXXVI) set forth herein (FIG. 43B).

FIG. 44 depicts a representation of further mass spectrometry data in the form of a chromatogram, notably a chromatogram obtained in the performance of an experiment to synthesize an example multi-substituent psilocybin derivative compound having the chemical formula (XXXVIII) set forth herein.

FIG. 45 depicts a representation of further mass spectrometry data in the form of a chromatogram, notably a chromatogram obtained in the performance of an experiment to synthesize an example multi-substituent psilocybin derivative compound having the chemical formula (XL) set forth herein.

FIG. 46 depicts a representation of further mass spectrometry data in the form of a chromatogram, notably a chromatogram obtained in the performance of an experiment to synthesize an example multi-substituent psilocybin derivative compound having the chemical formula (XXXIX) set forth herein.

FIG. 47 depicts a representation of further mass spectrometry data in the form of a chromatogram, notably a chromatogram obtained in the performance of an experiment to synthesize an example multi-substituent psilocybin derivative compound having the chemical formula (LII) set forth herein.

FIG. 48 depicts a representation of further mass spectrometry data in the form of a chromatogram, notably a chromatogram obtained in the performance of an experiment to synthesize an example multi-substituent psilocybin derivative compound having the chemical formula (LIII) set forth herein.

FIG. 49 depicts a representation of further mass spectrometry data in the form of a chromatogram, notably a chromatogram obtained in the performance of an experiment to synthesize an example multi-substituent psilocybin derivative compound having the chemical formula (LIV) set forth herein.

FIG. 50 depicts a representation of further mass spectrometry data in the form of a chromatogram, notably a chromatogram obtained in the performance of an experiment to synthesize an example multi-substituent psilocybin derivative compound having the chemical formula (XXX) set forth herein.

FIG. 51 depicts a representation of further mass spectrometry data in the form of a chromatogram, notably a chromatogram obtained in the performance of an experiment to synthesize an example multi-substituent psilocybin derivative compound having the chemical formula (XXXI) set forth herein.

FIG. 52 depicts a representation of further mass spectrometry data in the form of a chromatogram, notably a chromatogram obtained in the performance of an experiment to synthesize an example multi-substituent psilocybin derivative compound having the chemical formula (XLI) set forth herein.

FIG. 53 depicts a representation of further mass spectrometry data in the form of a chromatogram, notably a chromatogram obtained in the performance of an experiment to synthesize an example multi-substituent psilocybin derivative compound having the chemical formula (XLII) set forth herein.

FIG. 54 depicts a representation of further mass spectrometry data in the form of a chromatogram, notably a chromatogram obtained in the performance of an experiment to synthesize an example multi-substituent psilocybin derivative compound having the chemical formula (XLV) set forth herein.

FIGS. 55A and 55B depict a representation of further mass spectrometry data in the form of a chromatogram, notably a chromatogram obtained in the performance of an experiment to synthesize an example multi-substituent psilocybin derivative compound having the chemical formula (XLIV) set forth herein (FIG. 55A); and a representation of mass spectrometry data in the form of a further mass spectrometry spectrum obtained in the performance of an experiment to identify a multi-substituent psilocybin derivative compound having the chemical formula (XLIV) set forth herein (FIG. 55B).

FIG. 56 depicts a representation of further mass spectrometry data in the form of a chromatogram, notably a chromatogram obtained in the performance of an experiment to synthesize an example multi-substituent psilocybin derivative compound having the chemical formula (LXVVI) set forth herein.

The figures together with the following detailed description make apparent to those skilled in the art how the disclosure may be implemented in practice.

### DETAILED DESCRIPTION

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system, or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system, or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g., a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent, or patent application was specifically and individually indicated to be incorporated by reference in its entirety.

### Terms and Definitions

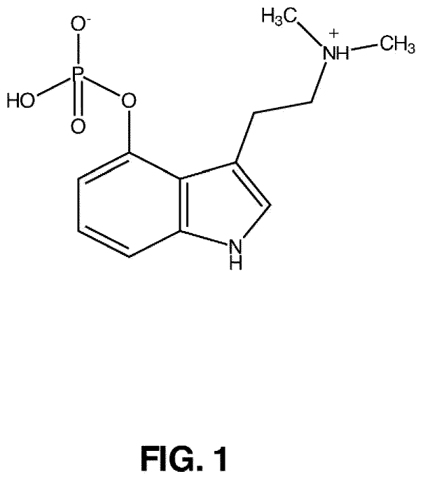

The term “psilocybin”, refers to a chemical compound having the structure set forth in FIG. 1.

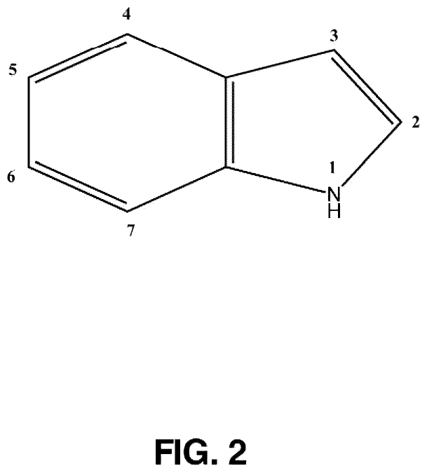

The term “indole prototype structure” refers to the chemical structure shown in FIG. 2. It is noted that specific carbon atoms and a nitrogen atom in the indole prototype structure are numbered. Reference may be made to these carbon and nitrogen numbers herein, for example C₂, C₄, N₁, and so forth. Furthermore, reference may be made to chemical groups attached to the indole prototype structure in accordance with the same numbering, for example, R₄ and R₆ reference chemical groups attached to the C₄ and C₆ atom, respectively. In addition, R_{3A} and R_{3B}, in this respect, reference chemical groups extending from the ethyl-amino group extending in turn from the C₃ atom of the prototype indole structure.

The terms “psilocybin derivative”, as used herein, refers to compounds that can be derivatized from psilocybin, wherein such compounds include an indole prototype structure and a C₃ ethylamine or ethylamine derivative group having the formula (LXXVIII):

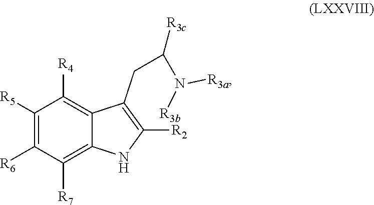

wherein R₃ₐ and R_{3b} are each independently a hydrogen atom, an alkyl group, a cycloalkyl group, an acyl group, or an aryl group, and wherein and R_{3c} is a hydrogen atom or a carboxyl group. Psilocybin derivatives include compounds containing one or more substituents at each of C₂, C₄, C₅, C₆ and C₇. Thus, in formula (LXXVIII), R₂, R₄, R₅, R₆ and R₇ can each be, for example, any of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, (x) an O-alkyl group, (xi) an (xii) O-acyl group, (xiii) a phosphate group, or (xiv) a hydrogen atom.

The term “multiple-substituent psilocybin derivative” refers to a psilocybin derivative compound wherein two or more substituent entities have been bonded to psilocybin or a psilocybin derivative. Reference may be made to specific carbon atoms which may be substituted. Furthermore the substituent entities may be referred to as S1, S2, S3 or S4, wherein each of S1, S2, S3 and S4 refer to a different substituent entity. For example, a 5,7-S1,S2-di-psilocybin derivative refers to a psilocybin derivative in which carbon atom number 5 and carbon number 7 (as identified in the indole prototype structure) each possess a different substituent entity, or, similarly, a 2,5,6-tri-S1,S2,S3-psilocybin derivative refers to a psilocybin derivative in which carbon atom number 2,5,6 (as identified in the indole prototype structure) possess a different substituent entity (or at least two of the three substituents are different). By way of another example, a 2,5,6-tri-S1,S2,S2-psilocybin derivative refers to a psilocybin derivative in which carbon atom number 2,5,6 (as identified in the indole prototype structure) each possess a substituent entity, the substituent entity possessed by carbon atom number 5 and 6 being the same. It is noted that S1, S2, S3 and S4 can herein additionally include numerical subscripts, such as S15, S36, S47 etc. Where such numerical values are included, they reference the numbered C atom of the prototype indole structure. Thus, for example, S15 is a substituent entity extending from the C₅ atom of the indole ring structure, S37 is a substituent entity extending the C₇ atom of the indole ring structure, and so forth. The term multiple-substituent psilocybin derivatives further includes chemical compounds having a chemical formula (I):

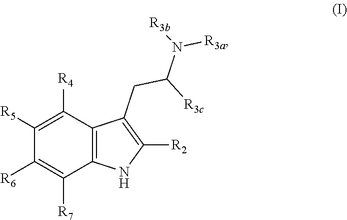

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group. The term multiple-substituent psilocybin derivatives, further also includes compounds having a formula (IV):

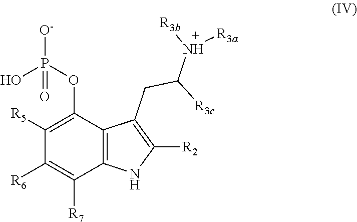

wherein, at least two of R₂, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom, wherein R₃ₐ and R_{3b} are each independently a hydrogen atom, an alkyl group, a cycloalkyl group, an acyl group, or an aryl group, and wherein and R_{3c} is a hydrogen atom or a carboxyl group. The term further includes salts of multiple-substituent psilocybins, such as a sodium salt, a potassium salt etc.

The terms “halogen”, “halogenated” and “halo-”, as used herein, refer to the class of chemical elements consisting of fluorine (F), chlorine (Cl), bromine (Br), and iodine (I). Accordingly, halogenated compounds can refer to “fluorinated”, “chlorinated”, “brominated”, or “iodinated” compounds.

The terms “phosphate group” or “phospho group”, as used herein, is a molecule containing one atom of phosphorus, covalently bound to four oxygen atoms (three single bonds and one double bond). Of the four oxygen atoms one oxygen atom may be a hydroxy group, and one of the non-hydroxylated oxygen atom may be chemically bonded to another entity.

The terms “hydroxy group”, and “hydroxy”, as used herein refers to a molecule containing one atom of oxygen bonded to one atom of hydrogen, and having the formula —OH. A hydroxy group through its oxygen atom may be chemically bonded to another entity.

The term “nitro group” and “nitro”, as used herein refers to a molecule containing one atom of nitrogen bonded to two atoms of oxygen and having the formula —NO₂. A nitro group through its nitrogen atom may be chemically bonded to another entity. Furthermore, it is noted that an entity attached to a nitro group may be referred to herein as a “nitrated” entity, e.g., a nitrated psilocybin derivative is a psilocybin derivative possessing a nitro group.

The term “amino group” and “amino”, as used herein refers to a molecule containing one atom of nitrogen bonded to hydrogen atoms and having the formula —NH₂. An amino group also may be protonated and having the formula —NH₃⁺. An amino group through its nitrogen atom may be chemically bonded to another entity. Furthermore, it is noted that an entity attached to an amino group may be referred to herein as an “aminated” entity, e.g., an aminated psilocybin derivative is a psilocybin derivative possessing either an amino group or a N-substituted amino group.

The term “N-substituted amino group”, as used herein, refers to an amino group wherein at least one of the hydrogen atoms has been substituted by another atom or group, such as, for example, an alkyl group, an acyl group, an aryl group a sulfonyl group etc. An N-substituted amino group also may be protonated, and the amino group through its nitrogen atom may be chemically bonded to another entity. Thus, N-substituted amino group may be represented herein as:

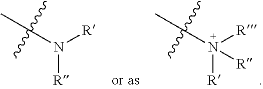

Furthermore N-substituted amino groups include:
chemical group (IV) (an alkyl group, an aryl group):

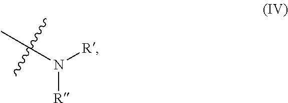

wherein R′ and R″ are each independently selected from a hydrogen atom, an alkyl group, and an aryl group, provided however that at least one of R′, and R″ is not a hydrogen atom;
chemical group (V):

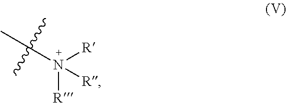

wherein R′, R″ and R′″ are each independently selected from a hydrogen atom, an alkyl group, and an aryl group, provided however that at least one of R′, R″, and R′″ is not a hydrogen atom;
chemical group (VI) (an acyl group):

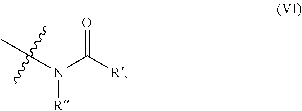

wherein R′ and R″ are each independently selected from a hydrogen atom, an alkyl group and an aryl group;
chemical group (VII) (a sulfonyl group):

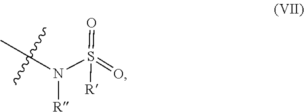

wherein R′, and R″ are each independently selected from a hydrogen atom, an alkyl group and an aryl group; and
chemical group (VIII) (a sulfonate group):

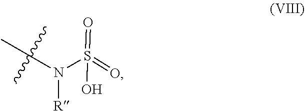

wherein R″ is selected from a hydrogen atom, an alkyl group, and an aryl group. The nitrogen atom of chemical groups (VI), (VII) and (VIII) can also be positively charged and be further substituted with H, or R′″. It is noted that R′, R″ and R′″ can herein additionally include numerical subscripts, such as ₅ₐ, _{6b}, _{7b} etc., and be represented, for example, as R′₅ₐ, R″_{6b} or R′″₇ₐ, respectively. Where such numerical values are included, they reference chemical entity extending from the amino group extending in turn from the thus numbered C atom of the prototype indole structure. Thus, for example, R′5a is a chemical entity extending from an aminated group attached to the C₅ atom of the indole ring structure, R′2a is a chemical entity extending from an aminated group attached to the C₂ atom of the indole ring structure, and so forth. Furthermore, it is noted that an entity attached to an N-substituted amino group may be referred to herein as an “aminated” entity, e.g., an aminated psilocybin derivative is a psilocybin derivative possessing either an amino group or a N-substituted amino group.

The terms “carboxyl group”, “carboxyl”, and “carboxy”, as used herein, refer to a molecule containing one atom of carbon bonded to an oxygen atom and a hydroxy group and having the formula —COOH. A carboxyl group includes a deprotonated carboxyl group, i.e., a carboxyl ion, having the formula —COO⁻. In its deprotonated form a carboxyl group may form a carboxyl salt, for example, a sodium or potassium carboxyl salt, or an organic carboxyl salt, all of which may be represented herein as COO⁻M⁺. It is further to be understood that a carboxyl group through its carbon atom may be chemically bonded to another entity. Furthermore, it is noted that an entity attached to a carboxyl group may be referred to herein as a “carboxylated” entity, e.g., a carboxylated psilocybin derivative is a psilocybin derivative possessing either a carboxyl group or a OH-substituted carboxyl group.

The term “carboxylic acid derivative”, as used herein, refers to a carboxyl group wherein the hydroxy group of the carboxyl group has been substituted by another atom or group, such as, for example, an —OR″ group or an —NR′R″ group. Thus a carboxylic acid derivative includes chemical group (III):

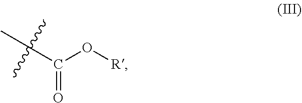

wherein, R′, is an alkyl group, an aryl group and a hydrogen atom. It is noted that chemical group (III) is an ester. It is noted that R′ can herein additionally include numerical subscripts, such as _{3c}, _{6b}, _{7b} etc., and be represented, for example, as R′_{3c}, R′_{6b} or R′₇ₐ, respectively. Where such numerical values are included, they reference chemical entity extending from the carboxyl group extending in turn from the thus numbered C atom of the prototype indole structure. Thus, for example, R′₅ₐ is a chemical entity extending from a carboxylated group attached to the C₅ atom of the indole ring structure, R′₂ₐ is a chemical entity extending from a carboxylated group attached to the C₂ atom of the indole ring structure, and so forth. Furthermore, it is noted that an entity attached to a carboxylic acid derivative may be referred to herein as an “carboxylated” entity, e.g., a carboxylated psilocybin derivative is a psilocybin derivative possessing either a carboxyl group or an OH-substituted carboxyl group.

The terms “aldehyde” or “aldehyde group”, as used herein, refers to a molecule containing one atom of carbon double bonded to an oxygen atom, and bonded to a hydrogen atom, and having the chemical formula:

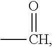

which may, further alternatively be represented herein as —CHO. A —CHO group may also by referred to herein as a formyl group. It is to be understood that an aldehyde through its carbon atom may be chemically bonded to another entity.

The terms “ketone” or “ketone group”, as used herein, refer to a molecule containing two atoms of carbon, a first carbon atom double bonded to an oxygen atom, and the first carbon further bonded to a second carbon atom, the molecule having the chemical formula:

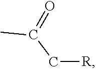

wherein R is any entity or plurality of entities which taken together allow the carbon atom bonded to R to achieve its ordinary valency. Thus, for example, R may represent 3 hydrogen atoms, or R may represent 2 hydrogen atoms and a methyl group. It is to be understood that a ketone through its first carbon atom may be chemically bonded to another entity, such as an alkylene group (C₁-C₆)-alkylene.

The term “nitrile group” and “nitrile”, as used herein, refer to a molecule containing one atom of carbon bonded to a nitrogen atom and having the formula

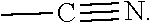

It is to be understood that a nitrile group through its carbon atom may be chemically bonded to another entity. Furthermore, it is noted that an entity attached to a nitrile group may be referred to herein as a “nitrilated” entity, e.g., a nitrilated psilocybin derivative is a psilocybin derivative possessing a nitrile group.

The terms “glycosylated” or “glycosyl”, as used herein, refer to a saccharide group, such as a mono-, di-, tri- oligo- or a poly-saccharide group, which can be or has been bonded from its anomeric carbon either in the pyranose or furanose form, either in the α or the β conformation. When bonded through its anomeric carbon via an oxygen atom to another entity, the bonded saccharide group, inclusive of the oxygen atom, may be referred to herein as a “glycosyloxy” group. Example monosaccharide groups include, but are not limited to, a pentosyl, a hexosyl, or a heptosyl group. The glycosyloxy group may also be substituted with various groups. Such substitutions may include lower alkyl, lower alkoxy, acyl, carboxy, carboxyamino, amino, acetamido, halo, thio, nitro, keto, and phosphatyl groups, wherein the substitution may be at one or more positions on the saccharide. Included in the term glycosyl are further stereoisomers, optical isomers, anomers, and epimers of the glycosyloxy group. Thus, a hexose group, for example, can be either an aldose or a ketose group, can be of D- or L-configuration, can assume either an a or p conformation, and can be a dextro- or levo-rotatory with respect to plane-polarized light. Example glycosyloxy groups further include, without limitation, glucosyl groups, glucuronic acid groups, galactosyl groups, fucosyl groups, xylose groups, arabinose groups, and rhamnose groups.

The terms “prenyl group”, and “prenyl”, as used herein refers to a chemical group having the structure (LVI):

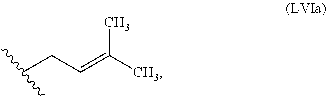 [SMILES: *CC=C(C)C]

and further includes poly-prenyl compounds having the structure:

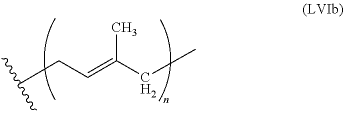

Wherein n is an integer having a value of 2 or more, e.g., 2, 3, 4, 5, etc. Furthermore, the term “prenyl compound” refers to a chemical compound being, substantially being, or possessing a reactive prenyl group, i.e., a prenyl group that may be received by another entity. Prenyl compounds include, for example, geranyl pyrophosphate (GPP), dimethylallyl diphosphate (DMAPP), farnesyl pyrophosphate (FPP) and geranylgeranyl pyrophosphate (GGPP).

The term “alkyl group”, as used herein, refers to a straight and/or branched chain, saturated alkyl radical containing from one to “p” carbon atoms (“C₁-Cₚ-alkyl”) and includes, depending on the identity of “p”, methyl, ethyl, propyl, isopropyl, n-butyl, s-butyl, isobutyl, t-butyl, 2,2-dimethylbutyl, n-pentyl, 2-methylpentyl, 3-methylpentyl 4-methylpentyl, n-hexyl, and the like, where the variable p is an integer representing the largest number of carbon atoms in the alkyl radical. Alkyl groups further include hydrocarbon groups arranged in a chain having the chemical formula —CₙH₂ₙ₊₁, including, without limitation, methyl groups (—CH₃), ethyl groups (—C₂H₅), propyl groups (—C₃H₇), and butyl groups (—C₄H₉), further also includes cyclic alkyl groups, including cyclo-propane, cyclo-butane, cyclo-pentane, cyclo-hexane, and cyclo-heptane.

The term “cycloalkyl” refers to cyclic alkyl groups, including (C₃-C₂₀), (C₃-C₁₀), and (C₃-C₆) cycloalkyl groups, and further including cyclo-propane, cyclo-butane, cyclo-pentane, cyclo-hexane, and cyclo-heptane.

The term “O-alkyl group”, as used herein, refers to a hydrocarbon group arranged in a chain having the chemical formula —O—CₙH₂ₙ₊₁. O-alkyl groups include, without limitation, O-methyl groups (—O—CH₃), O-ethyl groups (—O—C₂H₅), O-propyl groups (—O—C₃H₇) and O-butyl groups (—O—C₄H₉).

The term “aryl group”, as used herein, refers to a hydrocarbon group arranged in an aromatic ring and can, for example, be a C₆-C₁₄-aryl, a C₆-C₁₀-aryl. Aryl groups further include phenyl, naphthyl, tetrahydronaphthyl, phenanthrenyl, biphenylenyl, indanyl, tolyl, xylyl, or indenyl groups, and the like.

The term “acyl group”, as used herein, refers to a carbon atom double bonded to an oxygen and single bonded to an alkyl group. The carbon atom further can be bonded to another entity. An acyl group can be described by the chemical formula: —C(═O)—CₙH₂ₙ₊₁.

The term “O-acyl group”, as used herein, refers to an acyl group in which the carbon atom is single bonded to an additional oxygen atom. The additional oxygen atom can be bonded to another entity. An O-acyl group can be described by the chemical formula: —O—C(═O)—CₙH₂ₙ₊₁. Furthermore, depending on the carbon chain, length specific O-acyl groups may be termed an acetoxy group (n=1), a propanoyloxy group (n=2), butyryloxy group (n=3), a pentanoyloxy group (n=4) etc.

The term “alcohol group” or “hydroxylalkyl”, as used herein, refers to a hydrocarbon group arranged in a chain having the chemical formula CₙHₙ₊₁OH. Depending on the carbon chain, length specific alcohol groups may be termed a methanol group (n=1) or hydroxymethyl, an ethanol group (n=2) or hydroxyethyl, a propanol group (n=3) or hydroxypropyl, a butanol group (n=4) or hydroxybutyl etc.

The term “5-HT_{2A} receptor”, as used herein, refers to a subclass of a family of receptors for the neurotransmitter and peripheral signal mediator serotonin. 5-HT_{2A} receptors can mediate a plurality of central and peripheral physiologic functions of serotonin. Central nervous system effects can include mediation of hallucinogenic effects of hallucinogenic compounds.

The term “modulating 5-HT_{2A} receptors”, as used herein, refers to the ability of a compound disclosed herein to alter the function of 5-HT_{2A} receptors. A 5-HT_{2A} receptor modulator may activate the activity of a 5-HT_{2A} receptor, may activate or inhibit the activity of a 5-HT_{2A} receptor depending on the concentration of the compound exposed to the 5-HT_{2A} receptor, or may inhibit the activity of a 5-HT_{2A} receptor. Such activation or inhibition may be contingent on the occurrence of a specific event, such as activation of a signal transduction pathway, and/or maybe manifest only in particular cell types. The term “modulating 5-HT_{2A} receptors,” also refers to altering the function of a 5-HT_{2A} receptor by increasing or decreasing the probability that a complex forms between a 5-HT_{2A} receptor and a natural binding partner to form a multimer. A 5-HT_{2A} receptor modulator may increase the probability that such a complex forms between the 5-HT_{2A} receptor and the natural binding partner, may increase or decrease the probability that a complex forms between the 5-HT_{2A} receptor and the natural binding partner depending on the concentration of the compound exposed to the 5-HT_{2A} receptor, and or may decrease the probability that a complex forms between the 5-HT_{2A} receptor and the natural binding partner. It is further noted that the prenylated psilocybin derivatives may alter the function of a 5-HT_{2A} receptor by acting as an agonist or antagonist of the 5-HT_{1A} receptor, and that prenylated psilocybin derivatives according to the present disclosure may alter the function of a 5-HT_{2A} receptor by directly interacting therewith or binding thereto, or by indirectly interacting therewith through one or more other molecular entities.

The term “5-HT_{2A} receptor-mediated disorder”, as used herein, refers to a disorder that is characterized by abnormal 5-HT_{2A} receptor activity. A 5-HT_{2A} receptor-mediated disorder may be completely or partially mediated by modulating 5-HT_{2A} receptors. In particular, a 5-HT_{2A} receptor-mediated disorder is one in which modulation of 5-HT_{2A} receptors results in some effect on the underlying disorder e.g., administration of a 5-HT_{2A} receptor modulator results in some improvement in at least some of the subjects being treated.

The term “5-HT_{1A} receptor”, as used herein, refers to a subclass of a family of receptors for the neurotransmitter and peripheral signal mediator serotonin. 5-HT_{1A} receptors can mediate a plurality of central and peripheral physiologic functions of serotonin. Ligand activity at 5-HT_{1A} is generally not associated with hallucination, although many hallucinogenic compounds are known to modulate 5-HT_{1A} receptors to impart complex physiological responses (Inserra et al., 2020, Pharmacol Rev 73: 202).

The term “modulating 5-HT_{1A} receptors”, as used herein, refers to the ability of a compound disclosed herein to alter the function of 5-HT_{1A} receptors. A 5-HT_{1A} receptor modulator may activate the activity of a 5-HT_{1A} receptor, may activate or inhibit the activity of a 5-HT_{1A} receptor depending on the concentration of the compound exposed to the 5-HT_{1A} receptor, or may inhibit the activity of a 5-HT_{1A} receptor. Such activation or inhibition may be contingent on the occurrence of a specific event, such as activation of a signal transduction pathway, and/or maybe manifest only in particular cell types. The term “modulating 5-HT_{1A} receptors,” also refers to altering the function of a 5-HT_{1A} receptor by increasing or decreasing the probability that a complex forms between a 5-HT_{1A} receptor and a natural binding partner to form a multimer. A 5-HT_{1A} receptor modulator may increase the probability that such a complex forms between the 5-HT_{1A} receptor and the natural binding partner, may increase or decrease the probability that a complex forms between the 5-HT_{1A} receptor and the natural binding partner depending on the concentration of the compound exposed to the 5-HT_{1A} receptor, and or may decrease the probability that a complex forms between the 5-HT_{1A} receptor and the natural binding partner. It is further noted that the prenylated psilocybin derivatives may alter the function of a 5-HT_{1A} receptor by acting as an agonist or antagonist of the 5-HT_{1A} receptor, and that prenylated psilocybin derivatives according to the present disclosure may alter the function of a 5-HT_{1A} receptor by directly interacting therewith or binding thereto, or by indirectly interacting therewith through one or more other molecular entities.

The term “5-HT_{1A} receptor-mediated disorder”, as used herein, refers to a disorder that is characterized by abnormal 5-HT_{1A} receptor activity. A 5-HT_{1A} receptor-mediated disorder may be completely or partially mediated by modulating 5-HT_{1A} receptors. In particular, a 5-HT_{1A} receptor-mediated disorder is one in which modulation of 5-HT_{1A} receptors results in some effect on the underlying disorder e.g., administration of a 5-HT_{1A} receptor modulator results in some improvement in at least some of the subjects being treated.

The term “reactant psilocybin derivative compound”, as used herein, refers to a psilocybin derivative compound capable of reacting in a synthetic or biosynthetic reaction to thereby form another psilocybin derivative compound, and generally includes indole structure containing reactants. The term “reactant psilocybin derivative compound” includes the term “psilocybin derivative precursor compound”.

The term “psilocybin derivative precursor compound”, as used herein, refers to a chemical compound that may serve as a precursor compound in the synthesis or biosynthesis of a multi-substituent psilocybin derivative, and includes compounds comprising an indole prototype structure, including, for example, tryptophan and tryptamine, and further includes a psilocybin derivative precursor compound having a formula (LVII):

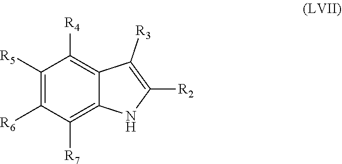

wherein at least one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, and wherein R₃ is a hydrogen atom or —CH₂—CHNH₂COOH or —CH₂—CH₂NH₂.

The term “psilocybin biosynthetic enzyme complement”, as used herein, refers to one or more polypeptides which alone or together are capable of facilitating the chemical conversion of a psilocybin derivative precursor compound, and form a multi-substituent psilocybin derivative compound. A psilocybin biosynthetic enzyme complement can include, for example, one or more of a tryptophan synthase B polypeptide, a tryptophan decarboxylase, an N-acetyl transferase, a N-methyl transferase and a prenyl transferase.

The term “tryptophan synthase B polypeptide”, as used herein, refers to any and all enzymes comprising a sequence of amino acid residues which is (i) substantially identical to the amino acid sequences constituting any tryptophan synthase B polypeptide set forth herein, including, for example, SEQ. ID NO: 2, or (ii) encoded by a nucleic acid sequence capable of hybridizing under at least moderately stringent conditions to any nucleic acid sequence encoding any tryptophan synthase B polypeptide set forth herein, but for the use of synonymous codons.

The term “tryptophan decarboxylase”, as used herein, refers to any and all enzymes comprising a sequence of amino acid residues which is (i) substantially identical to the amino acid sequences constituting any tryptophan decarboxylase polypeptide set forth herein, including, for example, SEQ. ID NO: 4, SEQ. ID NO: 6 and SEQ. ID NO: 8, or (ii) encoded by a nucleic acid sequence capable of hybridizing under at least moderately stringent conditions to any nucleic acid sequence encoding any tryptophan decarboxylase set forth herein, but for the use of synonymous codons.

The term “N-acetyl transferase”, as used herein, refers to any and all enzymes comprising a sequence of amino acid residues which is (i) substantially identical to the amino acid sequences constituting any acetyl transferase polypeptide set forth herein, including, for example, SEQ. ID NO: 10, or (ii) encoded by a nucleic acid sequence capable of hybridizing under at least moderately stringent conditions to any nucleic acid sequence encoding any acetyl transferase set forth herein, but for the use of synonymous codons.

The term “N-methyl transferase”, as used herein, refers to any and all enzymes comprising a sequence of amino acid residues which is (i) substantially identical to the amino acid sequences constituting any N-methyl transferase polypeptide set forth herein, including, for example, SEQ. ID NO: 12 and SEQ. ID NO: 14, or (ii) encoded by a nucleic acid sequence capable of hybridizing under at least moderately stringent conditions to any nucleic acid sequence encoding any N-methyl transferase set forth herein, but for the use of synonymous codons.

The term “prenyl transferase”, as used herein, refers to any and all enzymes comprising a sequence of amino acid residues which is (i) substantially identical to the amino acid sequences constituting any prenyl transferase polypeptide set forth herein, including, for example, SEQ. ID NO: 16, SEQ. ID NO: 18, SEQ. ID NO: 20, and SEQ. ID NO: 22, or (ii) encoded by a nucleic acid sequence capable of hybridizing under at least moderately stringent conditions to any nucleic acid sequence encoding any prenyl transferase set forth herein, but for the use of synonymous codons.

The term “PsiH”, as used herein, refers to any and all enzymes comprising a sequence of amino acid residues which is (i) substantially identical to the amino acid sequences constituting any PsiH polypeptide set forth herein, including, for example, SEQ. ID NO: 24, or (ii) encoded by a nucleic acid sequence capable of hybridizing under at least moderately stringent conditions to any nucleic acid sequence encoding any PsiH set forth herein, but for the use of synonymous codons.

The term “CPR”, as used herein, refers to any and all enzymes comprising a sequence of amino acid residues which is (i) substantially identical to the amino acid sequences constituting any CPR polypeptide set forth herein, including, for example, SEQ. ID NO: 26, or (ii) encoded by a nucleic acid sequence capable of hybridizing under at least moderately stringent conditions to any nucleic acid sequence encoding any CPR set forth herein, but for the use of synonymous codons.

The term “PsiK”, as used herein, refers to any and all enzymes comprising a sequence of amino acid residues which is (i) substantially identical to the amino acid sequences constituting any PsiK set forth herein, including, for example, SEQ. ID NO: 49, or (ii) encoded by a nucleic acid sequence capable of hybridizing under at least moderately stringent conditions to any nucleic acid sequence encoding any PsiK set forth herein, but for the use of synonymous codons.

The terms “nucleic acid sequence encoding tryptophan synthase B polypeptide”, and “nucleic acid sequence encoding a tryptophan synthase B polypeptide”, as may be used interchangeably herein, refer to any and all nucleic acid sequences encoding a tryptophan synthase B polypeptide, including, for example, SEQ. ID NO: 1. Nucleic acid sequences encoding a tryptophan synthase B polypeptide further include any and all nucleic acid sequences which (i) encode polypeptides that are substantially identical to the tryptophan synthase B polypeptide sequences set forth herein; or (ii) hybridize to any tryptophan synthase B polypeptide nucleic acid sequences set forth herein under at least moderately stringent hybridization conditions or which would hybridize thereto under at least moderately stringent conditions but for the use of synonymous codons.

The terms “nucleic acid sequence encoding tryptophan decarboxylase”, and “nucleic acid sequence encoding a tryptophan decarboxylase polypeptide”, as may be used interchangeably herein, refer to any and all nucleic acid sequences encoding a tryptophan decarboxylase polypeptide, including, for example, SEQ. ID NO: 3, SEQ. ID NO: 5 and SEQ. ID NO: 7. Nucleic acid sequences encoding a tryptophan decarboxylase polypeptide further include any and all nucleic acid sequences which (i) encode polypeptides that are substantially identical to the tryptophan decarboxylase polypeptide sequences set forth herein; or (ii) hybridize to any tryptophan decarboxylase nucleic acid sequences set forth herein under at least moderately stringent hybridization conditions or which would hybridize thereto under at least moderately stringent conditions but for the use of synonymous codons.

The terms “nucleic acid sequence encoding an N-acetyl transferase”, and “nucleic acid sequence encoding an N-acetyl transferase polypeptide”, as may be used interchangeably herein, refer to any and all nucleic acid sequences encoding an N-acetyl transferase polypeptide, including, for example, SEQ. ID NO: 9. Nucleic acid sequences encoding an N-acetyl transferase polypeptide further include any and all nucleic acid sequences which (i) encode polypeptides that are substantially identical to the N-acetyl transferase polypeptide sequences set forth herein; or (ii) hybridize to any N-acetyl transferase nucleic acid sequences set forth herein under at least moderately stringent hybridization conditions or which would hybridize thereto under at least moderately stringent conditions but for the use of synonymous codons.

The terms “nucleic acid sequence encoding N-methyl transferase”, and “nucleic acid sequence encoding a N-methyl transferase polypeptide”, as may be used interchangeably herein, refer to any and all nucleic acid sequences encoding a N-methyl transferase polypeptide, including, for example, SEQ. ID NO: 11 and SEQ. ID NO: 13. Nucleic acid sequences encoding a N-methyl transferase polypeptide further include any and all nucleic acid sequences which (i) encode polypeptides that are substantially identical to the N-methyl transferase polypeptide sequences set forth herein; or (ii) hybridize to any N-methyl transferase nucleic acid sequences set forth herein under at least moderately stringent hybridization conditions or which would hybridize thereto under at least moderately stringent conditions but for the use of synonymous codons.

The terms “nucleic acid sequence encoding a prenyl transferase”, and “nucleic acid sequence encoding a prenyl transferase polypeptide”, as may be used interchangeably herein, refer to any and all nucleic acid sequences encoding a prenyl transferase polypeptide, including, for example, SEQ. ID NO: 15, SEQ. ID NO: 17, SEQ. ID NO; 19 and SEQ. ID NO: 21. Nucleic acid sequences encoding a prenyl transferase polypeptide further include any and all nucleic acid sequences which (i) encode polypeptides that are substantially identical to the prenyl transferase polypeptide sequences set forth herein; or (ii) hybridize to any prenyl transferase nucleic acid sequences set forth herein under at least moderately stringent hybridization conditions or which would hybridize thereto under at least moderately stringent conditions but for the use of synonymous codons.

The terms “nucleic acid sequence encoding PsiH”, and “nucleic acid sequence encoding a PsiH polypeptide”, as may be used interchangeably herein, refer to any and all nucleic acid sequences encoding a PsiH, including, for example, SEQ. ID NO: 23. Nucleic acid sequences encoding a PsiH polypeptide further include any and all nucleic acid sequences which (i) encode polypeptides that are substantially identical to the PsiH polypeptide sequences set forth herein; or (ii) hybridize to any PsiH nucleic acid sequences set forth herein under at least moderately stringent hybridization conditions or which would hybridize thereto under at least moderately stringent conditions but for the use of synonymous codons.

The terms “nucleic acid sequence encoding CPR”, and “nucleic acid sequence encoding an CPR polypeptide”, as may be used interchangeably herein, refer to any and all nucleic acid sequences encoding a CPR, including, for example, SEQ. ID NO: 25. Nucleic acid sequences encoding a CPR polypeptide further include any and all nucleic acid sequences which (i) encode polypeptides that are substantially identical to the CPR polypeptide sequences set forth herein; or (ii) hybridize to any CPR nucleic acid sequences set forth herein under at least moderately stringent hybridization conditions or which would hybridize thereto under at least moderately stringent conditions but for the use of synonymous codons.

The terms “nucleic acid sequence encoding a PsiK”, and “nucleic acid sequence encoding a PsiK polypeptide”, as may be used interchangeably herein, refer to any and all nucleic acid sequences encoding PsiK, including, for example, SEQ. ID NO: 48. Nucleic acid sequences encoding a PsiK further include any and all nucleic acid sequences which (i) encode polypeptides that are substantially identical to the PsiK polypeptide sequences set forth herein; or (ii) hybridize to any PsiK nucleic acid sequences set forth herein under at least moderately stringent hybridization conditions or which would hybridize thereto under at least moderately stringent conditions but for the use of synonymous codons.

The terms “nucleic acid”, or “nucleic acid sequence”, as used herein, refer to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acids of the present disclosure may be deoxyribonucleic nucleic acids (DNA) or ribonucleic acids (RNA) and may include naturally occurring bases including adenine, guanine, cytosine, thymidine, and uracil. The nucleic acids may also contain modified bases. Examples of such modified bases include aza and deaza adenine, guanine, cytosine, thymidine and uracil, and xanthine and hypoxanthine. A sequence of nucleotide or nucleoside monomers may be referred to as a polynucleotide sequence, nucleic acid sequence, a nucleotide sequence, or a nucleoside sequence.

The term “polypeptide”, as used herein in conjunction with a reference SEQ. ID NO, refers to any and all polypeptides comprising a sequence of amino acid residues which is (i) substantially identical to the amino acid sequence constituting the polypeptide having such reference SEQ. ID NO, or (ii) encoded by a nucleic acid sequence capable of hybridizing under at least moderately stringent conditions to any nucleic acid sequence encoding the polypeptide having such reference SEQ. ID NO, but for the use of synonymous codons. A sequence of amino acid residues may be referred to as an amino acid sequence, or polypeptide sequence.

The term “nucleic acid sequence encoding a polypeptide”, as used herein in conjunction with a reference SEQ. ID NO, refers to any and all nucleic acid sequences encoding a polypeptide having such reference SEQ. ID NO. Nucleic acid sequences encoding a polypeptide, in conjunction with a reference SEQ. ID NO, further include any and all nucleic acid sequences which (i) encode polypeptides that are substantially identical to the polypeptide having such reference SEQ. ID NO; or (ii) hybridize to any nucleic acid sequences encoding polypeptides having such reference SEQ. ID NO under at least moderately stringent hybridization conditions or which would hybridize thereto under at least moderately stringent conditions but for the use of synonymous codons.

By the term “substantially identical” it is meant that two amino acid sequences preferably are at least 70% identical, and more preferably are at least 85% identical and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et al., J. Mol. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts, and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

The term “functional variant”, as used herein in reference to polynucleotides or polypeptides, refers to polynucleotides or polypeptides capable of performing the same function as a noted reference polynucleotide or polypeptide. Thus, for example, a functional variant of the polypeptide set forth in SEQ. ID NO: 2, refers to a polypeptide capable of performing the same function as the polypeptide set forth in SEQ. ID NO: 2. Functional variants include modified a polypeptide wherein, relative to a noted reference polypeptide, the modification includes a substitution, deletion, or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine, and valine, (iv) asparagine and glutamine, and (v) tryptophan, tyrosine, and phenylalanine. Functional variants further include polypeptides having retained or exhibiting an enhanced psilocybin biosynthetic bioactivity.

The term “chimeric”, as used herein in the context of nucleic acids, refers to at least two linked nucleic acids which are not naturally linked. Chimeric nucleic acids include linked nucleic acids of different natural origins. For example, a nucleic acid constituting a microbial promoter linked to a nucleic acid encoding a plant polypeptide is considered chimeric. Chimeric nucleic acids also may comprise nucleic acids of the same natural origin, provided they are not naturally linked. For example a nucleic acid constituting a promoter obtained from a particular cell-type may be linked to a nucleic acid encoding a polypeptide obtained from that same cell-type, but not normally linked to the nucleic acid constituting the promoter. Chimeric nucleic acids also include nucleic acids comprising any naturally occurring nucleic acids linked to any non-naturally occurring nucleic acids.

The term “pharmaceutical formulation”, as used herein, refers to a preparation in a form which allows an active ingredient, including a psychoactive ingredient, contained therein to provide effective treatment, and which does not contain any other ingredients which cause excessive toxicity, an allergic response, irritation, or other adverse response commensurate with a reasonable risk/benefit ratio. The pharmaceutical formulation may contain other pharmaceutical ingredients such as excipients, carriers, diluents, or auxiliary agents.

The term “recreational drug formulation”, as used herein, refers to a preparation in a form which allows a psychoactive ingredient contained therein to be effective for administration as a recreational drug, and which does not contain any other ingredients which cause excessive toxicity, an allergic response, irritation, or other adverse response commensurate with a reasonable risk/benefit ratio. The recreational drug formulation may contain other ingredients such as excipients, carriers, diluents, or auxiliary agents.

The term “effective for administration as a recreational drug”, as used herein, refers to a preparation in a form which allows a subject to voluntarily induce a psychoactive effect for non-medical purposes upon administration, generally in the form of self-administration. The effect may include an altered state of consciousness, satisfaction, pleasure, euphoria, perceptual distortion, or hallucination.

The term “effective amount”, as used herein, refers to an amount of an active agent, pharmaceutical formulation, or recreational drug formulation, sufficient to induce a desired biological or therapeutic effect, including a prophylactic effect, and further including a psychoactive effect. Such effect can include an effect with respect to the signs, symptoms or causes of a disorder, or disease or any other desired alteration of a biological system. The effective amount can vary depending, for example, on the health condition, injury stage, disorder stage, or disease stage, weight, or sex of a subject being treated, timing of the administration, manner of the administration, age of the subject, and the like, all of which can be determined by those of skill in the art.

The terms “treating” and “treatment”, and the like, as used herein, are intended to mean obtaining a desirable physiological, pharmacological, or biological effect, and includes prophylactic and therapeutic treatment. The effect may result in the inhibition, attenuation, amelioration, or reversal of a sign, symptom or cause of a disorder, or disease, attributable to the disorder, or disease, which includes mental and psychiatric diseases and disorders. Clinical evidence of the prevention or treatment may vary with the disorder, or disease, the subject, and the selected treatment.

The term “pharmaceutically acceptable”, as used herein, refers to materials, including excipients, carriers, diluents, or auxiliary agents, that are compatible with other materials in a pharmaceutical or recreational drug formulation and within the scope of reasonable medical judgement suitable for use in contact with a subject without excessive toxicity, allergic response, irritation, or other adverse response commensurate with a reasonable risk/benefit ratio.

The terms “substantially pure” and “isolated”, as may be used interchangeably herein describe a compound, e.g., a psilocybin derivative, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., by chromatography, gel electrophoresis or HPLC analysis.

The term “recovered”, as used herein in association with an enzyme, protein, or a chemical compound, refers to a more or less pure form of the enzyme, protein, or chemical compound.

The term “in vivo”, as used herein relation to a method of making a multi-substituent psilocybin derivative compound, refers to a method involving contacting a psilocybin derivative precursor compound with an enzyme capable of converting the psilocybin derivative precursor compound within a cell, for example, a cell or a microorganism, cultivated, for example, in a growth medium, to convert the psilocybin derivative precursor compound into a multi-substituent psilocybin derivative compound. The cell generally expresses a psilocybin biosynthetic enzyme complex, including a heterologously expressed tryptophan synthase B polypeptide, a tryptophan decarboxylase, an N-acetyl transferase, a N-methyl transferase and a prenyl transferase, for example.

The term “in vitro”, as used herein relation to a method of making a multi-substituent psilocybin derivative compound, refers to a method involving contacting a psilocybin derivative precursor compound with an enzyme capable of converting the psilocybin derivative precursor outside a cell, for example, in a microwell plate, a tube, a flask, a beaker, a tank, a reactor, or the like, to convert the psilocybin derivative precursor compound into a multi-substituent psilocybin derivative compound.

### General Implementation

As hereinbefore mentioned, the present disclosure relates to psilocybin derivatives. In particular, the present disclosure provides novel multiple-substituent psilocybin derivatives. In general, the herein provided compositions exhibit functional properties which deviate from the functional properties of psilocybin. Thus, for example, the multiple-substituent psilocybin derivatives, can exhibit pharmacological properties which deviate from psilocybin. Furthermore, the multiple-substituent derivatives may psilocybin derivatives may exhibit physico-chemical properties which differ from psilocybin. Thus, for example, multiple-substituent psilocybin derivatives may exhibit superior solubility in a solvent, for example, an aqueous solvent. The multiple-substituent psilocybin derivatives in this respect are useful in the formulation of pharmaceutical and recreational drug formulations. In one embodiment, the multiple-substituent psilocybin derivatives of the present disclosure can conveniently be chemically and/or biosynthetically produced. The practice of this method avoids the extraction of psilocybin from mushrooms and the performance of subsequent chemical reactions to achieve multiple-substituent derivatives. Furthermore, the growth of mushrooms can be avoided thus limiting the dependence on climate and weather, and potential legal and social challenges associated with the cultivation of mushrooms containing psychoactive compounds. The method can efficiently yield substantial quantities of multiple-substituent psilocybin derivatives.

In what follows selected embodiments are described with reference to the drawings.

Initially example multiple-substituent psilocybin derivatives will be described. Thereafter example methods of using and making the multiple-substituent psilocybin derivatives will be described.

Accordingly, in one aspect the present disclosure provides derivatives of a compound known as psilocybin of which the chemical structure is shown in FIG. 1. The derivatives herein provided are, in particular, multiple-substituent derivatives including psilocybin derivatives possessing two or more substituent entities.

Thus, in one aspect, the present disclosure provides, in accordance with the teachings herein, in at least one embodiment, a chemical compound having a formula (I):

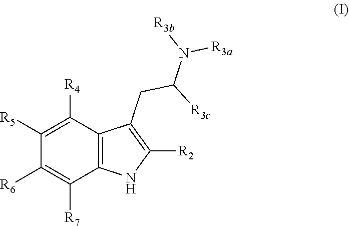

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

Thus, referring to the chemical compound having the formula (I), initially it is noted that, in an aspect hereof, at least two of R₂, R₄, R₅, R₆, or R₇ are substituent entities selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group. Thus, it is to be understood that, in accordance with an aspect of the present disclosure, at least two of R₂, R₄, R₅, R₆, or R₇ are substituent entities. The substituent entities are each independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and where two (but no more than two) substituent entities are selected, they are non-identical, and where three or more substituent entities are selected, at least two of the selected substituent entities are non-identical.

In an aspect hereof, in an embodiment, at least three of R₂, R₄, R₅, R₆, or R₇ can be substituents selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

In an aspect hereof, in an embodiment, at least three of R₂, R₄, R₅, R₆, or R₇ can be substituents selected from at least three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Continuing to refer to the chemical compound having the formula (I), in a further aspect hereof, R₄ can be a phosphate group or a hydrogen atom.

Continuing to refer to the chemical compound having the formula (I), in a further aspect hereof, R₃ₐ and R_{3b} can be a hydrogen atom, an alkyl group, an acyl group or an aryl group. Thus, R₃ₐ and R_{3b} can each be a hydrogen atom, or R₃ₐ and R_{3b} can each be an alkyl group, such as a methyl group, ethyl group, propyl group, or longer chain alkyl group, or R₃ₐ and R_{3b} can be each be an acyl group, or R₃ₐ and R_{3b} can each be an aryl group. Furthermore, one of R₃ₐ and R_{3b} can be a hydrogen atom, and one of R₃ₐ and R_{3b} can be an alkyl group. One of R₃ₐ and R_{3b} can be a hydrogen atom, and one of R₃ₐ and R_{3b} can be an acyl group. One of R₃ₐ and R_{3b} can be a hydrogen atom, and one of R₃ₐ and R_{3b} can be an aryl group. One of R₃ₐ and R_{3b} can be an alkyl group, and one of R₃ₐ and R_{3b} can be an aryl group. One of R₃ₐ and R_{3b} can be an alkyl group, and one of R₃ₐ and R_{3b} can be an acyl group. One of R₃ₐ and R_{3b} can be an acyl group, and one of R₃ₐ and R_{3b} can be an aryl group.

Continuing to refer to the chemical compound having the formula (I), in a further aspect hereof, each of the non-substituted groups R₂, R₅, R₆, or R₇ can be a hydrogen atom. Moreover, as hereinbefore noted, R₄ can also be a hydrogen atom.

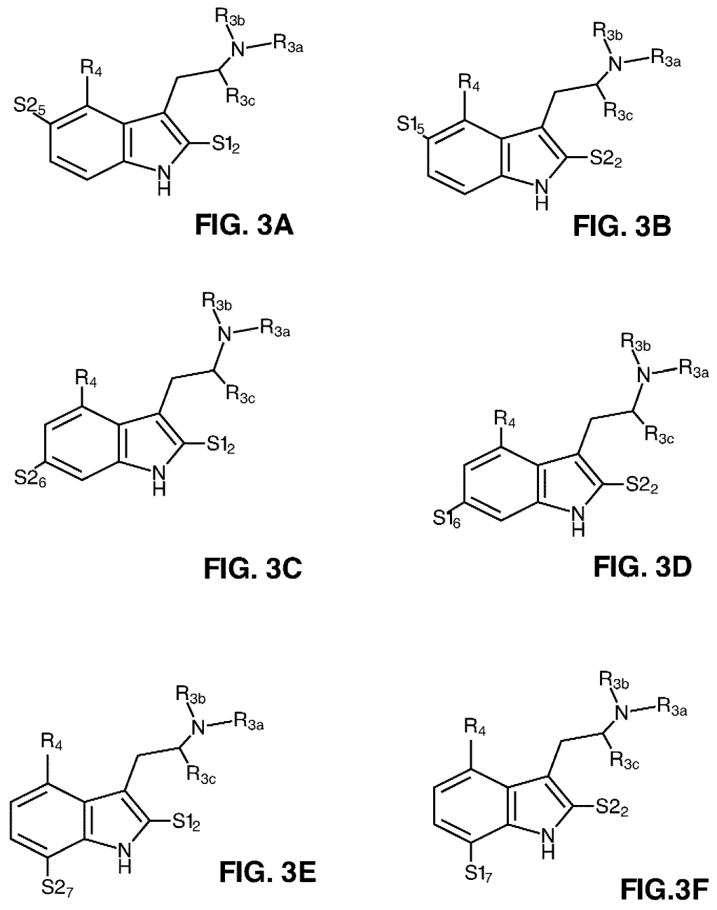

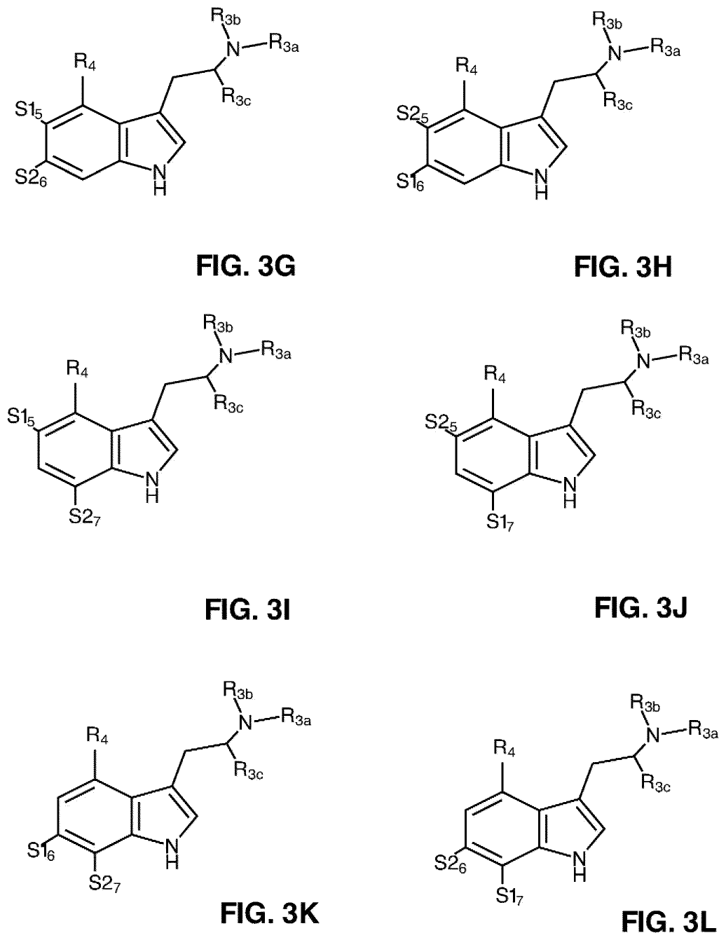

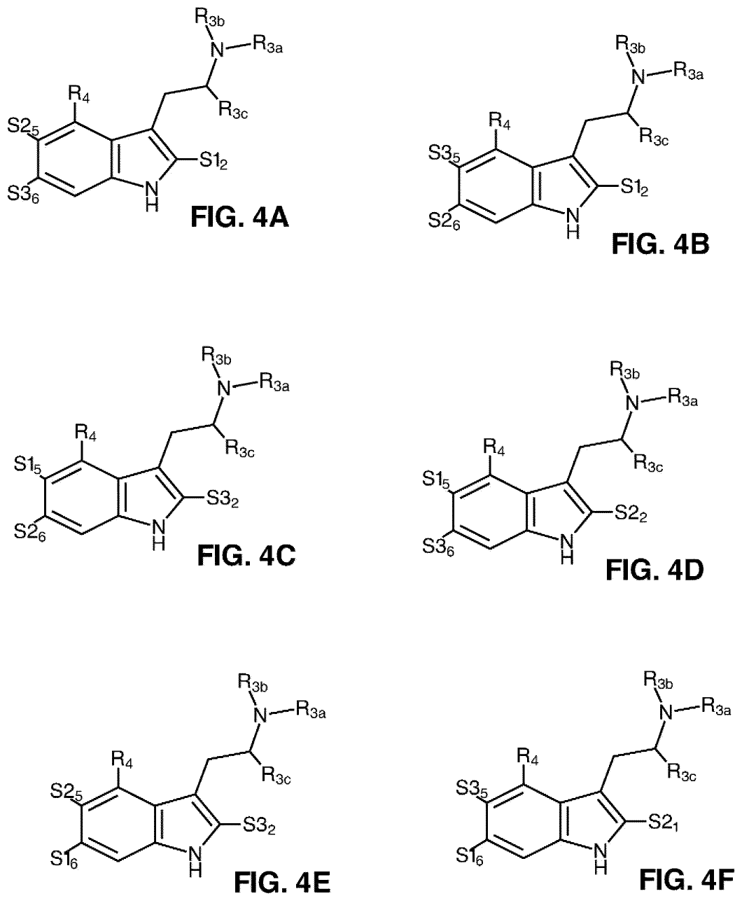

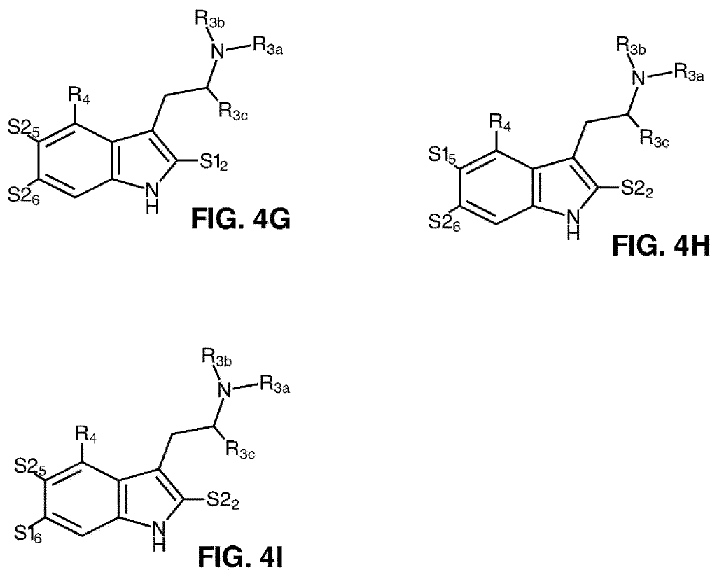

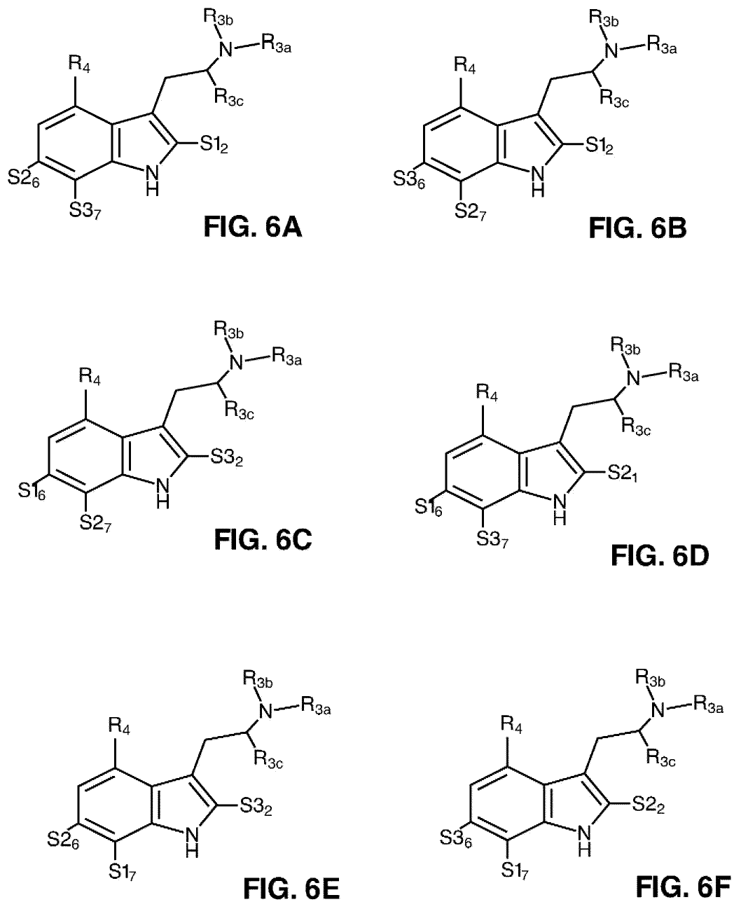

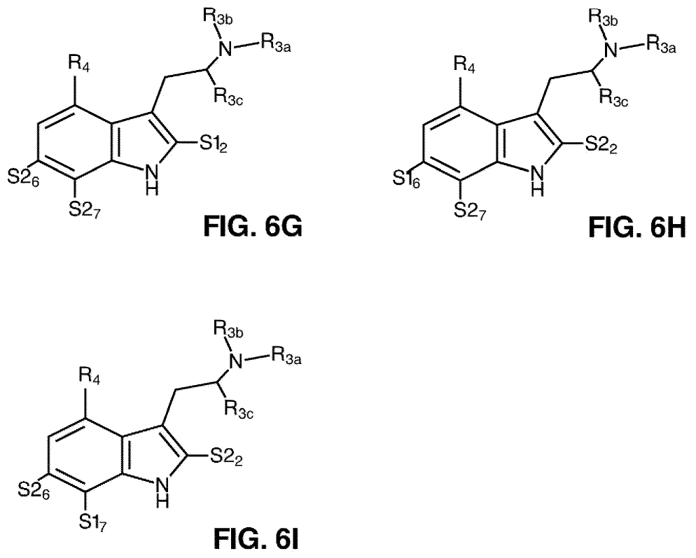

In accordance herewith disclosed herein, in an aspect, multiple-substituent psilocybin derivatives including two, or three substituent groups. Examples of each of these will next be discussed, by referring to selected figures. In particular, examples including multiple-substituent psilocybin derivatives including two substituent groups are discussed by referring to FIGS. 3A-3L; and examples of multiple-substituent psilocybins including three substituent groups are discussed by referring to FIGS. 4A-4I, 5A-5I, 6A-6I and 7A-7I. For clarity, it is noted that in each of these figures, notations such as S15, S25, and S17 for example, indicate, respectively, a first substituent S1 at the 5-position (C₅), a second substituent S2 at the 5-position (C₅), and a first substituent S1 at the 7-position (C₇). Similarly, in certain figures, notation within the same chemical compound, such as S2₂ and S2₆, for example, denote a second substituent S2 at the 2-position (C₂) and that same second substituent at the 6 position (C₆) of the compound (see: e.g., FIG. 6I).

Thus, referring next to FIGS. 3A-3L, examples of multiple-substituent psilocybin derivatives in accordance herewith, wherein two of R₂, R₅, R₆, or R₇ are substituents, are: the 2,5-di-S1,S2-psilocybin derivative depicted in FIG. 3A, the 2,5-di-S2,S1-psilocybin derivative depicted in FIG. 3B, the 2,6-di-S1,S2-psilocybin derivative depicted in FIG. 3C, the 2,6-di-S2,S1-psilocybin derivative depicted in FIG. 3D, the 2,7-di-S1,S2-psilocybin derivative depicted in FIG. 3E, the 2,7-di-S2,S1-psilocybin derivative depicted in FIG. 3F, the 5,6-di-S1,S2-psilocybin derivative depicted in FIG. 3G, the 5,6-di-S2,S1-psilocybin derivative depicted in FIG. 3H, the 57-di-S1,S2-psilocybin derivative depicted in FIG. 3I, the 5,7-di-S2,S1-psilocybin derivative depicted in FIG. 3J, the 6,7-di-S1,S2-psilocybin derivative depicted in FIG. 3K, and the 6,7-di-S2,S1-psilocybin derivative depicted in FIG. 3L. As will be clear from the foregoing, in each of FIGS. 3A-3L, S1 and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Thus, for example, referring to the chemical compound having the formula (I), in one example embodiment, two of R₄, R₅, R₆, or R₇ can be substituents, one of which can be selected from (i) a halogen atom, (ii) a prenyl group, and (iii) a nitrile group, and one of R₄, R₅, R₆, or R₇ can be a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, provided however the two substituents are non-identical, and R₂ and the non-substituted R₄, R₅, R₆ and R₇ are hydrogen atoms. Thus, in such example embodiment, referring to FIGS. 3A-3L, S1 can be selected from (i) a halogen atom, (ii) a prenyl group, and (iii) a nitrile group, and S2 can be selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, or, conversely, S2 can be selected from (i) a halogen atom, (ii) a prenyl group, and (iii) a nitrile group, and S1 can be selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Thus, for example, referring to the chemical compound having the formula (I), in a further example embodiment, two of R₄, R₅, R₆, or R₇ can be substituents, one of which is a halogen atom, and one of R₄, R₅, R₆, or R₇ can be a substituent selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a nitrile group, and R₂ and the non-substituted R₄, R₅, R₆ and R₇ are hydrogen atoms. Thus, in such example embodiment, referring to FIGS. 3A-3L, S1 can be a halogen atom, and S2 can be selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a nitrile group, or, conversely, S2 can be a halogen atom, and S1 can be (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a nitrile group.

Thus, for example, referring to the chemical compound having the formula (I), in a further example embodiment, two of R₄, R₅, R₆, or R₇ can be substituents, one of which is a halogen atom, and one of R₄, R₅, R₆, or R₇ can be a substituent selected from (i) an amino group, (ii) a nitrile group, (iii) a nitro group, (iv) a hydroxy group, and a (vii) a prenyl group, and R₂ and the non-substituted R₄, R₅, R₆ and R₇ are hydrogen atoms. Thus, in such example embodiment, referring to FIGS. 3A-3L, S1 can be a halogen atom, and S2 can be selected from (i) an amino group, (ii) a nitrile group, (iii) a nitro group, (iv) a hydroxy group, and a (vii) a prenyl group, or, conversely, S2 can be a halogen atom, and S1 can be (i) an amino group, (ii) a nitrile group, (iii) a nitro group, (iv) a hydroxy group, and a (v) a prenyl group.

Thus, for example, referring to the chemical compound having the formula (I), in a further example embodiment, two of R₄, R₅, R₆, or R₇ can be substituents, one of which is a halogen atom, and one of R₄, R₅, R₆, or R₇ can be a prenyl group, and R₂ and the non-substituted R₄, R₅, R₆ and R₇ are hydrogen atoms. Thus, in such example embodiment, referring to FIGS. 3A-3L, S1 can be a halogen atom, and S2 can be a prenyl group, or, conversely, S2 can be a prenyl group, and S1 can be a halogen atom.

Thus, for example, referring to the chemical compound having the formula (I), in a further example embodiment, two of R₄, R₅, R₆, or R₇ can be substituents, one of which is a prenyl group, and one of R₄, R₅, R₆, or R₇ can be a substituent selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a halogen atom, and (viii) a nitrile group, and R₂ and the non-substituted R₄, R₅, R₆ and R₇ are hydrogen atoms. Thus, in such example embodiment, referring to FIGS. 3A-3L, S1 can be a prenyl group, and S2 can be selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a halogen atom, and (viii) a nitrile group, or, conversely, S2 can be a prenyl group, and S1 can be (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a halogen atom, and (viii) a nitrile group.

Thus, for example, referring to the chemical compound having the formula (I), in a further example embodiment, two of R₄, R₅, R₆, or R₇ can be substituents, one of which is a prenyl group, and one of R₄, R₅, R₆, or R₇ can be a substituent selected from (i) a carboxyl group or a carboxylic acid derivative, (ii) a halogen atom, and (iii) a hydroxy group, and R₂ and the non-substituted R₄, R₅, R₆ and R₇ are hydrogen atoms. Thus, in such example embodiment, referring to FIGS. 3A-3L, S1 can be a prenyl group, and S2 can be selected from (i) a carboxyl group or a carboxylic acid derivative, (ii) a halogen atom, and (iii) a hydroxy group, or, conversely, S2 can be a prenyl group, and S1 can be (i) a carboxyl group or a carboxylic acid derivative, (ii) a halogen atom, and (iii) a hydroxy group.

Thus, for example, referring to the chemical compound having the formula (I), in a further example embodiment, two of R₄, R₅, R₆, or R₇ can be substituents, one of which is a nitrile group, and one of R₄, R₅, R₆, or R₇ can be a substituent selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a halogen atom, and R₂ and the non-substituted R₄, R₅, R₆ and R₇ are hydrogen atoms. Thus, in such example embodiment, referring to FIGS. 3A-3L, S1 can be a nitril group, and S2 can be selected from (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a halogen atom, or, conversely, S2 can be a nitrile group, and S1 can be (i) a hydroxy group, (ii) a nitro group, (iii) a glycosyloxy group, (iv) an amino group or an N-substituted amino group, (v) a carboxyl group or a carboxylic acid derivative, (vi) an aldehyde or a ketone group, (vii) a prenyl group, and (viii) a halogen atom.

Thus, for example, referring to the chemical compound having the formula (I), in a further example embodiment, two of R₄, R₅, R₆, or R₇ can be substituents, one of which is a nitrile group, and one of R₄, R₅, R₆, or R₇ can be an amino group or an N-substituted amino group, and R₂ and the non-substituted R₄, R₅, R₆ and R₇ are hydrogen atoms. Thus, in such example embodiment, referring to FIGS. 3A-3L, S1 can be a nitrile group, and S2 can be an amino group or an N-substituted amino group, conversely, S2 can be a nitrile group, and S1 can be an amino group or an N-substituted amino group.

Turning now to multiple-substituent psilocybin derivatives, including three substituent groups, and referring to FIGS. 4A-4F, 4G-4I, 5A-5F, 5G-5I, 6A-6F, 6G-6I, 7A-7F, 7G-7I, shown therein are examples of multiple-substituent psilocybin derivatives in accordance herewith, wherein three of R₂, R₅, R₆, or R₇ are substituents. It is noted that in the examples shown in FIGS. 4A-4F, 5A-5F, 6A-6F, 6G-6I, and 7A-7F, the three substituents are three different substituents S1, S2, S3. In the examples shown in FIGS. 4G-4I, 5G-5I, 6G-6I, and 7G-7I, the three substituents are selected such that two of the three substituents are the same, i.e., S1, S2, S2. Examples, in this respect, are in particular: the 2,5,6-tri-S1,S2,S3-psilocybin derivative shown in FIG. 4A, the 2,5,6-tri-S1,S3,S2-psilocybin derivative shown in FIG. 4B, the 2,5,6-tri-S3,S1,S2-psilocybin derivative shown in FIG. 4C, the 2,5,6-tri-S2,S1,S3-psilocybin derivative shown in FIG. 4D, the 2,5,6-tri-S3,S2,S1-psilocybin derivative shown in FIG. 4E, and the 2,5,6-tri-S2,83,S1-psilocybin derivative (FIG. 4F). As will be clear from the foregoing, in each of FIGS. 4A-4F S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Further examples, wherein three of R₂, R₅, R₆, or R₇ are substituents are shown in FIGS. 4G, 4H, and 4I, in particular the 2,5,6-tri-S1,S2,S2-psilocybin derivative shown in FIG. 4G, the 2,5,6-tri-S2,S1,S2-psilocybin derivative shown in FIG. 4H, and the 2,5,6-tri-S2,S2,S1-psilocybin derivative shown FIG. 4I. As will be clear from the foregoing, in each of FIGS. 4G-4I S1 and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

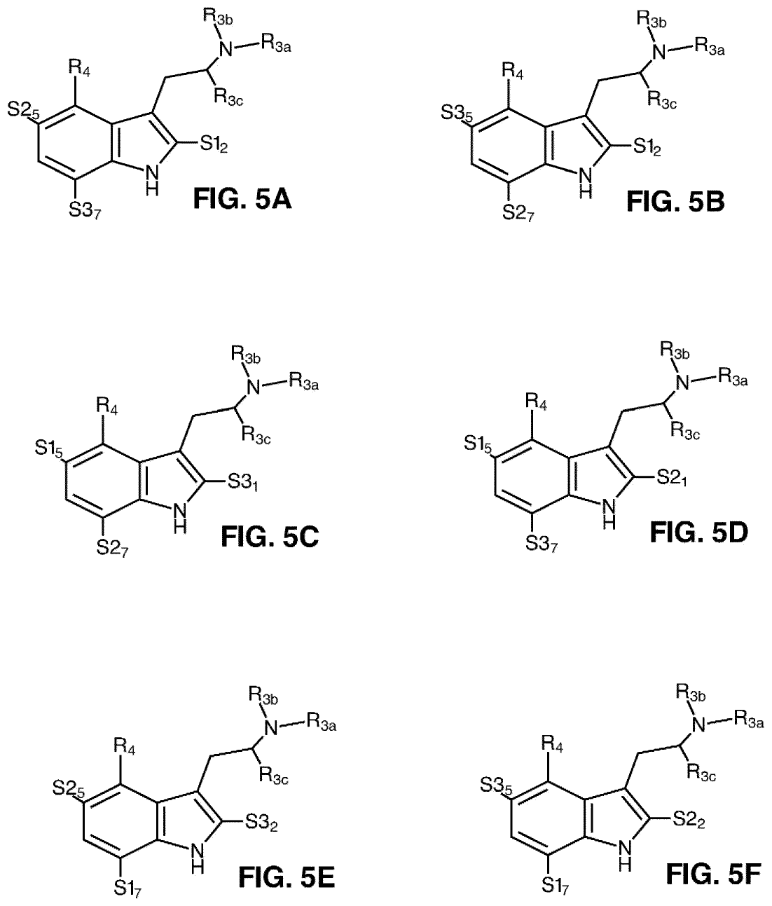

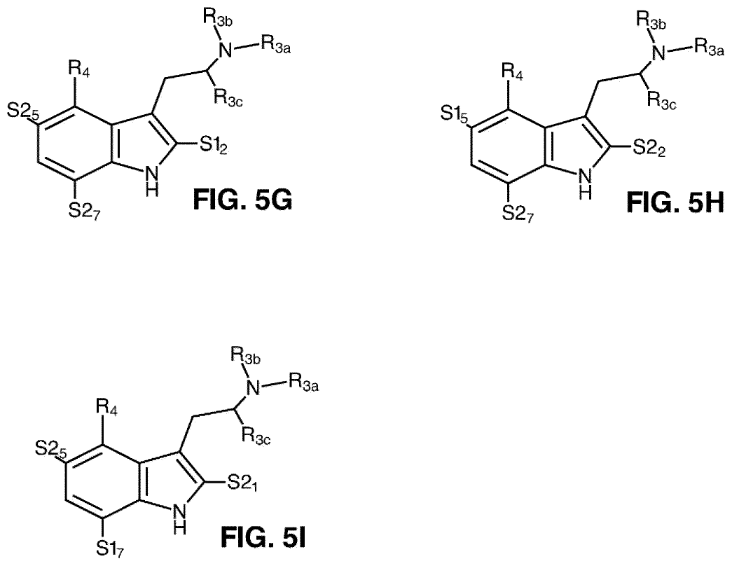

Further examples, wherein three of R₂, R₅, R₆, or R₇ are substituents are shown in FIGS. 5A, 5B, 5C, 5D, 5E, and 5F, in particular the 2,5,7-tri-S1,S2,S3-psilocybin derivative shown in FIG. 5A), the 2,5,7-tri-S1,S3,S2-psilocybin derivative shown in FIG. 5B, the 2,5,7-tri-S3,S1,82-psilocybin derivative shown in FIG. 5C, the 2,5,7-tri-S2,S1,S3-psilocybin derivative shown in FIG. 5D, the 2,5,7-tri-S3,S2,S1-psilocybin derivative shown in FIG. 5E, and the 2,5,7-tri-S2,S3,S1-psilocybin derivative shown in FIG. 5F. As will be clear from the foregoing in each of FIGS. 5A-5I S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Further examples, wherein three of R₂, R₅, R₆, or R₇ are substituents are shown in FIGS. 5G, 5H, and 5I, in particular the 2,5,7-tri-S1,S2,S2-psilocybin derivative shown in FIG. 5G, the 2,5,7-tri-S2,S1,82-psilocybin derivative shown in FIG. 5H), and the 2,5,7-tri-S2,S2,S1-psilocybin derivative shown in FIG. 5I. As will be clear from the foregoing in each of FIGS. 5G-5I, S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Further examples, wherein three of R₂, R₅, R₆, or R₇ are substituents are shown in FIGS. 6A, 6B, 6C, 6D, 6E, and 6F, in particular, the 2,6,7-tri-S1, S2, S3-psilocybin derivative shown in FIG. 6A, the 2,6,7-tri-S1,S3,S2-psilocybin derivative shown in FIG. 6B, the 2,6,7-tri-S3,S1,S2-psilocybin derivative shown in FIG. 6C, the 2,6,7-tri-S2,S1,S3-psilocybin derivative shown in FIG. 6D, the 2,6,7-tri-S3,S2,S1-psilocybin derivative shown in FIG. 6E, and the 2,6,7-tri-S2,S3,S1-psilocybin derivative (FIG. 6F). It will be clear from the foregoing, that in FIGS. 6A-6F, S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Further examples, wherein three of R₂, R₅, R₆, or R₇ are substituents are shown in FIGS. 6G, 6H, and 6I, in particular the 2,6,7-tri-S1,S2,S2-psilocybin derivative shown in FIG. 6G, the 2,6,7-tri-S2,S1,S2-psilocybin derivative shown in FIG. 6H, and the 2,6,7-tri-S2,S2,S1-psilocybin derivative shown in FIG. 6I. It will be clear from the foregoing that in FIGS. 6A-6I, S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix a nitrile group.

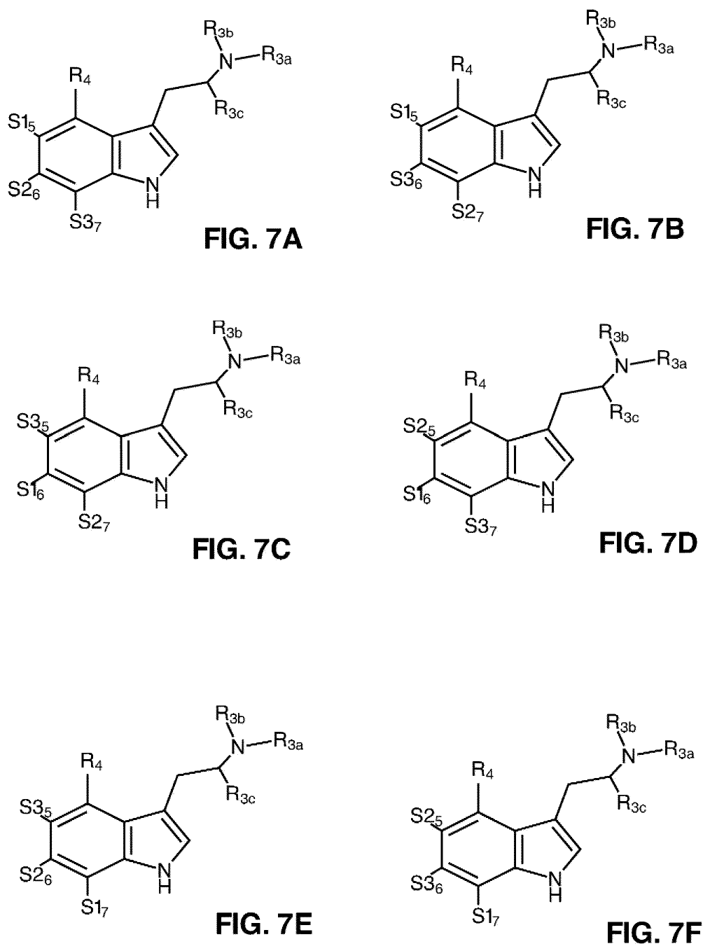

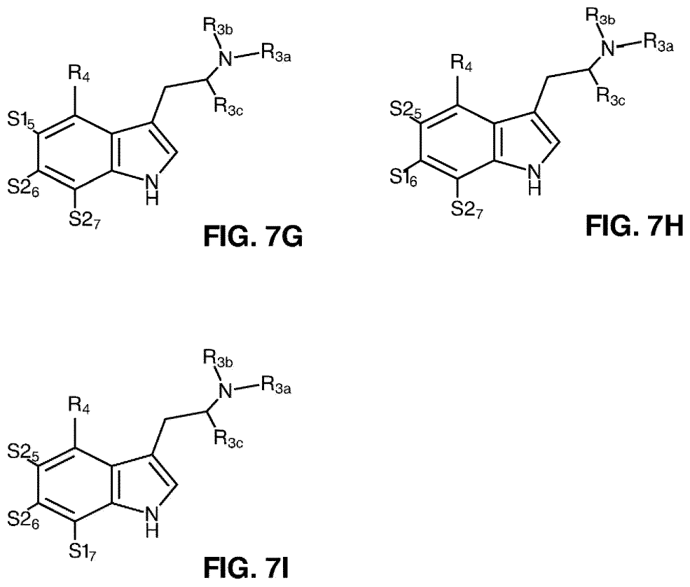

Further examples, wherein three of R₂, R₅, R₆, or R₇ are substituents are shown in FIGS. 7A, 7B, 7C, 7D, 7E, and 7F, in particular the 5,6,7-tri-S1, 2,S3-psilocybin derivative shown in FIG. 7A, the 5,6,7-tri-S1 S3,S2-psilocybin derivative shown in FIG. 7B, the 5,6,7-tri-S3,S1,S2-psilocybin derivative shown in FIG. 7C, the 5,6,7-tri-S2,S1,S3-psilocybin derivative shown in FIG. 7D, the 5,6,7-tri-S3,S2,S1-psilocybin derivative shown in FIG. 7E, and the 5,6,7-tri-S2,S3,S1-psilocybin derivative shown FIG. 7F. It will be clear from the foregoing that in FIGS. 7A-7F, S1, S2 and S3 are selected from three of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Yet further examples, wherein three of R₂, R₅, R₆, or R₇ are substituents are shown in FIGS. 7G, 7H, and 7I in particular the 5,6,7-tri-S1,S2,S2-psilocybin derivative shown in FIG. 7G, the 5,6,7-tri-S2,S1,S2-psilocybin derivative shown in FIG. 7H, and the 5,6,7-tri-S2,S2,S1-psilocybin derivative shown in FIG. 7I. It will be clear from the foregoing that in each of FIGS. 7G-7I, S1, and S2 are selected from two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group.

Furthermore, in each of the example embodiments shown in FIGS. 3A-3L, 4A-4I, 5A-5I, 6A-6I, and 7A-7I it is noted that R₃ₐ and R_{3b} can be a hydrogen atom, an alkyl group, an acyl group, or an aryl group. Thus, R₃ₐ and R_{3b} can each be a hydrogen atom, or R₃ₐ and R_{3b} can each be an alkyl group, such as a methyl group, ethyl group, propyl group, or longer chain alkyl group, or R₃ₐ and R_{3b} can be each be an acyl group, or R₃ₐ and R_{3b} can each be an aryl group. Furthermore, one of R₃ₐ and R_{3b} can be a hydrogen atom, and one of R₃ₐ and R_{3b} can be an alkyl group. One of R_{3A} and R_{3B} can be a hydrogen atom, and one of R₃ₐ and R_{3b} can be an acyl group. One of R₃ₐ and R_{3b} can be a hydrogen atom, and one of R₃ₐ and R_{3b} can be an aryl group. One of R₃ₐ and R_{3b} can be an alkyl group, and one of R_{3A} and R_{3B} can be an aryl group. One of R₃ₐ and R_{3b} can be an alkyl group, and one of R₃ₐ and R_{3b} can be an acyl group. One of R₃ₐ and R_{3b} can be an acyl group, and one of R₃ₐ and R_{3b} can be an aryl group.

It is noted that in a further aspect hereof in each of the example embodiments shown in FIGS. 3A-3L, 4A-4I, 5A-5I, 6A-6I, and 7A-7I, R_{3c} can be a hydrogen atom or a carboxy group.

It is noted that in a further aspect hereof in each of the example embodiments shown in FIGS. 3A-3L, 4A-4I, 5A-5I, 6A-6I, and 7A-7I, R₄ can be a hydrogen atom, an O-alkyl group, an O-acyl group, or a phosphate group.

Furthermore, in one example embodiment, a multi-substituent psilocybin derivative according to the present disclosure can be a chemical compound having a formula (IX):

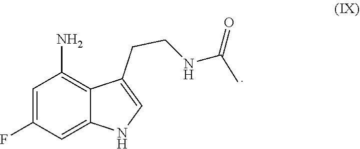 [SMILES: CC(=O)NCCc1c[nH]c2cc(F)cc(N)c12]

Furthermore, in one example embodiment, a multi-substituent psilocybin derivative according to the present disclosure can be a chemical compound having a formula (X):

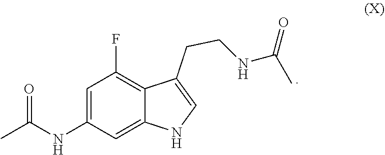 [SMILES: CC(=O)NCCc1c[nH]c2cc(NC(C)=O)cc(F)c12]

Furthermore, in one example embodiment, a multi-substituent psilocybin derivative according to the present disclosure can be a chemical compound having a formula (XI):

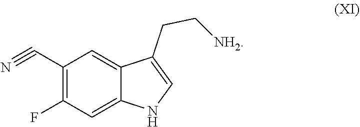 [SMILES: N#Cc1cc2c(CCN)c[nH]c2cc1F]

Furthermore, in one example embodiment, a multi-substituent psilocybin derivative according to the present disclosure can be a chemical compound having a formula (XII):

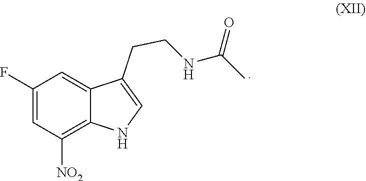 [SMILES: CC(=O)NCCc1c[nH]c2c([N+](=O)[O-])cc(F)cc12]

Furthermore, in one example embodiment, a multi-substituent psilocybin derivative according to the present disclosure can be a chemical compound having a formula (XIII):

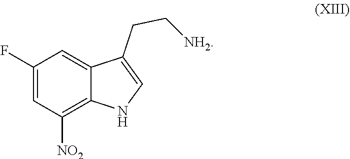 [SMILES: NCCc1c[nH]c2c([N+](=O)[O-])cc(F)cc12]

Furthermore, in one example embodiment, a multi-substituent psilocybin derivative according to the present disclosure can be a chemical compound having a formula (XIV):

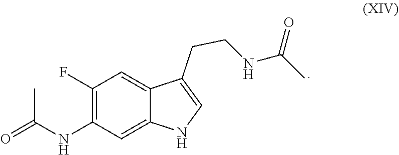 [SMILES: CC(=O)NCCc1c[nH]c2cc(NC(C)=O)c(F)cc12]

Furthermore, in one example embodiment, a multi-substituent psilocybin derivative according to the present disclosure can be a chemical compound having a formula (XV):

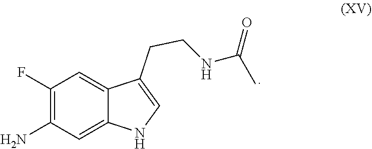 [SMILES: CC(=O)NCCc1c[nH]c2cc(N)c(F)cc12]

Furthermore, in one example embodiment, a multi-substituent psilocybin derivative according to the present disclosure can be a chemical compound having a formula (XV):

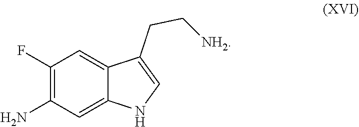 [SMILES: NCCc1c[nH]c2cc(N)c(F)cc12]

Furthermore, in one example embodiment, a multi-substituent psilocybin derivative according to the present disclosure can be a chemical compound having a formula (XVII):

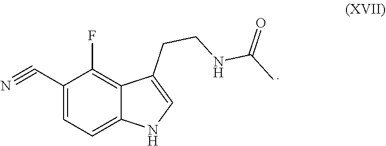 [SMILES: CC(=O)NCCc1c[nH]c2ccc(C#N)c(F)c12]

Furthermore, in one example embodiment, a multi-substituent psilocybin derivative according to the present disclosure can be a chemical compound having a formula (XVIII):

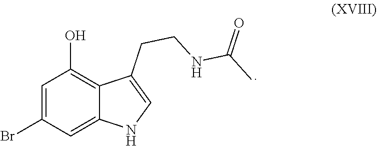 [SMILES: CC(=O)NCCc1c[nH]c2cc(Br)cc(O)c12]

Furthermore, in one example embodiment, a multi-substituent psilocybin derivative according to the present disclosure can be a chemical compound having a formula (XIX):

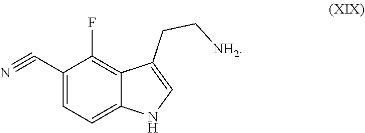 [SMILES: N#Cc1ccc2[nH]cc(CCN)c2c1F]

Furthermore, in one example embodiment, a multi-substituent psilocybin derivative according to the present disclosure can be a chemical compound having a formula (XX):

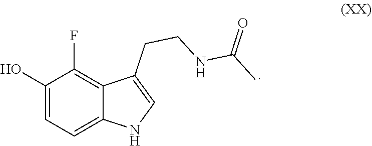 [SMILES: CC(=O)NCCc1c[nH]c2ccc(O)c(F)c12]

Furthermore, in one example embodiment, a multi-substituent psilocybin derivative according to the present disclosure can be a chemical compound having a formula (XXI):

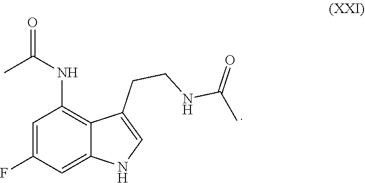 [SMILES: CC(=O)NCCc1c[nH]c2cc(F)cc(NC(C)=O)c12]

Furthermore, in one example embodiment, a multi-substituent psilocybin derivative according to the present disclosure can be a chemical compound having a formula (XXII):

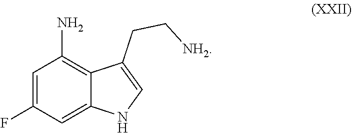 [SMILES: NCCc1c[nH]c2cc(F)cc(N)c12]

Furthermore, in one example embodiment, a multi-substituent psilocybin derivative according to the present disclosure can be a chemical compound having a formula (XXIII):

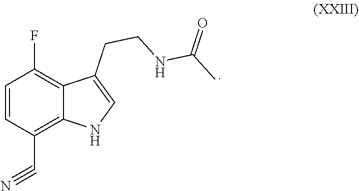 [SMILES: CC(=O)NCCc1c[nH]c2c(C#N)ccc(F)c12]

Furthermore, in one example embodiment, a multi-substituent psilocybin derivative according to the present disclosure can be a chemical compound having a formula (XXIV):

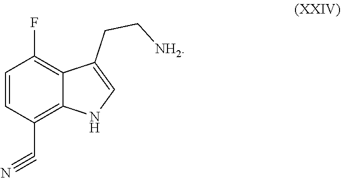 [SMILES: N#Cc1ccc(F)c2c(CCN)c[nH]c12]

Furthermore, in one example embodiment, a multi-substituent psilocybin derivative according to the present disclosure can be a chemical compound having a formula (XXV):

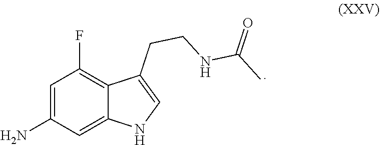 [SMILES: CC(=O)NCCc1c[nH]c2cc(N)cc(F)c12]

Furthermore, in one example embodiment, a multi-substituent psilocybin derivative according to the present disclosure can be a chemical compound having a formula (XVI):

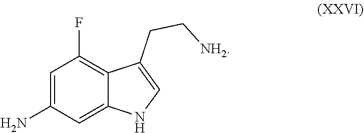 [SMILES: NCCc1c[nH]c2cc(N)cc(F)c12]

Furthermore, in one example embodiment, a multi-substituent psilocybin derivative according to the present disclosure can be a chemical compound having a formula (XXVII):

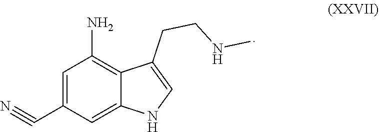 [SMILES: CNCCc1c[nH]c2cc(C#N)cc(N)c12]

Furthermore, in one example embodiment, a multi-substituent psilocybin derivative according to the present disclosure can be a chemical compound having a formula (XXVIII):

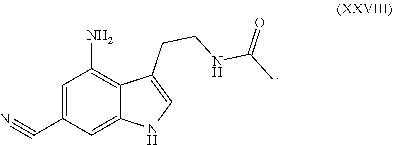 [SMILES: CC(=O)NCCc1c[nH]c2cc(C#N)cc(N)c12]

Furthermore, in one example embodiment, a multi-substituent psilocybin derivative according to the present disclosure can be a chemical compound having a formula (XXIX):

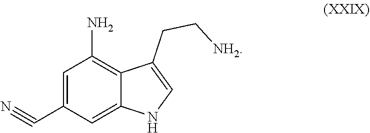 [SMILES: N#Cc1cc(N)c2c(CCN)c[nH]c2c1]

Furthermore, in one example embodiment, a multi-substituent psilocybin derivative according to the present disclosure can be a chemical compound having a formula (XXX):

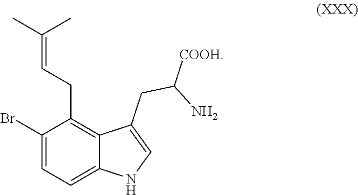 [SMILES: CC(C)=CCc1c(Br)ccc2[nH]cc(CC(N)C(=O)O)c12]

Furthermore, in one example embodiment, a multi-substituent psilocybin derivative according to the present disclosure can be a chemical compound having a formula (XXXI):

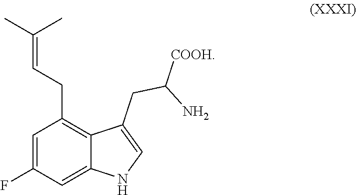 [SMILES: CC(C)=CCc1cc(F)cc2[nH]cc(CC(N)C(=O)O)c12]

Furthermore, in one example embodiment, a multi-substituent psilocybin derivative according to the present disclosure can be a chemical compound having a formula (XXXII):

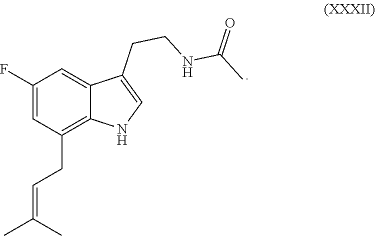 [SMILES: CC(=O)NCCc1c[nH]c2c(CC=C(C)C)cc(F)cc12]

Furthermore, in one example embodiment, a multi-substituent psilocybin derivative according to the present disclosure can be a chemical compound having a formula (XXXIII):

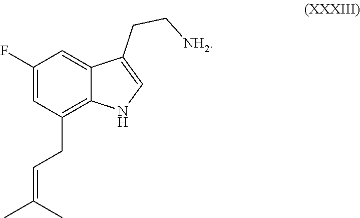 [SMILES: CC(C)=CCc1cc(F)cc2c(CCN)c[nH]c12]

Furthermore, in one example embodiment, a multi-substituent psilocybin derivative according to the present disclosure can be a chemical compound having a formula (XXXIV):

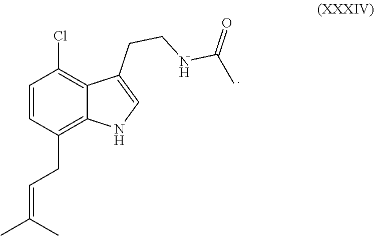 [SMILES: CC(=O)NCCc1c[nH]c2c(CC=C(C)C)ccc(Cl)c12]

Furthermore, in one example embodiment, a multi-substituent psilocybin derivative according to the present disclosure can be a chemical compound having a formula (XXXV):

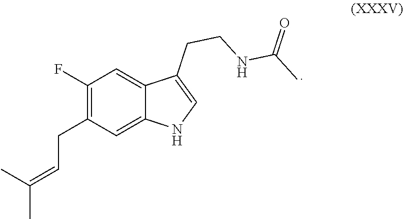 [SMILES: CC(=O)NCCc1c[nH]c2cc(CC=C(C)C)c(F)cc12]

Furthermore, in one example embodiment, a multi-substituent psilocybin derivative according to the present disclosure can be a chemical compound having a formula (XXXVI):

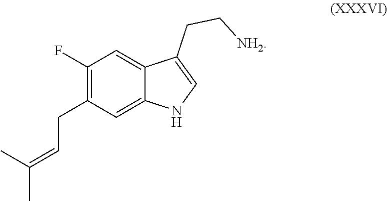 [SMILES: CC(C)=CCc1cc2[nH]cc(CCN)c2cc1F]

Furthermore, in one example embodiment, a multi-substituent psilocybin derivative according to the present disclosure can be a chemical compound having a formula (XXXVII):

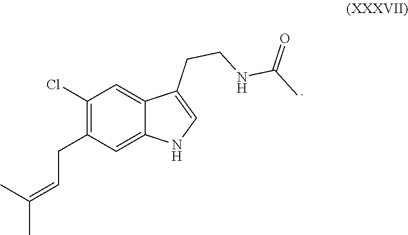 [SMILES: CC(=O)NCCc1c[nH]c2cc(CC=C(C)C)c(Cl)cc12]

Furthermore, in one example embodiment, a multi-substituent psilocybin derivative according to the present disclosure can be a chemical compound having a formula (XXXVIII):

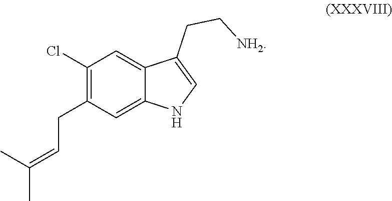 [SMILES: CC(C)=CCc1cc2[nH]cc(CCN)c2cc1Cl]

Furthermore, in one example embodiment, a multi-substituent psilocybin derivative according to the present disclosure can be a chemical compound having a formula (XXXIX):

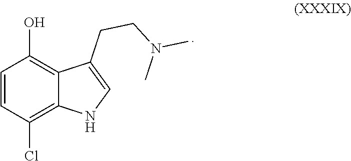 [SMILES: CN(C)CCc1c[nH]c2c(Cl)ccc(O)c12]

Furthermore, in one example embodiment, a multi-substituent psilocybin derivative according to the present disclosure can be a chemical compound having a formula (XL):

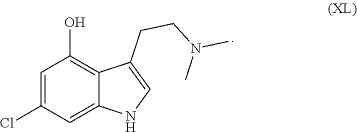 [SMILES: CN(C)CCc1c[nH]c2cc(Cl)cc(O)c12]

Furthermore, in one example embodiment, a multi-substituent psilocybin derivative according to the present disclosure can be a chemical compound having a formula (XLI):

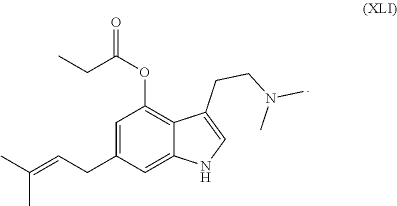 [SMILES: CCC(=O)Oc1cc(CC=C(C)C)cc2[nH]cc(CCN(C)C)c12]

Furthermore, in one example embodiment, a multi-substituent psilocybin derivative according to the present disclosure can be a chemical compound having a formula (XLII):

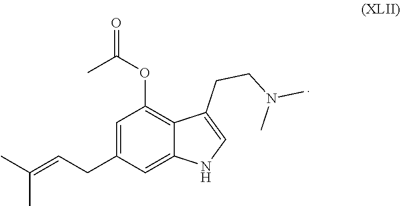 [SMILES: CC(=O)Oc1cc(CC=C(C)C)cc2[nH]cc(CCN(C)C)c12]

Furthermore, in one example embodiment, a multi-substituent psilocybin derivative according to the present disclosure can be a chemical compound having a formula (XLIII):

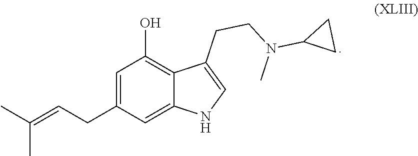 [SMILES: CC(C)=CCc1cc(O)c2c(CCN(C)C3CC3)c[nH]c2c1]

Furthermore, in one example embodiment, a multi-substituent psilocybin derivative according to the present disclosure can be a chemical compound having a formula (XLIV):

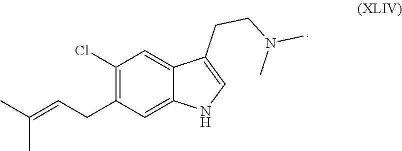 [SMILES: CC(C)=CCc1cc2[nH]cc(CCN(C)C)c2cc1Cl]

Furthermore, in one example embodiment, a multi-substituent psilocybin derivative according to the present disclosure can be a chemical compound having a formula (XLV):

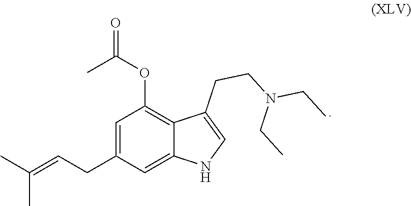 [SMILES: CCN(CC)CCc1c[nH]c2cc(CC=C(C)C)cc(OC(C)=O)c12]

Furthermore, in one example embodiment, a multi-substituent psilocybin derivative according to the present disclosure can be a chemical compound having a formula (XLVI):

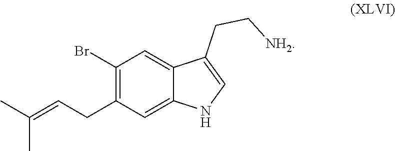 [SMILES: CC(C)=CCc1cc2[nH]cc(CCN)c2cc1Br]

Furthermore, in one example embodiment, a multi-substituent psilocybin derivative according to the present disclosure can be a chemical compound having a formula (XLVII):

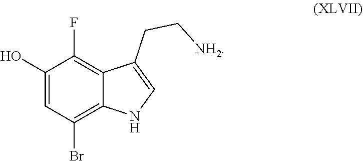 [SMILES: NCCc1c[nH]c2c(Br)cc(O)c(F)c12]

Furthermore, in one example embodiment, a multi-substituent psilocybin derivative according to the present disclosure can be a chemical compound having a formula (XLVIII):

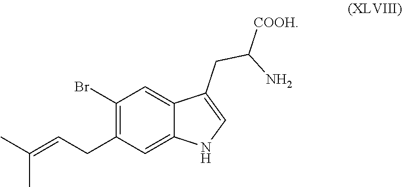 [SMILES: CC(C)=CCc1cc2[nH]cc(CC(N)C(=O)O)c2cc1Br]

Furthermore, in one example embodiment, a multi-substituent psilocybin derivative according to the present disclosure can be a chemical compound having a formula (XLIX):

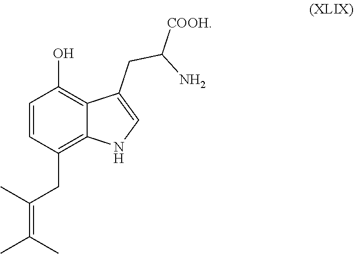 [SMILES: CC(C)=C(C)Cc1ccc(O)c2c(CC(N)C(=O)O)c[nH]c12]

Furthermore, in one example embodiment, a multi-substituent psilocybin derivative according to the present disclosure can be a chemical compound having a formula (L):

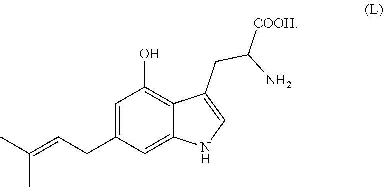 [SMILES: CC(C)=CCc1cc(O)c2c(CC(N)C(=O)O)c[nH]c2c1]

Furthermore, in one example embodiment, a multi-substituent psilocybin derivative according to the present disclosure can be a chemical compound having a formula (LI):

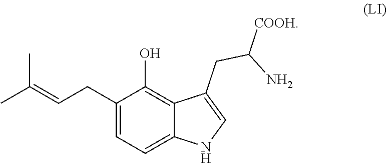 [SMILES: CC(C)=CCc1ccc2[nH]cc(CC(N)C(=O)O)c2c1O]

Furthermore, in one example embodiment, a multi-substituent psilocybin derivative according to the present disclosure can be a chemical compound having a formula (LII):

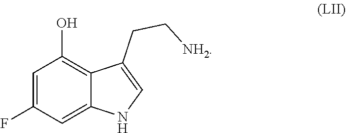 [SMILES: NCCc1c[nH]c2cc(F)cc(O)c12]

Furthermore, in one example embodiment, a multi-substituent psilocybin derivative according to the present disclosure can be a chemical compound having a formula (LIII):

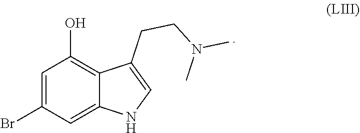 [SMILES: CN(C)CCc1c[nH]c2cc(Br)cc(O)c12]

Furthermore, in one example embodiment, a multi-substituent psilocybin derivative according to the present disclosure can be a chemical compound having a formula (LIV):

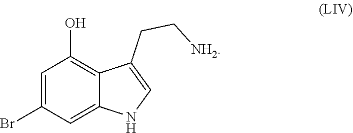 [SMILES: NCCc1c[nH]c2cc(Br)cc(O)c12]

Furthermore, in one example embodiment, a multi-substituent psilocybin derivative according to the present disclosure can be a chemical compound having a formula (LV):

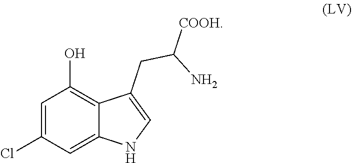 [SMILES: NC(Cc1c[nH]c2cc(Cl)cc(O)c12)C(=O)O]

Furthermore, in one example embodiment, a multi-substituent psilocybin derivative according to the present disclosure can be a chemical compound having a formula (LXXVI):

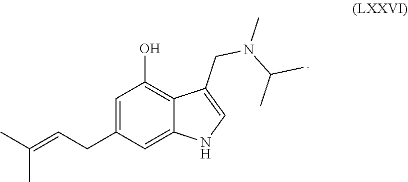 [SMILES: CC(C)=CCc1cc(O)c2c(CN(C)C(C)C)c[nH]c2c1]

Furthermore, it is noted that the multi-substituent psilocybin derivatives of the present disclosure include salts thereof, including pharmaceutically acceptable salts. Thus, the nitrogen atom of the ethyl-amino group extending in turn from the C₃ atom may be protonated, and the positive charge may be balanced by, for example, chloride or sulfate ions, to thereby form a chloride salt or a sulfate salt. Furthermore, in compounds wherein R₄ is a phosphate group, the phosphate group may be de-protonated, and the negative charge may be balanced by, for example, sodium ions or potassium ions, to thereby form a sodium salt or a potassium salt.

Furthermore, it is noted that when R₄ is a phosphate group, the term prenylated psilocybin derivative also includes compounds having a formula (IV):

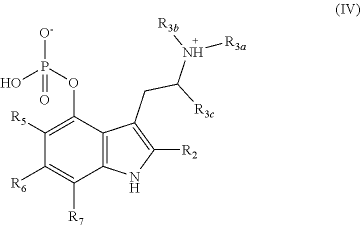

wherein, at least two of R₂, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom, wherein R₃ₐ and R_{3b} are each independently a hydrogen atom, an alkyl group, a cycloalkyl group, an acyl group, or an aryl group, and wherein and R_{3c} is a hydrogen atom or a carboxyl group. When R_{3c} is a carboxy group, further included are compounds having a formula (Iva):

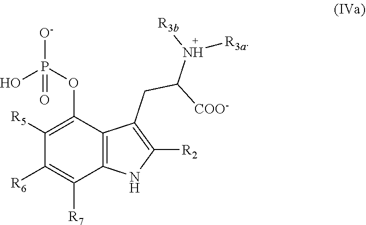

Further included are salts of prenylated psilocybin derivatives having a formula (IV) and (IVa), such as a sodium salt, a potassium salt, etc.

Thus, to briefly recap, the present disclosure provides multi-substituent psilocybin derivatives. The disclosure provides, in particular, a chemical compound having a formula (I):

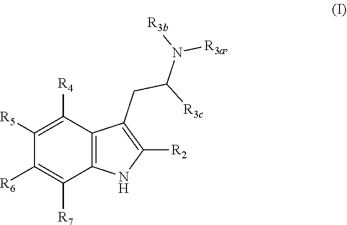

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

In another embodiment, R₃ₐ and R_{3b} are a hydrogen atom, a (C₁-C₂₀)-alkyl group, a (C₆-C₁₄)-aryl group, or a —C(═O)(C₁-C₂₀)-alkyl group. In another embodiment, R₃ₐ and R_{3b} are a hydrogen atom, a (C₁-C₁₀)-alkyl group, a (C₆-C₁₀)-aryl group, or a —C(═O)(C₁-C₁₀)-alkyl group. In another embodiment, R₃ₐ and R_{3b} are a hydrogen atom, a (C₁-C₆)-alkyl group, a phenyl group, or a —C(═O)(C₁-C₆)-alkyl group. In another embodiment, R₃ₐ and R_{3b} are a hydrogen atom, a methyl group, an ethyl group, a propyl group, a phenyl group, —C(═O)—CH₃, —C(═O)—CH₂CH₃, or —C(═O)—CH₂CH₂CH₃.

In another embodiment, R₃ₐ and/or R_{3b} are a (C₁-C₂₀)-cyclo-alkyl group, or a (C₁-C₁₀)-cyclo-alkyl group, a (C₁-C₁₀)-cyclo-alkyl group, or a (C₁-C₁₀)-cyclo-alkyl group. In one embodiment, R₃ₐ and/or R_{3b} are a cyclo-propane group, a cyclo-butane group, a cyclo-pentane group, or a cyclo-hexane group.

In one embodiment, the alkyl groups (including O-alkyl) in any of the definitions of the formulas of the disclosure is C₁-C₂₀-alkyl. In another embodiment, the alkyl group is C₁-C₁₀-alkyl. In another embodiment, the alkyl group is C₁-C₆-alkyl. In another embodiment, the alkyl group is methyl, ethyl, propyl, butyl or pentyl.

In one embodiment, the acyl groups (including O-acyl) in any of the definitions of the formulas of the disclosure is C₁-C₂₀-acyl (or C₁-C₂₀-acyl-O—). In another embodiment, the alkyl group is C₁-C₁₀-acyl (or C₁-C₁₀-acyl-O—). In another embodiment, the alkyl group is C₁-C₆-acyl (or C₁-C₆-acyl-O—). In another embodiment, the acyl group is an O-acyl group, a methanoyl, ethanoyl, propanoyl, butanoyl or pentanoyl.

In one embodiment, the aryl groups in any of the definitions of the formulas of the disclosure is optionally substituted C₆-C₁₄-aryl. In another embodiment, the aryl group is optionally substituted C₆-C₁₀-aryl, or phenyl. In another embodiment, the aryl group is phenyl, naphthyl, tetrahydronaphthyl, phenanthrenyl, biphenylenyl, indanyl, or indenyl and the like.

The multi-substituent psilocybin derivatives of the present disclosure may be used to prepare a pharmaceutical or recreational drug formulation. Thus in one embodiment, the present disclosure further provides in another aspect, pharmaceutical and recreational drug formulations comprising multi-substituent psilocybin derivatives. Accordingly, in one aspect, the present disclosure provides in a further embodiment a pharmaceutical or recreational drug formulation comprising a chemical compound having a formula (I):

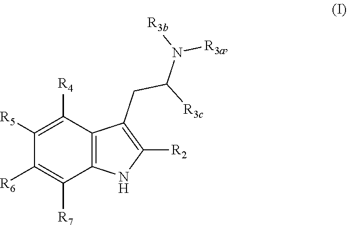

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

The pharmaceutical or recreational drug formulations may be prepared as liquids, tablets, capsules, microcapsules, nanocapsules, trans-dermal patches, gels, foams, oils, aerosols, nanoparticulates, powders, creams, emulsions, micellar systems, films, sprays, ovules, infusions, teas, decoctions, suppositories, etc. and include a pharmaceutically acceptable salt or solvate of the nitrilated psilocybin compound together with an excipient. The term “excipient” as used herein means any ingredient other than the chemical compound of the disclosure. As will readily be appreciated by those of skill in art, the selection of excipient may depend on factors such as the particular mode of administration, the effect of the excipient on solubility of the chemical compounds of the present disclosure and methods for their preparation will be readily apparent to those skilled in the art. Such compositions and methods for their preparation may be found, for example, in “Remington's Pharmaceutical Sciences”, 22^{nd} Edition (Pharmaceutical Press and Philadelphia College of Pharmacy at the University of the Sciences, 2012).

The dose when using the compounds of the present disclosure can vary within wide limits, and as is customary and is known to those of skill in the art, the dose can be tailored to the individual conditions in each individual case. The dose depends, for example, on the nature and severity of the illness to be treated, on the condition of the patient, on the compound employed or on whether an acute or chronic disease state is treated, or prophylaxis is conducted, on the mode of delivery of the compound, or on whether further active compounds are administered in addition to the compounds of the present disclosure. Representative doses of the present invention include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to about 100 mg, about 0.001 mg to about 50 mg, and about 0.001 mg to about 25 mg. Representative doses of the present disclosure include, but are not limited to, about 0.0001 to about 1,000 mg, about 10 to about 160 mg, about 10 mg, about 20 mg, about 40 mg, about 80 mg or about 160 mg. Multiple doses may be administered during the day, especially when relatively large amounts are deemed to be needed, for example 2, 3 or 4, doses. Depending on the subject and as deemed appropriate from the patient's physician or care giver it may be necessary to deviate upward or downward from the doses described herein.

The pharmaceutical and drug formulations comprising the multi-substituent psilocybin derivatives of the present disclosure may be administered orally. Oral administration may involve swallowing, so that the compound enters the gastrointestinal tract, or buccal or sublingual administration may be employed by which the compound enters the blood stream directly from the mouth. Formulations suitable for oral administration include both solid and liquid formulations.

Solid formulations include tablets, capsules (containing particulates, liquids, microcapsules, or powders), lozenges (including liquid-filled lozenges), chews, multi- and nano-particulates, gels, solid solutions, liposomal preparations, microencapsulated preparations, creams, films, ovules, suppositories, and sprays.

Liquid formulations include suspensions, solutions, syrups, and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose.

Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally comprise surface active agents, such as sodium lauryl sulfate and polysorbate 80. When present, surface active agents may comprise from 0.2% (w/w) to 5% (w/w) of the tablet.

Tablets may further contain lubricants such as magnesium stearate, calcium stearate, zinc stearate, sodium stearyl fumarate, and mixtures of magnesium stearate with sodium lauryl sulphate. Lubricants generally comprise from 0.25% (w/w) to 10% (w/w), from 0.5% (w/w) to 3% (w/w) of the tablet.

In addition to the multi-substituent psilocybin derivative, tablets may contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1% (w/w) to 25% (w/w) or from 5% (w/w) to 20% (w/w) of the dosage form.

Other possible auxiliary ingredients include anti-oxidants, colourants, flavouring agents, preservatives, and taste-masking agents.

For tablet dosage forms, depending on the desired effective amount of the chemical compound, the chemical compound of the present disclosure may make up from 1% (w/w) to 80% (w/w) of the dosage form, more typically from 5% (w/w) to 60% (w/w) of the dosage form.

Exemplary tablets contain up to about 80% (w/w) of the chemical compound, from about 10% (w/w) to about 90% (w/w) binder, from about 0% (w/w) to about 85% (w/w) diluent, from about 2% (w/w) to about 10% (w/w) disintegrant, and from about 0.25% (w/w) to about 10% (w/w) lubricant.

The formulation of tablets is discussed in “Pharmaceutical Dosage Forms: Tablets”, Vol. 1-Vol. 3, by CRC Press (2008).

The pharmaceutical and recreational drug formulations comprising the multi-substituent psilocybin derivatives of the present disclosure may also be administered directly into the blood stream, into muscle, or into an internal organ. Thus, the pharmaceutical and recreational drug formulations can be administered parenterally (for example, by subcutaneous, intravenous, intraarterial, intrathecal, intraventricular, intracranial, intramuscular, or intraperitoneal injection). Parenteral formulations are typically aqueous solutions which may contain excipients such as salts, carbohydrates and buffering agents (in one embodiment, to a pH of from 3 to 9), but, for some applications, they may be more suitably formulated as a sterile non-aqueous solution or as a dried form to be used in conjunction with a suitable vehicle such as sterile water.

Formulations comprising the multi-substituent psilocybin derivatives of the present disclosure for parenteral administration may be formulated to be immediate and/or modified release. Modified release formulations include delayed-, sustained-, pulsed-, controlled-, targeted and programmed release. Thus the chemical compounds of the disclosure may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The pharmaceutical or recreational drug formulations of the present disclosure also may be administered topically to the skin or mucosa, i.e., dermally or transdermally. Example pharmaceutical and recreational drug formulations for this purpose include gels, hydrogels, lotions, solutions, creams, ointments, dusting powders, cosmetics, oils, eye drops, dressings, foams, films, skin patches, wafers, implants, sponges, fibres, bandages and microemulsions. Liposomes may also be used. Example carriers include alcohol, water, mineral oil, liquid petrolatum, white petrolatum, glycerin, polyethylene glycol and propylene glycol. Penetration enhancers may be incorporate (see: for example, Finnin, B. and Morgan, T. M., 1999 J. Pharm. Sci, 88 (10), 955-958).

Other means of topical administration include delivery by electroporation, iontophoresis, phonophoresis, sonophoresis and microneedle or needle-free (e.g., Powderject™, Bioject™, etc.) injection.

Pharmaceutical and recreational drug formulations for inhalation or insufflation include solutions and suspensions in pharmaceutically acceptable, aqueous, or organic solvents, or mixtures thereof, and powders. The liquid or solid pharmaceutical compositions can contain suitable pharmaceutically acceptable excipients. In some embodiments, the pharmaceutical compositions are administered by the oral or nasal respiratory route for local or systemic effect. Pharmaceutical compositions in pharmaceutically acceptable solvents can be nebulized by use of inert gases. Nebulized solutions can be inhaled directly from the nebulizing device or the nebulizing device can be attached to a face mask tent, or intermittent positive pressure breathing machine. Solution, suspension, or powder pharmaceutical compositions can be administered, e.g., orally, or nasally, from devices that deliver the formulation in an appropriate manner.

In further embodiments, in which the multi-substituent psilocybin derivative compounds of present disclosure are used as a recreational drug, the compounds may be included in compositions such as a food or food product, a beverage, a food seasoning, a personal care product, such as a cosmetic, perfume or bath oil, or oils (both for topical administration as massage oil, or to be burned or aerosolized). The chemical compounds of the present disclosure may also be included in a “vape” product, which may also include other drugs, such as nicotine, and flavorings.

Thus it will be clear the multi-substituent psilocybin derivative compounds may be used as a pharmaceutical or recreational drug. Accordingly, in another aspect the present disclosure provides, in at least one embodiment, a use of a chemical compound having a formula (I):

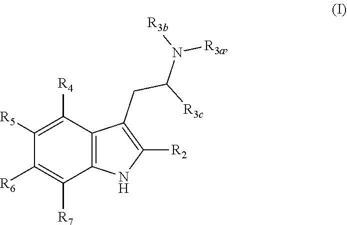

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

The pharmaceutical formulations comprising the chemical compounds of the present disclosure may be used to treat a subject, and in particular to treat a psychiatric disorder in a subject. Accordingly, the present disclosure includes in a further embodiment, a method for treating a psychiatric disorder, the method comprising administering to a subject in need thereof a pharmaceutical formulation comprising a chemical compound having a formula (I):

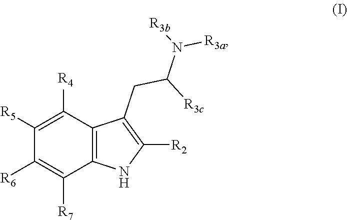

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

Psychiatric disorders that may be treated include, for example, neurodevelopmental disorders such as intellectual disability, global development delay, communication disorders, autism spectrum disorder, and attention-deficit hyperactivity disorder (ADHD); bipolar and related disorders, such as mania, and depressive episodes; anxiety disorder, such as generalized anxiety disorder (GAD), agoraphobia, social anxiety disorder, specific phobias (natural events, medical, animal, situational, for example), panic disorder, and separation anxiety disorder; stress disorders, such as acute stress disorder, adjustment disorders, post-traumatic stress disorder (PTSD), and reactive attachment disorder; dissociative disorders, such as dissociative amnesia, dissociative identity disorder, and depersonalization/derealization disorder; somatoform disorders, such as somatic symptom disorders, illness anxiety disorder, conversion disorder, and factitious disorder; eating disorders, such as anorexia nervosa, bulimia nervosa, rumination disorder, pica, and binge-eating disorder; sleep disorders, such as narcolepsy, insomnia disorder, hypersomnolence, breathing-related sleep disorders, parasomnias, and restless legs syndrome; disruptive disorders, such as kleptomania, pyromania, intermittent explosive disorder, conduct disorder, and oppositional defiant disorder; depressive disorders, such as disruptive mood dysregulation disorder, major depressive disorder, persistent depressive disorder (dysthymia), premenstrual dysphoric disorder, substance/medication-induced depressive disorder, postpartum depression, and depressive disorder caused by another medical condition, for example, psychiatric and existential distress within life-threatening cancer situations (ACS Pharmacol. Transl. Sci. 4: 553-562; J Psychiatr Res 137: 273-282); substance-related disorders, such as alcohol-related disorders, cannabis related disorders, inhalant-use related disorders, stimulant use disorders, and tobacco use disorders; neurocognitive disorders, such as delirium; schizophrenia; compulsive disorders, such as obsessive compulsive disorders (OCD), body dysmorphic disorder, hoarding disorder, trichotillomania disorder, excoriation disorder, substance/medication induced obsessive-compulsive disorder, and obsessive-compulsive disorder related to another medical condition; and personality disorders, such as antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder.

In an aspect, the compounds of the present disclosure may be used to be contacted with a 5-HT_{2A} receptor to thereby modulate the 5-HT_{2A} receptor. Such contacting includes bringing a compound of the present disclosure and 5-HT_{2A} receptor together under in vitro conditions, for example, by introducing the compounds in a sample containing a 5-HT_{2A} receptor, for example, a sample containing purified 5-HT_{2A} receptors, or a sample containing cells comprising 5-HT_{2A} receptors. In vitro conditions further include the conditions described in Example 1 hereof. Contacting further includes bringing a compound of the present disclosure and 5-HT_{2A} receptor together under in vivo conditions. Such in vivo conditions include the administration to an animal or human subject, for example, of a pharmaceutically effective amount of the compound of the present disclosure, when the compound is formulated together with a pharmaceutically active carrier, diluent, or excipient, as hereinbefore described, to thereby treat the subject. Upon having contacted the 5-HT_{2A} receptor, the compound may activate the 5-HT_{2A} receptor or inhibit the 5-HT_{2A} receptor.

Thus, in a further aspect, the condition that may be treated in accordance herewith can be any 5-HT_{2A} receptor mediated disorder. Such disorders include, but are not limited to schizophrenia, psychotic disorder, attention deficit hyperactivity disorder, autism, and bipolar disorder.

In an aspect, the compounds of the present disclosure may be used to be contacted with a 5-HT_{1A} receptor to thereby modulate the 5-HT_{1A} receptor. Such contacting includes bringing a compound of the present disclosure and 5-HT_{1A} receptor together under in vitro conditions, for example, by introducing the compounds in a sample containing a 5-HT_{1A} receptor, for example, a sample containing purified 5-HT_{1A} receptors, or a sample containing cells comprising 5-HT_{1A} receptors. In vitro conditions further include the conditions described in Example 1 hereof. Contacting further includes bringing a compound of the present disclosure and 5-HT_{1A} receptor together under in vivo conditions. Such in vivo conditions include the administration to an animal or human subject, for example, of a pharmaceutically effective amount of the compound of the present disclosure, when the compound is formulated together with a pharmaceutically active carrier, diluent, or excipient, as hereinbefore described, to thereby treat the subject. Upon having contacted the 5-HT_{1A} receptor, the compound may activate the 5-HT_{1A} receptor or inhibit the 5-HT_{1A} receptor.

Thus, in a further aspect, the condition that may be treated in accordance herewith can be any 5-HT_{1A} receptor mediated disorder. Such disorders include, but are not limited to schizophrenia, psychotic disorder, attention deficit hyperactivity disorder, autism, and bipolar disorder.

In some embodiments, upon having contacted a 5-HT_{1A} receptor and a 5-HT_{2A} receptor, the compound may modulate the 5-HT_{1A} receptor, e.g., activate or inhibit the 5-HT_{1A} receptor, however the compound may at the same time not modulate the 5-HT_{2A} receptor.

In some embodiments, upon having contacted a 5-HT_{2A} receptor and a 5-HT_{1A} receptor, the compound may modulate the 5-HT_{2A} receptor, e.g., activate or inhibit the 5-HT_{2A} receptor, however the compound may at the same time not modulate the 5-HT_{1A} receptor.

Turning now to methods of making the multi-substituent psilocybin derivatives of the present disclosure, it is initially noted that the multi-substituent psilocybin derivatives of the present disclosure may be prepared in any suitable manner, including by any organic chemical synthesis methods, biosynthetic methods, or a combination thereof. Next, initially example methods for chemically making the multi-substituent psilocybin derivatives of the present disclosure will be discussed. Thereafter, example biosynthetic methods for making the multi-substituent psilocybin derivatives will be discussed.

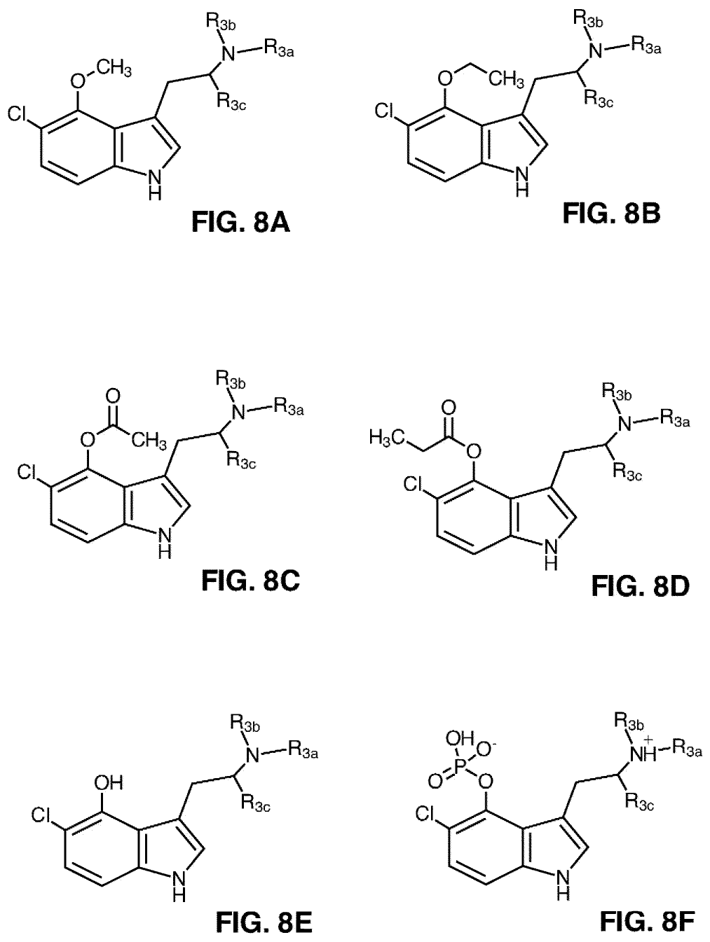

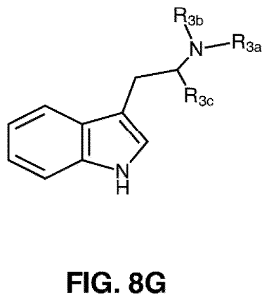

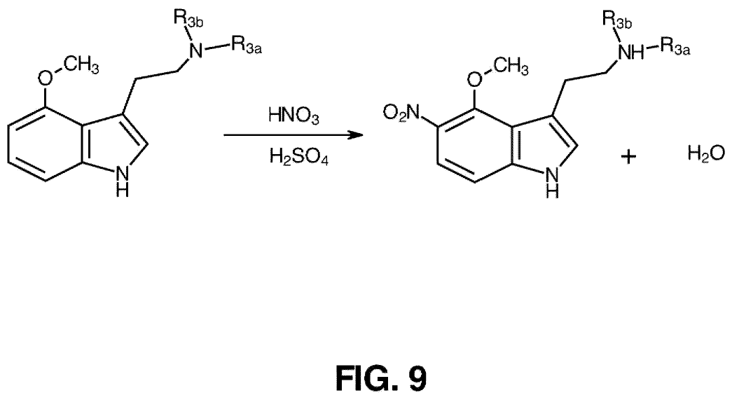

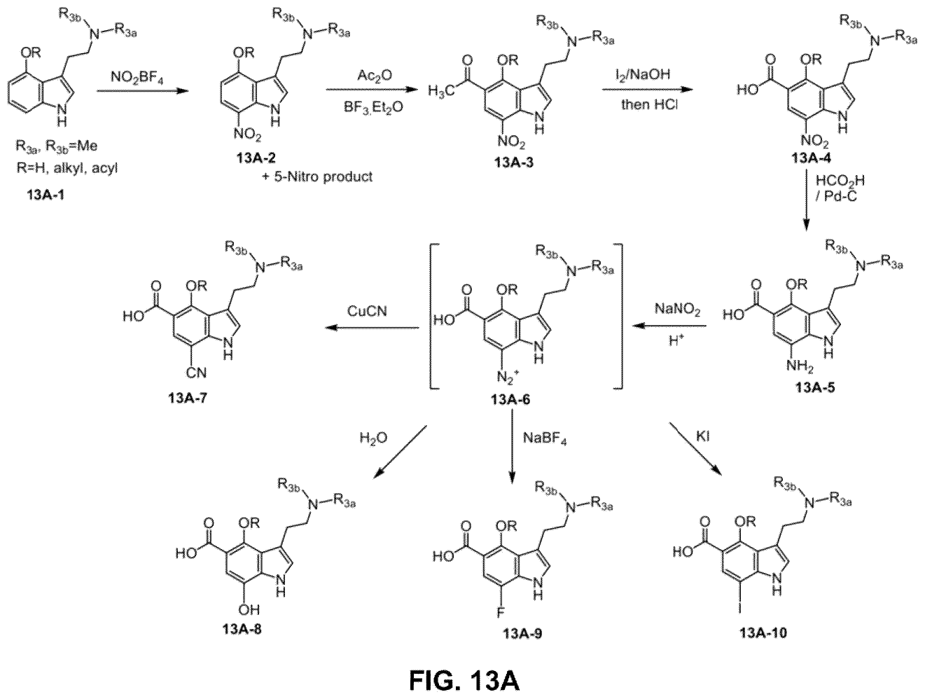

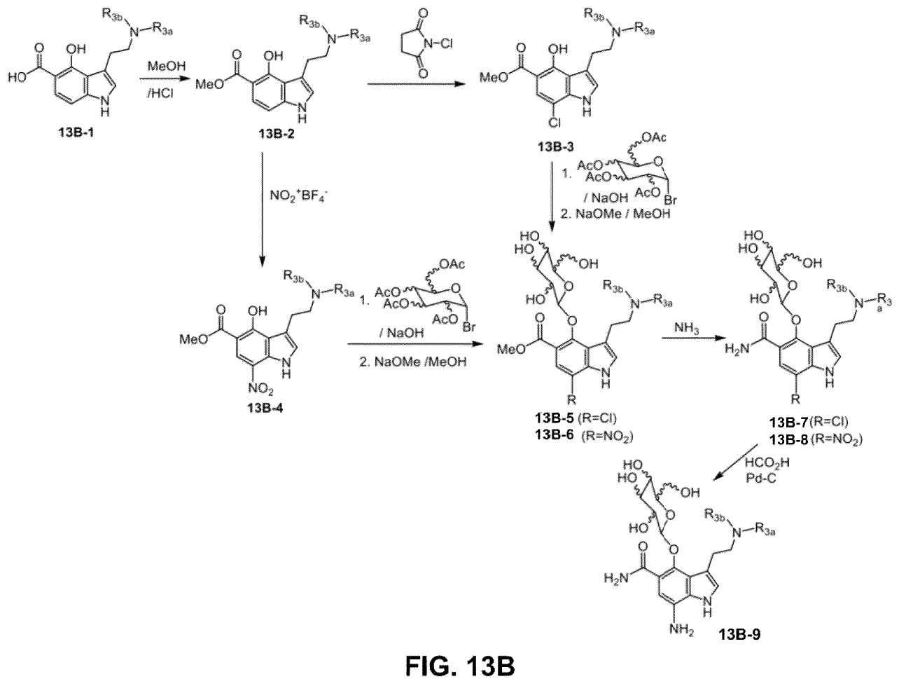

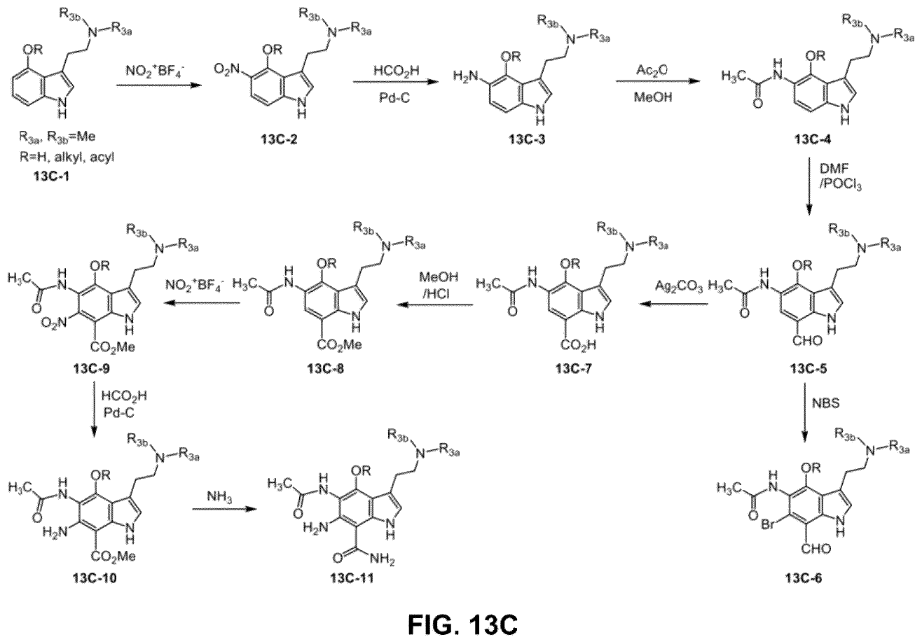

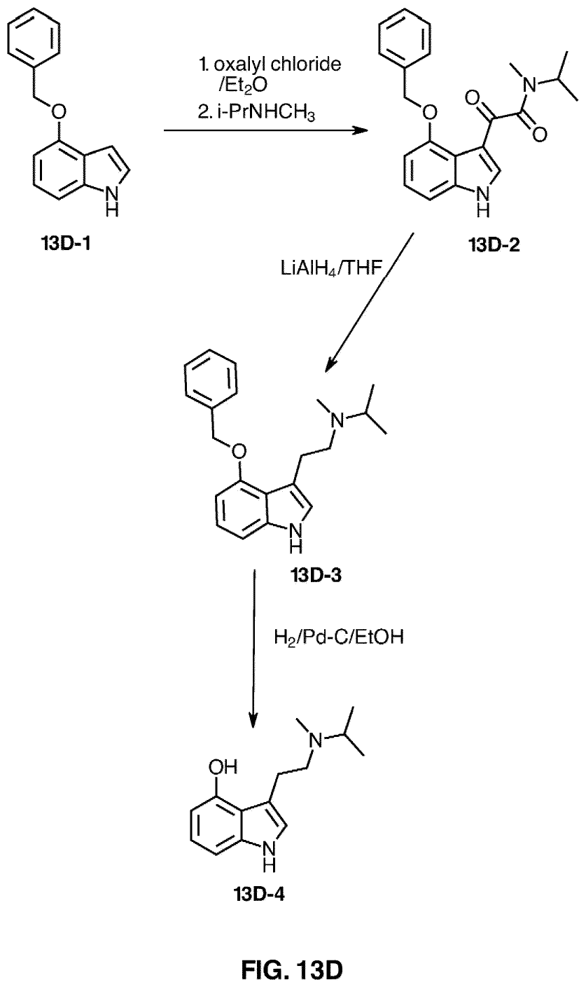

One suitable method of making the multi-substituent psilocybin derivatives of the present disclosure initially involves selecting and obtaining or preparing a reactant psilocybin derivative compound and selecting and obtaining or preparing a substituent group containing compound and, thereafter chemically or biochemically reacting the reactant psilocybin derivative compound and the substituent group containing compound to obtain a multi-substituent psilocybin derivative compound. It is noted that in embodiments hereof where the reactant psilocybin derivative compound does not already possess at least one substituent group, the non-substituent reactant psilocybin derivative compound (i.e., generally an indole structure containing reactant wherein R₂, R₅, R₆ and R₇ are each hydrogen atoms) can be reacted, generally sequentially, with at least two substituent groups containing compounds. Examples thereof are shown in FIGS. 9, 10, 11A-11C, and 12A-12D (depicting example reactions to form a first psilocybin derivative possessing a single substituent), in conjunction with FIGS. 13A-13C (depicting follow-on example reactions to form multi-substituent psilocybin derivatives possessing two or more substituents). In other embodiments, the reactant psilocybin derivative compound may possess at least one substituent group, and is reacted with at least one additional substituent group containing compound (such as depicted in FIGS. 8A-8G).

Accordingly, in one aspect, the present disclosure provides, in at least one embodiment, a method of making a psilocybin derivative or salt thereof having a formula (I):

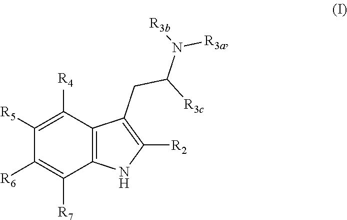

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and R_{3c} is a hydrogen atom or a carboxyl group, the method comprising:
reacting a reactant psilocybin derivative compound having a chemical formula (II):

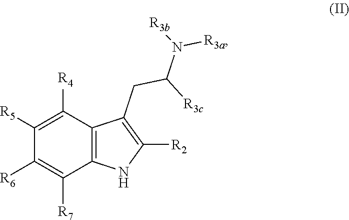

wherein, one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom or an alcohol group and when R₄ is not substituted with any of the foregoing substituents, R₄ is a hydrogen atom, a hydroxy group, an O-alkyl group, O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group
with
   a substituent containing compound, wherein the substituent in the substituent containing compound is selected from (i) a halogen containing compound, (ii) a hydroxy group containing compound, (iii) a nitro group containing compound, (iv) a glycosyloxy group containing compound, (v) an amino group or an N-substituted amino group containing compound, (vi) a carboxyl group or a carboxylic acid derivative containing compound, (vii) an aldehyde or a ketone group containing compound, (viii) a prenyl group containing compound, and (ix) a nitrile group containing compound under reaction conditions sufficient to form the psilocybin substituent or salt thereof having chemical formula (I).

Reactant psilocybin derivative compound having formula (II) encompasses a plurality of compounds. In general, a reactant psilocybin derivative compound having formula (II) can be selected, by initially identifying a desired multi-substituent psilocybin derivative compound, and determining the substituent groups therein, and by thereafter selecting an appropriate reactant psilocybin derivative compound having formula (II). Thus, for example, if it is desirable to prepare a S1₄, S2₆ multi-substituent psilocybin derivative, a S1₄ reactant psilocybin derivative compound may be selected and reacted with an S2 substituent containing compound to form the desired S1₄, S2₆ multi-substituent psilocybin derivative compound, or if it is desirable to prepare a S1₅, S2₆ multi-substituent psilocybin derivative, a S1₅ reactant psilocybin derivative compound may be selected and reacted with an S2 substituent containing compound to form the S1₅, S2₆ multi-substituent psilocybin derivative. Thus, furthermore it can be said that the performance of chemical reactions to make the compounds of the present disclosure, in general involves a substitution at different carbon atoms, i.e. the C₂, C₄, C₅, C₆ and/or C₇ atom.

Thus, in one example embodiment, to form a multi-substituent psilocybin derivative wherein S1₅ is a chlorine atom, and R₂, R₆, and R₇ are S2₂, S2₆ or S2₇, the reactant psilocybin derivative can be selected to be a chemical compound wherein R₄ is an O-alkyl group, R₂, R₆, and R₇ are a hydrogen atom, R₅ is a chlorine atom, and R_{3A} and R_{3B} are a hydrogen atom, an alkyl group, an acyl group, or an aryl group, such as, for example, the reactant psilocybin derivative shown in FIGS. 8A and 8B.

In one further example embodiment, to form a multi-substituent psilocybin derivative wherein S1₅ is a chlorine atom, and R₂, R₆, and R₇ are S2₂, S2₆ or S2₇, the reactant psilocybin derivative can be selected to be a chemical compound wherein R₄ is an O-acyl group, R₂, R₆, and R₇ are a hydrogen atom, R₅ is a chlorine atom, and R_{3A} and R_{3B} are a hydrogen atom, an alkyl group, an acyl group, or an aryl group, such as, for example, the reactant psilocybin derivative shown in FIGS. 8C and 8D.

In one further example embodiment, to form a multi-substituent psilocybin derivative wherein S1₅ is a chlorine atom, and R₂, R₆, and R₇ are S2₂, S2₆ or S2₇, the reactant psilocybin derivative can be selected to be a chemical compound wherein R₄ is a hydroxyl group, R₂, R₆, and R₇ are a hydrogen atom, R₅ is a chlorine atom, and R_{3A} and R_{3B} are a hydrogen atom, an alkyl group, an acyl group, or an aryl group, such as, for example, the reactant psilocybin derivative shown in FIG. 8E.

In one further example embodiment, to form a multi-substituent psilocybin derivative wherein S1₅ is a chlorine atom, and R₂, R₆, and R₇ are S2₂, S2₆ or S2₇, the reactant psilocybin derivative can be selected to be a chemical compound wherein R₄ is a phosphate group, R₂, R₆, and R₇ are a hydrogen atom, R₅ is a chlorine atom, and R_{3A} and R_{3B} are a hydrogen atom, an alkyl group, an acyl group, or an aryl group, such as, for example, the reactant psilocybin derivative shown in FIG. 8F.

In yet one further example embodiment, to form a multi-substituent psilocybin derivative wherein S1₅ is a chlorine atom, and R₂, R₆, and R₇ are S2₂, S2₆ or S2₇, the reactant psilocybin derivative can be selected to be a chemical compound wherein R₄ is a hydrogen atom, R₂, R₆, and R₇ are a hydrogen atom, R₅ is a chlorine atom, and R_{3A} and R_{3B} are a hydrogen atom, an alkyl group, an acyl group, or an aryl group, such as, for example, the reactant psilocybin derivative shown in FIG. 8G.

The reactant psilocybin derivative compounds may be provided in a more or less chemically pure form, for example, in the form of a psilocybin derivative preparation having a purity of at least about 95%, at least about 96%, at least about 97%, at least about 98%, at least about 99%, or at least 99.9%. The psilocybin derivative may be chemically synthesized, or obtained from a fine chemical manufacturer, such as, for example, Sigma-Aldrich® (St. Louis, MO, USA).

The substituent group containing compound can be any compound comprising a substituent group selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group capable of reacting with the selected reactant psilocybin derivative compound.

The substituent group containing compound may be provided in a more or less chemically pure form, for example, having a purity of at least about 95%, at least about 96%, at least about 97%, at least about 98%, at least about 99%, or at least 99.9%. The nitrile containing compound may be synthesized or purified, or can be conveniently obtained from a fine chemical manufacturer, such as, for example, Sigma-Aldrich® (St. Louis, MO, USA).

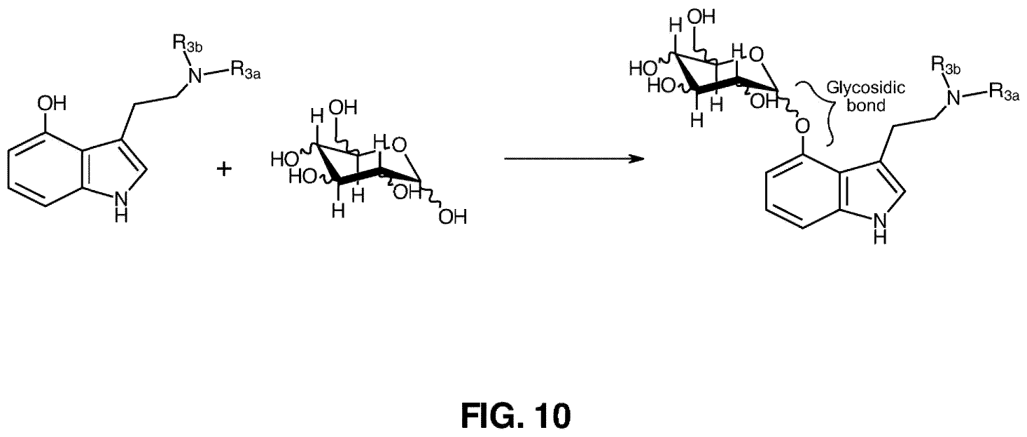

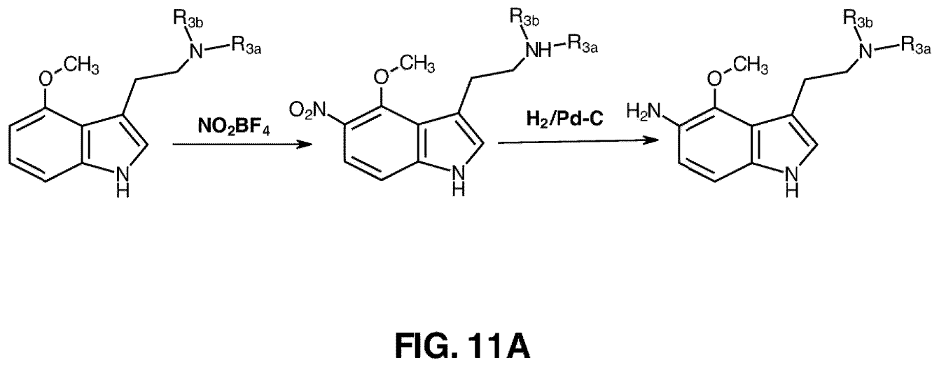

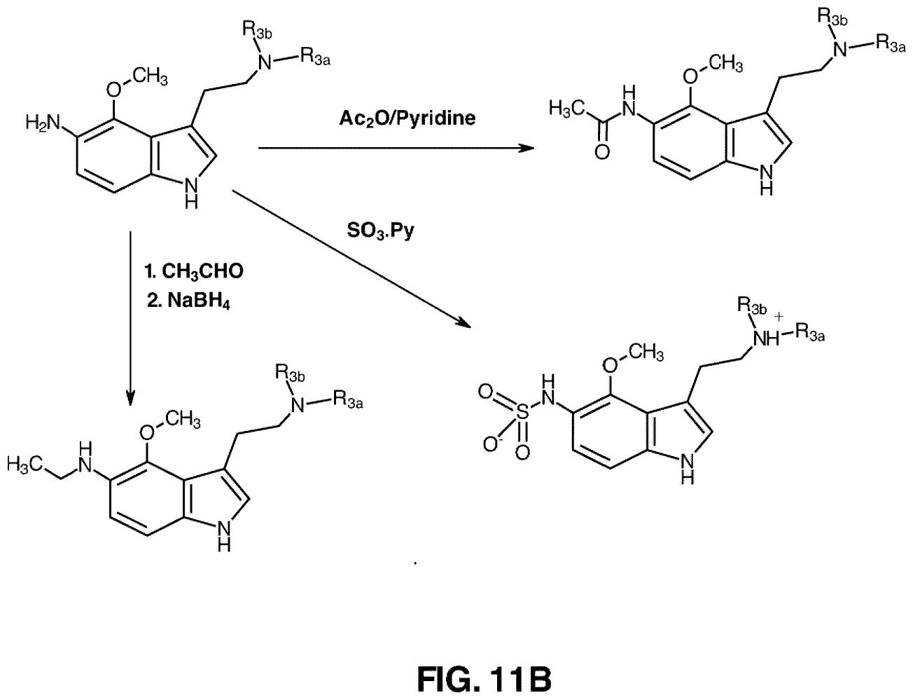

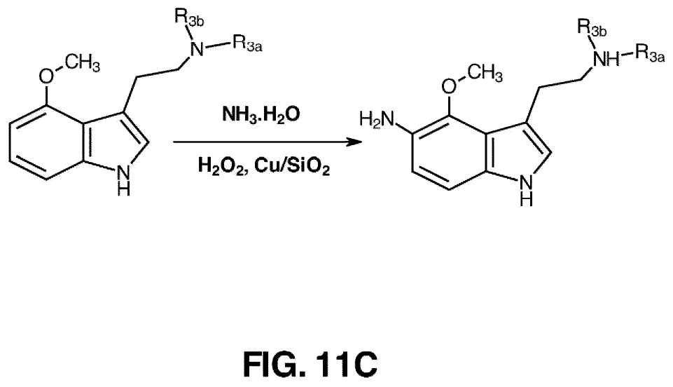

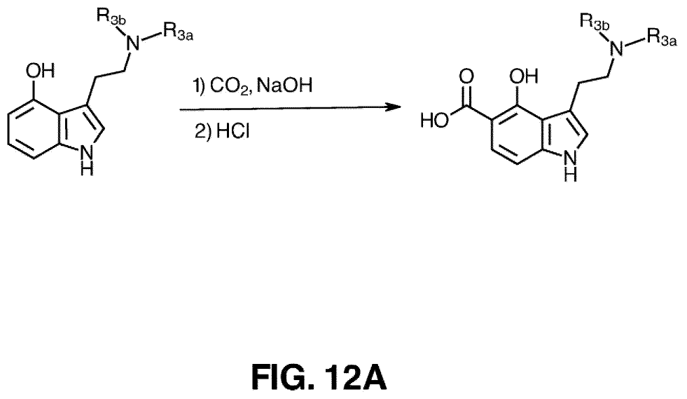

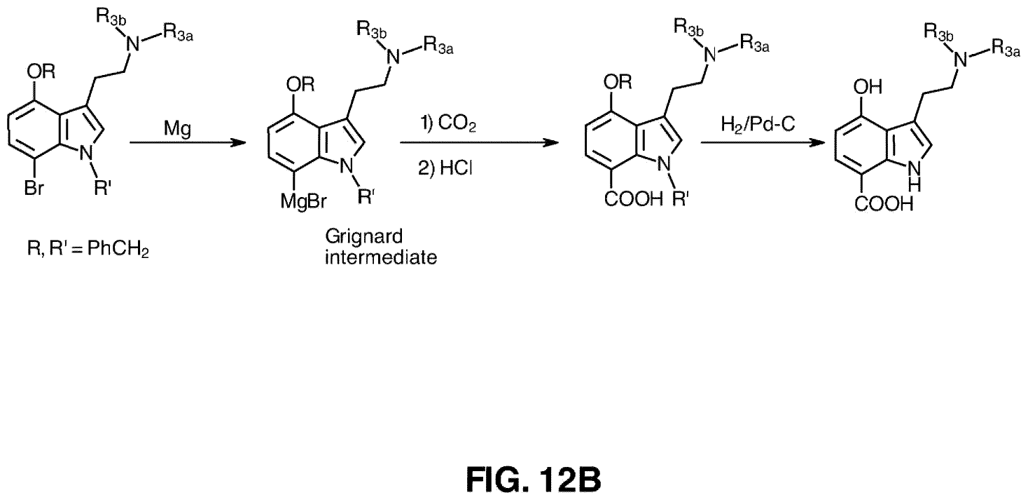

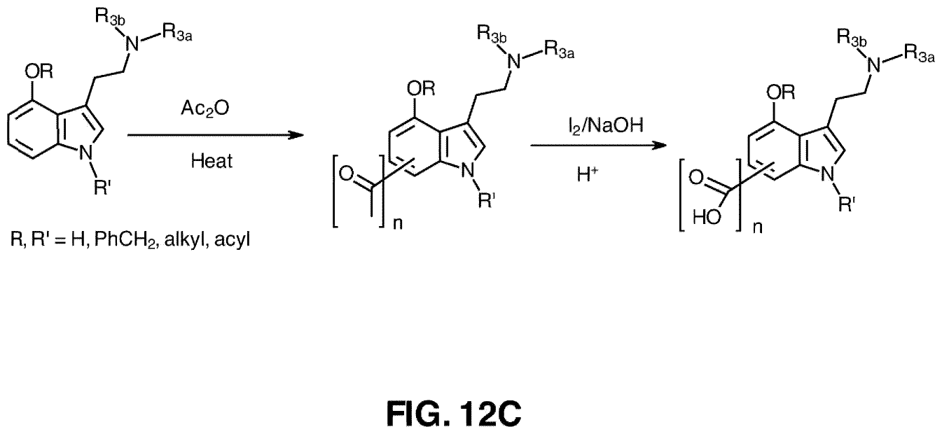

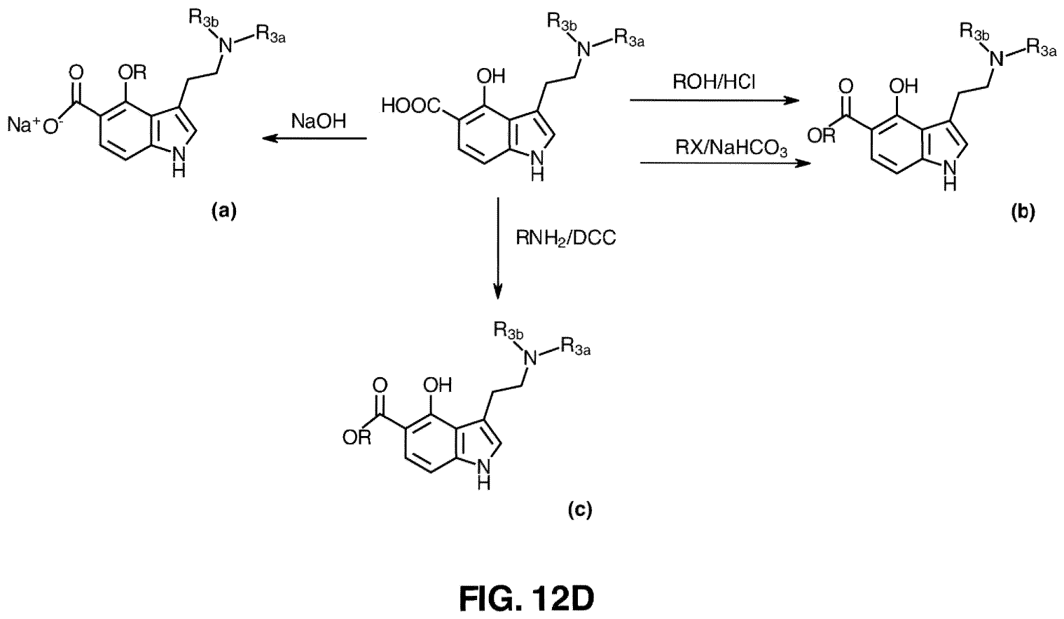

By way of an example, shown in FIGS. 9, 10, 11A-11C, 12A-12D, and 13A-13C are example reactions to form an initial nitrated (FIG. 9), glycosylated (FIG. 10), aminated (FIG. 11A-11C), and carboxylated (FIG. 12A-12D) psilocybin derivatives. Each of the obtained compounds may subsequently then be reacted with an additional substituent containing compound, as shown, by way of example, in FIGS. 13A-13C.

Thus, referring to FIG. 13A, shown therein are example chemical transformations for an initially synthesized 7-nitrated psilocybin derivative (see: compound 13A-2) to other 5,7-di-substituted psilocybin derivatives containing two types of groups at the C₅ and C₇ carbon atom. For example, a Friedel-Crafts acylation with compound 13A-2 will regioselectively install an acetyl group at the C₅ carbon to afford compound 13A-3, which, in turn, can be further oxidized to the corresponding 5-carboxy derivative (compound 13A-4) via an iodoform reaction. The 7-nitro group can then be reduced to the corresponding 7-amino-psilocibin derivative (compound 13A-5) through a palladium-mediated reduction using formic acid as the hydrogen source. The 7-amino group can be further converted to the reactive and versatile 7-diazonium salt (compound 13A-6) using sodium nitrite as a reagent under acidic condition. From the reactive 7-diazonium salt intermediate compound 13A-6, the corresponding 5-carboxy-psilocibin derivatives additionally containing a 7-nitrile (compound 13A-7), a 7-hydroxy group (compound 13A-8), a 7-fluoride group (compound 13A-9), or a 7-iodide group (compound 13A-10) can be obtained.

In one example embodiment, in an aspect, the formed first psilocybin derivative can be the compound possessing an acetyl group, such as, for example, the compound shown in FIG. 13A and labeled 13A-3. In other examples, R₂, R₄, or R₆ or R₇ can possess an acetyl group.

Thus, referring further to FIG. 13A, and the chemical compound having formula (I), it will be clear that a formed first multi-substituent psilocybin derivative compound can be further reacted to form additional multi-substituent psilocybin derivative compounds. Thus, for example, the formed first psilocybin derivative can be the compound shown in FIG. 13A and labeled 13A-3, or a compound wherein other combinations of R₂, R₄, R₅, R₆ and R₇ posses a nitro group and an acetyl group. This formed first psilocybin derivative can be reacted to oxidize the acetyl group and form a second psilocybin derivative having formula (I), wherein one or at least one of R₂, R₄, R₅, R₆, or R₇ is a nitro group, and wherein one or at least one of R₂, R₄, R₅, R₆, or R₇ is a carboxyl group, and form a second psilocybin derivative, for example, the compound shown in FIG. 13A and labeled 13A-4.

Continuing to refer to FIG. 13A, and the chemical compound having formula (I), the formed second psilocybin derivative can be reacted to reduce the nitro group and form an amino group, and a third psilocybin derivative, wherein one or at least one of R₂, R₄, R₅, R₆, or R₇ is an amino group, and wherein in one or at least one of R₂, R₄, R₅, R₆, or R₇ is a carboxyl group. The formed third psilocybin derivative can be the compound shown in FIG. 13A and labeled 13A-5.

Continuing to refer to FIG. 13A, and the chemical compound having formula (I), the formed third psilocybin derivative can be reacted with a nitrite to convert the amino group in a diazonium salt and form an intermediate psilocybin derivative, wherein one or at least one of R₂, R₄, R₅, R₆, or R₇ is a diazonium group, and one or at least one of R₂, R₄, R₅, R₆, or R₇ is a carboxyl group. The intermediate formed psilocybin derivative can, for example, be the compound shown in FIG. 13A and labeled 13A-6.

Continuing to refer to FIG. 13A, and the chemical compound having formula (I), the intermediate formed psilocybin derivative can be reacted with a nitrile containing compound to convert the diazonium group and form a fourth psilocybin derivative, wherein one or at least one of R₂, R₄, R₅, R₆, or R₇ is a nitrile group, and one or at least one of R₂, R₄, R₅, R₆, or R₇ is a carboxyl group, for example, the compound shown in FIG. 13A and labeled 13A-7.

Continuing to refer to FIG. 13A, and the chemical compound having formula (I), the intermediate formed psilocybin can be reacted with water to convert the diazonium group and form a fifth psilocybin, wherein one or at least one of R₂, R₄, R₅, R₆, or R₇ is a hydroxy group, and one or at least one of R₂, R₄, R₅, R₆, or R₇ is a carboxyl group, for example, the compound shown in FIG. 13A and labeled 13A-8.

Continuing to refer to FIG. 13A, and the chemical compound having formula (I), the intermediate formed psilocybin derivative can be reacted with a halogen containing compound to convert the diazonium group and form a sixth psilocybin derivative wherein one or at least one of R₂, R₄, R₅, R₆, or R₇ is a halogen atom, and one or at least one of R₂, R₄, R₅, R₆, or R₇ is a carboxyl group, for example, the compounds shown in FIG. 13A and labeled 13A-9 and 13A-10.

Referring next to FIG. 13B, shown therein are example chemical transformations using the initially synthesized 5-carboxy psilocybin derivative (see compound: 13B-1) to other O-4-glycosylated psilocybin derivatives containing two types of groups at carbon atoms C₅ and C₇. Thus, starting with compound 13B-1, the 5-carboxy functionality can be selectively esterified in methanol under Fisher esterification conditions to afford compound 13B-2 which can be used to synthesize the corresponding 7-chloro (compound 13B-3) and 7-nitro (compound 13B-4) derivatives, respectively. Both compounds 13B-3 and 13B-4 can be glycosylated at the O-4 position with a per-O-acetylated glycosyl bromide and after removing all the O-acetates, the corresponding 4-O-glycosylated derivative compounds 13A-5 and 13A-6 can be obtained. The 5-ester functionality of both compounds can be further converted to a 5-amido group via an aminolysis reaction to afford, respectively, 4-O-glycosylated psilocybin derivative compounds 13B-7 and 13B-8. The 7-nitro group of compound 13B-8 can be further reduced to afford compound 13B-9 through a palladium-mediated reduction using formic acid as the hydrogen source.

Referring further to FIG. 13B, and the chemical compound having formula (I), it will be clear that a reactant psilocybin derivative compound containing a substituent group can be used to react with a substituent containing compound and form an initial multi-substituent psilocybin derivative compound, which in turn can be used to form additional multi-substituent psilocybin derivative compounds. Thus, for example, the substituent in the reactant psilocybin derivative can be a methoxycarbonyl group, the substituent containing compound can be the halogen containing compound N-halo-succinimide, and the reactant psilocybin derivative and the substituent containing compound can be reacted to form a first psilocybin derivative, wherein one of R₂, R₄, R₅, R₆, or R₇ is a methoxy carbonyl group, and one or at least one of R₂, R₄, R₅, R₆, or R₇ is a halogen atom, for example, the compound shown in FIG. 13B and labeled 13B-3.

Continuing to refer to FIG. 13B, and the chemical compound having formula (I) and (II), the reactant psilocybin derivative can possess a methoxycarbonyl group, the substituent containing compound can be the nitro containing compound nitronium tetrafluoroborate, and the reactant psilocybin derivative and the substituent containing compound can be reacted to form a second psilocybin derivative wherein one or at least one of R₂, R₄, R₅, R₆, or R₇ is a methoxy carbonyl group, and one or at least one of R₂, R₄, R₅, R₆, or R₇ is a nitro group, and the formed second psilocybin derivative can be the compound shown in FIG. 13B and labeled 13B-4.

Continuing to refer to FIG. 13B, and the chemical compound having formula (I), the formed first psilocybin derivative can be reacted with an acetylated glycosyl compound and form a third psilocybin, wherein one or at least one of R₂, R₄, R₅, R₆, or R₇ is a methoxy carbonyl group, one or at least one of R₂, R₄, R₅, R₆, or R₇ is a glycosyloxy group, and one or at least one of R₂, R₄, R₅, R₆, or R₇ is a halogen atom, for example, the compound shown in FIG. 13B and labeled 13B-5.

Continuing to refer to FIG. 13B, and the chemical compound having formula (I), the formed second psilocybin derivative can be reacted with an acetylated glycosyl compound and form a fourth psilocybin derivative, wherein one or at least one of R₂, R₄, R₅, R₆, or R₇ is a methoxycarbonyl group, one or at least one of R₂, R₄, R₅, R₆, or R₇ is a glycosyloxy group, and one or at least one of R₂, R₄, R₅, R₆, or R₇ is a nitro group. For example, the formed fourth psilocybin derivative can be the compound shown in FIG. 13B and labeled 13B-6.

Continuing to refer to FIG. 13B, and the chemical compound having formula (I), the formed third psilocybin derivative can be reacted with ammonia to convert the methoxycarbonyl group in an amido group and form a fifth psilocybin derivative, wherein one or at least one of R₂, R₄, R₅, R₆, or R₇ is an amido group, one or at least one of R₂, R₄, R₅, R₆, or R₇ is a glycosyloxy group, and one or at least one of R₂, R₄, R₅, R₆, or R₇ is a halogen atom, for example the compound shown in FIG. 13B and labeled 13B-7.

Continuing to refer to FIG. 13B, and the chemical compound having formula (I), the formed third psilocybin derivative can be reacted with ammonia to convert the methoxycarbonyl group in an amido group and form a sixth psilocybin derivative, wherein one or at least one of R₂, R₄, R₅, R₆, or R₇ is an amido group, one or at least one of R₂, R₄, R₅, R₆, or R₇ is a glycosyloxy group, and one or at least one of R₂, R₄, R₅, R₆, or R₇ is a nitro group, for example, the compound shown in FIG. 13B and labeled 13B-8.

Continuing to refer to FIG. 13B, and the chemical compound having formula (I), the formed sixth psilocybin derivative can be reacted to reduce the nitro group to form an amino group and a seventh psilocybin derivative, wherein one or at least one of R₂, R₄, R₅, R₆, or R₇ is an amido group, one or at least one of R₂, R₄, R₅, R₆, or R₇ is a glycosyloxy group, and one or at least one of R₂, R₄, R₅, R₆, or R₇ is a nitro group, for example the compound shown in FIG. 13B and labeled 13B-9.

Thus, referring to FIG. 13C, shown therein are more example chemical transformations of synthesized 5-nitrated psilocybin derivatives (see: compound 13C-2) to 5,6,7-tri-substituted psilocybin derivatives containing up to three types of substituent groups. For example, the 5-nitro group can be reduced to produce 5-amino derivatives (compound: 13C-3) through a palladium-mediated reduction in the presence of formic acid as the hydrogen source. A chemo-selective N-acetylation can be carried out in methanol to afford the corresponding 5-acetamino psilocybin derivative compound 13C-4, which can then be formylated at carbon atom C₇ to obtain 7-aldehyde compound 13C-5 using DMF-POCl₃ as the reagent. A further bromination using N-bromosuccinimide can provide the 5-acetamido-6-bromo-7-formyl-psilocybin derivative compound 13C-6. Alternatively, using compound 13C-5 as a substrate, the 7-formyl group can be oxidized under mild conditions using silver carbonate as a reagent to afford the 7-carboxy derivative compound 13C-7 which can be esterified using Fisher esterification conditions to obtain compound 13C-8. By reacting compound 13C-8 with nitrosonium tetrafluoroborate, a nitro group can be installed at carbon atom C₆ to afford a 5,6,7-trisubstituted psilocybin derivative compound 13C-9 which can have its 6-nitro group reduced to form compound 13C-10 and subsequently undergo an aminolysis reaction to convert the 7-ester group to an amide. This affords the novel 5,6,7-tri-substituted psilocybin derivative compound 13C-11.

Referring further to FIG. 13C and the chemical compounds having formula (I) and (II), it will be clear that a reactant psilocybin derivative compound containing a substituent group can be used to react with a substituent containing compound and form an initial multi-substituent psilocybin derivative compound, which in turn can be used to form additional multi-substituent psilocybin derivative compounds. Thus, for example, the substituent in the reactant psilocybin derivative can be an acetamidyl group, the substituent containing compound can be the halogen containing compound N-halo-succinimide (e.g., N-bromo-succinimide (NBS)), and the reactant psilocybin derivative and the substituent containing compound can be reacted to form a first psilocybin derivative, wherein one or at least one of R₂, R₄, R₅, R₆, or R₇ is an acetamidyl group, and one or at least one of R₂, R₄, R₅, R₆, or R₇ is a halogen atom, for example, the compound shown in FIG. 13C and labeled 13C-6.

Referring further to FIG. 13C and the chemical compound having formula (I) and (II), the reactant psilocybin derivative compound can possess an acetamidyl group, the substituent containing compound can be dimethyl formamide, and the reactant psilocybin derivative and the substituent containing compound can be reacted to form an intermediate psilocybin derivative, wherein one or at least one of R₂, R₄, R₅, R₆, or R₇ is an acetamidyl group, and at least one or at least one of R₂, R₄, R₅, R₆, or R₇ is a methanol group, and wherein the intermediate psilocybin derivative is reacted to oxidize the methanol group, and form a second psilocybin derivative, wherein one or at least one of R₂, R₄, R₅, R₆, or R₇ is an acetamidyl group, and one or at least one of R₂, R₄, R₅, R₆, or R₇ is a carboxy group. For example, the intermediate psilocybin derivative can be the compound shown in FIG. 13C and labeled 13C-5, and the formed second psilocybin derivative can be the compound shown in FIG. 13C and labeled 13C-7.

Referring further to FIG. 13C and the chemical compound having formula (I) the formed second psilocybin derivative having formula (I) can be reacted with an alcohol to esterify the carboxy group to form an ester and a third psilocybin derivative, wherein one or at least one of R₂, R₄, R₅, R₆, or R₇ is an acetamidyl group, one or at least one of R₂, R₄, R₅, R₆, or R₇ is a carboxyl ester, for example, the compound shown in FIG. 13C and labeled 13C-8.

Referring further to FIG. 13C and the chemical compound having formula (I), the formed third psilocybin derivative can be reacted with a nitro group containing compound and form a fourth psilocybin derivative, wherein one or at least one of R₂, R₄, R₅, R₆, or R₇ is an acetamidyl group, one or at least one of R₂, R₄, R₅, R₆, or R₇ is a carboxyl ester, and at least one of R₂, R₄, R₅, R₆, or R₇ is a nitro group, for example, the compound shown in FIG. 13C and labeled 13C-9.

Referring further to FIG. 13C and the chemical compound having formula (I), the formed fourth psilocybin derivative can be reacted to reduce the nitro group to form an amino group and a fifth psilocybin derivative, wherein one or at least one of R₂, R₄, R₅, R₆, or R₇ is an acetamidyl group, one or at least one of R₂, R₄, R₅, R₆, or R₇ is a carboxyl ester, and one at least one of R₂, R₄, R₅, R₆, or R₇ is an amino group, for example, the compound shown in FIG. 13C and labeled 13C-10.

Referring further to FIG. 13C and the chemical compound having formula (I), the formed fifth psilocybin derivative can be reacted with ammonia to form an amido group and a sixth psilocybin derivative having formula (I), wherein one or at least one of R₂, R₄, R₅, R₆, or R₇ is an acetamidyl group, one or at least one of R₂, R₄, R₅, R₆, or R₇ is a carboxyester, and at least one of R₂, R₄, R₅, R₆, or R₇ is an amido group, for example, the compound shown in FIG. 13C and labeled 13C-11.

Thus, in general, a reactant psilocybin derivative is provided, and the reactant psilocybin derivative is employed to react in a chemical reaction resulting in the formation of a multiple-substituent psilocybin derivatives.

The reactions, such as the example reaction shown in FIGS. 9, 10, 11A-11C, 12A-12D, and 13A-13C, may be conducted in any suitable reaction vessel (e.g., a tube, bottle). Suitable solvents that may be used are for example, water, alcohol (such as methanol, ethanol, tetrahydrofuran (THF), dichloromethane, acetone, N,N-dimethylformamide (DMF), dimethylsulfoxide (DMSO), or a combination of solvents. Suitable temperatures may range from, for example, e.g., from about 20° C. to about 100° C. Furthermore, reaction times may be varied. As will readily be appreciated by those of skill in the art, the reaction conditions may be optimized, for example by preparing several psilocybin derivative reactants preparations and reacting these in different reaction vessels under different reaction conditions, for example, at different temperatures, using different solvents, using different catalysts etc., evaluating the obtained multiple-substituent psilocybin derivative reaction product, adjusting reaction conditions, and selecting a desired reaction condition.

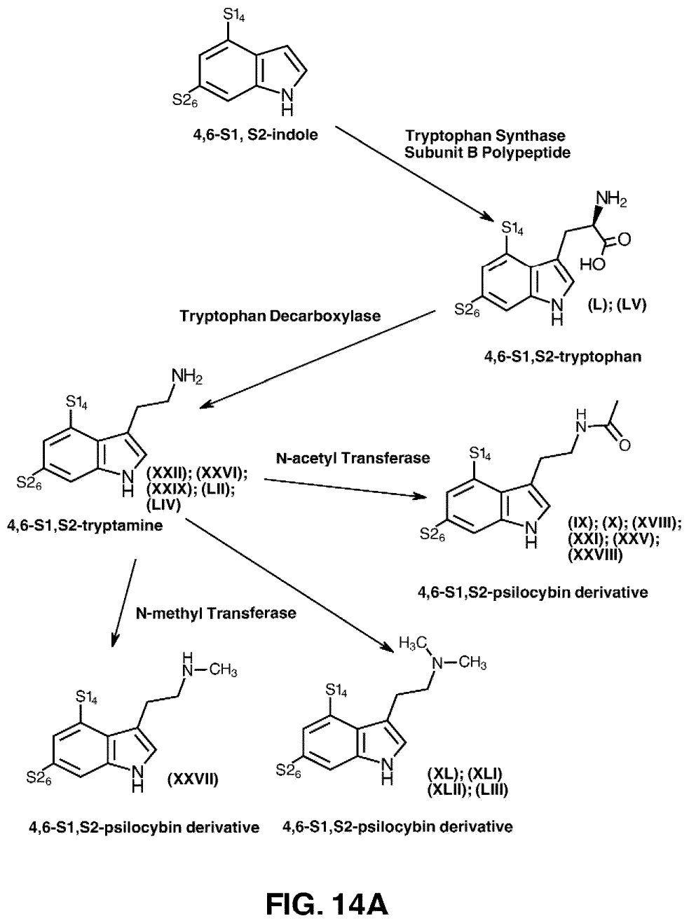

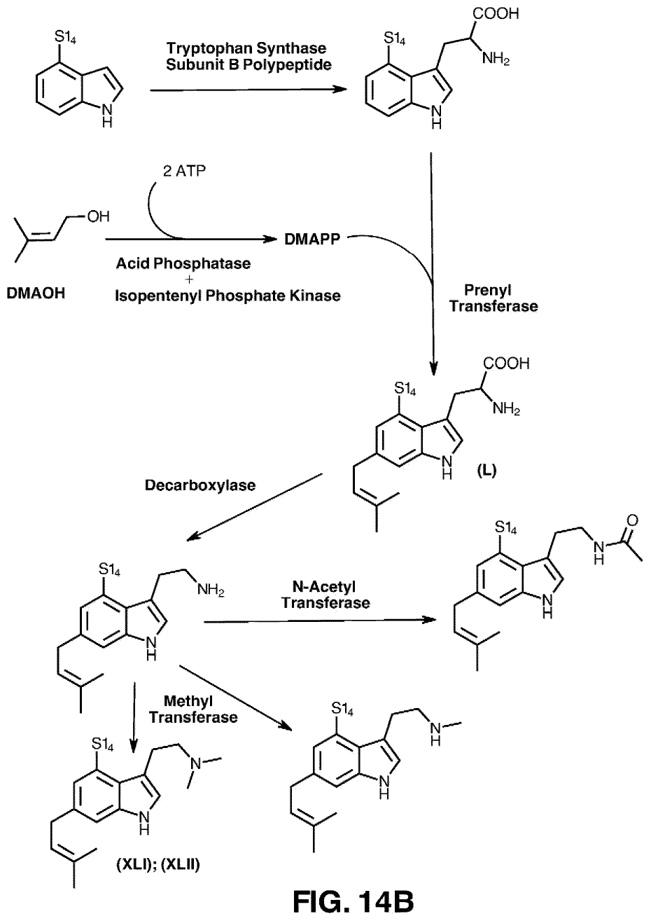

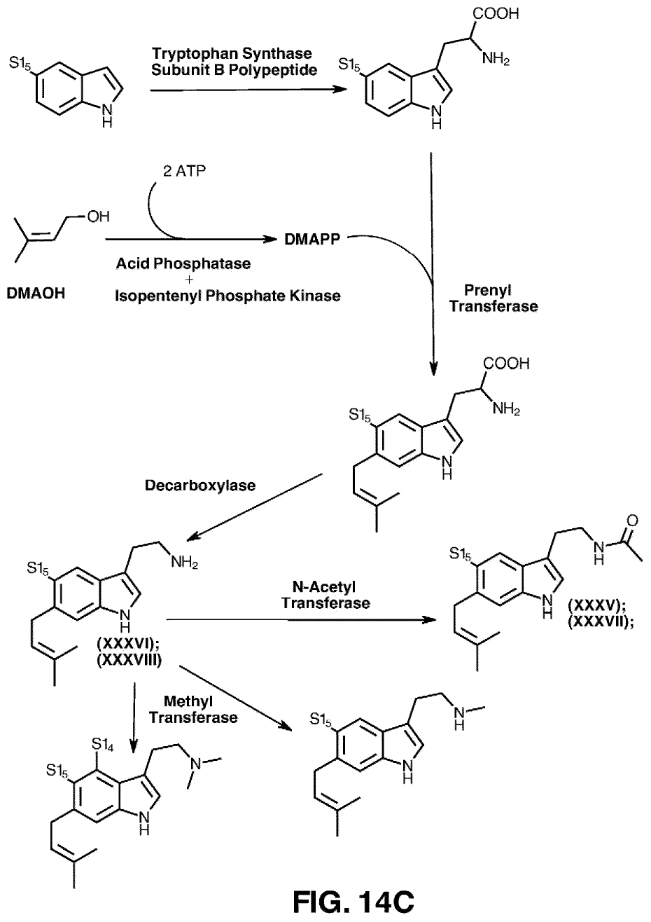

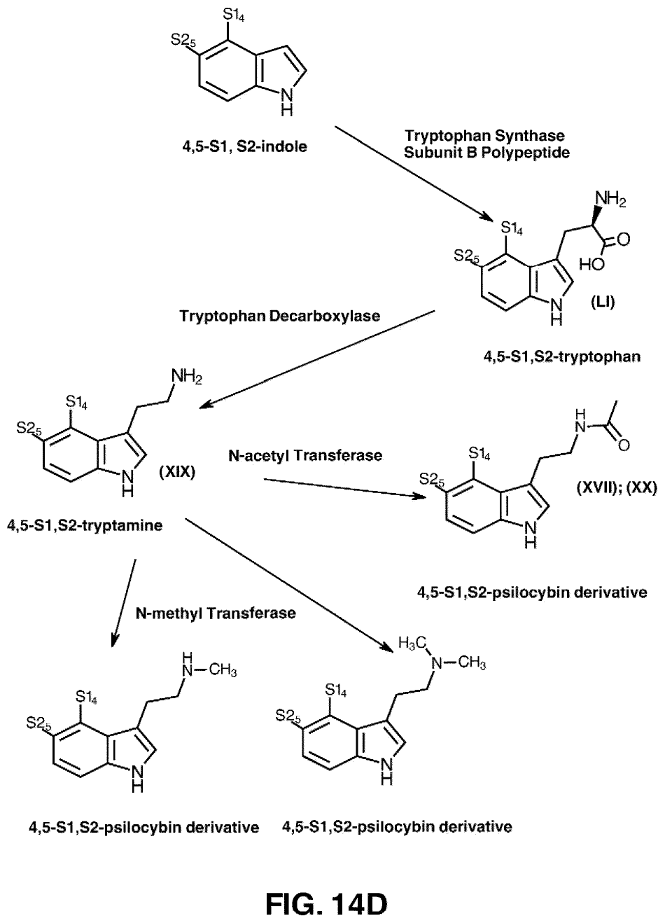

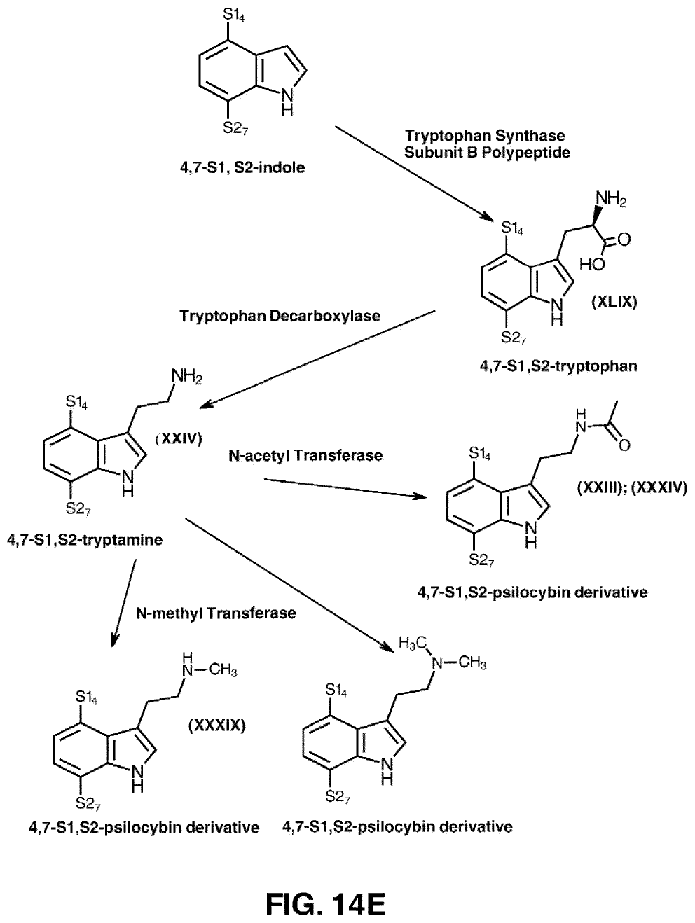

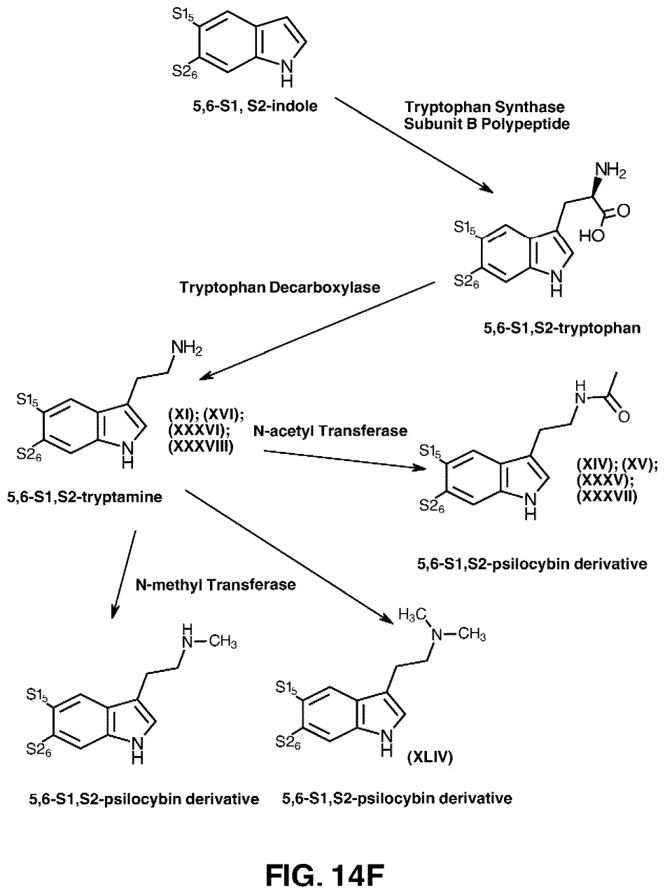

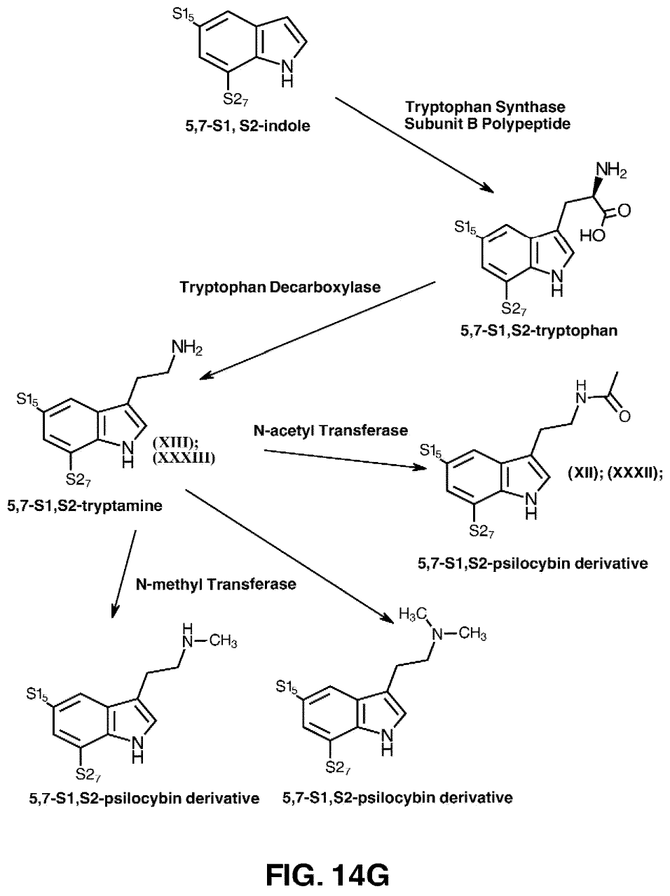

Turning next to biosynthetic methods to make the multiple-substituent compounds of the present disclosure, such methods, in general, involve the use of psilocybin biosynthetic enzyme complement to enzymatically catalyze the conversion of a psilocybin derivative precursor compound and form a multi-substituent psilocybin derivative compound. The enzymes included in the psilocybin biosynthetic enzyme complement may vary, as hereinafter will be discussed with reference to certain example enzymes and example compounds shown in FIGS. 14A, 14B, and 14C.

Thus, in one aspect, the present disclosure further provides a method of making a multi-substituent psilocybin derivative, the method comprising contacting a psilocybin derivative precursor compound having a formula (LVII):

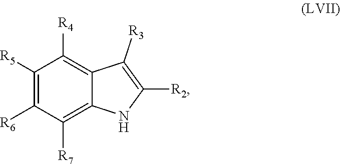

wherein at least one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, and wherein R₃ is a hydrogen atom or —CH₂—CHNH₂COOH or —CH₂—CH₂NH₂,
   with a catalytic quantity of a psilocybin biosynthetic enzyme complement under reaction conditions permitting an enzyme catalyzed conversion of the psilocybin derivative precursor compound to form a multi-substituent psilocybin derivative compound having a formula (I):

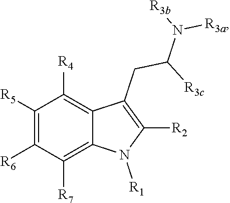

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.

The reaction conditions can be in vitro reaction conditions, or in vivo reaction conditions, or a combination thereof.

### In Vitro Synthesis

In vitro synthesis, in general, involves initially providing the reagents, including the precursor psilocybin derivative compound and other reactants, in a more or less pure form. Thus, the reactants may be provided as a particulate in a substantially pure form, or they may be dissolved, in a more or less pure form, in a suitable solvent or diluent, such as water or a buffer. The reagents can then be combined and contacted with one another in a suitable reaction vessel, such as a tube, beaker, flask, or the like, or, at a larger scale, in a tank or reactor, generally preferably in liquid form, which may be prepared by further including a diluent, such as water or a buffer, as necessary. The combined reagents may be mixed, by, in general, gentle stirring, using a suitable stirring or mixing device, such as a laboratory size magnetic stirrer (e.g., as manufactured by Fisher Scientific®), or a handheld or industrial mixer, for example, to form a mixture. Relative quantities and absolute quantities of reagents may be selected as desired. Absolute quantities will typically depend on the scale one wishes to perform the reaction at, such as, for example, at a laboratory scale (e.g., at a less than 1 L, a less than 100 mL, a less than 10 mL, or a less than 1 mL scale), or, for example, at a commercial production scale (e.g., at a more than 100 L, a more than 1,000 L, or a more than 10,000 L scale). Relative quantities of the reagents may vary. Thus, for example, in one embodiment, stoichiometric quantities of each of a precursor psilocybin derivative and a substituent containing compound can be mixed with catalytic quantities of enzymes. If desired, off-stoichiometric quantities of reagents, for example, a molar ratio of psilocybin precursor derivative to substituent containing compound of 1:0.95; 1:0.9; 1:0.75; or 1:1.05, 1:1.1 or 1:1.25, may be selected.

As will be understood by those of skill in the art, in molar quantity terms, small quantities of enzyme suffice to conduct the reaction, since the enzyme acts as a catalytic agent, and, unlike the precursor psilocybin derivatives and the substituent containing compound, the enzyme is not consumed in the reaction. Thus, in general terms, catalytic quantities can be thought of as the at least minimal quantity of enzyme required to convert precursor psilocybin derivatives and the substituent containing compound, and form desirable quantities of multi-substituent psilocybin derivatives. Thus, for example, from 0.1 to 1,000 enzyme units (e.g., 0.1 enzyme unit, 1 enzyme unit, 10 enzyme units, 50 enzyme units, 100 enzyme units, 250 enzyme units, 500 enzyme units, or 1,000 enzyme units) may be included in a reaction mixture, wherein, as is known to those of skill in the art, 1 enzyme unit is an amount of enzyme that catalyzes 1 μmole of substrate (i.e., psilocybin precursor compound) per minute. Furthermore, in vitro reaction conditions may vary and may include temperatures ranging from, for example, between about 18° C. and about 37° C., and a pH in the range of about pH 5.0 to about pH 8.5. Furthermore, other agents may be included to facilitate catalysis, for example, a diluent (e.g., water or a buffer), salts, and pH modifying agents. The in vitro reaction conditions may be adjusted and optimized, for example, by preparing a plurality of samples, each being reacted at a different operating condition, e.g., at a different temperature, a different pH, including a different quantity of enzyme, including different relative quantities of reagents, and so forth, and detecting the formed multi-substituent psilocybin derivative.

### In Vivo Synthesis

In one embodiment, the psilocybin derivative precursor compound and the substituent containing compound can be contacted with the psilocybin biosynthetic enzyme complement in a host cell, wherein the host cell comprises a chimeric nucleic acid sequence comprising as operably linked components:
(i) a nucleic acid sequence controlling expression in the host cell, and
(ii) a nucleic acid sequence encoding psilocybin biosynthetic enzyme complement,
   and the host cell is grown to express the psilocybin biosynthetic enzyme complement and to produce the multi-substituent psilocybin derivative compound.

Suitable chimeric nucleic acid sequences include any nucleic acid sequence comprising a nucleic acid sequence controlling expression in the host cell operably linked to a sequence encoding psilocybin biosynthetic enzyme complement, such as, for example, one or more of tryptophan synthase subunit B polypeptide, tryptophan decarboxylase, N-acetyl transferase, N-methyl transferase, and prenyl transferase, as herein after further described.

Nucleic acid sequences capable of controlling expression of a nucleic acid sequence encoding biosynthetic enzyme complement in host cells that can be used herein include any transcriptional promoter capable of controlling expression of polypeptides in host cells. Generally, promoters obtained from bacterial cells are used when a bacterial host is selected in accordance herewith, while a fungal promoter will be used when a fungal host cell is selected, a plant promoter will be used when a plant cell is selected, and so on. Specific examples that can be used, for example for expression in yeast cells include a galactose inducible promoter, such as a Gal10/Gal 1 promoter, or for expression in Escherichia coli cells, a beta-galactosidase promoter. Further nucleic acid elements capable elements of controlling expression in a host cell include transcriptional terminators, enhancers, and the like, all of which may be included in the chimeric nucleic acid sequences of the present disclosure.

The chimeric nucleic acid sequences can be integrated into a recombinant expression vector which ensures good expression in the host cell, wherein the expression vector is suitable for expression in a host cell. The term “suitable for expression in a host cell” means that the recombinant expression vector comprises the chimeric nucleic acid sequence linked to genetic elements required to achieve expression in a cell. Genetic elements that may be included in the expression vector in this regard include a transcriptional termination region, one or more nucleic acid sequences encoding marker genes, one or more origins of replication and the like. In preferred embodiments, the expression vector further comprises genetic elements required for the integration of the vector or a portion thereof in the host cell's genome, for example. If a plant host cell is used the T-DNA left and right border sequences which facilitate the integration into the plant's nuclear genome.

Pursuant to the present disclosure, the expression vector may further contain a marker gene. Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin or ampicillin, or an auxotrophic marker, for example, a leu marker (Sikorski and Hieter, 1989, Genetics 122(1): 19-2₇) or a ura marker (Rose and Winston, 1984, Mol. Gen. Genet. 193 (3): 557-560. Screenable markers that may be employed to identify transformants through visual inspection include P-glucuronidase (GUS) (U.S. Pat. Nos. 5,268,463 and 5,599,670) and green fluorescent protein (GFP) (Niedz et al., 1995, Plant Cell Rep., 14: 403).

A variety of host cells can be used in accordance herewith. The selected host cell may be able to naturally produce psilocybin compounds, or derivatives thereof or the cell may not be able to naturally produce psilocybin compounds or derivatives thereof. Host cells, upon the introduction of the chimeric nucleic acid sequence can be said to be able to heterologously express psilocybin biosynthetic enzyme complement.

In some embodiments, the host cell can be a microbial cell, for example, bacterial cell or a yeast cell. An example bacterial cell that can be used in accordance herewith is an Escherichia coli cell. Example yeast cells that can be in accordance herewith are a Saccharomyces cerevisiae cell or a Yarrowia lipolytica cell.

In a further embodiment, the host cell can be a plant cell or an algal cell.

A variety of techniques and methodologies to manipulate host cells to introduce nucleic acid sequences, including expression vectors comprising the chimeric nucleic acid sequences of the current disclosure, in cells and attain expression exists and are well known to the skilled artisan. These methods include, for example, cation based methods, for example, lithium ion or calcium ion based methods, electroporation, biolistics, and glass beads based methods. As will be known to those of skill in the art, depending on the host cell selected, the methodology to introduce nucleic acid material in the host cell may vary, and, furthermore, methodologies may be optimized for uptake of nucleic acid material by the host cell, for example, by comparing uptake of nucleic acid material using different conditions. Detailed guidance can be found, for example, in Sambrook et al., Molecular Cloning, a Laboratory Manual, Cold Spring Harbor Laboratory Press, 2012, Fourth Ed. It is noted that the chimeric nucleic acid is a non-naturally occurring chimeric nucleic acid sequence and can be said to be heterologous to the host cell.

One example host cell that conveniently may be used is Escherichia coli. The preparation of the E. coli vectors may be accomplished using commonly known techniques such as restriction digestion, ligation, gel electrophoresis, DNA sequencing, the polymerase chain reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Suitable promoter sequences for use in E. coli include, for example, the T7 promoter, the T5 promoter, tryptophan (trp) promoter, lactose (lac) promoter, tryptophan/lactose (tac) promoter, lipoprotein (Ipp) promoter, and λ phage PL promoter. Typically, cloning vectors contain a marker, for example, an antibiotic resistance marker, such as ampicillin or kanamycin resistance marker, allowing selection of transformed cells. Nucleic acid sequences may be introduced in these vectors, and the vectors may be introduced in E. coli by preparing competent cells, electroporation or using other well-known methodologies to a person of skill in the art. E. coli may be grown in an appropriate medium, such as Luria-Broth medium and harvested. Recombinant expression vectors may readily be recovered from cells upon harvesting and lysing of the cells.

Another example host cell that may be conveniently used is a yeast cell. Example yeast host cells that can be used are yeast cells belonging to the genus Candida, Kluyveromyces, Saccharomyces, Schizosaccharomyces, Pichia, Hansenula, and Yarrowia. In specific example embodiments, the yeast cell can be a Saccharomyces cerevisiae cell, a Yarrowia lipolytica cell, or Pichia pastoris cell.

A number of vectors exist for the expression of recombinant proteins in yeast host cells. Examples of vectors that may be used in yeast host cells include, for example, Yip type vectors, Yep type vectors, Yrp type vectors, Ycp type vectors, pGPD-2, pAO815, pGAPZ, pGAPZα, pHIL-D2, pHIL-S1, pPIC3.5K, pPIC9K, pPICZ, pPICZα, pPIC3K, pHWO10, pPUZZLE and 2 μm plasmids. Such vectors are known to the art and are, for example, described in Cregg et al, Mol Biotechnol (2000) 16(1): 23-52. Suitable promoter sequences for use in yeast host cells are also known and described, for example, in Mattanovich et al., Methods Mol. Biol., 2012, 824:329-58, and in Romanos et al., 1992, Yeast 8: 423-488. Examples of suitable promoters for use in yeast host cells include promoters of glycolytic enzymes, like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (PPGK), the glycerol aldehyde phosphate dehydrogenase promoter (PGAP), translation elongation factor promoter (PTEF), S. cerevisiae enolase (ENO-1), S. cerevisiae galactokinase (GAL1), S. cerevisiae alcohol dehydrogenase/glyceraldehyde-3-phosphate dehydrogenase (ADH1, ADH2/GAP), S. cerevisiae triose phosphate isomerase (TPI), S. cerevisiae metallothionein (CUP1), and S. cerevisiae 3-phosphoglycerate kinase (PGK), and the maltase gene promoter (MAL). Marker genes suitable for use in yeast host cells are also known to the art. Thus, antibiotic resistance markers, such as ampicillin resistance markers, can be used in yeast, as well as marker genes providing genetic functions for essential nutrients, for example, leucine (LEU2), tryptophan (TRP1 and TRP2), uracil (URA3, URA5, URA6), histidine (HIS3), and the like. Methods for introducing vectors into yeast host cells can, for example, be found in S Kawai et al, 2010, Bioeng. Bugs 1(6): 395-403.

Further, guidance with respect to the preparation of expression vectors and introduction thereof into host cells, including in E. coli cells, yeast cells, and other host cells, may be found in, for example: Sambrook et al., Molecular Cloning, a Laboratory Manual, Cold Spring Harbor Laboratory Press, 2012, Fourth Ed.

Referring next to FIGS. 14A-14G, shown therein are example biosynthetic pathways to make the multi-substituent psilocybin derivative compounds of the present disclosure, notably FIG. 14A, by way of example, illustrates how example S1₄, S2₆ multi-substituent psilocybin derivative compounds may be biosynthetically made. FIG. 14B, by way of further example, illustrates how further example S1₄, S2₆ multi-substituent psilocybin derivative compounds may be biosynthetically made. FIG. 14C by way of a further example, illustrates how further example S1₅, S2₆ multi-substituent psilocybin derivative compounds may be biosynthetically made. FIG. 14D, by way of example, illustrates how example S1₄, S25 multi-substituent psilocybin derivative compounds may be biosynthetically made. FIG. 14E, by way of example, illustrates how example S1₄, S2₇ multi-substituent psilocybin derivative compounds may be biosynthetically made. FIG. 14F, by way of example, illustrates how example S1₅, S2₆ multi-substituent psilocybin derivative compounds may be biosynthetically made. FIG. 14G, by way of yet a further example, illustrates how example S1₅, S2₇ multi-substituent psilocybin derivative compounds may be biosynthetically made. It is to be understood that, in addition to the pathways, enzymes and compounds shown in FIGS. 14A-14G other biosynthetic pathways may be used to make other multi-substituent psilocybin derivative compounds, comprising multiple substituents, wherein two or more of the carbon atoms C₂, C₄, C₅, C₆ and C₇ possess a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, including further, for example, the multi-substituent compounds shown in FIGS. 3A-3L, 4A-4I, 5A-5I, 6A-6I, and 7A-7F, and further including any multi-substituent psilocybin derivative compounds having a formula (I):

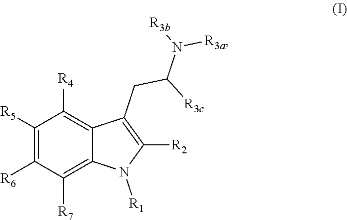

wherein, at least two of R₂, R₄, R₅, R₆, or R₇ are substituents independently selected from at least two of (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, and wherein each non-substituted R₂, R₅, R₆, or R₇ is a hydrogen atom and when R₄ is not substituted with any of the foregoing substituents, R₄ is a hydrogen atom, an O-alkyl group, an O-acyl group, or a phosphate group, wherein R₃ₐ and R_{3b} are each independently a hydrogen atom, an alkyl group, a cycloalkyl group, an acyl group, or an aryl group, and wherein and R_{3c} is a hydrogen atom or a carboxyl group.
   Following the teachings set forth herein, including by referring to the examples shown in FIGS. 14A-14G, those of skill In the art will be able to select appropriate psilocybin precursor compounds, psilocybin biosynthetic enzyme complement enzymes to biosynthetically make the multi-substituent psilocybin derivative compounds of the present disclosure.

Thus, referring further to FIG. 14A, and a psilocybin derivative precursor compound (LVII), the psilocybin biosynthetic enzyme complement can, for example, comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f).
   A first psilocybin derivative precursor compound having formula (I) can be used wherein two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom. A first multi-substituent psilocybin derivative compound having formula (I) can be formed wherein R_{3c} is a carboxyl group. For example, a psilocybin derivative precursor compound can be a chemical compound having a formula (LVIII):

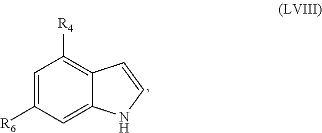

wherein R₄ is a hydroxy group, and wherein R₆ is a chlorine atom, and a first multi-substituent psilocybin derivative compound has a formula (LV):

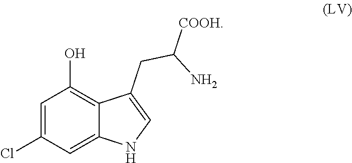 [SMILES: NC(Cc1c[nH]c2cc(Cl)cc(O)c12)C(=O)O]

can be formed in an in vivo or in vitro reaction catalyzed by a tryptophan synthase subunit B polypeptide.

Continuing to refer to FIG. 14A, a psilocybin biosynthetic enzyme complement can further comprise a tryptophan decarboxylase to decarboxylate the R₃—CH₂—CHNH₂COOH group of a first multi-substituent psilocybin derivative compound to thereby form a second multi-substituent psilocybin derivative having formula (I) wherein R₃ₐ and R_{3b} each are a hydrogen atom, a tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, and SEQ. ID NO 8;
(f) a nucleic acid sequence that encodes a functional variant of any one of the amino acid sequences set forth in SEQ. ID NO: 4, SEQ. ID NO: 6, and SEQ. ID NO 8; and
(g) a nucleic acid sequence that hybridizes under stringent conditions to any one of the nucleic acid sequences set forth in (a), (b), (c), (d), (e) or (f).

Continuing to refer to FIG. 14A, in an example embodiment, a psilocybin derivative precursor compound can be a chemical compound having a formula (LVIII):

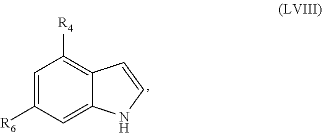

wherein R₄ is a fluorine atom, an amino group, or a hydroxy group, wherein R₆ is a fluorine atom, an amino group, a nitrile, or a bromine atom, and a first multi-substituent psilocybin derivative compound has a formula (LIX):

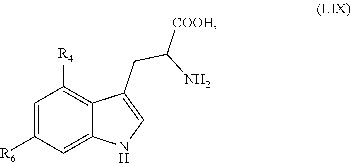

wherein R₄ is a fluorine atom, an amino group, or a hydroxy group, wherein R₆ is a fluorine atom, an amino group, a nitrile, or a bromine atom can be formed. The first multi-substituent psilocybin can be decarboxylated, and a second multi-substituent psilocybin can be formed, for example, a second multi-substituent psilocybin derivative having a formula (XXII), (XXVI), (XXIX), (LII), or (LIV):

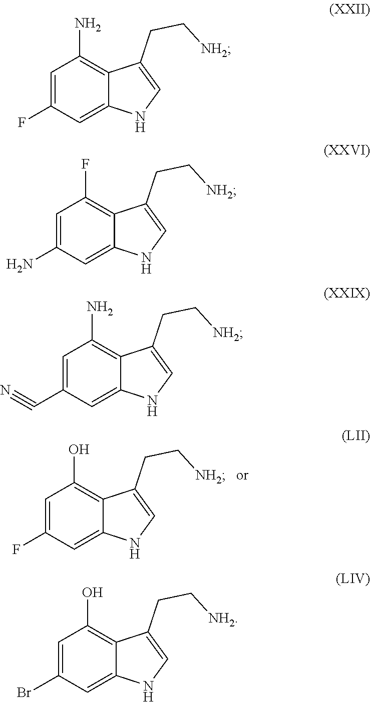

A psilocybin biosynthetic enzyme complement can further, for example, comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group. A N-acetyl transferase can be encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of the amino acid sequence set forth in SEQ. ID NO: 10; and
(g) a nucleic acid sequence that hybridizes under stringent conditions to any one of the nucleic acid sequences set forth in (a), (b), (c), (d), (e) or (f).

Thus, continuing to refer to FIG. 14A, in an example embodiment, a psilocybin derivative precursor compound can be a chemical compound having a formula (LVIII):

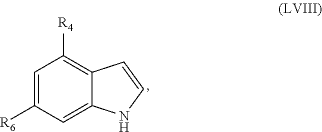

wherein R₄ is an acetamidyl group, a fluorine atom, an amino group, or a hydroxy group, wherein R₆ is a fluorine atom, an amino group, a nitrile, or a bromine atom, and a first multi-substituent psilocybin derivative compound having a formula (LIX):

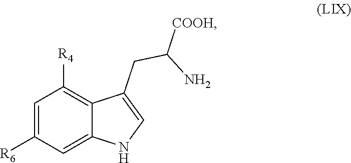

wherein R₄ is an acetamidyl group, a fluorine atom, an amino group, or a hydroxy group, wherein R₆ is a fluorine atom, an amino group, a nitrile, or a bromine atom can be formed. Then the first multi-substituent psilocybin derivative can be decarboxylated and a second multi-substituent psilocybin derivative having a formula (LX):

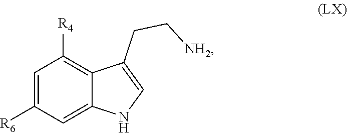

can be formed. Thereafter, the second multi-substituent psilocybin derivative can be acetylated, and a third multi-substituent psilocybin derivative can be formed, for example, a third multi-substituent psilocybin derivative having a formula (IX), (X), (XVIII), (XXI), (XXV), or (XXVIII):

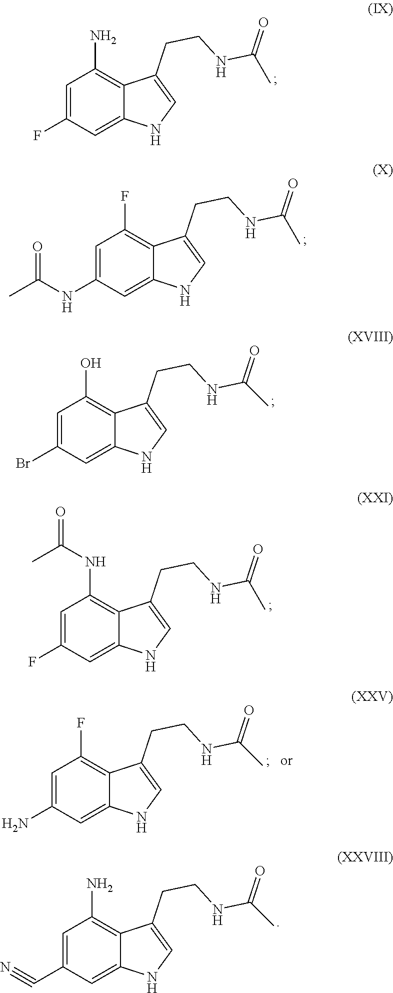

A psilocybin biosynthetic enzyme complement, in accordance herewith can further, for example, comprise an N-methyl transferase to methylate the R₃ amino group at R₃ and form a fourth multi-substituent psilocybin derivative having a chemical formula (I), wherein R₃ₐ and R_{3b} are each a methyl group, or wherein R₃ₐ is a hydrogen atom and R_{3b} is a methyl group. A N-methyl transferase can be encoded by a nucleic acid sequence selected from:
(a) SEQ. ID NO: 11 and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 1₄;
(f) a nucleic acid sequence that encodes a functional variant of any one of the amino acid sequences set forth in SEQ. ID NO: 12 and SEQ. ID NO 14; and
(g) a nucleic acid sequence that hybridizes under stringent conditions to any one of the nucleic acid sequences set forth in (a), (b), (c), (d), (e) or (f).

Continuing to refer to FIG. 14A, in an example embodiment, a psilocybin derivative precursor compound can be a chemical compound having a formula (LVIII):

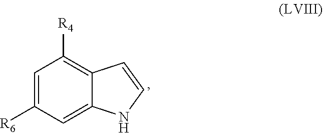

wherein R₄ is an amino group or a hydroxy group, wherein R₆ is a chlorine atom, a nitrile group, or a bromine atom, and a first multi-substituent psilocybin derivative compound having a formula (LIX):

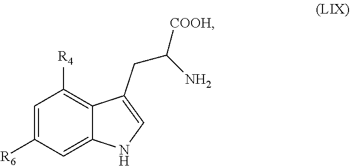

wherein R₄ is an amino group or a hydroxy group, wherein R₆ is a chlorine atom, a nitrile group, or a bromine atom can be formed. The first multi-substituent psilocybin derivative compound can be decarboxylated to form a second multi-substituent psilocybin derivative compound, wherein the second multi-substituent psilocybin derivative has a formula (LX):

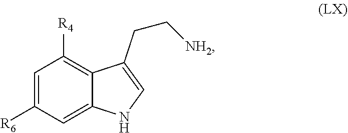

The third multi-substituent psilocybin derivative compound can be methylated to form a fourth multi-substituent psilocybin derivative, for example, a fourth multi-substituent psilocybin derivative compound having a formula (XXVII), (XL), or (LIII):

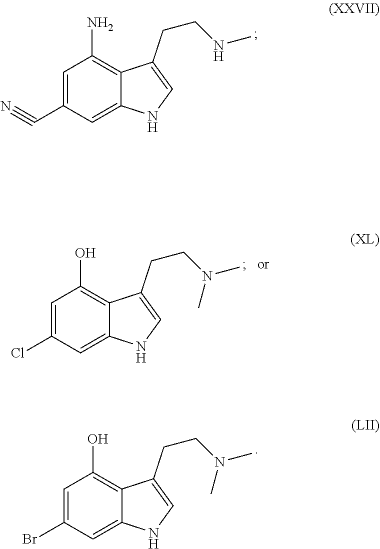 [SMILES: CNCCc1c[nH]c2cc(C#N)cc(N)c12 ; CN(C)CCc1c[nH]c2cc(Cl)cc(O)c12 ; CN(C)CCc1c[nH]c2cc(Br)cc(O)c12]

In accordance herewith, a psilocybin biosynthetic enzyme complement can, in a further example embodiment, comprise a prenyl transferase, encoded by a nucleic acid selected from:
(a) SEQ. ID NO: 15, SEQ. ID NO: 17, SEQ. ID NO: 19, and SEQ. ID NO 21;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 16, SEQ. ID NO: 18, SEQ. ID NO: 20, and SEQ. ID NO 22;
(f) a nucleic acid sequence that encodes a functional variant of any one of the amino acid sequences set forth in SEQ. ID NO: 16, SEQ. ID NO: 18, SEQ. ID NO: 20, and SEQ. ID NO 22; and
(g) a nucleic acid sequence that hybridizes under stringent conditions to any one of the nucleic acid sequences set forth in (a), (b), (c), (d), (e) or (f).

Referring, in this respect, next to FIG. 14B, in a further example embodiment, a psilocybin derivative precursor compound can be a chemical compound having a formula (LXI):

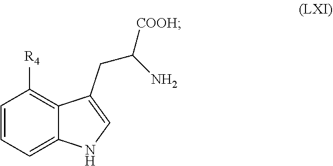

wherein R₄ is a hydroxy group, and a first multi-substituent psilocybin derivative compound can be formed, for example, a first formed multi-substituent psilocybin derivative compound having a formula (L):

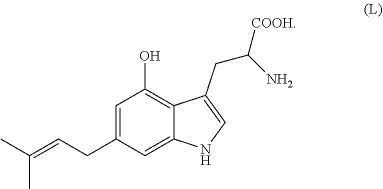 [SMILES: CC(C)=CCc1cc(O)c2c(CC(N)C(=O)O)c[nH]c2c1]

Continuing to referring FIG. 14B, a psilocybin biosynthetic enzyme complement can, in a further example embodiment, comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom. A tryptophan decarboxylase can be encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

A psilocybin biosynthetic enzyme complement can in a further example embodiment, comprise an N-methyl transferase to methylate the R₃ amino group at R₃ and form a fourth multi-substituent psilocybin derivative having a chemical formula (I), wherein R₃ₐ and R_{3b} are each a methyl group, or wherein R₃ₐ is a hydrogen atom and R_{3b} is a methyl group. A N-methyl transferase encoded by a nucleic acid sequence can be selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14;
(f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

Continuing to referring FIG. 14B, in an example embodiment, a psilocybin derivative precursor compound can be a chemical compound having a formula (LXI):

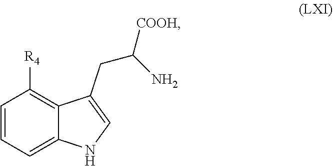

wherein R₄ is a propionyloxy or an acetoxy group, and a first formed multi-substituent psilocybin derivative compound having a formula (LIX):

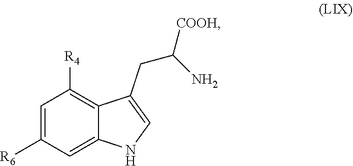

wherein R₄ is a propionyloxy or an acetoxy group, wherein R₆ is a prenyl group is formed. The first multi-substituent psilocybin derivative compound can be decarboxylated to form a second multi-substituent psilocybin derivative having a formula:

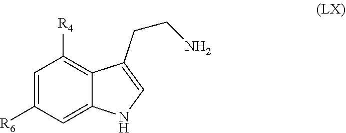

wherein R₄ is a propionyloxy or an acetoxy group, wherein R₆ is a prenyl group. The second multi-substituent psilocybin derivative can be methylated to for a third multi-substituent psilocybin derivative having a formula (XLI) or (XLII):

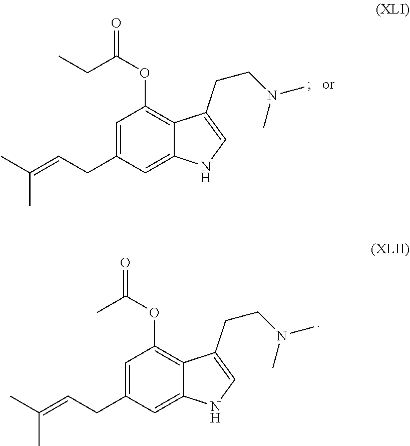

Referring further to FIG. 14B, its noted in order to prenylate the S1₄ psilocybin derivative precursor compound, dimethylallyl pyrophosphate (DMAPP) can be used as a substituent containing compound. Other prenyl containing compounds, such as geranyl pyrophosphate (GPP), farnesyl pyrophosphate (FPP), and geranylgeranyl pyrophosphate (GGPP) may alternatively be used as substituent containing compounds. As further shown in FIG. 14B, DMAPP itself may optionally be formed biosynthetically (in vitro or in vivo) from dimethylallyl alcohol DMAOH using acid phosphatase and isopentenyl phosphate kinase as catalyzing enzymes, as further described by Couillaud et al., 2019, ACS, Omega, 4, 7838-7859.

Referring next to FIG. 14C, in a further example embodiment, a psilocybin derivative precursor compound can be a chemical compound having a formula (LXII):

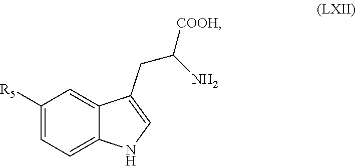

wherein R₅ is a chlorine or a fluorine atom, and a first multi-substituent psilocybin derivative compound having a formula (LXIII):

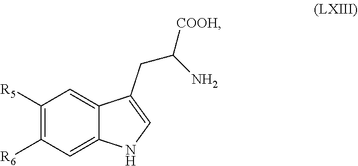

wherein R₅ is a chlorine or a fluorine atom, and wherein R₆ is a prenyl group can be formed. The first multi-substituent psilocybin derivative can be decarboxylated to form a second multi-substituent psilocybin derivative compound having a formula (XXXVI) or (XXXVIII):

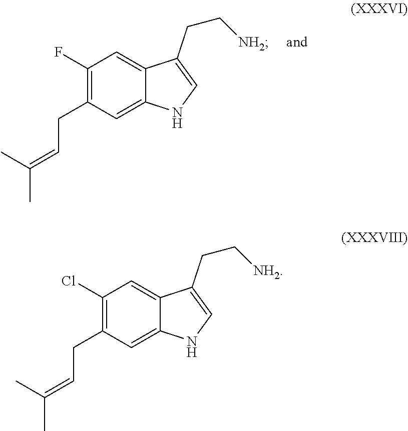 [SMILES: CC(C)=CCc1cc2[nH]cc(CCN)c2cc1F ; CC(C)=CCc1cc2[nH]cc(CCN)c2cc1Cl]

In one further example embodiment, a psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

Continuing to refer to FIG. 14C, a psilocybin derivative precursor compound can, in a further example embodiment, be a chemical compound having a formula (LXII):

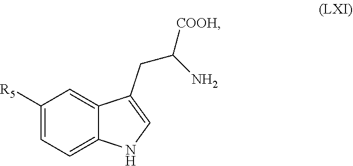

wherein R₅ is a chlorine or a fluorine atom, and a first multi-substituent psilocybin derivative compound having a formula (LXIII):

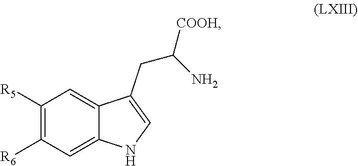

wherein R₅ is a chlorine or a fluorine atom, and wherein R₆ is a prenyl group can be formed. The first multi-substituent psilocybin derivative compound can be decarboxylated to form a second formed multi-substituent psilocybin derivative compound having a formula (LXIV):

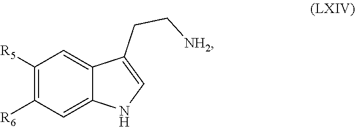

wherein R₅ is a chlorine or a fluorine atom, and wherein R₆ is a prenyl group. The second multi-substituent psilocybin derivative compound can be acetylated to form a wherein third multi-substituent psilocybin derivative having a formula (XXXV) or (XXXVII):

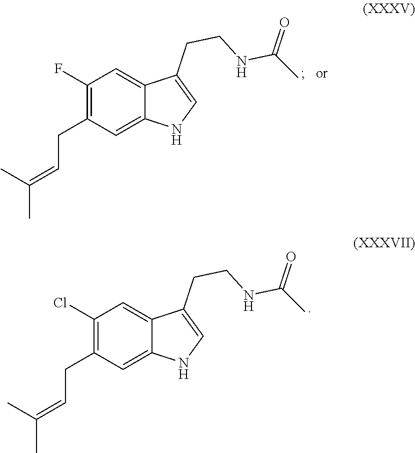

Referring further to FIG. 14C, similar to the biosynthetic pathway depicted in FIG. 14B, in order to prenylate the S15 psilocybin derivative precursor compound shown in FIG. 14C, dimethylallyl pyrophosphate (DMAPP) can be used as a substituent containing compound.

Referring next to FIG. 14D, and a psilocybin derivative precursor compound having formula (LVII), the psilocybin biosynthetic enzyme complement can, for example, comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f).

A first psilocybin derivative precursor compound having formula (I) can be used wherein two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom. A first multi-substituent psilocybin derivative compound having formula (I) can be formed wherein R_{3c} is a carboxyl group. For example, a psilocybin derivative precursor compound can be a chemical compound having a formula: (LXV):

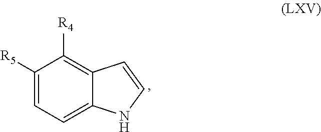

wherein R₄ is a hydroxy group, and wherein R₅ is a prenyl group, and a first formed multi-substituent psilocybin derivative compound having formula (LI):

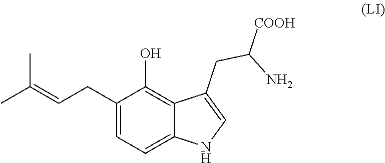 [SMILES: CC(C)=CCc1ccc2[nH]cc(CC(N)C(=O)O)c2c1O]

can be formed.

The psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

Continuing to refer to FIG. 14D, for example, a psilocybin derivative precursor compound can be a chemical compound having a formula (LXV):

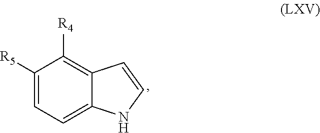

wherein R₄ is a fluorine atom and R₅ is nitrile group, a first formed multi-substituent psilocybin derivative compound having a formula (LXIX):

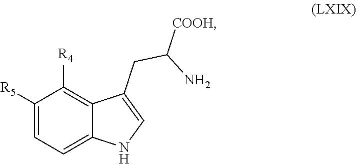

wherein R₄ is a fluorine atom and wherein R₅ is a nitrile group can be formed. A second formed multi-substituent psilocybin derivative compound can then be formed by decarboxylating the first multi-substituent derivative compound, the second multi-substituent psilocybin derivative compound having a formula (XIX):

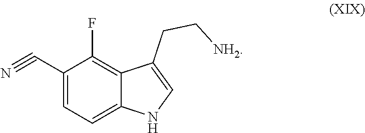 [SMILES: N#Cc1ccc2[nH]cc(CCN)c2c1F]

In a further embodiment, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

Continuing to refer to FIG. 14D, for example, the psilocybin derivative precursor compound can be a chemical compound having a formula (LXV):

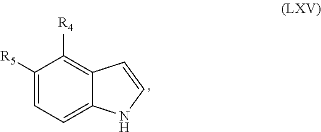

wherein R₄ is a fluorine atom and R₅ is a hydroxy group or a nitrile group, and a first formed multi-substituent psilocybin derivative compound having a formula (LXIX):

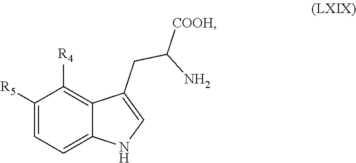

wherein R₄ is a fluorine atom and wherein R₅ is a hydroxy group or a nitrile group can be formed. The first formed multi-substituent psilocybin derivative can be decarboxylated to form a second multi-substituent psilocybin derivative compound having a formula (LXXII):

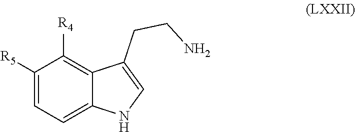

wherein R₄ is a fluorine atom, and wherein R₅ is a hydroxy group or a nitrile group. The second formed multi-substituent psilocybin derivative can then be acetylated to form a third multi-substituent psilocybin derivative having a formula (XVII) or (XX):

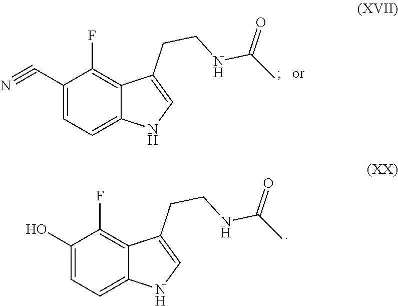 [SMILES: CC(=O)NCCc1c[nH]c2ccc(C#N)c(F)c12 ; CC(=O)NCCc1c[nH]c2ccc(O)c(F)c12]

Referring next to FIG. 14E, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group. For example, the psilocybin derivative precursor compound can be a chemical compound having a formula (LXVI):

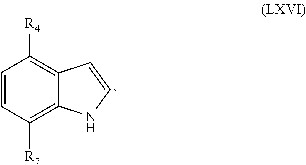

wherein R₄ is a hydroxy group, and wherein R₇ is a prenyl group, and a first formed multi-substituent psilocybin derivative compound having a formula (XLIX):

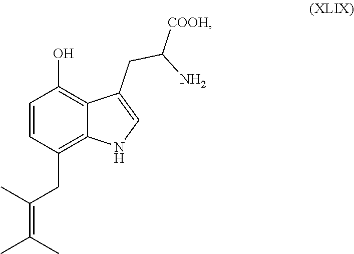 [SMILES: CC(C)=C(C)Cc1ccc(O)c2c(CC(N)C(=O)O)c[nH]c12]

can be formed.

In a further embodiment, the psilocybin biosynthetic enzyme complement can further comprise a tryptophan decarboxylase to decarboxylate the R₃—CH₂—CHNH₂COOH group, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

Continuing to refer to FIG. 14E, the psilocybin derivative precursor compound can be a chemical compound having a formula (LXVI):

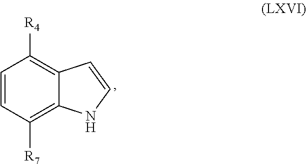

wherein R₄ is a fluorine atom and R₇ is nitrile group, and a first multi-substituent psilocybin derivative compound having a formula (LXX):

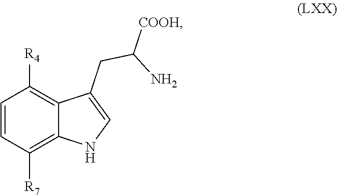

wherein R₄ is a fluorine atom and wherein R₇ is a nitrile group, can be formed. A second formed multi-substituent psilocybin derivative compound having a formula (XXIV):

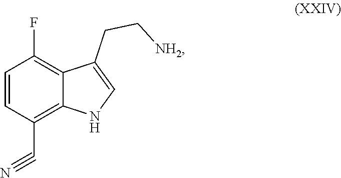 [SMILES: N#Cc1ccc(F)c2c(CCN)c[nH]c12]

can be formed by decarboxylating the first multi-substituent psilocybin derivative compound.

In a further embodiment, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9,
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

Continuing to refer to FIG. 14E, the psilocybin derivative precursor compound can be a chemical compound having a formula (LXVI):

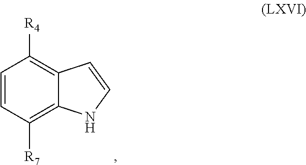

wherein R₄ is a fluorine atom or a chlorine atom and R₇ is a prenyl group or a nitrile group, and a multi-substituent psilocybin derivative compound having a formula (LXX):

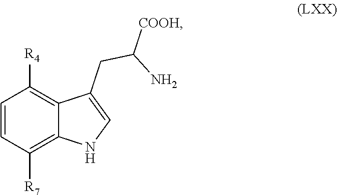

wherein R₄ is a fluorine atom or a chlorine atom and wherein R₇ is a prenyl group or a nitrile group can be formed. Upon decarboxylation of the first multi-substituent psilocybin derivative compound, a second multi-substituent psilocybin derivative compound having a formula (LXXIII):

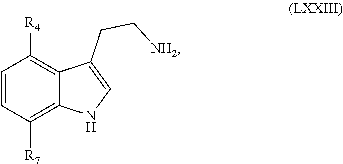

wherein R₄ is a fluorine atom or a chlorine atom, and wherein R₇ is a prenyl group or a nitrile group, can be formed. Following acetylation, a third multi-substituent psilocybin derivative having a formula (XXIII) or (XX):

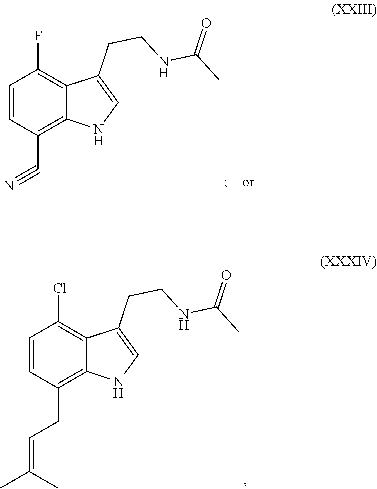

can be formed.

The psilocybin biosynthetic enzyme complement can further comprise an N-methyl transferase to methylate the R₃ amino group at R₃ and form a further multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14; (f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

In one embodiment, for example, the psilocybin derivative precursor compound can be a chemical compound having a formula (LXVI):

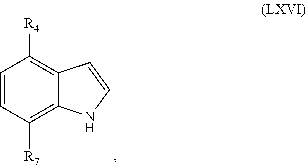

wherein R₄ is a chlorine atom and R₇ is a hydroxy group, and a first formed multi-substituent psilocybin derivative compound has a formula (LXX):

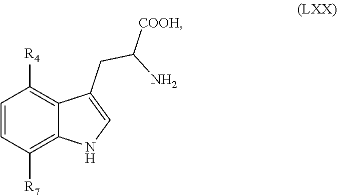

wherein R₄ is a hydroxy group and wherein R₇ is a chlorine atom can be formed. Following decarboxylation, a second formed multi-substituent psilocybin derivative compound having a formula (LXXIII):

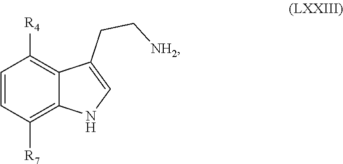

wherein R₄ is a hydroxy group, and wherein R₇ is a chlorine atom can be formed Following methylation a third multi-substituent psilocybin derivative having a formula (XXXIX):

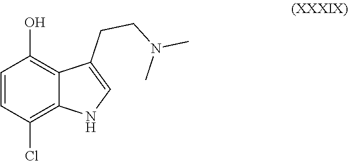 [SMILES: CN(C)CCc1c[nH]c2c(Cl)ccc(O)c12]

can be formed.

Referring next to FIG. 14F, in another embodiment, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group, and the psilocybin biosynthetic enzyme complement further comprises a tryptophan decarboxylase to decarboxylate a R₃—CH₂—CHNH₂COOH group of the first multi-substituent psilocybin derivative compound, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

Continuing to refer to FIG. 14F, the psilocybin derivative precursor compound can be a chemical compound having a formula (LXVII):

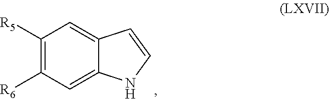

wherein R₅ is a fluorine atom, a chlorine atom, or a nitrile group and R₆ is a fluorine atom, an amino group or a prenyl group, and a first formed multi-substituent psilocybin derivative compound has a formula (LXIII):

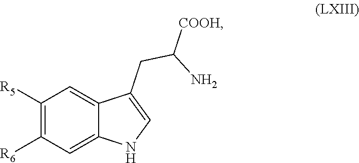

wherein R₅ is a fluorine atom, a chlorine atom, or a nitrile group and wherein R₆ is a is a fluorine atom, an amino group or a prenyl group, can be formed. Following decarboxylation, a second formed multi-substituent psilocybin derivative compound having a formula (XI), (XVI), (XXXVI) or (XXXVIII):

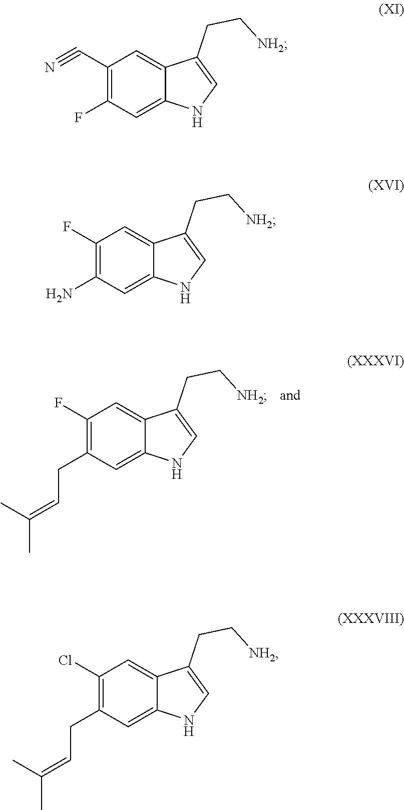 [SMILES: N#Cc1cc2c(CCN)c[nH]c2cc1F ; NCCc1c[nH]c2cc(N)c(F)cc12 ; CC(C)=CCc1cc2[nH]cc(CCN)c2cc1F ; CC(C)=CCc1cc2[nH]cc(CCN)c2cc1Cl]

can be formed.

The psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

Continuing to refer to FIG. 14F, in one embodiment, for example, the psilocybin derivative precursor compound can be a chemical compound having a formula (LXVII):

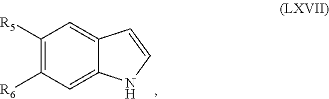

wherein R₅ is a fluorine atom or a chlorine atom and R₆ is an amino group, an acetamidyl group, or a prenyl group, and a first formed multi-substituent psilocybin derivative compound having a formula (LXIII):

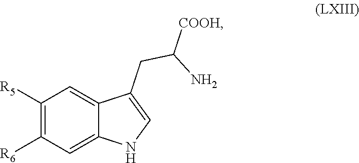

wherein R₅ is a fluorine atom or a chlorine atom and wherein R₆ is an amino group, an acetamidyl group, or a prenyl group can be formed. Following decarboxylation a second multi-substituent psilocybin derivative compound having a formula (LXXIV):

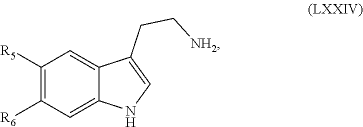

wherein R₅ is a fluorine atom or a chlorine atom, and wherein R₆ is an amino group, an acetamidyl group, or a prenyl group can be formed. Following acetylation, a third multi-substituent psilocybin derivative has a formula (XIV), (XV), (XXXV), or (XXXVII):

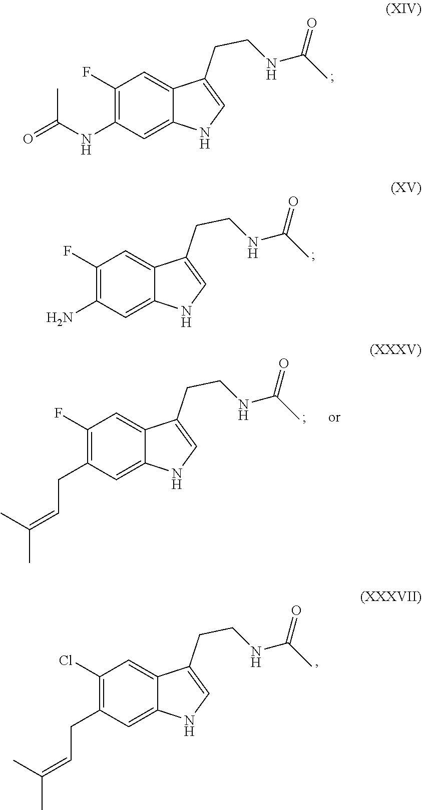

can be formed.

The psilocybin biosynthetic enzyme complement can further comprise an N-methyl transferase to methylate the R₃ amino group at R₃ and form a further multi-substituent psilocybin derivative having a chemical formula (I), wherein R₃ₐ and R_{3b} are each a methyl group, or wherein R₃ₐ is a hydrogen atom and R_{3b} is a methyl group, the N-methyl transferase encoded by a nucleic acid sequence selected from:
(a) SEQ. ID NO: 11, and SEQ. ID NO 13;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 12 and SEQ. ID NO 14;
(f) a nucleic acid sequence that encodes a functional variant of any one of the amino acid sequences set forth in SEQ. ID NO: 12, and SEQ. ID NO 14; and
(g) a nucleic acid sequence that hybridizes under stringent conditions to any one of the nucleic acid sequences set forth in (a), (b), (c), (d), (e) or (f).

Continuing to refer to FIG. 14F, the psilocybin derivative precursor compound can be a chemical compound having a formula (LXVII):

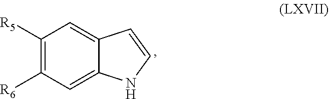

wherein R₅ is a chlorine atom and R₆ is a prenyl group, and a first formed multi-substituent psilocybin derivative compound has a formula (LXIII):

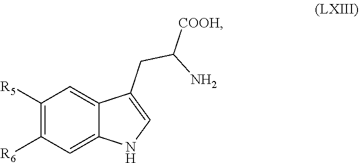

wherein R₅ is a chlorine atom and wherein R₆ is a prenyl group can be formed. Following decarboxylation, a second multi-substituent psilocybin derivative compound having a formula (LXXIV):

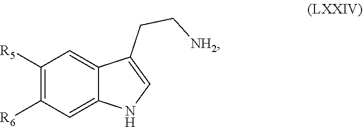

wherein R₅ is a chlorine atom, and wherein R₆ is a prenyl group can be formed. Following methylation, a third multi-substituent psilocybin derivative having a formula (XLIV):

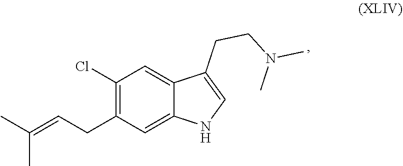 [SMILES: CC(C)=CCc1cc2[nH]cc(CCN(C)C)c2cc1Cl]

can be formed.

Referring next to FIG. 14G, in another embodiment, the psilocybin biosynthetic enzyme complement can comprise a tryptophan synthase subunit B polypeptide, encoded by a nucleic acid selected from:
(a) SEQ. ID NO: 1;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequences of (a);
(e) a nucleic acid sequence encoding a polypeptide having an amino acid sequences set forth in SEQ. ID NO: 2;
(f) a nucleic acid sequence that encodes a functional variant of the amino acid sequence set forth in SEQ. ID NO: 2; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), two of R₂, R₄, R₅, R₆, or R₇ are a substituent independently selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group, and the psilocybin biosynthetic enzyme complement further comprises a tryptophan decarboxylase to decarboxylate a R₃—CH₂—CHNH₂COOH group of the first multi-substituent psilocybin derivative compound, and thereby form a second multi-substituent psilocybin derivative having formula (I) wherein an R₃ₐ and R_{3b} each are a hydrogen atom, the tryptophan decarboxylase encoded by a nucleic acid sequence selected from:
(a) SEQ. ID NO: 3, SEQ. ID NO: 5, and SEQ. ID NO: 7;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 4, SEQ. ID NO: 6, SEQ. ID NO: and SEQ. ID NO 8;
(f) a nucleic acid sequence that encodes a functional variant of any one of the amino acid sequences set forth in SEQ. ID NO: 4, SEQ. ID NO: 6 and SEQ. ID NO 8; and
(g) a nucleic acid sequence that hybridizes under stringent conditions to any one of the nucleic acid sequences set forth in (a), (b), (c), (d), (e) or (f).

Continuing to refer to FIG. 14G, the psilocybin derivative precursor compound can, for example, be a chemical compound having a formula (LXVIII):

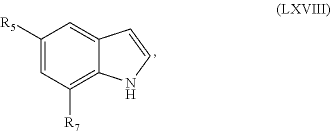

wherein R₅ is a fluorine atom, and R₇ is a nitro group or a prenyl group, a first formed multi-substituent psilocybin derivative compound having a formula (LXXI):

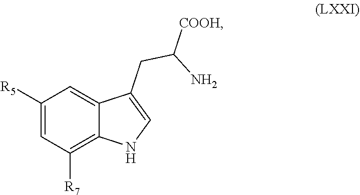

wherein R₅ is a fluorine atom, and wherein R₇ is a nitro group atom or a prenyl group can be formed. Following decarboxylation, a second formed multi-substituent psilocybin derivative compound having a formula (XIII) or (XXXIII):

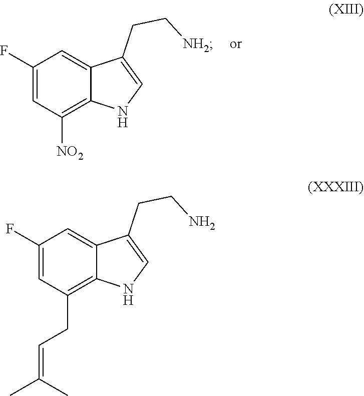 [SMILES: NCCc1c[nH]c2c([N+](=O)[O-])cc(F)cc12 ; CC(C)=CCc1cc(F)cc2c(CCN)c[nH]c12]

can be formed.

In one embodiment, the psilocybin biosynthetic enzyme complement can further comprise an N-acetyl transferase to acetylate the second psilocybin derivative having chemical formula (I) and thereby form a third multi-substituent psilocybin having chemical formula (I), wherein R₃ₐ is a hydrogen atom and R_{3b} is an acetyl group, the N-acetyl transferase encoded by a nucleic acid sequence selected from:
(a) SEQ. ID NO: 9;
(b) a nucleic acid sequence that is substantially identical to the nucleic acid sequence of (a);
(c) a nucleic acid sequence that is substantially identical to the nucleic acid sequence of (a) but for the degeneration of the genetic code;
(d) a nucleic acid sequence that is complementary to the nucleic acid sequence of (a);
(e) a nucleic acid sequence encoding a polypeptide having the amino acid sequence set forth in SEQ. ID NO: 10;
(f) a nucleic acid sequence that encodes a functional variant of any one of the amino acid sequences set forth in SEQ. ID NO: 10; and
(g) a nucleic acid sequence that hybridizes under stringent conditions to any one of the nucleic acid sequences set forth in (a), (b), (c), (d), (e) or (f).

Continuing to refer to FIG. 14G, the psilocybin derivative precursor compound can, for example, be a chemical compound having a formula (LXVIII):

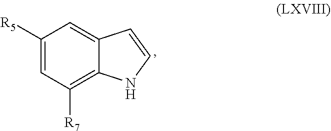

wherein R₅ is a fluorine atom, and R₇ is a nitro group or a prenyl group, a first formed multi-substituent psilocybin derivative compound having a formula (LXXI):

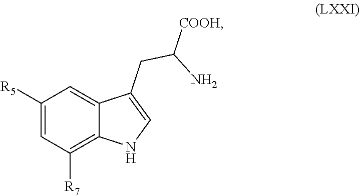

wherein R₅ is a fluorine atom, and wherein R₇ is a nitro group atom or a prenyl group can be formed. Following decarboxylation thereof a second formed multi-substituent psilocybin derivative compound has a formula (LXXV):

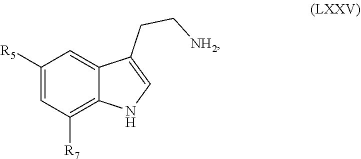

wherein R₅ is a fluorine atom, and wherein R₇ is a nitro group or a prenyl group can be formed. Following acetylation thereof a third multi-substituent psilocybin derivative has a formula (XII) or (XXXII):

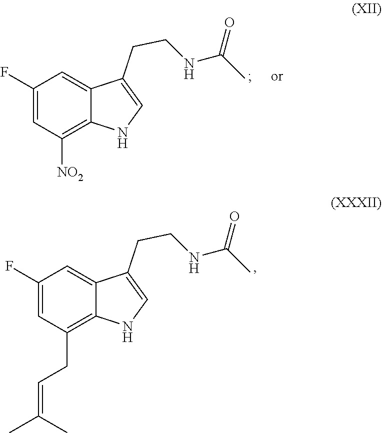 [SMILES: CC(=O)NCCc1c[nH]c2c([N+](=O)[O-])cc(F)cc12 ; CC(=O)NCCc1c[nH]c2c(CC=C(C)C)cc(F)cc12]

can be formed.

As hereinbefore noted, in some embodiments, a multi-substituent psilocybin derivative may be made by a employing a combination of synthetic and biosynthetic methods. Thus, for example, referring to FIG. 13D, compound 13-D4, may be made synthetically in accordance with the synthesis schematic shown in FIG. 13D (and as herein further described in Example 42). Compound 13-D4 then be prenylated, either in vitro or in vivo, using a prenyl transferase to form, for example, a C₆ prenylated derivative of compound 13-D4.

Thus, in one embodiment, a psilocybin biosynthetic enzyme complement can contain a prenyl transferase encoded by a nucleic acid selected from:
(a) SEQ. ID NO: 15, SEQ. ID NO: 17, SEQ. ID NO: 19, and SEQ. ID NO 21;
(b) a nucleic acid sequence that is substantially identical to any one of the nucleic acid sequences of (a);
(c) a nucleic acid sequence that is substantially identical to any one of the nucleic acid sequences of (a) but for the degeneration of the genetic code;
(d) a nucleic acid sequence that is complementary to any one of the nucleic acid sequences of (a);
(e) a nucleic acid sequence encoding a polypeptide having any one of the amino acid sequences set forth in SEQ. ID NO: 16, SEQ. ID NO: 18, SEQ. ID NO: 20, and SEQ. ID NO 22;
(f) a nucleic acid sequence that encodes a functional variant of any one of the amino acid sequences set forth in SEQ. ID NO: 16, SEQ. ID NO: 18, SEQ. ID NO: 20, and SEQ. ID NO 22; and
(g) a nucleic acid sequence that hybridizes under stringent conditions to any one of the nucleic acid sequences set forth in (a), (b), (c), (d), (e) or (f),
   wherein in the psilocybin derivative precursor compound having formula (LVIII), one of R₂, R₄, R₅, R₆, or R₇ is a substituent selected from (i) a halogen atom, (ii) a hydroxy group, (iii) a nitro group, (iv) a glycosyloxy group, (v) an amino group or an N-substituted amino group, (vi) a carboxyl group or a carboxylic acid derivative, (vii) an aldehyde or a ketone group, (viii) a prenyl group, and (ix) a nitrile group, wherein R₃ is a hydrogen atom, and wherein a first multi-substituent psilocybin derivative compound having formula (I) is formed wherein R_{3c} is a carboxyl group or a hydrogen atom.

In one embodiment, the psilocybin derivative precursor compound can have a formula (LXXVII):

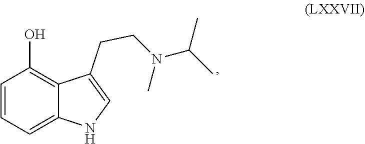 [SMILES: CC(C)N(C)CCc1c[nH]c2cccc(O)c12]

and a multi-substituent psilocybin derivative compound having the formula (LXXVI):

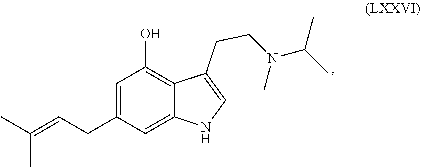 [SMILES: CC(C)=CCc1cc(O)c2c(CCN(C)C(C)C)c[nH]c2c1]

can be formed.

It will be clear to those of skill in the art that a significant variety of different psilocybin precursor compounds may be selected. FIGS. 14A and 14G in this respect provide guidance and allow a person of skill in the art to select appropriate psilocybin derivative precursor compounds and a matching a psilocybin biosynthetic enzyme complement.

Upon production by the host cells of a multi-substituent psilocybin compound in accordance with the methods of the present disclosure, the multi-substituent psilocybin derivative compounds may be extracted from the host cell suspension, and separated from other constituents within the host cell suspension, such as media constituents and cellular debris. Separation techniques will be known to those of skill in the art and include, for example, solvent extraction (e.g., butane, chloroform, ethanol), column chromatography based techniques, high-performance liquid chromatography (HPLC), for example, and/or countercurrent separation (CCS) based systems. The recovered multi-substituent psilocybin derivative compounds may be obtained in a more or less pure form, for example, a preparation of multi-substituent derivative psilocybin compounds of at least about 60% (w/w), about 70% (w/w), about 80% (w/w), about 90% (w/w), about 95% (w/w), about 96% (w/w), about 97% (w/w), about 98% (w/w) or about 99% (w/w) purity may be obtained. Thus, in this manner, multi-substituent psilocybin derivatives in more or less pure form may be prepared.

It will now be clear from the foregoing that novel multiple-substituent psilocybin derivatives are disclosed herein, as well as methods of making multiple-substituent psilocybin derivatives. The multiple-substituent psilocybin compounds may be formulated for use as a pharmaceutical drug or recreational drug.

### SUMMARY OF SEQUENCES

SEQ. ID NO: 1 sets forth a Pyrococcus furiosus nucleic acid sequence encoding tryptophan synthase subunit B polypeptide, named PfTrpB-BOA9.

SEQ. ID NO: 2 sets forth a deduced amino acid sequence of a Pyrococcus furiosus subunit B tryptophan synthase subunit B polypeptide, named PfTrpB-B0A9.

SEQ. ID NO: 3 sets forth a Bacillus atrophaeaus nucleic acid sequence encoding a tryptophan decarboxylase polypeptide, named BaTDC.

SEQ. ID NO: 4 sets forth a deduced amino acid sequence of Bacillus atrophaeaus tryptophan decarboxylase polypeptide, named BaTDC.

SEQ. ID NO: 5 sets forth a Clostridium sporidium nucleic acid sequence encoding a tryptophan decarboxylase polypeptide, named ClostSporTDC.

SEQ. ID NO: 6 sets forth a deduced amino acid sequence of Clostridium sporidium tryptophan decarboxylase polypeptide, named ClostSporTDC.

SEQ. ID NO: 7 sets forth a Psilocybe cubensis nucleic acid sequence encoding a PsiD polypeptide.

SEQ. ID NO: 8 sets forth a deduced amino acid sequence of a Psilocybe cubensis PsiD polypeptide.

SEQ. ID NO: 9 sets forth a Streptomyces griseofuscus nucleic acid sequence encoding an N-acetyl transferase, named PmsF.

SEQ. ID NO: 10 sets forth a deduced amino acid sequence of a Streptomyces griseofuscus an N-acetyl polypeptide, named PmsF.

SEQ. ID NO: 11 sets forth an Ephedra sinica nucleic acid sequence encoding an N-methyl transferase, named EsNMT.

SEQ. ID NO: 12 sets forth a deduced amino acid sequence of an Ephedra sinica an N-methyl transferase polypeptide, named EsNMT.

SEQ. ID NO: 13 sets forth a Psilocybe cubensis nucleic acid sequence encoding a PsiM polypeptide.

SEQ. ID NO: 1₄ sets forth a deduced amino acid sequence of a Psilocybe cubensis PsiM polypeptide.

SEQ. ID NO: 15 sets forth an Aspergillus fumigatus nucleic acid sequence encoding a tryptophan 7-prenyl transferase polypeptide, named 7DMATS.

SEQ. ID NO: 16 sets forth a deduced amino acid sequence of an Aspergillus fumigatus tryptophan 7-prenyl transferase polypeptide, named 7DMATS.

SEQ. ID NO: 17 sets forth a Streptomyces sp. RM-5-8 nucleic acid sequence encoding a 6-prenyl transferase polypeptide, named PriB.

SEQ. ID NO: 18 sets forth a deduced amino acid sequence of a Streptomyces sp. RM-5-8 6-prenyl transferase polypeptide, named PriB.

SEQ. ID NO: 19 sets forth a Streptomyces coelicolor nucleic acid sequence encoding a tryptophan 5-prenyl transferase polypeptide, named SCO7467.

SEQ. ID NO: 20 sets forth a deduced amino acid sequence of a Streptomyces coelicolor tryptophan 5-prenyl transferase polypeptide, named SCO7467.

SEQ. ID NO: 21 sets forth an Aspergillus fumigatus nucleic acid sequence encoding a tryptophan 4-prenyl transferase polypeptide, named FgaPT2.

SEQ. ID NO: 22 sets forth a deduced amino acid sequence of an Aspergillus fumigatus tryptophan 4-prenyl transferase polypeptide, named FgaPT2.

SEQ. ID NO: 23 sets forth a Psilocybe cubensis nucleic acid sequence encoding a PsiH polypeptide.

SEQ. ID NO: 24 sets forth a deduced amino acid sequence of a Psilocybe cubensis PsiH polypeptide.

SEQ. ID NO: 25 sets forth a Psilocybe cubensis nucleic acid sequence encoding a CPR polypeptide.

SEQ. ID NO: 26 sets forth a deduced amino acid sequence of a Psilocybe cubensis CPR polypeptide.

SEQ. ID NO: 27 sets forth an artificial nucleic acid useful as an integration cassette, named XII-4::TADH1-PsiH-HA-PPGK1-PTDH3-CPR-c-myc-TCYC1.

SEQ. ID NO: 28 sets forth an artificial nucleic acid useful as an integration cassette, named XII-5::TADH1-PsiK-V5-PPGK1-PTDH3-PsiM-FLAG-TCYC1.

SEQ. ID NO: 29 sets forth an artificial nucleic acid useful as an integration cassette, named pMM1-PTDH3-ClostSporTDC-His-TCYC1.

SEQ. ID NO: 30 sets forth an artificial nucleic acid useful as a promoter, named PGK1_promoter.

SEQ. ID NO: 31 sets forth an artificial nucleic acid useful as a promoter, named TDH3_promoter.

SEQ. ID NO: 32 sets forth an artificial nucleic acid useful as a promoter, named CLN1_promoter.

SEQ. ID NO: 33 sets forth an artificial nucleic acid useful as a promoter, named UGA1_promoter.

SEQ. ID NO: 34 sets forth an artificial nucleic acid useful as a vector, named pMM1.

SEQ. ID NO: 35 sets forth an artificial nucleic acid useful as a vector, named pCDM4.

SEQ. ID NO: 36 sets forth an artificial nucleic acid useful as a vector, named pET28a(+).

SEQ. ID NO: 37 sets forth an artificial nucleic acid useful as a vector, named pET23(+).

SEQ. ID NO: 38 sets forth an artificial nucleic acid encoding a polypeptide sequence useful as a tag, named HA-tag.

SEQ. ID NO: 39 sets forth an artificial polypeptide sequence useful as a tag, named HA-tag.

SEQ. ID NO: 40 sets forth an artificial nucleic acid sequence encoding a polypeptide sequence useful as a tag, named c-myc-tag.

SEQ. ID NO: 41 sets forth an artificial polypeptide sequence useful as a tag, named c-myc-tag.

SEQ. ID NO: 42 sets forth an artificial nucleic acid sequence encoding a polypeptide sequence useful as a tag, named FLAG-tag.

SEQ. ID NO: 43 sets forth an artificial polypeptide sequence useful as a tag, named FLAG-tag.

SEQ. ID NO: 44 sets forth an artificial nucleic acid sequence encoding a polypeptide sequence useful as a tag, named V5-tag.

SEQ. ID NO: 45 sets forth an artificial polypeptide sequence useful as a tag, named V5-tag.

SEQ. ID NO: 46 sets forth an artificial nucleic acid sequence encoding a polypeptide sequence useful as a tag, named His-tag.

SEQ. ID NO: 47 sets forth an artificial polypeptide sequence useful as a tag, named His-tag.

SEQ. ID NO: 48 sets forth a Psilocybe cubensis nucleic acid sequence encoding a PsiK polypeptide.

SEQ. ID NO: 49 sets forth a deduced amino acid sequence of a Psilocybe cubensis PsiK polypeptide.

SEQ. ID NO: 50 sets forth an artificial nucleic acid useful as an integration cassette, named X-3::TADH1-BaTDC-Flag-PPGK1-PTDH3-CPR-c-myc-TCYC1.

SEQ. ID NO: 51 sets forth an artificial nucleic acid useful as an integration cassette, named Xii-2::TADH1-PPGK1-PTDH3-PriB-His-TCYC1.

SEQ. ID NO: 52 sets forth an artificial nucleic acid useful as an integration cassette, named X-3::TADH1-ClostSporTDC-Flag-PPGK1-PTDH3-CPR-c-myc-TCYC1.

SEQ. ID NO: 53 sets forth an artificial nucleic acid useful as an integration cassette, named Xii-2::TADH1-PPGK1-PTDH3-Af-7DMATS-His-TCYC1.

SEQ. ID NO: 54 sets forth an artificial nucleic acid useful as a vector, named pET26b(+).

SEQ. ID NO: 1
ATGTGGTTCGGTGAGTTTGGTGGACAATATGTGCCAGAGACTTTAGTGGGTCCTCTTAA
GGAATTGGAAAAGGCATATAAAAGGTTCAAGGACGATGAGGAGTTCAACAGGCAACTAA
ACTATTATTTGAAGACATGGGCCGGTAGACCAACGCCCTTGTATTATGCTAAGAGGTTA
ACTGAAAAGATTGGCGGCGCGAAAGTGTATCTGAAAAGAGAAGACCTAGTTCATGGTGG
AGCACACAAGACAAATAATGCCATTGGACAAGCACTATTGGCAAAGCTAATGGGTAAAA
CTAGATTGATAGCTGAGACAGGAGCGGGTCAACATGGGGTCGCGACAGCGATGGCTGGT
GCACTACTGGGGATGAAGGTAGATATTTACATGGGTGCTGAGGACGTTGAGCGTCAGAA
ACTAAATGTCTTCAGGATGAAGCTATTAGGTGCCAATGTTATACCTGTAAATTCTGGCT
CAAGAACACTAAAGGACGCCTTCGACGAGGCTCTTAGAGACTGGGTTGCCACTTTCGAG
TATACTCATTACTTGATCGGTTCAGTGGTTGGACCACATCCATACCCAACCATCGTTAG
GGACTTTCAGAGCGTGATTGGTAGAGAGGCTAAGGCACAGATCTTAGAAGCAGAGGGAC
AGCTACCTGACGTCATAGTTGCCTGCGTCGGCGGTGGCTCTAACGCAATGGGTATATTC
TATCCATTCGTTAATGACAAGAAGGTTAAATTAGTAGGAGTCGAAGCTGGCGGAAAGGG
GTTAGAGTCGGGTAAACACTCAGCAAGCTTAAATGCAGGACAGGTAGGGGTGTCCCACG
GCATGTTGTCGTATTTCTTGCAAGACGAGGAAGGTCAGATAAAGCCAAGTCATTCAATT
GCTCCAGGCCTTGACCACCCCGGTGTTGGTCCAGAGCACGCTTACTTAAAGAAGATTCA
AAGGGCCGAGTACGTCGCTGTAACAGACGAAGAGGCATTGAAAGCTTTCCATGAGCTAT
CCAGAACTGAGGGGATTATACCCGCCCTTGAGTCTGCCCATGCTGTGGCGTACGCCATG
AAGTTAGCTAAAGAGATGTCCCGTGACGAAATCATCATTGTAAATCTATCAGGGAGAGG
AGACAAGGATTTGGACATTGTATTGAAGGCAAGCGGAAATGTTTGA
SEQ. ID NO: 2
MWFGEFGGQYVPETLVGPLKELEKAYKRFKDDEEFNRQLNYYLKTWAGRPTPLYYAKRL
TEKIGGAKVYLKREDLVHGGAHKTNNAIGQALLAKLMGKTRLIAETGAGQHGVATAMAG
ALLGMKVDIYMGAEDVERQKLNVERMKLLGANVIPVNSGSRTLKDAFDEALRDWVATFE
YTHYLIGSVVGPHPYPTIVRDFQSVIGREAKAQILEAEGQLPDVIVACVGGGSNAMGIF
YPFVNDKKVKLVGVEAGGKGLESGKHSASLNAGQVGVSHGMLSYFLQDEEGQIKPSHSI
APGLDHPGVGPEHAYLKKIQRAEYVAVTDEEALKAFHELSRTEGIIPALESAHAVAYAM
KLAKEMSRDEIIIVNLSGRGDKDLDIVLKASGNV
SEQ. ID NO: 3
ATGATGTCTGAAAATTTGCAATTGTCAGCTGAAGAAATGAGACAATTGGGTTACCAAGC
AGTTGATTTGATCATCGATCACATGAACCATTTGAAGTCTAAGCCAGTTTCAGAAACAA
TCGATTCTGATATCTTGAGAAATAAGTTGACTGAATCTATCCCAGAAAATGGTTCAGAT
CCAAAGGAATTGTTGCATTTCTTGAACAGAAACGTTTTTAATCAAATTACACATGTTGA
TCATCCACATTTCTTGGCTTTTGTTCCAGGTCCAAATAATTACGTTGGTGTTGTTGCAG
ATTTCTTGGCTTCTGGTTTTAATGTTTTTCCAACTGCATGGATTGCTGGTGCAGGTGCT
GAACAAATCGAATTGACTACAATTAATTGGTTGAAATCTATGTTGGGTTTTCCAGATTC
AGCTGAAGGTTTATTTGTTTCTGGTGGTTCAATGGCAAATTTGACAGCTTTGACTGTTG
CAAGACAGGCTAAGTTGAACAACGATATCGAAAATGCTGTTGTTTACTTCTCTGATCAA
ACACATTTCTCAGTTGATAGAGCATTGAAGGTTTTAGGTTTTAAACATCATCAAATCTG
TAGAATCGAAACAGATGAACATTTGAGAATCTCTGTTTCAGCTTTGAAGAAACAAATTA
AAGAAGATAGAACTAAGGGTAAAAAGCCATTCTGTGTTATTGCAAATGCTGGTACTACA
AATTGTGGTGCTGTTGATTCTTTGAACGAATTAGCAGATTTGTGTAACGATGAAGATGT
TTGGTTGCATGCTGATGGTTCTTATGGTGCTCCAGCTATCTTGTCTGAAAAGGGTTCAG
CTATGTTGCAAGGTATTCATAGAGCAGATTCTTTGACTTTAGATCCACATAAGTGGTTG
TTCCAACCATACGATGTTGGTTGTGTTTTGATCAGAAACTCTCAATATTTGTCAAAGAC
TTTTAGAATGATGCCAGAATACATCAAGGATTCAGAAACTAACGTTGAAGGTGAAATTA
ATTTCGGTGAATGTGGTATCGAATTGTCAAGAAGATTCAGAGCTTTGAAGGTTTGGTTG
TCTTTTAAAGTTTTCGGTGTTGCTGCTTTTAGACAAGCAATCGATCATGGTATCATGTT
AGCAGAACAAGTTGAAGCATTTTTGGGTAAAGCAAAAGATTGGGAAGTTGTTACACCAG
CTCAATTGGGTATCGTTACTTTTAGATACATTCCATCTGAATTGGCATCAACAGATACT
ATTAATGAAATTAATAAGAAATTGGTTAAGGAAATCACACATAGAGGTTTCGCTATGTT
ATCTACTACAGAATTGAAGGAAAAGGTTGTTATTAGATTGTGTTCAATTAATCCAAGAA
CTACAACTGAAGAAATGTTGCAAATCATGATGAAGATTAAAGCATTGGCTGAAGAAGTT
TCTATTTCATACCCATGTGTTGCTGAATAA
SEQ. ID NO: 4
MMSENLQLSAEEMRQLGYQAVDLIIDHMNHLKSKPVSETIDSDILRNKLTESIPENGSD
PKELLHFLNRNVENQITHVDHPHFLAFVPGPNNYVGVVADFLASGENVFPTAWIAGAGA
EQIELTTINWLKSMLGFPDSAEGLFVSGGSMANLTALTVARQAKLNNDIENAVVYESDQ
THFSVDRALKVLGFKHHQICRIETDEHLRISVSALKKQIKEDRTKGKKPFCVIANAGTT
NCGAVDSLNELADLCNDEDVWLHADGSYGAPAILSEKGSAMLQGIHRADSLTLDPHKWL
FQPYDVGCVLIRNSQYLSKTERMMPEYIKDSETNVEGEINFGECGIELSRRFRALKVWL
SFKVFGVAAFRQAIDHGIMLAEQVEAFLGKAKDWEVVTPAQLGIVTFRYIPSELASTDT
INEINKKLVKEITHRGFAMLSTTELKEKVVIRLCSINPRTTTEEMLQIMMKIKALAEEV
SISYPCVAE
SEQ. ID NO: 5
ATGAAGTTCTGGAGAAAGTACACACAACAAGAAATGGATGAAAAGATTACTGAATCTTT
GGAAAAGACTTTGAACTACGATAACACTAAGACAATCGGTATTCCAGGTACTAAGTTGG
ATGATACAGTTTTCTATGATGATCATTCTTTCGTTAAGCATTCACCATACTTGAGAACT
TTTATTCAAAACCCAAACCATATCGGTTGTCATACTTATGATAAGGCTGATATCTTGTT
CGGTGGTACATTCGATATCGAAAGAGAATTAATCCAATTGTTAGCAATCGATGTTTTGA
ACGGTAACGATGAAGAATTTGATGGTTACGTTACTCAAGGTGGTACAGAAGCTAACATC
CAAGCAATGTGGGTTTACAGAAACTACTTCAAGAAAGAAAGAAAGGCTAAGCATGAAGA
AATCGCTATCATCACTTCAGCAGATACACATTACTCTGCATACAAAGGTTCAGATTTGT
TGAACATCGATATTATTAAGGTTCCAGTTGATTTTTATTCAAGAAAAATTCAAGAAAAT
ACATTGGATTCAATTGTTAAAGAAGCTAAAGAAATTGGTAAAAAGTACTTCATCGTTAT
CTCTAACATGGGTACTACAATGTTTGGTTCAGTTGATGATCCAGATTTGTACGCTAACA
TCTTCGATAAGTACAATTTGGAATACAAAATTCATGTTGATGGTGCATTTGGTGGTTTT
ATATATCCAATTGATAATAAGGAATGTAAAACTGATTTCTCTAATAAGAACGTTTCTTC
AATCACATTAGATGGTCATAAGATGTTGCAAGCTCCATACGGTACTGGTATCTTCGTTT
CAAGAAAGAATTTGATCCATAACACTTTGACAAAGGAAGCAACTTACATCGAAAATTTG
GATGTTACATTGTCTGGTTCAAGATCTGGTTCAAATGCTGTTGCAATTTGGATGGTTTT
AGCTTCTTATGGTCCATACGGTTGGATGGAAAAGATTAATAAGTTGAGAAATAGAACTA
AATGGTTGTGTAAGCAATTGAACGATATGAGAATTAAATATTACAAAGAAGATTCAATG
AATATTGTTACAATTGAAGAACAATATGTTAATAAGGAAATCGCTGAAAAGTACTTTTT
AGTTCCAGAAGTTCATAACCCAACTAACAACTGGTACAAGATCGTTGTTATGGAACATG
TTGAATTGGATATCTTGAACTCTTTGGTTTACGATTTGAGAAAGTTTAATAAGGAACAT
TTGAAGGCAATGTAA
SEQ. ID NO: 6
MKFWRKYTQQEMDEKITESLEKTLNYDNTKTIGIPGTKLDDTVFYDDHSFVKHSPYLRT
FIQNPNHIGCHTYDKADILFGGTFDIERELIQLLAIDVLNGNDEEFDGYVTQGGTEANI
QAMWVYRNYFKKERKAKHEEIAIITSADTHYSAYKGSDLLNIDIIKVPVDFYSRKIQEN
TLDSIVKEAKEIGKKYFIVISNMGTTMFGSVDDPDLYANIFDKYNLEYKIHVDGAFGGF
IYPIDNKECKTDFSNKNVSSITLDGHKMLQAPYGTGIFVSRKNLIHNTLTKEATYIENL
DVTLSGSRSGSNAVAIWMVLASYGPYGWMEKINKLRNRTKWLCKQLNDMRIKYYKEDSM
NIVTIEEQYVNKEIAEKYFLVPEVHNPTNNWYKIVVMEHVELDILNSLVYDLRKENKEH
LKAM
SEQ. ID NO: 7
ATGCAGGTGATACCCGCGTGCAACTCGGCAGCAATAAGATCACTATGTCCTACTCCCGA
GTCTTTTAGAAACATGGGATGGCTCTCTGTCAGCGATGCGGTCTACAGCGAGTTCATAG
GAGAGTTGGCTACCCGCGCTTCCAATCGAAATTACTCCAACGAGTTCGGCCTCATGCAA
CCTATCCAGGAATTCAAGGCTTTCATTGAAAGCGACCCGGTGGTGCACCAAGAATTTAT
TGACATGTTCGAGGGCATTCAGGACTCTCCAAGGAATTATCAGGAACTATGTAATATGT
TCAACGATATCTTTCGCAAAGCTCCCGTCTACGGAGACCTTGGCCCTCCCGTTTATATG
ATTATGGCCAAATTAATGAACACCCGAGCGGGCTTCTCTGCATTCACGAGACAAAGGTT
GAACCTTCACTTCAAAAAACTTTTCGATACCTGGGGATTGTTCCTGTCTTCGAAAGATT
CTCGAAATGTTCTTGTGGCCGACCAGTTCGACGACAGACATTGCGGCTGGTTGAACGAG
CGGGCCTTGTCTGCTATGGTTAAACATTACAATGGACGCGCATTTGATGAAGTCTTCCT
CTGCGATAAAAATGCCCCATACTACGGCTTCAACTCTTACGACGACTTCTTTAATCGCA
GATTTCGAAACCGAGATATCGACCGACCTGTAGTCGGTGGAGTTAACAACACCACCCTC
ATTTCTGCTGCTTGCGAATCACTTTCCTACAACGTCTCTTATGACGTCCAGTCTCTCGA
CACTTTAGTTTTCAAAGGAGAGACTTATTCGCTTAAGCATTTGCTGAATAATGACCCTT
TCACCCCACAATTCGAGCATGGGAGTATTCTACAAGGATTCTTGAACGTCACCGCTTAC
CACCGATGGCACGCACCCGTCAATGGGACAATCGTCAAAATCATCAACGTTCCAGGTAC
CTACTTTGCGCAAGCCCCGAGCACGATTGGCGACCCTATCCCGGATAACGATTACGACC
CACCTCCTTACCTTAAGTCTCTTGTCTACTTCTCTAATATTGCCGCAAGGCAAATTATG
TTTATTGAAGCCGACAACAAGGAAATTGGCCTCATTTTCCTTGTGTTCATCGGCATGAC
CGAAATCTCGACATGTGAAGCCACGGTGTCCGAAGGTCAACACGTCAATCGTGGCGATG
ACTTGGGAATGTTCCATTTCGGTGGTTCTTCGTTCGCGCTTGGTCTGAGGAAGGATTGC
AGGGCAGAGATCGTTGAAAAGTTCACCGAACCCGGAACAGTGATCAGAATCAACGAAGT
CGTCGCTGCTCTAAAGGCTTAG
SEQ. ID NO: 8
MQVIPACNSAAIRSLCPTPESFRNMGWLSVSDAVYSEFIGELATRASNRNYSNEFGLMQ
PIQEFKAFIESDPVVHQEFIDMFEGIQDSPRNYQELCNMENDIFRKAPVYGDLGPPVYM
IMAKLMNTRAGESAFTRQRLNLHFKKLFDTWGLFLSSKDSRNVLVADQFDDRHCGWLNE
RALSAMVKHYNGRAFDEVELCDKNAPYYGENSYDDFFNRRERNRDIDRPVVGGVNNTTL
ISAACESLSYNVSYDVQSLDTLVFKGETYSLKHLLNNDPFTPQFEHGSILQGELNVTAY
HRWHAPVNGTIVKIINVPGTYFAQAPSTIGDPIPDNDYDPPPYLKSLVYFSNIAARQIM
FIEADNKEIGLIFLVFIGMTEISTCEATVSEGQHVNRGDDLGMFHFGGSSFALGLRKDC
RAEIVEKFTEPGTVIRINEVVAALKA
SEQ. ID NO: 9
ATGAACACCTTCAGAACAGCCACTGCCAGAGACATACCTGATGTAGCAGCAACTCTTAC
GGAAGCCTTCGCAACTGATCCACCCACGCAGTGGGTGTTCCCCGACGGTACTGCCGCCG
TCAGCAGGTTCTTTACACATGTTGCAGATAGGGTTCACACGGCCGGTGGTATTGTTGAG
CTACTACCAGACAGAGCCGCCATGATTGCATTGCCACCACACGTGAGGCTGCCAGGAGA
AGCTGCCGACGGAAGGCAGGCGGAAATTCAGAGAAGGCTGGCAGACAGGCACCCGCTGA
CACCTCACTACTACCTGCTGTTTTACGGAGTTAGAACGGCACACCAGGGTTCGGGATTG
GGCGGAAGAATGCTGGCCAGATTAACTAGCAGAGCTGATAGGGACAGGGTGGGTACATA
TACTGAGGCATCCACCTGGCGTGGCGCTAGACTGATGCTGAGACATGGATTCCATGCTA
CAAGGCCACTAAGATTGCCAGATGGACCCAGCATGTTTCCACTTTGGAGAGATCCAATC
CATGATCATTCTGAT
SEQ. ID NO: 10
MNTFRTATARDIPDVAATLTEAFATDPPTQWVFPDGTAAVSRFFTHVADRVHTAGGIVE
LLPDRAAMIALPPHVRLPGEAADGRQAEIQRRLADRHPLTPHYYLLFYGVRTAHQGSGL
GGRMLARLTSRADRDRVGTYTEASTWRGARLMLRHGFHATRPLRLPDGPSMFPLWRDPI
HDHSD
SEQ. ID NO: 11
ATGGGATCCATGGAAGAAGCAAAAATGGCGACCCTGGGCGGTGCGTCCTATGCGATGAT
TGTGAAAACGATGATGCGCTCTCTGGAAGCAAACCTGATTCCGGATTTTGTGCTGCGTC
GCCTGACGCGTATCCTGCTGGCTAGTCGCCTGAAACTGGGTTATAAGCAGACCGCTGAA
CTGCAACTGGCGGATCTGATGTCATTCGTTGCGTCGCTGAAAACGATGCCGATTGCCCT
GTGCACCGAAGAAGCAAAGGGTCAGCATTACGAACTGCCGACCAGCTTTTTCAAACTGG
TCCTGGGCAAACATCTGAAGTATAGCTCTGCCTACTTTTCTGAACACACCCGTACGCTG
GATGAAGCGGAAGAAGCCATGCTGGCACTGTATTGCGAACGCGCCAAAATTGAAGATGG
TCAGAAGATTCTGGACATCGGCTGTGGTTGGGGCAGTTTTTCCCTGTATGTGGCAGAAC
GTTACCCGAAATGCGAAATTACGGGCCTGTGTAACAGTTCCACCCAAAAAGCCTTCATC
GAACAGCAATGCAGCGAACGTCGCCTGTGTAATGTTACCATTTATGCAGATGACATCAG
CACCTTTGATACGGAATCTACCTACGACCGCATTATCAGCATCGAAATGTTCGAACACA
TGAAGAACTACAGTACGCTGCTGAAGAAAATTAGCAAGTGGATGAATCAGGAATGCCTG
CTGTTTGTCCATTATTTCTGTCACAAAACCTTTGCGTACCACTTCGAAGATGTGGACGA
AGATGACTGGATGGCTCGTTATTTCTTTACCGGCGGCACCATGCCGGCGTCATCGCTGC
TGCTGTACTTTCAGGATGACGTCTCAGTGGTTGATCATTGGCTGATTAACGGTAAACAC
TATGCTCAAACCTCGGAAGAATGGCTGAAGCGTATGGACCACAATCTGAGCTCTATTCT
GCCGATCTTTAACGAAACGTATGGCGAAAATGCGGCCAAAAAGTGGCTGGCATACTGGC
GCACCTTTTTCATCGCAGTTGCTGAACTGTTCAAATACAACGATGGCGAAGAATGGATG
GTGTCCCACTTCCTGTTCAAAAAGAAATAA
SEQ. ID NO: 12
MGSMEEAKMATLGGASYAMIVKTMMRSLEANLIPDFVLRRLTRILLASRLKLGYKQTAE
LQLADLMSFVASLKTMPIALCTEEAKGQHYELPTSFFKLVLGKHLKYSSAYFSEHTRTL
DEAEEAMLALYCERAKIEDGQKILDIGCGWGSFSLYVAERYPKCEITGLCNSSTQKAFI
EQQCSERRLCNVTIYADDISTEDTESTYDRIISIEMFEHMKNYSTLLKKISKWMNQECL
LFVHYFCHKTFAYHFEDVDEDDWMARYFFTGGTMPASSLLLYFQDDVSVVDHWLINGKH
YAQTSEEWLKRMDHNLSSILPIFNETYGENAAKKWLAYWRTFFIAVAELFKYNDGEEWM
VSHFLEKKK
SEQ. ID NO: 13
ATGCATATCAGAAATCCTTACCGTACACCAATTGACTATCAAGCACTTTCAGAGGCCTT
CCCTCCCCTCAAGCCATTTGTGTCTGTCAATGCAGATGGTACCAGTTCTGTTGACCTCA
CTATCCCAGAAGCCCAGAGGGCGTTCACGGCCGCTCTTCTTCATCGTGACTTCGGGCTC
ACCATGACCATACCAGAAGACCGTCTGTGCCCAACAGTCCCCAATAGGTTGAACTACGT
TCTGTGGATTGAAGATATTTTCAACTACACGAACAAAACCCTCGGCCTGTCGGATGACC
GTCCTATTAAAGGCGTTGATATTGGTACAGGAGCCTCCGCAATTTATCCTATGCTTGCC
TGTGCTCGGTTCAAGGCATGGTCTATGGTTGGAACAGAGGTCGAGAGGAAGTGCATTGA
CACGGCCCGCCTCAATGTCGTCGCGAACAATCTCCAAGACCGTCTCTCGATATTAGAGA
CATCCATTGATGGTCCTATTCTCGTCCCCATTTTCGAGGCGACTGAAGAATACGAATAC
GAGTTTACTATGTGTAACCCTCCATTCTACGACGGTGCTGCCGATATGCAGACTTCGGA
TGCTGCCAAAGGATTTGGATTTGGCGTGGGCGCTCCCCATTCTGGAACAGTCATCGAAA
TGTCGACTGAGGGAGGTGAATCGGCTTTCGTCGCTCAGATGGTCCGTGAGAGCTTGAAG
CTTCGAACACGATGCAGATGGTACACGAGTAACTTGGGAAAGCTGAAATCCTTGAAAGA
AATAGTGGGGCTGCTGAAAGAACTTGAGATAAGCAACTATGCCATTAACGAATACGTTC
AGGGGTCCACACGTCGTTATGCCGTTGCGTGGTCTTTCACTGATATTCAACTGCCTGAG
GAGCTTTCTCGTCCCTCTAACCCCGAGCTCAGCTCTCTTTTCTAG
SEQ. ID NO: 14
MHIRNPYRTPIDYQALSEAFPPLKPFVSVNADGTSSVDLTIPEAQRAFTAALLHRDFGL
TMTIPEDRLCPTVPNRLNYVLWIEDIFNYTNKTLGLSDDRPIKGVDIGTGASAIYPMLA
CARFKAWSMVGTEVERKCIDTARLNVVANNLQDRLSILETSIDGPILVPIFEATEEYEY
EFTMCNPPFYDGAADMQTSDAAKGFGFGVGAPHSGTVIEMSTEGGESAFVAQMVRESLK
LRTRCRWYTSNLGKLKSLKEIVGLLKELEISNYAINEYVQGSTRRYAVAWSFTDIQLPE
ELSRPSNPELSSLF
SEQ. ID NO: 15
ATGTCCATCGGAGCCGAGATCGATTCGCTGGTTCCTGCTCCACCGGGCCTCAACGGCAC
CGCTGCGGGCTATCCAGCCAAGACGCAGAAGGAGTTAAGCAACGGAGACTTTGACGCGC
ACGATGGTCTTTCTCTTGCACAACTGACACCGTACGATGTCTTGACGGCTGCACTTCCG
CTGCCGGCTCCGGCTTCGAGCACAGGGTTCTGGTGGCGGGAGACGGGCCCTGTTATGAG
CAAGCTTTTGGCCAAGGCGAACTACCCTCTTTACACTCATTACAAGTACCTTATGTTAT
ACCATACCCATATTCTCCCATTGTTGGGACCTCGACCGCCGCTCGAGAACTCGACGCAC
CCGTCGCCGAGTAACGCGCCGTGGAGGTCCTTCCTGACAGACGACTTCACTCCGCTCGA
GCCGAGCTGGAACGTGAACGGGAACTCGGAAGCACAGAGCACAATCCGTCTTGGTATTG
AACCTATAGGCTTTGAAGCCGGGGCTGCAGCGGACCCATTCAACCAAGCTGCCGTGACG
CAGTTCATGCACTCATACGAGGCAACCGAAGTCGGTGCCACGCTGACGCTGTTCGAGCA
CTTCCGCAACGACATGTTTGTTGGCCCAGAAACGTACGCTGCGTTAAGAGCGAAGATAC
CAGAAGGCGAGCATACCACACAGAGTTTCCTGGCGTTCGACCTGGACGCGGGTCGTGTC
ACCACAAAGGCGTACTTTTTCCCGATTCTCATGTCGTTGAAAACTGGACAGAGCACAAC
AAAGGTGGTCTCTGATTCCATTCTGCATCTAGCGCTGAAGAGTGAGGTGTGGGGTGTGC
AGACCATCGCCGCGATGTCGGTCATGGAGGCGTGGATAGGTAGCTACGGTGGCGCGGCA
AAGACGGAGATGATCAGCGTCGATTGCGTGAACGAGGCAGACTCTCGGATCAAGATATA
CGTGCGGATGCCACATACATCCTTGCGGAAGGTAAAAGAGGCGTACTGCTTAGGTGGGC
GGTTGACAGACGAGAACACAAAGGAGGGCCTGAAGCTGCTGGACGAGCTGTGGAGGACG
GTCTTCGGCATCGACGACGAGGACGCGGAGCTGCCACAGAATAGCCATCGCACCGCAGG
CACAATATTCAATTTCGAGCTGAGGCCAGGGAAATGGTTCCCCGAGCCCAAGGTATACC
TGCCCGTCCGACACTACTGTGAAAGTGATATGCAGATTGCTAGTCGGCTACAAACGTTC
TTTGGAAGGCTCGGATGGCACAACATGGAGAAAGATTATTGCAAGCATCTGGAAGATTT
GTTTCCCCATCATCCACTGTCCTCGTCAACGGGCACACACACCTTTCTCTCATTTTCGT
ATAAGAAGCAGAAGGGGGTCTATATGACCATGTATTATAATCTCCGGGTGTACAGCACC
TAA
SEQ. ID NO: 16
MSIGAEIDSLVPAPPGLNGTAAGYPAKTQKELSNGDFDAHDGLSLAQLTPYDVLTAALP
LPAPASSTGEWWRETGPVMSKLLAKANYPLYTHYKYLMLYHTHILPLLGPRPPLENSTH
PSPSNAPWRSELTDDFTPLEPSWNVNGNSEAQSTIRLGIEPIGFEAGAAADPENQAAVT
QFMHSYEATEVGATLTLFEHERNDMFVGPETYAALRAKIPEGEHTTQSFLAFDLDAGRV
TTKAYFFPILMSLKTGQSTTKVVSDSILHLALKSEVWGVQTIAAMSVMEAWIGSYGGAA
KTEMISVDCVNEADSRIKIYVRMPHTSLRKVKEAYCLGGRLTDENTKEGLKLLDELWRT
VFGIDDEDAELPQNSHRTAGTIFNFELRPGKWFPEPKVYLPVRHYCESDMQIASRLQTF
FGRLGWHNMEKDYCKHLEDLFPHHPLSSSTGTHTFLSFSYKKQKGVYMTMYYNLRVYST
SEQ. ID NO: 17
ATGGGAGGTCCGATGAGCGGTTTCCATTCGGGGGAGGCGCTGCTCGGTGACCTCGCCAC
CGGTCAGCTGACCAGGCTGTGCGAGGTGGCGGGGCTGACCGAGGCCGACACGGCGGCCT
ACACGGGGGTGCTGATCGAAAGTCTGGGGACGTCGGCCGGACGGCCGTTGTCCCTGCCA
CCCCCGTCGCGGACCTTTCTCTCCGACGACCACACCCCCGTGGAGTTCTCCCTGGCCTT
CCTGCCGGGACGCGCACCGCACCTGCGGGTCCTGGTGGAACCGGGCTGCTCCAGCGGCG
ACGACCTGGCGGAAAACGGCCGGGCCGGTCTGCGGGCGGTCCACACCATGGCGGACCGC
TGGGGATTCTCCACCGAGCAACTCGACCGGCTGGAGGACCTGTTCTTCCCCTCCTCCCC
CGAGGGCCCGCTGGCCCTGTGGTGCGCCCTGGAGCTCCGCTCCGGTGGGGTGCCGGGGG
TGAAGGTCTACCTCAACCCCGCGGCGAATGGCGCCGACCGGGCCGCCGAGACGGTACGC
GAGGCGCTGGCCAGGCTGGGCCACCTGCAGGCGTTCGACGCGCTGCCCCGGGCGGACGG
CTTCCCGTTCCTCGCCCTGGACCTCGGCGACTGGGACGCCCCGCGGGTGAAGATCTACC
TCAAACACCTCGGCATGTCCGCCGCCGACGCGGGCTCCCTCCCCCGGATGTCGCCCGCA
CCGAGCCGGGAGCAGCTGGAGGAGTTCTTCCGCACCGCCGGTGACCTCCCGGCCCCGGG
AGACCCGGGGCCCACCGAGGACACCGGCCGGCTCGCCGGGCGCCCCGCCCTCACCTGCC
ACTCCTTCACGGAGACGGCGACCGGGCGGCCCAGCGGCTACACCCTCCACGTGCCGGTC
CGCGACTACGTCCGGCACGACGGCGAGGCACGGGACCGGGCGGTGGCCGTGCTGCGCGA
ACATGACATGGACAGTGCGGCACTGGACCGGGCGCTGGCCGCCGTGAGCCCCCGCCCGC
TGAGTGACGGGGTGGGCCTGATCGCCTATCTGGCACTGGTCCACCAGCGCGGCCGGCCG
ACACGGGTGACCGTCTACGTCTCCTCCGAGGCGTACGAGGTGCGGCCGCCCCGCGAGAC
GGTCCCCACCCGCGACCGGGCGCGGGCACGGCTGTGA
SEQ. ID NO: 18
MGGPMSGFHSGEALLGDLATGQLTRLCEVAGLTEADTAAYTGVLIESLGTSAGRPLSLP
PPSRTFLSDDHTPVEFSLAFLPGRAPHLRVLVEPGCSSGDDLAENGRAGLRAVHTMADR
WGESTEQLDRLEDLFFPSSPEGPLALWCALELRSGGVPGVKVYLNPAANGADRAAETVR
EALARLGHLQAFDALPRADGEPFLALDLGDWDAPRVKIYLKHLGMSAADAGSLPRMSPA
PSREQLEEFFRTAGDLPAPGDPGPTEDTGRLAGRPALTCHSFTETATGRPSGYTLHVPV
RDYVRHDGEARDRAVAVLREHDMDSAALDRALAAVSPRPLSDGVGLIAYLALVHQRGRP
TRVTVYVSSEAYEVRPPRETVPTRDRARARL
SEQ. ID NO: 19
ATGAGGGCCGCGTCGACGGGCGCGGACCCGCAGGACGCATCCACGCTCGGCTCTTTCAC
CGGCGGCCAGTTGCGAAGACTCGGCTCGGTCGCCGGTCTGTCCCGCGCCGACGTCGAGA
CCTACGCACAGGTCCTGACCGACGCATTGGGCCCGGTGGCCCAGCGGCCGCTGAGCCTG
GCGCCGCCCACCCGCACCTTCCTGTCGGACGACCACACCCCCGTGGAGTTCTCCCTCTC
CTTCCGGCCCGGGGCGGCGCCCGCCATGCGGGTCCTCGTGGAACCGGGCTGCGGTGCGA
CCAGCCTGGCCGACAACGGCCGTGCCGGTCTTGAGGCGGTCCGCACGATGGCGCGGCGC
TGGCACTTCACCACCGACGCCCTCGACGAACTCCTGGACCTGTTCCTGCCGCCCGCTCC
GCAGGGCCCCCTCGCCCTGTGGTGCGCCCTGGAACTCAGGCCCGGGGGTGTACCGGGCG
TCAAGGTCTATCTGAACCCTGCGGTGGGCGGGGAGGAACGTTCCGCCGCGACGGTGCGC
GAGGCCCTGCGCCGGCTCGGGCACCACCAGGCCTTCGACAGCCTCCCCCAGGGCAGTGG
ATACCCGTTCCTCGCCCTGGACCTCGGGAACTGGACGGAGCCCCGGGCGAAGGTCTACC
TGCGCCACGACAACCTCACGGCCGGTCGGGCCGCACGGCTGTCCCGGACGGACTCGGGC
CTCGTGCCGACCGCGGTCGAGGGTTTCTTCCGCACCGCCGCGGGTCCCGGCTCCGACGC
GGGTGGGCTCGACGGGCGGCCTGCTCAGTCCTGCCACTCCTTCACCGACCCCGGCGCGG
AGCGGCCGAGCGGCTTCACCCTGTACATCCCGGTTCGTGACTACGTCCGGCATGACGGG
GAGGCCCTGGCGCGGGCGTCCACCGTGCTGCACCACCACGGCATGGACGCCTCCGTGCT
CCACCGCGCCCTGGCCGCCCTCACCGAGCGGCGGCCCGAGGACGGGGTGGGCCTGATCG
CCTACCTGGCCCTCGCCGGCCAACGGGACCAGCCGCCGCGGGTGACGGCCTACCTCTCC
TCGGAGGCCTACACGGTCCGGCCGCCGGTCGTGGAGACCGTCCGCCAACCGCTGTCGGT
CGGCTGA
SEQ. ID NO: 20
MRAASTGADPQDASTLGSFTGGQLRRLGSVAGLSRADVETYAQVLTDALGPVAQRPLSL
APPTRTFLSDDHTPVEFSLSFRPGAAPAMRVLVEPGCGATSLADNGRAGLEAVRTMARR
WHFTTDALDELLDLFLPPAPQGPLALWCALELRPGGVPGVKVYLNPAVGGEERSAATVR
EALRRLGHHQAFDSLPQGSGYPFLALDLGNWTEPRAKVYLRHDNLTAGRAARLSRTDSG
LVPTAVEGFFRTAAGPGSDAGGLDGRPAQSCHSFTDPGAERPSGFTLYIPVRDYVRHDG
EALARASTVLHHHGMDASVLHRALAALTERRPEDGVGLIAYLALAGQRDQPPRVTAYLS
SEAYTVRPPVVETVRQPLSVG
SEQ. ID NO: 21
ATGAAGGCAGCCAATGCCTCCAGTGCGGAGGCCTATCGAGTTCTTAGTCGCGCCTTTAG
ATTCGATAATGAAGATCAGAAGCTGTGGTGGCACAGCACTGCCCCGATGTTTGCAAAAA
TGCTGGAAACTGCCAACTACACCACACCTTGTCAGTATCAATACCTCATCACCTATAAG
GAGTGCGTAATTCCCAGTCTCGGATGCTATCCGACCAACAGCGCCCCCCGCTGGTTGAG
CATCCTCACTCGATACGGCACTCCGTTCGAATTGAGCCTAAATTGCTCTAATTCAATAG
TGAGATACACATTCGAGCCGATCAATCAACATACCGGAACAGATAAAGACCCATTCAAT
ACGCACGCCATCTGGGAGAGCCTGCAGCACCTGCTTCCACTGGAGAAGAGCATTGATCT
GGAGTGGTTCCGCCACTTCAAGCACGATCTCACCCTCAACAGTGAAGAATCTGCTTTTC
TGGCTCATAATGATCGCCTCGTGGGCGGCACTATCAGGACGCAGAACAAGCTCGCGCTC
GATCTGAAGGATGGCCGCTTTGCACTTAAGACGTACATATACCCGGCTCTCAAAGCTGT
CGTCACCGGCAAGACAATTCATGAGTTGGTCTTTGGCTCAGTCCGCCGGCTGGCAGTGA
GGGAGCCCCGAATCTTGCCCCCACTCAACATGCTGGAGGAATACATCCGATCACGCGGT
TCCAAGAGCACTGCCAGTCCCCGCCTAGTGTCCTGTGATCTGACCAGTCCTGCCAAGTC
GAGAATCAAGATCTACCTGCTGGAGCAGATGGTTTCACTAGAAGCCATGGAGGACCTGT
GGACTCTGGGCGGACGGCGCCGAGACGCTTCCACTTTAGAGGGGCTCTCTCTGGTGCGT
GAGCTTTGGGATCTGATCCAACTGTCGCCGGGATTGAAGTCCTATCCGGCGCCGTATCT
GCCTCTCGGGGTTATCCCAGACGAGAGGCTGCCGCTTATGGCCAATTTCACCCTGCACC
AGAATGACCCGGTCCCAGAGCCGCAAGTATATTTCACAACCTTCGGCATGAACGACATG
GCGGTGGCGGATGCCCTGACGACGTTCTTCGAGCGCCGGGGTTGGAGTGAAATGGCCCG
CACCTACGAAACTACTTTGAAGTCGTACTACCCCCATGCGGATCATGACAAACTTAACT
ACCTCCACGCCTACATATCCTTCTCCTACAGGGACCGTACCCCTTATCTGAGTGTCTAT
CTTCAATCCTTCGAGACAGGGGACTGGGCAGTTGCAAACTTATCCGAATCAAAGGTCAA
GTGTCAGGATGCGGCCTGTCAACCCACAGCTTTACCTCCAGATCTGTCAAAGACAGGGG
TATATTATTCCGGTCTCCACTGA
SEQ. ID NO: 22
MKAANASSAEAYRVLSRAFREDNEDQKLWWHSTAPMFAKMLETANYTTPCQYQYLITYK
ECVIPSLGCYPTNSAPRWLSILTRYGTPFELSLNCSNSIVRYTFEPINQHTGTDKDPFN
THAIWESLQHLLPLEKSIDLEWFRHFKHDLTLNSEESAFLAHNDRLVGGTIRTQNKLAL
DLKDGRFALKTYIYPALKAVVTGKTIHELVEGSVRRLAVREPRILPPLNMLEEYIRSRG
SKSTASPRLVSCDLTSPAKSRIKIYLLEQMVSLEAMEDLWTLGGRRRDASTLEGLSLVR
ELWDLIQLSPGLKSYPAPYLPLGVIPDERLPLMANFTLHQNDPVPEPQVYFTTFGMNDM
AVADALTTFFERRGWSEMARTYETTLKSYYPHADHDKLNYLHAYISFSYRDRTPYLSVY
LQSFETGDWAVANLSESKVKCQDAACQPTALPPDLSKTGVYYSGLH
SEQ. ID NO: 23
ATGATCGCTGTACTATTCTCCTTCGTCATTGCAGGATGCATATACTACATCGTTTCTCG
TAGAGTGAGGCGGTCGCGCTTGCCACCAGGGCCGCCTGGCATTCCTATTCCCTTCATTG
GGAACATGTTTGATATGCCTGAAGAATCTCCATGGTTAACATTTCTACAATGGGGACGG
GATTACAGTCTGTCTTGCCGCGTTGACTTCTAATATATGAACAGCTAATATATTGTCAG
ACACCGATATTCTCTACGTGGATGCTGGAGGGACAGAAATGGTTATTCTTAACACGTTG
GAGACCATTACCGATCTATTAGAAAAGCGAGGGTCCATTTATTCTGGCCGGTGAGCTGA
TGTTGAGTTTTTTGCAATTGAATTTGTGGTCACACGTTTCCAGACTTGAGAGTACAATG
GTCAACGAACTTATGGGGTGGGAGTTTGACTTAGGGTTCATCACATACGGCGACAGGTG
GCGCGAAGAAAGGCGCATGTTCGCCAAGGAGTTCAGTGAGAAGGGCATCAAGCAATTTC
GCCATGCTCAAGTGAAAGCTGCCCATCAGCTTGTCCAACAGCTTACCAAAACGCCAGAC
CGCTGGGCACAACATATTCGCCAGTAAGTACTACTTGAGGAAAATAGCGTACGCTTCGC
TGACCGGTCCGTACATCAAAGTCAGATAGCGGCAATGTCACTGGATATTGGTTATGGAA
TTGATCTTGCAGAAGACGACCCTTGGCTGGAAGCGACCCATTTGGCTAATGAAGGCCTC
GCCATAGCATCAGTGCCGGGCAAATTTTGGGTCGATTCGTTCCCTTCTCGTGAGCATCC
TTCTTCTATGTAGGAAGGGAAGGAGTCTAACAAGTGTTAGTAAAATACCTTCCTGCTTG
GTTCCCAGGTGCTGTCTTCAAGCGCAAAGCGAAGGTCTGGCGAGAAGCCGCCGACCATA
TGGTTGACATGCCTTATGAAACTATGAGGAAATTAGCAGTTAGTCAAATGCGTTCTCCC
CGTATTTTTTCAATACTCTAACTTCAGCTCACAGCCTCAAGGATTGACTCGTCCGTCGT
ATGCTTCAGCTCGTCTGCAAGCCATGGATCTCAACGGTGACCTTGAGCATCAAGAACAC
GTAATCAAGAACACAGCCGCAGAGGTTAATGTCGGTAAGTCAAAAGCGTCCGTCGGCAA
TTCAAAATTCAGGCGCTAAAGTGGGTCTTCTCACCAAGGTGGAGGCGATACTGTAAGGA
TTTCTCAATCGTTAGAGTATAAGTGTTCTAATGCAGTACATACTCCACCAACCAGACTG
TCTCTGCTATGTCTGCGTTCATCTTGGCCATGGTGAAGTACCCTGAGGTCCAGCGAAAG
GTTCAAGCGGAGCTTGATGCTCTGACCAATAACGGCCAAATTCCTGACTATGACGAAGA
AGATGACTCCTTGCCATACCTCACCGCATGTATCAAGGAGCTTTTCCGGTGGAATCAAA
TCGCACCCCTCGCTATACCGCACAAATTAATGAAGGACGACGTGTACCGCGGGTATCTG
ATTCCCAAGAACACTCTAGTCTTCGCAAACACCTGGTGAGGCTGTCCATTCATTCCTAG
TACATCCGTTGCCCCACTAATAGCATCTTGATAACAGGGCAGTATTAAACGATCCAGAA
GTCTATCCAGATCCCTCTGTGTTCCGCCCAGAAAGATATCTTGGTCCTGACGGGAAGCC
TGATAACACTGTACGCGACCCACGTAAAGCGGCATTTGGCTATGGACGACGAAATTGGT
AAGTGCGCTTTCAGAACCCCCCCTTCCGTTGACTAGTGCCATGCGCGCATACAATATCG
CTATTGATCTGATATAACTTCCCTGCGGCATTTATTTTGGCATTCCTTTAGTCCCGGAA
TTCATCTAGCGCAGTCGACGGTTTGGATTGCAGGGGCAACCCTCTTATCAGCGTTCAAT
ATCGAGCGACCTGTCGATCAGAATGGGAAGCCCATTGACATACCGGCTGATTTTACTAC
AGGATTCTTCAGGTAGCTAATTTCCGTCTTTGTGTGCATAATACCCCTAACGACGCACG
TTTACCTTTTTGTAAAGACACCCAGTGCCTTTCCAGTGCAGGTTTGTTCCTCGAACAGA
GCAAGTCTCACAGTCGGTATCCGGACCCTGA
SEQ. ID NO: 24
MIAVLFSFVIAGCIYYIVSRRVRRSRLPPGPPGIPIPFIGNMEDMPEESPWLTFLQWGR
DYNTDILYVDAGGTEMVILNTLETITDLLEKRGSIYSGRLESTMVNELMGWEFDLGFIT
YGDRWREERRMFAKEFSEKGIKQFRHAQVKAAHQLVQQLTKTPDRWAQHIRHQIAAMSL
DIGYGIDLAEDDPWLEATHLANEGLAIASVPGKFWVDSFPSLKYLPAWFPGAVEKRKAK
VWREAADHMVDMPYETMRKLAPQGLTRPSYASARLQAMDLNGDLEHQEHVIKNTAAEVN
VGGGDTTVSAMSAFILAMVKYPEVQRKVQAELDALTNNGQIPDYDEEDDSLPYLTACIK
ELFRWNQIAPLAIPHKLMKDDVYRGYLIPKNTLVFANTWAVLNDPEVYPDPSVFRPERY
LGPDGKPDNTVRDPRKAAFGYGRRNCPGIHLAQSTVWIAGATLLSAFNIERPVDQNGKP
IDIPADFTTGFFRHPVPFQCRFVPRTEQVSQSVSGP
SEQ. ID NO: 25
ATGGCTTCTAGTTCTTCCGATGTCTTCGTTTTGGGTCTAGGTGTTGTTTTGGCTGCCTT
GTATATCTTCAGAGACCAATTATTCGCTGCTTCTAAGCCAAAGGTGGCTCCAGTTTCCA
CTACGAAGCCTGCCAACGGTTCCGCTAACCCAAGAGACTTCATCGCCAAGATGAAACAA
GGTAAGAAGAGAATCGTAATCTTCTACGGTTCTCAAACTGGTACCGCTGAAGAATATGC
TATTCGTTTGGCTAAGGAAGCTAAGCAAAAGTTCGGTCTAGCCTCCTTGGTTTGTGATC
CAGAAGAATACGATTTTGAAAAGTTGGACCAATTGCCAGAAGATTCTATTGCTTTCTTC
GTCGTTGCTACCTATGGTGAAGGTGAACCTACAGACAACGCTGTCCAATTGTTGCAAAA
CTTGCAAGATGAAAGCTTCGAATTCTCCTCTGGTGAGAGAAAGTTGTCAGGTTTGAAGT
ACGTTGTTTTTGGTCTGGGTAACAAGACCTACGAACATTACAACCTCATTGGGAGAACT
GTTGACGCTCAATTGGCCAAGATGGGTGCTATCAGAATCGGTGAAAGAGGTGAAGGTGA
TGATGACAAGTCCATGGAAGAAGACTACTTGGAATGGAAGGATGGTATGTGGGAAGCGT
TTGCCACTGCTATGGGTGTTGAAGAAGGTCAAGGTGGTGACTCCGCTGATTTCGTCGTT
TCCGAATTGGAATCTCACCCACCAGAAAAGGTTTACCAAGGTGAATTTTCTGCTAGAGC
TTTAACCAAAACCAAGGGTATTCACGACGCTAAGAATCCTTTTGCTGCTCCAATTGCGG
TTGCTAGAGAATTGTTCCAATCTGTTGTCGATAGAAACTGTGTCCACGTCGAATTCAAC
ATTGAAGGCTCTGGTATCACCTATCAACACGGTGACCACGTTGGTTTGTGGCCATTGAA
TCCAGATGTTGAAGTCGAACGGTTGTTGTGTGTTTTAGGTTTAGCTGAAAAGAGAGATG
CTGTCATCTCCATTGAATCCTTAGACCCGGCTTTGGCTAAGGTTCCATTCCCAGTCCCA
ACTACTTACGGTGCTGTGTTGAGACACTACATTGACATCTCTGCTGTCGCCGGTAGACA
AATCTTGGGTACTTTGTCCAAATTCGCTCCAACCCCAGAAGCTGAAGCTTTCTTGAGAA
ACTTGAACACTAACAAGGAAGAATACCACAACGTCGTCGCTAACGGTTGTTTGAAATTG
GGTGAAATTTTGCAAATCGCTACCGGTAACGACATTACTGTCCCACCAACTACTGCCAA
CACCACCAAATGGCCAATTCCATTCGACATCATTGTTTCTGCCATCCCAAGATTGCAAC
CAAGATACTACTCTATCTCTTCTTCCCCAAAAATTCATCCAAACACCATCCACGCTACC
GTTGTTGTGCTCAAATACGAAAACGTTCCAACCGAACCAATCCCAAGAAAGTGGGTTTA
CGGTGTCGGTAGTAACTTCTTGTTGAATTTAAAGTACGCTGTTAACAAGGAACCAGTTC
CATACATCACTCAAAATGGCGAACAAAGAGTCGGTGTCCCGGAATACTTGATTGCTGGT
CCACGTGGTTCTTACAAGACTGAATCTTTCTACAAGGCTCCAATCCATGTTAGACGTTC
TACTTTCCGTTTGCCAACCAACCCAAAGTCTCCAGTCATCATGATTGGTCCAGGTACTG
GTGTCGCCCCATTCAGAGGCTTCGTTCAAGAAAGAGTTGCCTTGGCCAGAAGATCCATC
GAAAAGAACGGTCCTGACTCTTTGGCTGACTGGGGTCGTATTTCCTTGTTCTACGGTTG
TAGAAGATCCGACGAAGACTTCTTGTACAAGGACGAATGGCCACAATACGAAGCTGAGT
TGAAGGGTAAGTTCAAGTTGCACTGTGCTTTCTCCAGACAAAACTACAAGCCAGACGGT
TCTAAGATTTACGTCCAAGATTTGATCTGGGAAGACAGAGAACACATTGCCGATGCCAT
CTTAAACGGTAAGGGTTACGTCTACATCTGCGGTGAAGCTAAGTCCATGTCTAAACAAG
TTGAAGAAGTTCTAGCCAAGATCTTGGGCGAAGCCAAAGGTGGTTCCGGTCCAGTTGAA
GGTGTTGCTGAAGTCAAGTTACTGAAGGAACGGTCCAGATTGATGTTGGATGTCTGGTC
TAGG
SEQ. ID NO: 26
MASSSSDVFVLGLGVVLAALYIFRDQLFAASKPKVAPVSTTKPANGSANPRDFIAKMKQ
GKKRIVIFYGSQTGTAEEYAIRLAKEAKQKFGLASLVCDPEEYDFEKLDQLPEDSIAFF
VVATYGEGEPTDNAVQLLQNLQDESFEFSSGERKLSGLKYVVFGLGNKTYEHYNLIGRT
VDAQLAKMGAIRIGERGEGDDDKSMEEDYLEWKDGMWEAFATAMGVEEGQGGDSADFVV
SELESHPPEKVYQGEFSARALTKTKGIHDAKNPFAAPIAVARELFQSVVDRNCVHVEFN
IEGSGITYQHGDHVGLWPLNPDVEVERLLCVLGLAEKRDAVISIESLDPALAKVPFPVP
TTYGAVLRHYIDISAVAGRQILGTLSKFAPTPEAEAFLRNLNTNKEEYHNVVANGCLKL
GEILQIATGNDITVPPTTANTTKWPIPEDIIVSAIPRLQPRYYSISSSPKIHPNTIHAT
VVVLKYENVPTEPIPRKWVYGVGSNFLLNLKYAVNKEPVPYITQNGEQRVGVPEYLIAG
PRGSYKTESFYKAPIHVRRSTFRLPTNPKSPVIMIGPGTGVAPERGFVQERVALARRSI
EKNGPDSLADWGRISLFYGCRRSDEDFLYKDEWPQYEAELKGKFKLHCAFSRQNYKPDG
SKIYVQDLIWEDREHIADAILNGKGYVYICGEAKSMSKQVEEVLAKILGEAKGGSGPVE
GVAEVKLLKERSRLMLDVWS
SEQ. ID NO: 27
GTATCCGGCTGTTCCTTCATAGCCCTTTCAATGAACGTTGCAGCCCTTTGAAGATTGGC
CATTTTGTCAGGACTCGAGCCTGACAGTTGGACCAACGCAACTTTAATTTTTTGTGAAA
GAATCTTCGAAGCACTCATACTGGCGATCTTCACGCCCTCCTGCTATTACAAAAGCTGT
GTTTTTACAAGAATCAAATTAAGTTAGCAAGATATTATACAACATTATTGATAATTTCA
ATATCGTGTTCGTACCTGATGACGTATCTGTGCATTGATAAGGCCCGCATGGTTTCAGA
AAGCAGAGCGGAACGATTCCAAATTAGTGGCCTTGTGCTTTGCATGTCAATTGTGTTAC
CTTCAGCTCGTGGATTTGTTTTATCAATACACAGTCTACAGTCAAGAATTTTTTTTATC
AAATTTTGCGTTCGAGCGTATAAAATAGCCGCTGTAGCTACTTAAGTTCCTGTTCAGCG
ATAGTTTTTTTCCATCACACGTACTATGGCAATTAAGTCCTCAGCGAGCTCGCATGGAA
TGCGTGCGATGAGCGACCTCATGCTATACCTGAGAAAGCAACCTGACCTACAGGAAAGA
GTTACTCAAGAATAAGAATTTTCGTTTTAAAACCTAAGAGTCACTTTAAAATTTGTATA
CACTTATTTTTTTTATAACTTATTTAATAATAAAAATCATAAATCATAAGAAATTCGCT
TATTTAGAAGTGTCAACAACGTATCTACCAACGGAATGCGTGCGATTCAGGCGTAGTCT
GGAACGTCGTATGGGTATGGACCAGAGACGGATTGAGAAACTTGTTCGGTTCTTGGGAC
GAATCTACATTGGAATGGAACTGGGTGCCTGAAGAAACCAGTGGTGAAATCAGCTGGGA
TGTCAATTGGCTTACCGTTTTGGTCAACTGGTCTTTCAATGTTGAAAGCAGACAACAAA
GTAGCACCGGCAATCCAAACAGTAGATTGAGCTAGGTGAATACCTGGGCAGTTTCTTCT
ACCGTAACCGAAAGCAGCCTTTCTTGGGTCTCTAACAGTGTTGTCTGGTTTACCATCAG
GACCCAAGTATCTTTCTGGACGGAAAACGGATGGATCTGGATAGACTTCTGGGTCATTC
AAAACTGCCCAGGTGTTAGCAAAAACCAATGTGTTCTTTGGAATCAAATAACCTCTGTA
AACATCATCCTTCATCAATTTATGAGGGATGGCTAATGGAGCAATTTGGTTCCATCTGA
ATAATTCCTTGATACAAGCGGTCAAATATGGGAGTGAGTCGTCTTCCTCATCGTAGTCA
GGGATTTGACCGTTGTTGGTCAAAGCATCCAATTCAGCTTGAACCTTTCTTTGCACTTC
TGGGTATTTAACCATGGCCAATATGAAAGCGGACATAGCAGAGACGGTAGTGTCGCCAC
CACCAACGTTAACTTCAGCAGCAGTGTTCTTGATAACATGTTCTTGGTGTTCTAAATCA
CCGTTCAAGTCCATAGCTTGTAATCTAGCAGAAGCGTAAGATGGTCTGGTTAGACCTTG
TGGAGCCAACTTTCTCATAGTTTCGTATGGCATGTCAACCATGTGGTCAGCGGCTTCTC
TCCAGACCTTGGCCTTACGCTTGAAGACGGCACCTGGGAACCAAGCTGGCAAGTACTTC
AAAGATGGGAAGGAGTCAACCCAGAACTTACCTGGAACACTAGCAATGGCCAAACCTTC
GTTGGCTAAGTGGGTAGCTTCTAACCATGGGTCATCTTCGGCTAGATCGATACCGTAAC
CAATGTCCAAAGACATAGCAGCGATTTGATGTCTGATGTGTTGAGCCCACCGGTCCGGA
GTCTTGGTCAATTGTTGAACCAATTGGTGAGCAGCCTTGACTTGAGCGTGTCTGAATTG
CTTGATACCCTTTTCAGAGAATTCCTTAGCAAACATTCTTCTTTCTTCTCTCCAACGGT
CACCGTAAGTGATAAAACCCAGATCAAATTCCCAACCCATCAATTCATTGACCATAGTG
GATTCCAATCTACCGGAGTAGATGGAACCACGTTTTTCCAAGAGATCCGTGATAGTTTC
CAAGGTGTTTAAAATGACCATTTCGGTACCACCAGCATCTACGTACAAGATATCAGTGT
TGTAGTCACGACCCCATTGCAAGAAAGTCAACCATGGAGATTCTTCTGGCATGTCGAAC
ATGTTACCAATAAATGGGATTGGAATTCCTGGTGGACCAGGTGGCAATCTGGATCTACG
AACTCTTCTGGAGACGATGTAGTAAATACAACCAGCAATGACGAAAGAGAACAAGACAG
CAATCATTGTTTTATATTTGTTGTAAAAAGTAGATAATTACTTCCTTGATGATCTGTAA
AAAAGAGAAAAAGAAAGCATCTAAGAACTTGAAAAACTACGAATTAGAAAAGACCAAAT
ATGTATTTCTTGCATTGACCAATTTATGCAAGTTTATATATATGTAAATGTAAGTTTCA
CGAGGTTCTACTAAACTAAACCACCCCCTTGGTTAGAAGAAAAGAGTGTGTGAGAACAG
GCTGTTGTTGTCACACGATTCGGACAATTCTGTTTGAAAGAGAGAGAGTAACAGTACGA
TCGAACGAACTTTGCTCTGGAGATCACAGTGGGCATCATAGCATGTGGTACTAAACCCT
TTCCCGCCATTCCAGAACCTTCGATTGCTTGTTACAAAACCTGTGAGCCGTCGCTAGGA
CCTTGTTGTGTGACGAAATTGGAAGCTGCAATCAATAGGAAGACAGGAAGTCGAGCGTG
TCTGGGTTTTTTCAGTTTTGTTCTTTTTGCAAACAACAGTTTATTCCTGGCATCCACTA
AATATAATGGAGCCCGCTTTTTAAGCTGGCATCCAGAAAAAAAAAGAATCCCAGCACCA
AAATATTGTTTTCTTCACCAACCATCAGTTCATAGGTCCATTCTCTTAGCGCAACTACA
GAGAACAGGGGCACAAACAGGCAAAAAACGGGCACAACCTCAATGGAGTGATGCAACCT
GCCTGGAGTAAATGATGACACAAGGCAATTGACCCACGCATGTATCTATCTCATTTTCT
TACACCTTCTATTACCTTCTGCTCTCTCTGATTTGGAAAAAGCTGAAAAAAAAGGTTGA
AACCAGTTCCCTGAAATTATTCCCCTACTTGACTAATAAGTATATAAAGACGGTAGGTA
TTGATTGTAATTCTGTAAATCTATTTCTTAAACTTCTTAAATTCTACTTTTATAGTTAG
TCTTTTTTTTAGTTTTAAAACACCAAGAACTTAGTTTCGAATAAACACACATAAACAAA
CAAAATGGCTTCTAGTTCTTCCGATGTCTTCGTTTTGGGTCTAGGTGTTGTTTTGGCTG
CCTTGTATATCTTCAGAGACCAATTATTCGCTGCTTCTAAGCCAAAGGTGGCTCCAGTT
TCCACTACGAAGCCTGCCAACGGTTCCGCTAACCCAAGAGACTTCATCGCCAAGATGAA
ACAAGGTAAGAAGAGAATCGTAATCTTCTACGGTTCTCAAACTGGTACCGCTGAAGAAT
ATGCTATTCGTTTGGCTAAGGAAGCTAAGCAAAAGTTCGGTCTAGCCTCCTTGGTTTGT
GATCCAGAAGAATACGATTTTGAAAAGTTGGACCAATTGCCAGAAGATTCTATTGCTTT
CTTCGTCGTTGCTACCTATGGTGAAGGTGAACCTACAGACAACGCTGTCCAATTGTTGC
AAAACTTGCAAGATGAAAGCTTCGAATTCTCCTCTGGTGAGAGAAAGTTGTCAGGTTTG
AAGTACGTTGTTTTTGGTCTGGGTAACAAGACCTACGAACATTACAACCTCATTGGGAG
AACTGTTGACGCTCAATTGGCCAAGATGGGTGCTATCAGAATCGGTGAAAGAGGTGAAG
GTGATGATGACAAGTCCATGGAAGAAGACTACTTGGAATGGAAGGATGGTATGTGGGAA
GCGTTTGCCACTGCTATGGGTGTTGAAGAAGGTCAAGGTGGTGACTCCGCTGATTTCGT
CGTTTCCGAATTGGAATCTCACCCACCAGAAAAGGTTTACCAAGGTGAATTTTCTGCTA
GAGCTTTAACCAAAACCAAGGGTATTCACGACGCTAAGAATCCTTTTGCTGCTCCAATT
GCGGTTGCTAGAGAATTGTTCCAATCTGTTGTCGATAGAAACTGTGTCCACGTCGAATT
CAACATTGAAGGCTCTGGTATCACCTATCAACACGGTGACCACGTTGGTTTGTGGCCAT
TGAATCCAGATGTTGAAGTCGAACGGTTGTTGTGTGTTTTAGGTTTAGCTGAAAAGAGA
GATGCTGTCATCTCCATTGAATCCTTAGACCCGGCTTTGGCTAAGGTTCCATTCCCAGT
CCCAACTACTTACGGTGCTGTGTTGAGACACTACATTGACATCTCTGCTGTCGCCGGTA
GACAAATCTTGGGTACTTTGTCCAAATTCGCTCCAACCCCAGAAGCTGAAGCTTTCTTG
AGAAACTTGAACACTAACAAGGAAGAATACCACAACGTCGTCGCTAACGGTTGTTTGAA
ATTGGGTGAAATTTTGCAAATCGCTACCGGTAACGACATTACTGTCCCACCAACTACTG
CCAACACCACCAAATGGCCAATTCCATTCGACATCATTGTTTCTGCCATCCCAAGATTG
CAACCAAGATACTACTCTATCTCTTCTTCCCCAAAAATTCATCCAAACACCATCCACGC
TACCGTTGTTGTGCTCAAATACGAAAACGTTCCAACCGAACCAATCCCAAGAAAGTGGG
TTTACGGTGTCGGTAGTAACTTCTTGTTGAATTTAAAGTACGCTGTTAACAAGGAACCA
GTTCCATACATCACTCAAAATGGCGAACAAAGAGTCGGTGTCCCGGAATACTTGATTGC
TGGTCCACGTGGTTCTTACAAGACTGAATCTTTCTACAAGGCTCCAATCCATGTTAGAC
GTTCTACTTTCCGTTTGCCAACCAACCCAAAGTCTCCAGTCATCATGATTGGTCCAGGT
ACTGGTGTCGCCCCATTCAGAGGCTTCGTTCAAGAAAGAGTTGCCTTGGCCAGAAGATC
CATCGAAAAGAACGGTCCTGACTCTTTGGCTGACTGGGGTCGTATTTCCTTGTTCTACG
GTTGTAGAAGATCCGACGAAGACTTCTTGTACAAGGACGAATGGCCACAATACGAAGCT
GAGTTGAAGGGTAAGTTCAAGTTGCACTGTGCTTTCTCCAGACAAAACTACAAGCCAGA
CGGTTCTAAGATTTACGTCCAAGATTTGATCTGGGAAGACAGAGAACACATTGCCGATG
CCATCTTAAACGGTAAGGGTTACGTCTACATCTGCGGTGAAGCTAAGTCCATGTCTAAA
CAAGTTGAAGAAGTTCTAGCCAAGATCTTGGGCGAAGCCAAAGGTGGTTCCGGTCCAGT
TGAAGGTGTTGCTGAAGTCAAGTTACTGAAGGAACGGTCCAGATTGATGTTGGATGTCT
GGTCTGAACAAAAGTTAATTTCTGAAGAAGATTTGGAATGAATCGCGTGCATTCATCCG
CTCTAACCGAAAAGGAAGGAGTTAGACAACCTGAAGTCTAGGTCCCTATTTATTTTTTT
ATAGTTATGTTAGTATTAAGAACGTTATTTATATTTCAAATTTTTCTTTTTTTTCTGTA
CAGACGCGTGTACGCATGTAACATTATACTGAAAACCTTGCTTGAGAAGGTTTTGGGAC
GCTCGAAGATCGCGTCCCAATTCGCCCTATAGTGAGTCGTATTACGCGCGCTCACTGGC
CGTCGTTTTACAACGTCGTGACTGGGAAAACCCTGGCGTTACCCCTGCAGGACTAGTGC
TGAGGCATTAATAGTTTACTCAATTCTTGAAGCCAATTTGTACAATTCCCCATTAGAGT
CAAATAAAAGGATGCCTCACGGAGGTATGTTACCCGCGCTATTTCACATGGCTCATTGA
ATTAGAGGTGGAATTTGGTGTACCCTCCCCTCCTCATCTGATGAAGTAGTGATCCGACA
ATTCTTAAAAGTTGTAGACATTACTTTTACCACCAACTAAGTTGTATTTATATTGCTAC
CCTTATCCTTTTATATCTAACTAGCGCTCATAAGGTTGGGGCAATACTAAAACTGTGTT
CTTATTCAACTCATTAAATACGTGGCAGTACGTACCCTATTAGAAACAATAGGAAACAG
CAGAGTCGGAAGAAGCCAAATGCCAGATTTGAAGTCCAAAACCTTGTCAAGCCAATCTT
TGGGAGCGGCTATTCCTCCAGAAATTGTGTACCAAATACTTACATACCAGTTTAGGGAT
TTGTTAAGAAATGACCATCCAGGTACGGCAGAAA
SEQ. ID NO: 28
CAATCTGGCGGCTTGAGTTCTCAACATGTTTTATTTTTTACTTATATTGCTGGTAGGGT
AAAAAAATATAACTCCTAGGAATAGGTTGTCTATATGTTTTTGTCTTGCTTCTATAATT
GTAACAAACAAGGAAAGGGAAAATACTGGGTGTAAAAGCCATTGAGTCAAGTTAGGTCA
TCCCTTTTATACAAAATTTTTCAATTTTTTTTCCAAGATTCTTGTACGATTAATTATTT
TTTTTTTGCGTCCTACAGCGTGATGAAAATTTCGCCTGCTGCAAGATGAGCGGGAACGG
GCGAAATGTGCACGCGCACAACTTACGAAACGCGGATGAGTCACTGACAGCCACCGCAG
AGGTTCTGACTCCTACTGAGCTCTATTGGAGGTGGCAGAACCGGTACCGGAGGAGGCCG
CTATAACCGGTTTGAATTTATTGTCACAGTGTCACATCAGCATTAAGTCCTCAGCGAGC
TCGCATGGAATGCGTGCGATGAGCGACCTCATGCTATACCTGAGAAAGCAACCTGACCT
ACAGGAAAGAGTTACTCAAGAATAAGAATTTTCGTTTTAAAACCTAAGAGTCACTTTAA
AATTTGTATACACTTATTTTTTTTATAACTTATTTAATAATAAAAATCATAAATCATAA
GAAATTCGCTTATTTAGAAGTGTCAACAACGTATCTACCAACGGAATGCGTGCGATTCA
GGTGGAGTCCAAACCCAACAAAGGATTTGGAATTGGCTTACCGGCAGTGGAGGATTCCT
TTAGCAACGTGGAAGTGATTTCACCGTTGTCGTTGTTACCTCTAGCATCGTGGAAAGCA
GCAACACCCTTCTTAACAAAGTTGATTCTTTCTTCTTCAGAACCCCATTGCATGAAGTC
AGTCCACATAACAATGTGAGCGGCGATACCAGCGGTAACCTTGGCGTAGTTGATGGAAT
GCTTGGAAGTACGGGCGTAAGATTGCAAGTAAGCTTGTCTCATGGTTGTACCAACTTGT
TCGTCTTGGAATCTGCTAATCAAGTAACAGTCACCCAAGAAGTAACCCAAATCCAATGA
AGCTGGACCGTACTTACACAATTCCCAGTCTAAGATGTAGATCTTTTGCAACTTAGATG
GGTTACCTTCTTCAAGTTGCAACAAGATGTTCCCAGACCACAAGTCAGCCATGACCAAA
GTTTCTTCGGAGTGCATAACATCGTCAACTAGATCCTTGACAACAGTTGGCAACAATGG
ATCATCGACGCCGTATTTAGCGGCGTTTGGGATAATAGTTTGGTACAATTGGTCAGAGG
TGGTTCTACCGACAATGTTACCAGAGAAGAACTTGAATTCTGGGTCGTCTCTTCTTTCT
CTACCTATGTTGTGCAATCTGGCGACGAAACCACCAATCTCGGTACCAACCAATCTAGC
AATATCGGTAGCCAAAGGTGGCTTAGCAGTAACGTAGTCTAATAAGGTCTTCATTTTAC
CGACATCTTGCATAATCAAAGCATTGTTTTCCAAGTCATAGTTGAGACCTTCTGGAACA
GAGACAATACCATCAACACCACCCAAAACTTCTCTGTTAGCCATCATCAACTTGATAGC
TTGGTATTCGTAGACAGAACGTTCAACACCGATTTTGAAATCTTCATCAGTAGACATGT
GTGGTTGAGCGTGCTTCAAAATGATAGAAGTGTGACCTTGGTATGGAGCGTTCAATTTA
ATACGCCAGGTGACGTTAACGAAACCACCGGATAATCTCTTGACACCAGAAGTGTCAAC
ATCTAAAGACAAATGCTTGGTCAAATAGGTGATTAAACCGTCTTCAGTCTTCAAGTCAA
AGGCCATTGTTTTATATTTGTTGTAAAAAGTAGATAATTACTTCCTTGATGATCTGTAA
AAAAGAGAAAAAGAAAGCATCTAAGAACTTGAAAAACTACGAATTAGAAAAGACCAAAT
ATGTATTTCTTGCATTGACCAATTTATGCAAGTTTATATATATGTAAATGTAAGTTTCA
CGAGGTTCTACTAAACTAAACCACCCCCTTGGTTAGAAGAAAAGAGTGTGTGAGAACAG
GCTGTTGTTGTCACACGATTCGGACAATTCTGTTTGAAAGAGAGAGAGTAACAGTACGA
TCGAACGAACTTTGCTCTGGAGATCACAGTGGGCATCATAGCATGTGGTACTAAACCCT
TTCCCGCCATTCCAGAACCTTCGATTGCTTGTTACAAAACCTGTGAGCCGTCGCTAGGA
CCTTGTTGTGTGACGAAATTGGAAGCTGCAATCAATAGGAAGACAGGAAGTCGAGCGTG
TCTGGGTTTTTTCAGTTTTGTTCTTTTTGCAAACAACAGTTTATTCCTGGCATCCACTA
AATATAATGGAGCCCGCTTTTTAAGCTGGCATCCAGAAAAAAAAAGAATCCCAGCACCA
AAATATTGTTTTCTTCACCAACCATCAGTTCATAGGTCCATTCTCTTAGCGCAACTACA
GAGAACAGGGGCACAAACAGGCAAAAAACGGGCACAACCTCAATGGAGTGATGCAACCT
GCCTGGAGTAAATGATGACACAAGGCAATTGACCCACGCATGTATCTATCTCATTTTCT
TACACCTTCTATTACCTTCTGCTCTCTCTGATTTGGAAAAAGCTGAAAAAAAAGGTTGA
AACCAGTTCCCTGAAATTATTCCCCTACTTGACTAATAAGTATATAAAGACGGTAGGTA
TTGATTGTAATTCTGTAAATCTATTTCTTAAACTTCTTAAATTCTACTTTTATAGTTAG
TCTTTTTTTTAGTTTTAAAACACCAAGAACTTAGTTTCGAATAAACACACATAAACAAA
CAAAATGCATATCAGAAACCCATATAGAACTCCAATTGACTACCAAGCTTTGTCTGAAG
CTTTCCCACCATTGAAGCCATTTGTTTCCGTTAACGCTGATGGTACCTCCTCAGTTGAC
TTGACCATTCCAGAAGCCCAAAGAGCTTTTACCGCTGCCCTTTTGCACAGAGACTTCGG
CTTGACTATGACTATCCCAGAAGATCGTTTGTGTCCAACCGTTCCAAACAGATTGAACT
ACGTTTTGTGGATTGAAGACATTTTCAACTACACCAACAAGACTTTGGGTTTATCTGAC
GACCGTCCAATCAAGGGTGTTGATATCGGTACCGGTGCTTCTGCCATTTACCCAATGTT
GGCTTGCGCCAGATTCAAGGCTTGGTCCATGGTTGGTACTGAAGTTGAAAGAAAGTGTA
TCGACACTGCTAGATTAAACGTTGTTGCTAACAACTTGCAAGATCGTCTATCCATCTTG
GAAACCTCTATTGACGGTCCAATTTTAGTCCCAATTTTCGAAGCTACCGAAGAATACGA
ATACGAATTCACCATGTGTAACCCACCTTTCTACGATGGTGCCGCTGACATGCAAACTA
GCGATGCTGCAAAGGGTTTTGGTTTCGGTGTCGGTGCTCCACACTCTGGTACAGTCATC
GAAATGTCTACTGAAGGTGGTGAATCCGCTTTCGTGGCTCAAATGGTTAGAGAATCTCT
TAAGTTGAGAACCAGATGTAGATGGTACACTTCTAACTTAGGTAAGTTGAAATCTTTGA
AGGAAATTGTCGGTTTGTTGAAGGAATTAGAAATCTCTAATTACGCCATCAACGAATAT
GTCCAAGGTTCCACTAGAAGATACGCTGTCGCTTGGAGTTTCACTGATATCCAATTGCC
AGAAGAATTGTCCAGACCATCTAATCCTGAATTGTCCTCTTTGTTCGACTACAAGGATG
ACGATGACAAATGAATCGCGTGCATTCATCCGCTCTAACCGAAAAGGAAGGAGTTAGAC
AACCTGAAGTCTAGGTCCCTATTTATTTTTTTATAGTTATGTTAGTATTAAGAACGTTA
TTTATATTTCAAATTTTTCTTTTTTTTCTGTACAGACGCGTGTACGCATGTAACATTAT
ACTGAAAACCTTGCTTGAGAAGGTTTTGGGACGCTCGAAGATCGCGTACCCAATTCGCC
CTATAGTGAGTCGTATTACGCGCGCTCACTGGCCGTCGTTTTACAACGTCGTGACTGGG
AAAACCCTGGCGTTACCCCTGCAGGACTAGTGCTGAGGCATTAATGCAACTCAGAAGTT
TGACAGCAAGCAAGTTCATCATTCGAACTAGCCTTATTGTTTTAGTTCAGTGACAGCGA
ACTGCCGTACTCGATGCTTTATTTCTCACGGTAGAGCGGAAGAACAGATAGGGGCAGCG
TGAGAAGAGTTAGAAAGTAAATTTTTATCACGTCTGAAGTATTCTTATTCATAGGAAAT
TTTGCAAGGTTTTTTAGCTCAATAACGGGCTAAGTTATATAAGGTGTTCACGCGATTTT
CTTGTTATGTATACCTCTTCTCTGAGGAATGGTACTACTGTCCTGATGTAGGCTCCTTA
AATTGGTGGGCAAGAATAACTTATCGATATTTTGTATATTGGTCTTGGAGTTCACCACG
TAATGCCTGTTTAAGACCATCAGTTAACTCTAGTATTATTTGGTCTTGGCTACTGGCCG
TTTGCTATTATTCAAGTCTTTTGTGCCTTCCCGTCGGGTAAGGGAGTTATTTAGGGATA
CAGAATCTAACGAAAACTAAATCTCAATGATTAACTCTATTTAATCCTTTTTTGAAAGG
CAAAAGAGGTCCCTTGTTCACTTACAACGTTCTTAGCCAAATTCGCTTATCACTTACTA
CTTCACGATATACAGAAGTAAAAACATATAAAAAGATGTCTGTTTGTTTAGCCATCACA
AAAGGTATCGCAG
SEQ. ID NO: 29
TGTTTTATATTTGTTGTAAAAAGTAGATAATTACTTCCTTGATGATCTGTAAAAAAGAG
AAAAAGAAAGCATCTAAGAACTTGAAAAACTACGAATTAGAAAAGACCAAATATGTATT
TCTTGCATTGACCAATTTATGCAAGTTTATATATATGTAAATGTAAGTTTCACGAGGTT
CTACTAAACTAAACCACCCCCTTGGTTAGAAGAAAAGAGTGTGTGAGAACAGGCTGTTG
TTGTCACACGATTCGGACAATTCTGTTTGAAAGAGAGAGAGTAACAGTACGATCGAACG
AACTTTGCTCTGGAGATCACAGTGGGCATCATAGCATGTGGTACTAAACCCTTTCCCGC
CATTCCAGAACCTTCGATTGCTTGTTACAAAACCTGTGAGCCGTCGCTAGGACCTTGTT
GTGTGACGAAATTGGAAGCTGCAATCAATAGGAAGACAGGAAGTCGAGCGTGTCTGGGT
TTTTTCAGTTTTGTTCTTTTTGCAAACAACAGTTTATTCCTGGCATCCACTAAATATAA
TGGAGCCCGCTTTTTAAGCTGGCATCCAGAAAAAAAAAGAATCCCAGCACCAAAATATT
GTTTTCTTCACCAACCATCAGTTCATAGGTCCATTCTCTTAGCGCAACTACAGAGAACA
GGGGCACAAACAGGCAAAAAACGGGCACAACCTCAATGGAGTGATGCAACCTGCCTGGA
GTAAATGATGACACAAGGCAATTGACCCACGCATGTATCTATCTCATTTTCTTACACCT
TCTATTACCTTCTGCTCTCTCTGATTTGGAAAAAGCTGAAAAAAAAGGTTGAAACCAGT
TCCCTGAAATTATTCCCCTACTTGACTAATAAGTATATAAAGACGGTAGGTATTGATTG
TAATTCTGTAAATCTATTTCTTAAACTTCTTAAATTCTACTTTTATAGTTAGTCTTTTT
TTTAGTTTTAAAACACCAAGAACTTAGTTTCGAATAAACACACATAAACAAACAAAGGA
TCCATGATGAAGTTCTGGAGAAAGTACACACAACAAGAAATGGATGAAAAGATTACTGA
ATCTTTGGAAAAGACTTTGAACTACGATAACACTAAGACAATCGGTATTCCAGGTACTA
AGTTGGATGATACAGTTTTCTATGATGATCATTCTTTCGTTAAGCATTCACCATACTTG
AGAACTTTTATTCAAAACCCAAACCATATCGGTTGTCATACTTATGATAAGGCTGATAT
CTTGTTCGGTGGTACATTCGATATCGAAAGAGAATTAATCCAATTGTTAGCAATCGATG
TTTTGAACGGTAACGATGAAGAATTTGATGGTTACGTTACTCAAGGTGGTACAGAAGCT
AACATCCAAGCAATGTGGGTTTACAGAAACTACTTCAAGAAAGAAAGAAAGGCTAAGCA
TGAAGAAATCGCTATCATCACTTCAGCAGATACACATTACTCTGCATACAAAGGTTCAG
ATTTGTTGAACATCGATATTATTAAGGTTCCAGTTGATTTTTATTCAAGAAAAATTCAA
GAAAATACATTGGATTCAATTGTTAAAGAAGCTAAAGAAATTGGTAAAAAGTACTTCAT
CGTTATCTCTAACATGGGTACTACAATGTTTGGTTCAGTTGATGATCCAGATTTGTACG
CTAACATCTTCGATAAGTACAATTTGGAATACAAAATTCATGTTGATGGTGCATTTGGT
GGTTTTATATATCCAATTGATAATAAGGAATGTAAAACTGATTTCTCTAATAAGAACGT
TTCTTCAATCACATTAGATGGTCATAAGATGTTGCAAGCTCCATACGGTACTGGTATCT
TCGTTTCAAGAAAGAATTTGATCCATAACACTTTGACAAAGGAAGCAACTTACATCGAA
AATTTGGATGTTACATTGTCTGGTTCAAGATCTGGTTCAAATGCTGTTGCAATTTGGAT
GGTTTTAGCTTCTTATGGTCCATACGGTTGGATGGAAAAGATTAATAAGTTGAGAAATA
GAACTAAATGGTTGTGTAAGCAATTGAACGATATGAGAATTAAATATTACAAAGAAGAT
TCAATGAATATTGTTACAATTGAAGAACAATATGTTAATAAGGAAATCGCTGAAAAGTA
CTTTTTAGTTCCAGAAGTTCATAACCCAACTAACAACTGGTACAAGATCGTTGTTATGG
AACATGTTGAATTGGATATCTTGAACTCTTTGGTTTACGATTTGAGAAAGTTTAATAAG
GAACATTTGAAGGCAATGCATCATCATCATCATCATTAACCGCGGCTAGCTAAGATCCG
CTCTAACCGAAAAGGAAGGAGTTAGACAACCTGAAGTCTAGGTCCCTATTTATTTTTTT
ATAGTTATGTTAGTATTAAGAACGTTATTTATATTTCAAATTTTTCTTTTTTTTCTGTA
CAGACGCGTGTACGCATGTAACATTATACTGAAAACCTTGCTTGAGAAGGTTTTGGGAC
GCTCGAAGATCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGACG
CTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCC
TTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTAC
AGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAA
CAAACCACCGCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGATC
CTTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGTT
CATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGT
TGTGCAAAAAAGCGGTTAGCTCCTTCGGTCCTCCGATCGTTGTCAGAAGTAAGTTGGCC
GCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTA
TGCGGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCCTTTTTCAATA
TTATTGAAGCATTTATCAGGGTTATTGTCTCATGAGCGGATACATATTTGAATGTATTT
AGAAAAATAAACAAATAGGGGTTCCGCGCACATTTCCCCGAAAAGTGCCACCTGAACGA
AGCATCTGTGCTTCATTTTGTAGAACAAAAATGCAACGCGAGAGCGCTAATTTTTCAAA
CAAAGAATCTGAGCTGCATTTTTACAGAACAGAAATGCAACGCGAAAGCGCTATTTTAC
CAACGAAGAATCTGTGCTTCATTTTTGTAAAACAAAAATGCAACGCGAGAGCGCTAATT
TTTCAAACAAAGAATCTGAGCTGCATTTTTACAGAACAGAAATGCAACGCGAGAGCGCT
ATTTTACCAACAAAGAATCTATACTTCTTTTTTGTTCTACAAAAATGCATCCCGAGAGC
GCTATTTTTCTAACAAAGCATCTTAGATTACTTTTTTTCTCCTTTGTGCGCTCTATAAT
GCAGTCTCTTGATAACTTTTTGCACTGTAGGTCCGTTAAGGTTAGAAGAAGGCTACTTT
GGTGTCTATTTTCTCTTCCATAAAAAAAGCCTGACTCCACTTCCCGCGTTTACTGATTA
CTAGCGAAGCTGCGGGTGCATTTTTTCAAGATAAAGGCATCCCCGATTATATTCTATAC
CGATGTGGATTGCGCATACTTTGTGAACAGAAAGTGATAGCGTTGATGATTCTTCATTG
GTCAGAAAATTATGAACGGTTTCTTCTATTTTGTCTCTATATACTACGTATAGGAAATG
TTTACATTTTCGTATTGTTTTCGATTCACTCTATGAATAGTTCTTACTACAATTTTTTT
GTCTAAAGAGTAATACTAGAGATAAACATAAAAAATGTAGAGGTCGAGTTTAGATGCAA
GTTCAAGGAGCGAAAGGTGGATGGGTAGGTTATATAGGGATATAGCACAGAGATATATA
GCAAAGAGATACTTTTGAGCAATGTTTGTGGAAGCGGTATTCGCAATATTTTAGTAGCT
CGTTACAGTCCGGTGCGTTTTTGGTTTTTTGAAAGTGCGTCTTCAGAGCGCTTTTGGTT
TTCAAAAGCGCTCTGAAGTTCCTATACTTTCTAGAGAATAGGAACTTCGGAATAGGAAC
TTCAAAGCGTTTCCGAAAACGAGCGCTTCCGAAAATGCAACGCGAGCTGCGCACATACA
GCTCACTGTTCACGTCGCACCTATATCTGCGTGTTGCCTGTATATATATATACATGAGA
AGAACGGCATAGTGCGTGTTTATGCTTAAATGCGTACTTATATGCGTCTATTTATGTAG
GATGAAAGGTAGTCTAGTACCTCCTGTGATATTATCCCATTCCATGCGGGGTATCGTAT
GCTTCCTTCAGCACTACCCTTTAGCTGTTCTATATGCTGCCACTCCTCAATTGGATTAG
TCTCATCCTTCAATGCTATCATTTCCTTTGATATTGGATCATACTAAGAAACCATTATT
ATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTC
TGTAAGCGGATGCCGGGAGCAGACAAGCCCGTCAGGGCGCGTCAGCGGGTGTTGGCGGG
TGTCGGGGCTGGCTTAACTATGCGGCATCAGAGCAGATTGTACTGAGAGTGCACCATAT
TTCACACCGCATAGATCCGTCGAGTTCAAGAGAAAAAAAAAGAAAAAGCAAAAAGAAAA
AAGGAAAGCGCGCCTCGTTCAGAATGACACGTATAGAATGATGCATTACCTTGTCATCT
TCAGTATCATACTGTTCGTATACATACTTACTGACATTCATAGGTATACATATATACAC
ATGTATATATATCGTATGCTGCAGCTTTAAATAATCGGTGTCACTACATAAGAACACCT
TTGGTGGAGGGAACATCGTTGGTACCATTGGGCGAGGTGGCTTCTCTTATGGCAACCGC
AAGAGCCTTGAACGCACTCTCACTACGGTGATGATCATTCTTGCCTCGCAGACAATCAA
CGTGGAGGGTAATTCTGCTAGCCTCTGCAAAGCTTTCAAGAAAATGCGGGATCATCTCG
CAAGAGAGATCTCCTACTTTCTCCCTTTGCAAACCAAGTTCGACAACTGCGTACGGCCT
GTTCGAAAGATCTACCACCGCTCTGGAAAGTGCCTCATCCAAAGGCGCAAATCCTGATC
CAAACCTTTTTACTCCACGCACGGCCCCTAGGGCCTCTTTAAAAGCTTGACCGAGAGCA
ATCCCGCAGTCTTCAGTGGTGTGATGGTCGTCTATGTGTAAGTCACCAATGCACTCAAC
GATTAGCGACCAGCCGGAATGCTTGGCCAGAGCATGTATCATATGGTCCAGAAACCCTA
TACCTGTGTGGACGTTAATCACTTGCGATTGTGTGGCCTGTTCTGCTACTGCTTCTGCC
TCTTTTTCTGGGAAGATCGAGTGCTCTATCGCTAGGGGACCACCCTTTAAAGAGATCGC
AATCTGAATCTTGGTTTCATTTGTAATACGCTTTACTAGGGCTTTCTGCTCTGTCATCT
TTGCCTTCGTTTATCTTGCCTGCTCATTTTTTAGTATATTCTTCGAAGAAATCACATTA
CTTTATATAATGTATAATTCATTATGTGATAATGCCAATCGCTAAGAAAAAAAAAGAGT
CATCCGCTAGGTGGAAAAAAAAAAATGAAAATCATTACCGAGGCATAAAAAAATATAGA
GTGTACTAGAGGAGGCCAAGAGTAATAGAAAAAGAAAATTGCGGGAAAGGACTGTGTTA
TGACTTCCCTGACTAATGCCGTGTTCAAACGATACCTGGCAGTGACTCCTAGCGCTCAC
CAAGCTCTTAAAACGGGAATTTATGGTGCACTCTCAGTACACGCGCCAGATCTGTTTAG
CTTGCCTCGTCCCCGCCGGGTCACCCGGCCAGCGACATGGAGGCCCAGAATACCCTCCT
TGACAGTCTTGACGTGCGCAGCTCAGGGGCATGATGTGACTGTCGCCCGTACATTTAGC
CCATACATCCCCATGTATAATCATTTGCATCCATACATTTTGATGGCCGCACGGCGCGA
AGCAAAAATTACGGCTCCTCGCTGCAGACCTGCGAGCAGGGAAACGCTCCCCTCACAGA
CGCGTTGAATTGTCCCCACGCCGCGCCCCTGTAGAGAAATATAAAAGGTTAGGATTTGC
CACTGAGGTTCTTCTTTCATATACTTCCTTTTAAAATCTTGCTAGGATACAGTTCTCAC
ATCACATCCGAACATAAACAACCATGGGTAAGGAAAAGACTCACGTTTCGAGGCCGCGA
TTAAATTCCAACATGGATGCTGATTTATATGGGTATAAATGGGCTCGCGATAATGTCGG
GCAATCAGGTGCGACAATCTATCGATTGTATGGGAAGCCCGATGCGCCAGAGTTGTTTC
TGAAACATGGCAAAGGTAGCGTTGCCAATGATGTTACAGATGAGATGGTCAGACTAAAC
TGGCTGACGGAATTTATGCCTCTTCCGACCATCAAGCATTTTATCCGTACTCCTGATGA
TGCATGGTTACTCACCACTGCGATCCCCGGCAAAACAGCATTCCAGGTATTAGAAGAAT
ATCCTGATTCAGGTGAAAATATTGTTGATGCGCTGGCAGTGTTCCTGCGCCGGTTGCAT
TCGATTCCTGTTTGTAATTGTCCTTTTAACAGCGATCGCGTATTTCGTCTCGCTCAGGC
GCAATCACGAATGAATAACGGTTTGGTTGATGCGAGTGATTTTGATGACGAGCGTAATG
GCTGGCCTGTTGAACAAGTCTGGAAAGAAATGCATAAGCTTTTGCCATTCTCACCGGAT
TCAGTCGTCACTCATGGTGATTTCTCACTTGATAACCTTATTTTTGACGAGGGGAAATT
AATAGGTTGTATTGATGTTGGACGAGTCGGAATCGCAGACCGATACCAGGATCTTGCCA
TCCTATGGAACTGCCTCGGTGAGTTTTCTCCTTCATTACAGAAACGGCTTTTTCAAAAA
TATGGTATTGATAATCCTGATATGAATAAATTGCAGTTTCATTTGATGCTCGATGAGTT
TTTCTAATCAGTACTGACAATAAAAAGATTCTTGTTTTCAAGAACTTGTCATTTGTATA
GTTTTTTTATATTGTAGTTGTTCTATTTTAATCAAATGTTAGCGTGATTTATATTTTTT
TTCGCCTCGACATCATCTGCCCAGATGCGAAGTTAAGTGCGCAGAAAGTAATATCATGC
GTCAATCGTATGTGAATGCTGGTCGCTATACTGCTGTCGATTCGATACTAACGCCGCCA
TCCAGTGTCGAATTCGCCATTCAGGCTGCGCAACTGTTGGGAAGGGCGATCGGTGCGGG
CCTCTTCGCTATTACGCCAGCTGAATTGGAGCGACCTCATGCTATACCTGAGAAAGCAA
CCTGACCTACAGGAAAGAGTTACTCAAGAATAAGAATTTTCGTTTTAAAACCTAAGAGT
CACTTTAAAATTTGTATACACTTATTTTTTTTATAACTTATTTAATAATAAAAATCATA
AATCATAAGAAATTCGCTTATTTAGAAGTGTCAACAACGTATCTACCAACGATTTGACC
CTTTTCCATCTTTTCGTAAATTTCTGGCAAGGTAGACAAGCCGACAACCTTGATTGGAG
ACTTGACCAAACCTCTGGCGAAGAATTGTTAATTAAGAGCTCAGATCTTTTGCGGCCGC
SEQ. ID NO: 30
GTTTGCAAAAAGAACAAAACTGAAAAAACCCAGACACGCTCGACTTCCTGTCTTCCTAT
TGATTGCAGCTTCCAATTTCGTCACACAACAAGGTCCTAGCGACGGCTCACAGGTTTTG
TAACAAGCAATCGAAGGTTCTGGAATGGCGGGAAAGGGTTTAGTACCACATGCTATGAT
GCCCACTGTGATCTCCAGAGCAAAGTTCGTTCGATCGTACTGTTACTCTCTCTCTTTCA
AACAGAATTGTCCGAATCGTGTGACAACAACAGCCTGTTCTCACACACTCTTTTCTTCT
AACCAAGGGGGTGGTTTAGTTTAGTAGAACCTCGTGAAACTTACATTTACATATATATA
AACTTGCATAAATTGGTCAATGCAAGAAATACATATTTGGTCTTTTCTAATTCGTAGTT
TTTCAAGTTCTTAGATGCTTTCTTTTTCTCTTTTTTACAGATCATCAAGGAAGTAATTA
TCTACTTTTTACAACAAATATAAAACA
SEQ. ID NO: 31
ACAGTTTATTCCTGGCATCCACTAAATATAATGGAGCCCGCTTTTTAAGCTGGCATCCA
GAAAAAAAAAGAATCCCAGCACCAAAATATTGTTTTCTTCACCAACCATCAGTTCATAG
GTCCATTCTCTTAGCGCAACTACAGAGAACAGGGGCACAAACAGGCAAAAAACGGGCAC
AACCTCAATGGAGTGATGCAACCTGCCTGGAGTAAATGATGACACAAGGCAATTGACCC
ACGCATGTATCTATCTCATTTTCTTACACCTTCTATTACCTTCTGCTCTCTCTGATTTG
GAAAAAGCTGAAAAAAAAGGTTGAAACCAGTTCCCTGAAATTATTCCCCTACTTGACTA
ATAAGTATATAAAGACGGTAGGTATTGATTGTAATTCTGTAAATCTATTTCTTAAACTT
CTTAAATTCTACTTTTATAGTTAGTCTTTTTTTTAGTTTTAAAACACCAAGAACTTAGT
TTCGAATAAACACACATAAACAAACAAA
SEQ. ID NO: 32
GCTGGATTGAGCTGAATGGTGCCAGGTCGAGGCTGGGAGGGAGACTAACTCGAAAGTGA
CGAAGACTCGAAAATTAAAAAAAAAGATACTGCAGAAGGCAAGATTGAGAATGGAGTAA
AGGCAGCGTGGGTCCCCTGTGGAAACCGCAGTTTTCCTGCGCCAAGTGGTACCGGTGCG
AGTGCAGCAATTAATCTCTCGATATTTTCTTAGTATCTCTTTTTATATAAGAATATATT
TTGGAATTGGTAATGCTTATCTTCAATAGTTTCTTAGTTGAATGCACACTTAAGAGCAA
ATTGGCCAAGGAGTTCTTCGTTCGCTTTAATTTATTTCCTGGTTATTGTCAATTTATTC
ATCCCATCTCCCCAGGATAGAAGAAATTAGTGTAATTTTGCTGACAATACATTTTAACG
ACGATAACAATAATAGCAATTAAATAAAATAGCACTACCACCACTCCACTGCTCGTTAG
CTATTTCTGTAAAATAAATAAAAAGATC
SEQ. ID NO: 33
ATTCGCGCTATCTCGATTTCTACCTATATAGTTAATCTCTGTACAAAAACAATCTTTCC
AACTATCCATTAATCATAGTATATTATCAGCGTCGGCGATTTTACCACGCTTGACAAAA
GCCGCGGGCGGGATTCCTGTGGGTAGTGGCACCGGCAGTTAATCTAATCAAAGGCGCTT
GAAGGAAGAGATAGATAATAGAACAAAGCAATCGCCGCTTTGGACGGCAAATATGTTTA
TCCATTGGTGCGGTGATTGGATATGATTTGTCTCCAGTAGTATAAGCAAGCGCCAGATC
TGTTTACTGTAAAATTAAGTGAGTAATCTCGCGGGATGTAATGATTTAAGGGAATCTGG
TTCAGGTTTTCACATATATTTGTATATAAGGCCATTTGTAATTTCAATAGTTTTAGGAT
TTTTCCTTCTCCCAAAATACTCACTTACTGTGTTACATTACAGAAAGAACAGACAAGAA
ACCGTCAATAAGAAATATAACTAAGAACA
SEQ. ID NO: 34
CAGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATATTG
AAAAAGGAAGAGTATGAGTATTCAACATTTCCGTGTCGCCCTTATTCCCTTTTTTGCGG
CATTTTGCCTTCCTGTTTTTGCTCACCCAGAAACGCTGGTGAAAGTAAAAGATGCTGAA
GATCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCTAT
GTGGCGCGGTATTATCCCGTATTGACGCCGGGCAAGAGCAACTCGGTCGCCGCATACAC
TATTCTCAGAATGACTTGGTTGAGTACTCACCAGTCACAGAAAAGCATCTTACGGATGG
CATGACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTATTAA
CTGGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGATAA
ATCTGGAGCCGGTGAGCGTGGGTCTCGCGGTATCATTGCAGCACTGGGGCCAGATGGTA
AGCCCTCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCAGACCA
AGTTTACTCATATATACTTTAGATTGATTTAAAACTTCATTTTTAATTTAAAAGGATCT
AGGTGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGATA
CCAAATACTGTTCTTCTAGTGTAGCCGTAGTTAGGCCACCACTTCAAGAACTCTGTAGC
ACCGCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACT
GAGATACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTTGAGCGTCG
ATTTTTGTGATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTGCGTTATCC
CCTGATTCTGTGGATAACCGTATTACCGCCTTTGAGTGAGCTGATACCGCTCGCCGCAG
CCGAACGACCGAGCGCAGCGAGTCAGTGAGCGAGGAAGCGGAAGAGCGCCCAATACGCA
AACCGCCTCTCCCCGCGCGTTGGCCGATTCATTAATGCAGCTGGATCTTCGAGCGTCCC
AAAACCTTCTCAAGCAAGGTTTTCAGTATAATGTTACATGCGTACACGCGTCTGTACAG
AAAAAAAAGAAAAATTTGAAATATAAATAACGTTCTTAATACTAACATAACTATAAAAA
AATAAATAGGGACCTAGACTTCAGGTTGTCTAACTCCTTCCTTTTCGGTTAGAGCGGAT
CTTAGCTAGCCGCGGTACCAAGCTGGTGGATCCTTTGTTTGTTTATGTGTGTTTATTCG
AAACTAAGTTCTTGGTGTTTTAAAACTAAAAAAAAGACTAACTATAAAAGTAGAATTTA
AGAAGTTTAAGAAATAGATTTACAGAATTACAATCAATACCTACCGTCTTTATATACTT
ATTAGTCAAGTAGGGGAATAATTTCAGGGAACTGGTTTCAACCTTTTTTTTCAGCTTTT
TCCAAATCAGAGAGAGCAGAAGGTAATAGAAGGTGTAAGAAAATGAGATAGATACATGC
GTGGGTCAATTGCCTTGTGTCATCATTTACTCCAGGCAGGTTGCATCACTCCATTGAGG
TTGTGCCCGTTTTTTGCCTGTTTGTGCCCCTGTTCTCTGTAGTTGCGCTAAGAGAATGG
ACCTATGAACTGATGGTTGGTGAAGAAAACAATATTTTGGTGCTGGGATTCTTTTTTTT
TCTGGATGCCAGCTTAAAAAGCGGGCTCCATTATATTTAGTGGATGCCAGGAATAAACT
GTTGTTTGCAAAAAGAACAAAACTGAAAAAACCCAGACACGCTCGACTTCCTGTCTTCC
TATTGATTGCAGCTTCCAATTTCGTCACACAACAAGGTCCTAGCGACGGCTCACAGGTT
TTGTAACAAGCAATCGAAGGTTCTGGAATGGCGGGAAAGGGTTTAGTACCACATGCTAT
GATGCCCACTGTGATCTCCAGAGCAAAGTTCGTTCGATCGTACTGTTACTCTCTCTCTT
TCAAACAGAATTGTCCGAATCGTGTGACAACAACAGCCTGTTCTCACACACTCTTTTCT
TCTAACCAAGGGGGTGGTTTAGTTTAGTAGAACCTCGTGAAACTTACATTTACATATAT
ATAAACTTGCATAAATTGGTCAATGCAAGAAATACATATTTGGTCTTTTCTAATTCGTA
GTTTTTCAAGTTCTTAGATGCTTTCTTTTTCTCTTTTTTACAGATCATCAAGGAAGTAA
TTATCTACTTTTTACAACAAATATAAAACAGCGGCCGCAAAAGATCTGAGCTCTTAATT
AACAATTCTTCGCCAGAGGTTTGGTCAAGTCTCCAATCAAGGTTGTCGGCTTGTCTACC
TTGCCAGAAATTTACGAAAAGATGGAAAAGGGTCAAATCGTTGGTAGATACGTTGTTGA
CACTTCTAAATAAGCGAATTTCTTATGATTTATGATTTTTATTATTAAATAAGTTATAA
AAAAAATAAGTGTATACAAATTTTAAAGTGACTCTTAGGTTTTAAAACGAAAATTCTTA
TTCTTGAGTAACTCTTTCCTGTAGGTCAGGTTGCTTTCTCAGGTATAGCATGAGGTCGC
TCCAATTCAGCTGGCGTAATAGCGAAGAGGCCCGCACCGATCGCCCTTCCCAACAGTTG
CGCAGCCTGAATGGCGAATTCGACACTGGATGGCGGCGTTAGTATCGAATCGACAGCAG
TATAGCGACCAGCATTCACATACGATTGACGCATGATATTACTTTCTGCGCACTTAACT
TCGCATCTGGGCAGATGATGTCGAGGCGAAAAAAAATATAAATCACGCTAACATTTGAT
TAAAATAGAACAACTACAATATAAAAAAACTATACAAATGACAAGTTCTTGAAAACAAG
AATCTTTTTATTGTCAGTACTGATTAGAAAAACTCATCGAGCATCAAATGAAACTGCAA
TTTATTCATATCAGGATTATCAATACCATATTTTTGAAAAAGCCGTTTCTGTAATGAAG
GAGAAAACTCACCGAGGCAGTTCCATAGGATGGCAAGATCCTGGTATCGGTCTGCGATT
CCGACTCGTCCAACATCAATACAACCTATTAATTTCCCCTCGTCAAAAATAAGGTTATC
AAGTGAGAAATCACCATGAGTGACGACTGAATCCGGTGAGAATGGCAAAAGCTTATGCA
TTTCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCGCT
GTTAAAAGGACAATTACAAACAGGAATCGAATGCAACCGGCGCAGGAACACTGCCAGCG
CATCAACAATATTTTCACCTGAATCAGGATATTCTTCTAATACCTGGAATGCTGTTTTG
CCGGGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAACAT
CATTGGCAACGCTACCTTTGCCATGTTTCAGAAACAACTCTGGCGCATCGGGCTTCCCA
TACAATCGATAGATTGTCGCACCTGATTGCCCGACATTATCGCGAGCCCATTTATACCC
ATATAAATCAGCATCCATGTTGGAATTTAATCGCGGCCTCGAAACGTGAGTCTTTTCCT
TACCCATGGTTGTTTATGTTCGGATGTGATGTGAGAACTGTATCCTAGCAAGATTTTAA
AAGGAAGTATATGAAAGAAGAACCTCAGTGGCAAATCCTAACCTTTTATATTTCTCTAC
AGGGGCGCGGCGTGGGGACAATTCAACGCGTCTGTGAGGGGAGCGTTTCCCTGCTCGCA
GGTCTGCAGCGAGGAGCCGTAATTTTTGCTTCGCGCCGTGCGGCCATCAAAATGTATGG
ATGCAAATGATTATACATGGGGATGTATGGGCTAAATGTACGGGCGACAGTCACATCAT
GCCCCTGAGCTGCGCACGTCAAGACTGTCAAGGAGGGTATTCTGGGCCTCCATGTCGCT
GGCCGGGTGACCCGGCGGGGACGAGGCAAGCTAAACAGATCTGGCGCGTGTACTGAGAG
TGCACCATAAATTCCCGTTTTAAGAGCTTGGTGAGCGCTAGGAGTCACTGCCAGGTATC
GTTTGAACACGGCATTAGTCAGGGAAGTCATAACACAGTCCTTTCCCGCAATTTTCTTT
TTCTATTACTCTTGGCCTCCTCTAGTACACTCTATATTTTTTTATGCCTCGGTAATGAT
TTTCATTTTTTTTTTTCCACCTAGCGGATGACTCTTTTTTTTTCTTAGCGATTGGCATT
ATCACATAATGAATTATACATTATATAAAGTAATGTGATTTCTTCGAAGAATATACTAA
AAAATGAGCAGGCAAGATAAACGAAGGCAAAGATGACAGAGCAGAAAGCCCTAGTAAAG
CGTATTACAAATGAAACCAAGATTCAGATTGCGATCTCTTTAAAGGGTGGTCCCCTAGC
GATAGAGCACTCGATCTTCCCAGAAAAAGAGGCAGAAGCAGTAGCAGAACAGGCCACAC
AATCGCAAGTGATTAACGTCCACACAGGTATAGGGTTTCTGGACCATATGATACATGCT
CTGGCCAAGCATTCCGGCTGGTCGCTAATCGTTGAGTGCATTGGTGACTTACACATAGA
CGACCATCACACCACTGAAGACTGCGGGATTGCTCTCGGTCAAGCTTTTAAAGAGGCCC
TAGGGGCCGTGCGTGGAGTAAAAAGGTTTGGATCAGGATTTGCGCCTTTGGATGAGGCA
CTTTCCAGAGCGGTGGTAGATCTTTCGAACAGGCCGTACGCAGTTGTCGAACTTGGTTT
GCAAAGGGAGAAAGTAGGAGATCTCTCTTGCGAGATGATCCCGCATTTTCTTGAAAGCT
TTGCAGAGGCTAGCAGAATTACCCTCCACGTTGATTGTCTGCGAGGCAAGAATGATCAT
CACCGTAGTGAGAGTGCGTTCAAGGCTCTTGCGGTTGCCATAAGAGAAGCCACCTCGCC
CAATGGTACCAACGATGTTCCCTCCACCAAAGGTGTTCTTATGTAGTGACACCGATTAT
TTAAAGCTGCAGCATACGATATATATACATGTGTATATATGTATACCTATGAATGTCAG
TAAGTATGTATACGAACAGTATGATACTGAAGATGACAAGGTAATGCATCATTCTATAC
GTGTCATTCTGAACGAGGCGCGCTTTCCTTTTTTCTTTTTGCTTTTTCTTTTTTTTTCT
CTTGAACTCGACGGATCTATGCGGTGTGAAATATGGTGCACTCTCAGTACAATCTGCTC
TGATGCCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCGTGAT
ACGCCTATTTTTATAGGTTAATGTCATGATAATAATGGTTTCTTAGTATGATCCAATAT
CAAAGGAAATGATAGCATTGAAGGATGAGACTAATCCAATTGAGGAGTGGCAGCATATA
GAACAGCTAAAGGGTAGTGCTGAAGGAAGCATACGATACCCCGCATGGAATGGGATAAT
ATCACAGGAGGTACTAGACTACCTTTCATCCTACATAAATAGACGCATATAAGTACGCA
TTTAAGCATAAACACGCACTATGCCGTTCTTCTCATGTATATATATATACAGGCAACAC
GCAGATATAGGTGCGACGTGAACAGTGAGCTGTATGTGCGCAGCTCGCGTTGCATTTTC
GGAAGCGCTCGTTTTCGGAAACGCTTTGAAGTTCCTATTCCGAAGTTCCTATTCTCTAG
AAAGTATAGGAACTTCAGAGCGCTTTTGAAAACCAAAAGCGCTCTGAAGACGCACTTTC
AAAAAACCAAAAACGCACCGGACTGTAACGAGCTACTAAAATATTGCGAATACCGCTTC
CACAAACATTGCTCAAAAGTATCTCTTTGCTATATATCTCTGTGCTATATCCCTATATA
ACCTACCCATCCACCTTTCGCTCCTTGAACTTGCATCTAAACTCGACCTCTACATTTTT
TATGTTTATCTCTAGTATTACTCTTTAGACAAAAAAATTGTAGTAAGAACTATTCATAG
AGTGAATCGAAAACAATACGAAAATGTAAACATTTCCTATACGTAGTATATAGAGACAA
AATAGAAGAAACCGTTCATAATTTTCTGACCAATGAAGAATCATCAACGCTATCACTTT
CTGTTCACAAAGTATGCGCAATCCACATCGGTATAGAATATAATCGGGGATGCCTTTAT
CTTGAAAAAATGCACCCGCAGCTTCGCTAGTAATCAGTAAACGCGGGAAGTGGAGTCAG
GCTTTTTTTATGGAAGAGAAAATAGACACCAAAGTAGCCTTCTTCTAACCTTAACGGAC
CTACAGTGCAAAAAGTTATCAAGAGACTGCATTATAGAGCGCACAAAGGAGAAAAAAAG
TAATCTAAGATGCTTTGTTAGAAAAATAGCGCTCTCGGGATGCATTTTTGTAGAACAAA
AAAGAAGTATAGATTCTTTGTTGGTAAAATAGCGCTCTCGCGTTGCATTTCTGTTCTGT
AAAAATGCAGCTCAGATTCTTTGTTTGAAAAATTAGCGCTCTCGCGTTGCATTTTTGTT
TTACAAAAATGAAGCACAGATTCTTCGTTGGTAAAATAGCGCTTTCGCGTTGCATTTCT
GTTCTGTAAAAATGCAGCTCAGATTCTTTGTTTGAAAAATTAGCGCTCTCGCGTTGCAT
TTTTGTTCTACAAAATGAAGCACAGATGCTTCGTT
SEQ. ID NO: 35
GCTAACAGCGCGATTTGCTGGTGACCCAATGCGACCAGATGCTCCACGCCCAGTCGCGT
ACCGTCTTCATGGGAGAAAATAATACTGTTGATGGGTGTCTGGTCAGAGACATCAAGAA
ATAACGCCGGAACATTAGTGCAGGCAGCTTCCACAGCAATGGCATCCTGGTCATCCAGC
GGATAGTTAATGATCAGCCCACTGACGCGTTGCGCGAGAAGATTGTGCACCGCCGCTTT
ACAGGCTTCGACGCCGCTTCGTTCTACCATCGACACCACCACGCTGGCACCCAGTTGAT
CGGCGCGAGATTTAATCGCCGCGACAATTTGCGACGGCGCGTGCAGGGCCAGACTGGAG
GTGGCAACGCCAATCAGCAACGACTGTTTGCCCGCCAGTTGTTGTGCCACGCGGTTGGG
AATGTAATTCAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCATACTCTGCG
ACATCGTATAACGTTACTGGTTTCACATTCACCACCCTGAATTGACTCTCTTCCGGGCG
CTATCATGCCATACCGCGAAAGGTTTTGCGCCATTCGATGGTGTCCGGGATCTCGACGC
TCTCCCTTATGCGACTCCTGCATTAGGAAGCAGCCCAGTAGTAGGTTGAGGCCGTTGAG
CACCGCCGCCGCAAGGAATGGTGCATGCAAGGAGATGGCGCCCAACAGTCCCCCGGCCA
CGGGGCCTGCCACCATACCCACGCCGAAACAAGCGCTCATGAGCCCGAAGTGGCGAGCC
CGATCTTCCCCATCGGTGATGTCGGCGATATAGGCGCCAGCAACCGCACCTGTGGCGCC
GGTGATGCCGGCCACGATGCGTCCGGCGTAGCCTAGGATCGAGATCGATCTCGATCCCG
CGAAATTAATACGACTCACTATAGGGGAATTGTGAGCGGATAACAATTCCCCTCTAGAA
ATAATTTTGTTTAACTTTAAGAAGGAGATATACATATGGCAGATCTCAATTGGATATCG
GCCGGCCACGCGATCGCTGACGTCGGTACCCTCGAGTCTGGTAAAGAAACCGCTGCTGC
GAAATTTGAACGCCAGCACATGGACTCGTCTACTAGTCGCAGCTTAATTAACCTAAACT
GCTGCCACCGCTGAGCAATAACTAGCATAACCCCTTGGGGCCTCTAAACGGGTCTTGAG
GGGTTTTTTGCTAGCGAAAGGAGGAGTCGACACTGCTTCCGGTAGTCAATAAACCGGTA
AACCAGCAATAGACATAAGCGGCTATTTAACGACCCTGCCCTGAACCGACGACCGGGTC
ATCGTGGCCGGATCTTGCGGCCCCTCGGCTTGAACGAATTGTTAGACATTATTTGCCGA
CTACCTTGGTGATCTCGCCTTTCACGTAGTGGACAAATTCTTCCAACTGATCTGCGCGC
GAGGCCAAGCGATCTTCTTCTTGTCCAAGATAAGCCTGTCTAGCTTCAAGTATGACGGG
CTGATACTGGGCCGGCAGGCGCTCCATTGCCCAGTCGGCAGCGACATCCTTCGGCGCGA
TTTTGCCGGTTACTGCGCTGTACCAAATGCGGGACAACGTAAGCACTACATTTCGCTCA
TCGCCAGCCCAGTCGGGCGGCGAGTTCCATAGCGTTAAGGTTTCATTTAGCGCCTCAAA
TAGATCCTGTTCAGGAACCGGATCAAAGAGTTCCTCCGCCGCTGGACCTACCAAGGCAA
CGCTATGTTCTCTTGCTTTTGTCAGCAAGATAGCCAGATCAATGTCGATCGTGGCTGGC
TCGAAGATACCTGCAAGAATGTCATTGCGCTGCCATTCTCCAAATTGCAGTTCGCGCTT
AGCTGGATAACGCCACGGAATGATGTCGTCGTGCACAACAATGGTGACTTCTACAGCGC
GGAGAATCTCGCTCTCTCCAGGGGAAGCCGAAGTTTCCAAAAGGTCGTTGATCAAAGCT
CGCCGCGTTGTTTCATCAAGCCTTACGGTCACCGTAACCAGCAAATCAATATCACTGTG
TGGCTTCAGGCCGCCATCCACTGCGGAGCCGTACAAATGTACGGCCAGCAACGTCGGTT
CGAGATGGCGCTCGATGACGCCAACTACCTCTGATAGTTGAGTCGATACTTCGGCGATC
ACCGCTTCCCTCATACTCTTCCTTTTTCAATATTATTGAAGCATTTATCAGGGTTATTG
TCTCATGAGCGGATACATATTTGAATGTATTTAGAAAAATAAACAAATAGCCAGCTCAC
TCGGTCGCTACGCTCCGGGCGTGAGACTGCGGCGGGCGCTGCGGACACATACAAAGTTA
CCCACAGATTCCGTGGATAAGCAGGGGACTAACATGTGAGGCAAAACAGCAGGGCCGCG
CCGGTGGCGTTTTTCCATAGGCTCCGCCCTCCTGCCAGAGTTCACATAAACAGACGCTT
TTCCGGTGCATCTGTGGGAGCCGTGAGGCTCAACCATGAATCTGACAGTACGGGCGAAA
CCCGACAGGACTTAAAGATCCCCACCGTTTCCGGCGGGTCGCTCCCTCTTGCGCTCTCC
TGTTCCGACCCTGCCGTTTACCGGATACCTGTTCCGCCTTTCTCCCTTACGGGAAGTGT
GGCGCTTTCTCATAGCTCACACACTGGTATCTCGGCTCGGTGTAGGTCGTTCGCTCCAA
GCTGGGCTGTAAGCAAGAACTCCCCGTTCAGCCCGACTGCTGCGCCTTATCCGGTAACT
GTTCACTTGAGTCCAACCCGGAAAAGCACGGTAAAACGCCACTGGCAGCAGCCATTGGT
AACTGGGAGTTCGCAGAGGATTTGTTTAGCTAAACACGCGGTTGCTCTTGAAGTGTGCG
CCAAAGTCCGGCTACACTGGAAGGACAGATTTGGTTGCTGTGCTCTGCGAAAGCCAGTT
ACCACGGTTAAGCAGTTCCCCAACTGACTTAACCTTCGATCAAACCACCTCCCCAGGTG
GTTTTTTCGTTTACAGGGCAAAAGATTACGCGCAGAAAAAAAGGATCTCAAGAAGATCC
TTTGATCTTTTCTACTGAACCGCTCTAGATTTCAGTGCAATTTATCTCTTCAAATGTAG
CACCTGAAGTCAGCCCCATACGATATAAGTTGTAATTCTCATGTTAGTCATGCCCCGCG
CCCACCGGAAGGAGCTGACTGGGTTGAAGGCTCTCAAGGGCATCGGTCGAGATCCCGGT
GCCTAATGAGTGAGCTAACTTACATTAATTGCGTTGCGCTCACTGCCCGCTTTCCAGTC
GGGAAACCTGTCGTGCCAGCTGCATTAATGAATCGGCCAACGCGCGGGGAGAGGCGGTT
TGCGTATTGGGCGCCAGGGTGGTTTTTCTTTTCACCAGTGAGACGGGCAACAGCTGATT
GCCCTTCACCGCCTGGCCCTGAGAGAGTTGCAGCAAGCGGTCCACGCTGGTTTGCCCCA
GCAGGCGAAAATCCTGTTTGATGGTGGTTAACGGCGGGATATAACATGAGCTGTCTTCG
GTATCGTCGTATCCCACTACCGAGATGTCCGCACCAACGCGCAGCCCGGACTCGGTAAT
GGCGCGCATTGCGCCCAGCGCCATCTGATCGTTGGCAACCAGCATCGCAGTGGGAACGA
TGCCCTCATTCAGCATTTGCATGGTTTGTTGAAAACCGGACATGGCACTCCAGTCGCCT
TCCCGTTCCGCTATCGGCTGAATTTGATTGCGAGTGAGATATTTATGCCAGCCAGCCAG
ACGCAGACGCGCCGAGACAGAACTTAATGGGCCC
SEQ. ID NO: 36
ATCCGGATATAGTTCCTCCTTTCAGCAAAAAACCCCTCAAGACCCGTTTAGAGGCCCCA
AGGGGTTATGCTAGTTATTGCTCAGCGGTGGCAGCAGCCAACTCAGCTTCCTTTCGGGC
TTTGTTAGCAGCCGGATCTCAGTGGTGGTGGTGGTGGTGCTCGAGTGCGGCCGCAAGCT
TGTCGACGGAGCTCGAATTCGGATCCGCGACCCATTTGCTGTCCACCAGTCATGCTAGC
CATATGGCTGCCGCGCGGCACCAGGCCGCTGCTGTGATGATGATGATGATGGCTGCTGC
CCATGGTATATCTCCTTCTTAAAGTTAAACAAAATTATTTCTAGAGGGGAATTGTTATC
CGCTCACAATTCCCCTATAGTGAGTCGTATTAATTTCGCGGGATCGAGATCTCGATCCT
CTACGCCGGACGCATCGTGGCCGGCATCACCGGCGCCACAGGTGCGGTTGCTGGCGCCT
ATATCGCCGACATCACCGATGGGGAAGATCGGGCTCGCCACTTCGGGCTCATGAGCGCT
TGTTTCGGCGTGGGTATGGTGGCAGGCCCCGTGGCCGGGGGACTGTTGGGCGCCATCTC
CTTGCATGCACCATTCCTTGCGGCGGCGGTGCTCAACGGCCTCAACCTACTACTGGGCT
GCTTCCTAATGCAGGAGTCGCATAAGGGAGAGCGTCGAGATCCCGGACACCATCGAATG
GCGCAAAACCTTTCGCGGTATGGCATGATAGCGCCCGGAAGAGAGTCAATTCAGGGTGG
TGAATGTGAAACCAGTAACGTTATACGATGTCGCAGAGTATGCCGGTGTCTCTTATCAG
ACCGTTTCCCGCGTGGTGAACCAGGCCAGCCACGTTTCTGCGAAAACGCGGGAAAAAGT
GGAAGCGGCGATGGCGGAGCTGAATTACATTCCCAACCGCGTGGCACAACAACTGGCGG
GCAAACAGTCGTTGCTGATTGGCGTTGCCACCTCCAGTCTGGCCCTGCACGCGCCGTCG
CAAATTGTCGCGGCGATTAAATCTCGCGCCGATCAACTGGGTGCCAGCGTGGTGGTGTC
GATGGTAGAACGAAGCGGCGTCGAAGCCTGTAAAGCGGCGGTGCACAATCTTCTCGCGC
AACGCGTCAGTGGGCTGATCATTAACTATCCGCTGGATGACCAGGATGCCATTGCTGTG
GAAGCTGCCTGCACTAATGTTCCGGCGTTATTTCTTGATGTCTCTGACCAGACACCCAT
CAACAGTATTATTTTCTCCCATGAAGACGGTACGCGACTGGGCGTGGAGCATCTGGTCG
CATTGGGTCACCAGCAAATCGCGCTGTTAGCGGGCCCATTAAGTTCTGTCTCGGCGCGT
CTGCGTCTGGCTGGCTGGCATAAATATCTCACTCGCAATCAAATTCAGCCGATAGCGGA
ACGGGAAGGCGACTGGAGTGCCATGTCCGGTTTTCAACAAACCATGCAAATGCTGAATG
AGGGCATCGTTCCCACTGCGATGCTGGTTGCCAACGATCAGATGGCGCTGGGCGCAATG
CGCGCCATTACCGAGTCCGGGCTGCGCGTTGGTGCGGATATCTCGGTAGTGGGATACGA
CGATACCGAAGACAGCTCATGTTATATCCCGCCGTTAACCACCATCAAACAGGATTTTC
GCCTGCTGGGGCAAACCAGCGTGGACCGCTTGCTGCAACTCTCTCAGGGCCAGGCGGTG
AAGGGCAATCAGCTGTTGCCCGTCTCACTGGTGAAAAGAAAAACCACCCTGGCGCCCAA
TACGCAAACCGCCTCTCCCCGCGCGTTGGCCGATTCATTAATGCAGCTGGCACGACAGG
TTTCCCGACTGGAAAGCGGGCAGTGAGCGCAACGCAATTAATGTAAGTTAGCTCACTCA
TTAGGCACCGGGATCTCGACCGATGCCCTTGAGAGCCTTCAACCCAGTCAGCTCCTTCC
GGTGGGCGCGGGGCATGACTATCGTCGCCGCACTTATGACTGTCTTCTTTATCATGCAA
CTCGTAGGACAGGTGCCGGCAGCGCTCTGGGTCATTTTCGGCGAGGACCGCTTTCGCTG
GAGCGCGACGATGATCGGCCTGTCGCTTGCGGTATTCGGAATCTTGCACGCCCTCGCTC
AAGCCTTCGTCACTGGTCCCGCCACCAAACGTTTCGGCGAGAAGCAGGCCATTATCGCC
GGCATGGCGGCCCCACGGGTGCGCATGATCGTGCTCCTGTCGTTGAGGACCCGGCTAGG
CTGGCGGGGTTGCCTTACTGGTTAGCAGAATGAATCACCGATACGCGAGCGAACGTGAA
GCGACTGCTGCTGCAAAACGTCTGCGACCTGAGCAACAACATGAATGGTCTTCGGTTTC
CGTGTTTCGTAAAGTCTGGAAACGCGGAAGTCAGCGCCCTGCACCATTATGTTCCGGAT
CTGCATCGCAGGATGCTGCTGGCTACCCTGTGGAACACCTACATCTGTATTAACGAAGC
GCTGGCATTGACCCTGAGTGATTTTTCTCTGGTCCCGCCGCATCCATACCGCCAGTTGT
TTACCCTCACAACGTTCCAGTAACCGGGCATGTTCATCATCAGTAACCCGTATCGTGAG
CATCCTCTCTCGTTTCATCGGTATCATTACCCCCATGAACAGAAATCCCCCTTACACGG
AGGCATCAGTGACCAAACAGGAAAAAACCGCCCTTAACATGGCCCGCTTTATCAGAAGC
CAGACATTAACGCTTCTGGAGAAACTCAACGAGCTGGACGCGGATGAACAGGCAGACAT
CTGTGAATCGCTTCACGACCACGCTGATGAGCTTTACCGCAGCTGCCTCGCGCGTTTCG
GTGATGACGGTGAAAACCTCTGACACATGCAGCTCCCGGAGACGGTCACAGCTTGTCTG
TAAGCGGATGCCGGGAGCAGACAAGCCCGTCAGGGCGCGTCAGCGGGTGTTGGCGGGTG
TCGGGGCGCAGCCATGACCCAGTCACGTAGCGATAGCGGAGTGTATACTGGCTTAACTA
TGCGGCATCAGAGCAGATTGTACTGAGAGTGCACCATATATGCGGTGTGAAATACCGCA
CAGATGCGTAAGGAGAAAATACCGCATCAGGCGCTCTTCCGCTTCCTCGCTCACTGACT
CGCTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGACTA
TAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGCTAC
ACTAGAAGGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAACAA
TAAAACTGTCTGCTTACATAAACAGTAATACAAGGGGTGTTATGAGCCATATTCAACGG
GAAACGTCTTGCTCTAGGCCGCGATTAAATTCCAACATGGATGCTGATTTATATGGGTA
TAAATGGGCTCGCGATAATGTCGGGCAATCAGGTGCGACAATCTATCGATTGTATGGGA
AGCCCGATGCGCCAGAGTTGTTTCTGAAACATGGCAAAGGTAGCGTTGCCAATGATGTT
ACAGATGAGATGGTCAGACTAAACTGGCTGACGGAATTTATGCCTCTTCCGACCATCAA
GCATTTTATCCGTACTCCTGATGATGCATGGTTACTCACCACTGCGATCCCCGGGAAAA
CAGCATTCCAGGTATTAGAAGAATATCCTGATTCAGGTGAAAATATTGTTGATGCGCTG
GCAGTGTTCCTGCGCCGGTTGCATTCGATTCCTGTTTGTAATTGTCCTTTTAACAGCGA
TCGCGTATTTCGTCTCGCTCAGGCGCAATCACGAATGAATAACGGTTTGGTTGATGCGA
GTGATTTTGATGACGAGCGTAATGGCTGGCCTGTTGAACAAGTCTGGAAAGAAATGCAT
AAACTTTTGCCATTCTCACCGGATTCAGTCGTCACTCATGGTGATTTCTCACTTGATAA
CCTTATTTTTGACGAGGGGAAATTAATAGGTTGTATTGATGTTGGACGAGTCGGAATCG
CAGACCGATACCAGGATCTTGCCATCCTATGGAACTGCCTCGGTGAGTTTTCTCCTTCA
TTACAGAAACGGCTTTTTCAAAAATATGGTATTGATAATCCTGATATGAATAAATTGCA
GTTTCATTTGATGCTCGATGAGTTTTTCTAAGAATTAATTCATGAGCGGATACATATTT
GAATGTATTTAGAAAAATAAACAAATAGGGGTTCCGCGCACATTTCCCCGAAAAGTGCC
ACCTGAAATTGTAAACGTTAATATTTTGTTAAAATTCGCGTTAAATTTTTGTTAAATCA
GCTCATTTTTTAACCAATAGGCCGAAATCGGCAAAATCCCTTATAAATCAAAAGAATAG
ACCGAGATAGGGTTGAGTGTTGTTCCAGTTTGGAACAAGAGTCCACTATTAAAGAACGT
GGACTCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAACCCT
AAAGGGAGCCCCCGATTTAGAGCTTGACGGGGAAAGCCGGCGAACGTGGCGAGAAAGGA
AGGGAAGAAAGCGAAAGGAGCGGGCGCTAGGGCGCTGGCAAGTGTAGCGGTCACGCTGC
GCGTAACCACCACACCCGCCGCGCTTAATGCGCCGCTACAGGGCGCGTCCCATTCGCCA
SEQ. ID NO: 37
ATCCGGATATAGTTCCTCCTTTCAGCAAAAAACCCCTCAAGACCCGTTTAGAGGCCCCA
AGGGGTTATGCTAGTTATTGCTCAGCGGTGGCAGCAGCCAACTCAGCTTCCTTTCGGGC
TTTGTTAGCAGCCGGATCTCAGTGGTGGTGGTGGTGGTGCTCGAGTGCGGCCGCAAGCT
TGTCGACGGAGCTCGAATTCGGATCCTAGAGGGAAACCGTTGTGGTCTCCCTATAGTGA
GTCGTATTAATTTCGCGGGATCGAGATCTCGGGCAGCGTTGGGTCCTGGCCACGGGTGC
GCATGATCGTGCTCCTGTCGTTGAGGACCCGGCTAGGCTGGCGGGGTTGCCTTACTGGT
TAGCAGAATGAATCACCGATACGCGAGCGAACGTGAAGCGACTGCTGCTGCAAAACGTC
TGCGACCTGAGCAACAACATGAATGGTCTTCGGTTTCCGTGTTTCGTAAAGTCTGGAAA
CGCGGAAGTCAGCGCCCTGCACCATTATGTTCCGGATCTGCATCGCAGGATGCTGCTGG
CTACCCTGTGGAACACCTACATCTGTATTAACGAAGCGCTGGCATTGACCCTGAGTGAT
TTTTCTCTGGTCCCGCCGCATCCATACCGCCAGTTGTTTACCCTCACAACGTTCCAGTA
ACCGGGCATGTTCATCATCAGTAACCCGTATCGTGAGCATCCTCTCTCGTTTCATCGGT
ATCATTACCCCCATGAACAGAAATCCCCCTTACACGGAGGCATCAGTGACCAAACAGGA
AAAAACCGCCCTTAACATGGCCCGCTTTATCAGAAGCCAGACATTAACGCTTCTGGAGA
AACTCAACGAGCTGGACGCGGATGAACAGGCAGACATCTGTGAATCGCTTCACGACCAC
GCTGATGAGCTTTACCGCAGCTGCCTCGCGCGTTTCGGTGATGACGGTGAAAACCTCTG
ACACATGCAGCTCCCGGAGACGGTCACAGCTTGTCTGTAAGCGGATGCCGGGAGCAGAC
AAGCCCGTCAGGGCGCGTCAGCGGGTGTTGGCGGGTGTCGGGGCGCAGCCATGACCCAG
TCACGTAGCGATAGCGGAGTGTATACTGGCTTAACTATGCGGCATCAGAGCAGATTGTA
CTGAGAGTGCACCATATATGCGGTGTGAAATACCGCACAGATGCGTAAGGAGAAAATAC
CGCATCAGGCGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAATCG
ACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACACGACTTA
TCGCCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTACGCGCAG
AAAAAAAGGATCTCAAGAAGATCCTTTGATCTTTTCTACGGGGTCTGACGCTCAGTGGA
ACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTG
GTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTT
AATAGTTTGCGCAACGTTGTTGCCATTGCTGCAGGCATCGTGGTGTCACGCTCGTCGTT
TGGTATGGCTTCATTCAGCTCCGGTTCCCAACGATCAAGGCGAGTTACATGATCCCCCA
TGTTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTT
CAGCATCTTTTACTTTCACCAGCGTTTCTGGGTGAGCAAAAACAGGAAGGCAAAATGCC
GCAAAAAAGGGAATAAGGGCGACACGGAAATGTTGAATACTCATACTCTTCCTTTTTCA
ATATTATTGAAGCATTTATCAGGGTTATTGTCTCATGAGCGGATACATATTTGAATGTA
TTTAGAAAAATAAACAAATAGGGGTTCCGCGCACATTTCCCCGAAAAGTGCCACCTGAA
ATTGTAAACGTTAATATTTTGTTAAAATTCGCGTTAAATTTTTGTTAAATCAGCTCATT
TTTTAACCAATAGGCCGAAATCGGCAAAATCCCTTATAAATCAAAAGAATAGACCGAGA
TAGGGTTGAGTGTTGTTCCAGTTTGGAACAAGAGTCCACTATTAAAGAACGTGGACTCC
AACGTCAAAGGGCGAAAAACCGTCTATCAGGGCGATGGCCCACTACGTGAACCATCACC
CTAATCAAGTTTTTTGGGGTCGAGGTGCCGTAAAGCACTAAATCGGAACCCTAAAGGGA
GCCCCCGATTTAGAGCTTGACGGGGAAAGCCGGCGAACGTGGCGAGAAAGGAAGGGAAG
AAAGCGAAAGGAGCGGGCGCTAGGGCGCTGGCAAGTGTAGCGGTCACGCTGCGCGTAAC
CACCACACCCGCCGCGCTTAATGCGCCGCTACAGGGCGCGTCCCATTCGCCA
SEQ. ID NO: 38
TACCCATACGACGTTCCAGACTACGCC
SEQ. ID NO: 39
YPYDVPDYA
SEQ. ID NO: 40
GAACAAAAGTTAATTTCTGAAGAAGATTTGGAA
SEQ. ID NO: 41
EQKLISEEDL
SEQ. ID NO: 42
GACTACAAGGATGACGATGACAAA
SEQ. ID NO: 43
DYKDDDDK
SEQ. ID NO: 44
GGTAAGCCAATTCCAAATCCTTTGTTGGGTTTGGACTCCACC
SEQ. ID NO: 45
GKPIPNPLLGLDST
SEQ. ID NO: 46
CATCATCATCATCATCAT
SEQ. ID NO: 47
HHHHHH
SEQ. ID NO: 48
ATGGCGTTCGATCTCAAGACTGAAGACGGCCTCATCACATATCTCACTAAACATCTTTC
TTTGGACGTCGACACGAGCGGAGTGAAGCGCCTTAGCGGAGGCTTTGTCAATGTAACCT
GGCGCATTAAGCTCAATGCTCCTTATCAAGGTCATACGAGCATCATCCTGAAGCATGCT
CAGCCGCACATGTCTACGGATGAGGATTTTAAGATAGGTGTAGAACGTTCGGTTTACGA
ATACCAGGCTATCAAGCTCATGATGGCCAATCGGGAGGTTCTGGGAGGCGTGGATGGCA
TAGTTTCTGTGCCAGAAGGCCTGAACTACGACTTAGAGAATAATGCATTGATCATGCAA
GATGTCGGGAAGATGAAGACCCTTTTAGATTATGTCACCGCCAAACCGCCACTTGCGAC
GGATATAGCCCGCCTTGTTGGGACAGAAATTGGGGGGTTCGTTGCCAGACTCCATAACA
TAGGCCGCGAGAGGCGAGACGATCCTGAGTTCAAATTCTTCTCTGGAAATATTGTCGGA
AGGACGACTTCAGACCAGCTGTATCAAACCATCATACCCAACGCAGCGAAATATGGCGT
CGATGACCCCTTGCTGCCTACTGTGGTTAAGGACCTTGTGGACGATGTCATGCACAGCG
AAGAGACCCTTGTCATGGCGGACCTGTGGAGTGGAAATATTCTTCTCCAGTTGGAGGAG
GGAAACCCATCGAAGCTGCAGAAGATATATATCCTGGATTGGGAACTTTGCAAGTACGG
CCCAGCGTCGTTGGACCTGGGCTATTTCTTGGGTGACTGCTATTTGATATCCCGCTTTC
AAGACGAGCAGGTCGGTACGACGATGCGGCAAGCCTACTTGCAAAGCTATGCGCGTACG
AGCAAGCATTCGATCAACTACGCCAAAGTCACTGCAGGTATTGCTGCTCATATTGTGAT
GTGGACCGACTTTATGCAGTGGGGGAGCGAGGAAGAAAGGATAAATTTTGTGAAAAAGG
GGGTAGCTGCCTTTCACGACGCCAGGGGCAACAACGACAATGGGGAAATTACGTCTACC
TTACTGAAGGAATCATCCACTGCGTAA
SEQ. ID NO: 49
MAFDLKTEDGLITYLTKHLSLDVDTSGVKRLSGGFVNVTWRIKLNAPYQGHTSIILKHA
QPHMSTDEDFKIGVERSVYEYQAIKLMMANREVLGGVDGIVSVPEGLNYDLENNALIMQ
DVGKMKTLLDYVTAKPPLATDIARLVGTEIGGFVARLHNIGRERRDDPEFKFFSGNIVG
RTTSDQLYQTIIPNAAKYGVDDPLLPTVVKDLVDDVMHSEETLVMADLWSGNILLQLEE
GNPSKLQKIYILDWELCKYGPASLDLGYFLGDCYLISRFQDEQVGTTMRQAYLQSYART
SKHSINYAKVTAGIAAHIVMWTDFMQWGSEEERINFVKKGVAAFHDARGNNDNGEITST
LLKESSTA
SEQ. ID NO: 50
CGAGATCTTTGTGTTCGGTTACCCGGCTCAGATCCTAACTTCTTCTTTTGGTATGTTTA
TTCGTATAAGTTACTGTTGTCCACAGGCAATACTCTGCAGAAAATTAAAACGGCATTAA
TGCTAGGACAACCAGAATTGTTACTACTGTATGTGCGATAGTTGATAACTGCAACATTA
TGCCCGGTATATTCTCAAAAAACCCTATTACTGCATACGAAGAAATCGCAAGAGAAATC
TTTCGGTTTGGAAAAGCTCACTGTGAGGTTCCTTGGAGCCAATAGTAATACAGCACAAT
CCAAGGAAAAATCTGGCCTATATGCAAGGAAGGAGAGATAGTCAAAAGCATTCTTTCCC
CTAGAAGTTGGTGCATATATGGCATCGTTAAAACATATTACCCCCAAAATTTCTTCTCT
AAACGATGTGCTTGGCCTTTGTTTTGGTTTTTGATGTCGGTCGTTTGAGGCCCCTTGCG
GAAAATCGAGATCGCCGAATGGCACGCGAGGGAAGGGAAATAAGGTTTAAAGGCACTGA
AACAATAGGCAAGAAGTAGGCGAGAGCCGACATACGAGACTAAATTAAGTCCTCAGCGA
GCTCGCATGGAATGCGTGCGATGAGCGACCTCATGCTATACCTGAGAAAGCAACCTGAC
CTACAGGAAAGAGTTACTCAAGAATAAGAATTTTCGTTTTAAAACCTAAGAGTCACTTT
AAAATTTGTATACACTTATTTTTTTTATAACTTATTTAATAATAAAAATCATAAATCAT
AAGAAATTCGCTTATTTAGAAGTGTCAACAACGTATCTACCAACGGAATGCGTGCGATT
CATTTGTCATCGTCATCCTTGTAGTCTTCAGCAACACATGGGTATGAAATAGAAACTTC
TTCAGCCAATGCTTTAATCTTCATCATGATTTGCAACATTTCTTCAGTTGTAGTTCTTG
GATTAATTGAACACAATCTAATAACAACCTTTTCCTTCAATTCTGTAGTAGATAACATA
GCGAAACCTCTATGTGTGATTTCCTTAACCAATTTCTTATTAATTTCATTAATAGTATC
TGTTGATGCCAATTCAGATGGAATGTATCTAAAAGTAACGATACCCAATTGAGCTGGTG
TAACAACTTCCCAATCTTTTGCTTTACCCAAAAATGCTTCAACTTGTTCTGCTAACATG
ATACCATGATCGATTGCTTGTCTAAAAGCAGCAACACCGAAAACTTTAAAAGACAACCA
AACCTTCAAAGCTCTGAATCTTCTTGACAATTCGATACCACATTCACCGAAATTAATTT
CACCTTCAACGTTAGTTTCTGAATCCTTGATGTATTCTGGCATCATTCTAAAAGTCTTT
GACAAATATTGAGAGTTTCTGATCAAAACACAACCAACATCGTATGGTTGGAACAACCA
CTTATGTGGATCTAAAGTCAAAGAATCTGCTCTATGAATACCTTGCAACATAGCTGAAC
CCTTTTCAGACAAGATAGCTGGAGCACCATAAGAACCATCAGCATGCAACCAAACATCT
TCATCGTTACACAAATCTGCTAATTCGTTCAAAGAATCAACAGCACCACAATTTGTAGT
ACCAGCATTTGCAATAACACAGAATGGCTTTTTACCCTTAGTTCTATCTTCTTTAATTT
GTTTCTTCAAAGCTGAAACAGAGATTCTCAAATGTTCATCTGTTTCGATTCTACAGATT
TGATGATGTTTAAAACCTAAAACCTTCAATGCTCTATCAACTGAGAAATGTGTTTGATC
AGAGAAGTAAACAACAGCATTTTCGATATCGTTGTTCAACTTAGCCTGTCTTGCAACAG
TCAAAGCTGTCAAATTTGCCATTGAACCACCAGAAACAAATAAACCTTCAGCTGAATCT
GGAAAACCCAACATAGATTTCAACCAATTAATTGTAGTCAATTCGATTTGTTCAGCACC
TGCACCAGCAATCCATGCAGTTGGAAAAACATTAAAACCAGAAGCCAAGAAATCTGCAA
CAACACCAACGTAATTATTTGGACCTGGAACAAAAGCCAAGAAATGTGGATGATCAACA
TGTGTAATTTGATTAAAAACGTTTCTGTTCAAGAAATGCAACAATTCCTTTGGATCTGA
ACCATTTTCTGGGATAGATTCAGTCAACTTATTTCTCAAGATATCAGAATCGATTGTTT
CTGAAACTGGCTTAGACTTCAAATGGTTCATGTGATCGATGATCAAATCAACTGCTTGG
TAACCCAATTGTCTCATTTCTTCAGCTGACAATTGCAAATTTTCAGACATTGTTTTATA
TTTGTTGTAAAAAGTAGATAATTACTTCCTTGATGATCTGTAAAAAAGAGAAAAAGAAA
GCATCTAAGAACTTGAAAAACTACGAATTAGAAAAGACCAAATATGTATTTCTTGCATT
GACCAATTTATGCAAGTTTATATATATGTAAATGTAAGTTTCACGAGGTTCTACTAAAC
TAAACCACCCCCTTGGTTAGAAGAAAAGAGTGTGTGAGAACAGGCTGTTGTTGTCACAC
GATTCGGACAATTCTGTTTGAAAGAGAGAGAGTAACAGTACGATCGAACGAACTTTGCT
CTGGAGATCACAGTGGGCATCATAGCATGTGGTACTAAACCCTTTCCCGCCATTCCAGA
ACCTTCGATTGCTTGTTACAAAACCTGTGAGCCGTCGCTAGGACCTTGTTGTGTGACGA
AATTGGAAGCTGCAATCAATAGGAAGACAGGAAGTCGAGCGTGTCTGGGTTTTTTCAGT
TTTGTTCTTTTTGCAAACAACAGTTTATTCCTGGCATCCACTAAATATAATGGAGCCCG
CTTTTTAAGCTGGCATCCAGAAAAAAAAAGAATCCCAGCACCAAAATATTGTTTTCTTC
ACCAACCATCAGTTCATAGGTCCATTCTCTTAGCGCAACTACAGAGAACAGGGGCACAA
ACAGGCAAAAAACGGGCACAACCTCAATGGAGTGATGCAACCTGCCTGGAGTAAATGAT
GACACAAGGCAATTGACCCACGCATGTATCTATCTCATTTTCTTACACCTTCTATTACC
TTCTGCTCTCTCTGATTTGGAAAAAGCTGAAAAAAAAGGTTGAAACCAGTTCCCTGAAA
TTATTCCCCTACTTGACTAATAAGTATATAAAGACGGTAGGTATTGATTGTAATTCTGT
AAATCTATTTCTTAAACTTCTTAAATTCTACTTTTATAGTTAGTCTTTTTTTTAGTTTT
AAAACACCAAGAACTTAGTTTCGAATAAACACACATAAACAAACAAAATGGCTTCTAGT
TCTTCCGATGTCTTCGTTTTGGGTCTAGGTGTTGTTTTGGCTGCCTTGTATATCTTCAG
AGACCAATTATTCGCTGCTTCTAAGCCAAAGGTGGCTCCAGTTTCCACTACGAAGCCTG
CCAACGGTTCCGCTAACCCAAGAGACTTCATCGCCAAGATGAAACAAGGTAAGAAGAGA
ATCGTAATCTTCTACGGTTCTCAAACTGGTACCGCTGAAGAATATGCTATTCGTTTGGC
TAAGGAAGCTAAGCAAAAGTTCGGTCTAGCCTCCTTGGTTTGTGATCCAGAAGAATACG
ATTTTGAAAAGTTGGACCAATTGCCAGAAGATTCTATTGCTTTCTTCGTCGTTGCTACC
TATGGTGAAGGTGAACCTACAGACAACGCTGTCCAATTGTTGCAAAACTTGCAAGATGA
AAGCTTCGAATTCTCCTCTGGTGAGAGAAAGTTGTCAGGTTTGAAGTACGTTGTTTTTG
GTCTGGGTAACAAGACCTACGAACATTACAACCTCATTGGGAGAACTGTTGACGCTCAA
TTGGCCAAGATGGGTGCTATCAGAATCGGTGAAAGAGGTGAAGGTGATGATGACAAGTC
CATGGAAGAAGACTACTTGGAATGGAAGGATGGTATGTGGGAAGCGTTTGCCACTGCTA
TGGGTGTTGAAGAAGGTCAAGGTGGTGACTCCGCTGATTTCGTCGTTTCCGAATTGGAA
TCTCACCCACCAGAAAAGGTTTACCAAGGTGAATTTTCTGCTAGAGCTTTAACCAAAAC
CAAGGGTATTCACGACGCTAAGAATCCTTTTGCTGCTCCAATTGCGGTTGCTAGAGAAT
TGTTCCAATCTGTTGTCGATAGAAACTGTGTCCACGTCGAATTCAACATTGAAGGCTCT
GGTATCACCTATCAACACGGTGACCACGTTGGTTTGTGGCCATTGAATCCAGATGTTGA
AGTCGAACGGTTGTTGTGTGTTTTAGGTTTAGCTGAAAAGAGAGATGCTGTCATCTCCA
TTGAATCCTTAGACCCGGCTTTGGCTAAGGTTCCATTCCCAGTCCCAACTACTTACGGT
GCTGTGTTGAGACACTACATTGACATCTCTGCTGTCGCCGGTAGACAAATCTTGGGTAC
TTTGTCCAAATTCGCTCCAACCCCAGAAGCTGAAGCTTTCTTGAGAAACTTGAACACTA
ACAAGGAAGAATACCACAACGTCGTCGCTAACGGTTGTTTGAAATTGGGTGAAATTTTG
CAAATCGCTACCGGTAACGACATTACTGTCCCACCAACTACTGCCAACACCACCAAATG
GCCAATTCCATTCGACATCATTGTTTCTGCCATCCCAAGATTGCAACCAAGATACTACT
CTATCTCTTCTTCCCCAAAAATTCATCCAAACACCATCCACGCTACCGTTGTTGTGCTC
AAATACGAAAACGTTCCAACCGAACCAATCCCAAGAAAGTGGGTTTACGGTGTCGGTAG
TAACTTCTTGTTGAATTTAAAGTACGCTGTTAACAAGGAACCAGTTCCATACATCACTC
AAAATGGCGAACAAAGAGTCGGTGTCCCGGAATACTTGATTGCTGGTCCACGTGGTTCT
TACAAGACTGAATCTTTCTACAAGGCTCCAATCCATGTTAGACGTTCTACTTTCCGTTT
GCCAACCAACCCAAAGTCTCCAGTCATCATGATTGGTCCAGGTACTGGTGTCGCCCCAT
TCAGAGGCTTCGTTCAAGAAAGAGTTGCCTTGGCCAGAAGATCCATCGAAAAGAACGGT
CCTGACTCTTTGGCTGACTGGGGTCGTATTTCCTTGTTCTACGGTTGTAGAAGATCCGA
CGAAGACTTCTTGTACAAGGACGAATGGCCACAATACGAAGCTGAGTTGAAGGGTAAGT
TCAAGTTGCACTGTGCTTTCTCCAGACAAAACTACAAGCCAGACGGTTCTAAGATTTAC
GTCCAAGATTTGATCTGGGAAGACAGAGAACACATTGCCGATGCCATCTTAAACGGTAA
GGGTTACGTCTACATCTGCGGTGAAGCTAAGTCCATGTCTAAACAAGTTGAAGAAGTTC
TAGCCAAGATCTTGGGCGAAGCCAAAGGTGGTTCCGGTCCAGTTGAAGGTGTTGCTGAA
GTCAAGTTACTGAAGGAACGGTCCAGATTGATGTTGGATGTCTGGTCTGAACAAAAGTT
AATTTCTGAAGAAGATTTGGAATGAATCGCGTGCATTCATCCGCTCTAACCGAAAAGGA
AGGAGTTAGACAACCTGAAGTCTAGGTCCCTATTTATTTTTTTATAGTTATGTTAGTAT
TAAGAACGTTATTTATATTTCAAATTTTTCTTTTTTTTCTGTACAGACGCGTGTACGCA
TGTAACATTATACTGAAAACCTTGCTTGAGAAGGTTTTGGGACGCTCGAAGATCGCGTC
CCAATTCGCCCTATAGTGAGTCGTATTACGCGCGCTCACTGGCCGTCGTTTTACAACGT
CGTGACTGGGAAAACCCTGGCGTTACCCCTGCAGGACTAGTGCTGAGGCATTAATACCA
CTTTTCAATGAAACGGATATTGATATGCTAGTAAAAGGACGAGCTCAAGAGCGAAAATA
TAAGTAAAGAATTCGAGTGCACTTGTCTCCATGCAGCAAGATTTCATATGAGTCTTTTT
TATCTTTTTACTTTTTACATTACACGATATGCACTTTATGAAAATTTAACGAGGTTGGA
AGCCGGATAATCAACCAAAATCAGGCACGAAGGCACACTCGTATATGCATGTTGTTGAA
ACTCTGTTACGCTGAACTAACAATCACACATGTAGAGGTCACCGGGAAAAGTTGCGACC
CCATGGAAGGTCGATCTCTTCGTTTGGCTTTGCTTGGCTGGCGGCATTGCGCTTCTTCG
CTTATACCCGTCTCTTGACGCTCGAGCTCGTTCATTGAGATACCTTTATTCTTGCACAT
TTTCTGGCTTTTTTCGCTACTCGGGTACATGTAATCATGCACACAGAAGGTGCTGTAGG
GTGAAAGTTCCTTTGTGCTGTCGTTTGTTTTTAATGCCAAACTTTCCGGTGATCAATAA
CCACCTC
SEQ. ID NO: 51
CGGCATGCAAACATCTACACAATTAGCAAGGGCAATCCATATTTTGTCTTTTCGCGCCC
TGGAAAGGCCTAAGTAATGTCGTAAACGCATTCTATCTGTACTTCAACTCTCCTCTGTG
CATTGGTTTGTGCAAATCACATTTTACGATACTGCCAGATATATGCAAAAAGAGAAAAC
CAAGGGACCAGAACAAAGCAAAATTACGATATTCTTCGAATTCCTTCGTGCTTGACTAA
GACAAAGGGATGGACGTAGCGATTTTTAGCGGGCCAAGAACTGGTTCCGAAAAAGCACA
GGTACACCGAACCCTCAGCTAAGGAGGGACAGCACCGATGCGGAAGGACAAACTTTCTT
TTTGCCTATCACAGTATCTTATCGAGCTAACTATTTTCGACACACATGAAAAAGCAGAA
ATATTAACGAAAAAGAAAAGAAAGACCATGTCATGTACGGGCAATCAGAATCTGTAACA
AGCGCCATTTTTTTTTCTGTATCGGGCCCTCCTTACTGCTCTCCTTCCGTGTAACGCGT
TATGAAATTAAGTCCTCAGCGAGCTCGCATGGAATGCGTGCGATGAGCGACCTCATGCT
ATACCTGAGAAAGCAACCTGACCTACAGGAAAGAGTTACTCAAGAATAAGAATTTTCGT
TTTAAAACCTAAGAGTCACTTTAAAATTTGTATACACTTATTTTTTTTATAACTTATTT
AATAATAAAAATCATAAATCATAAGAAATTCGCTTATTTAGAAGTGTCAACAACGTATC
TACCAACGGAATGCGTGCGATTGTTTTATATTTGTTGTAAAAAGTAGATAATTACTTCC
TTGATGATCTGTAAAAAAGAGAAAAAGAAAGCATCTAAGAACTTGAAAAACTACGAATT
AGAAAAGACCAAATATGTATTTCTTGCATTGACCAATTTATGCAAGTTTATATATATGT
AAATGTAAGTTTCACGAGGTTCTACTAAACTAAACCACCCCCTTGGTTAGAAGAAAAGA
GTGTGTGAGAACAGGCTGTTGTTGTCACACGATTCGGACAATTCTGTTTGAAAGAGAGA
GAGTAACAGTACGATCGAACGAACTTTGCTCTGGAGATCACAGTGGGCATCATAGCATG
TGGTACTAAACCCTTTCCCGCCATTCCAGAACCTTCGATTGCTTGTTACAAAACCTGTG
AGCCGTCGCTAGGACCTTGTTGTGTGACGAAATTGGAAGCTGCAATCAATAGGAAGACA
GGAAGTCGAGCGTGTCTGGGTTTTTTCAGTTTTGTTCTTTTTGCAAACAACAGTTTATT
CCTGGCATCCACTAAATATAATGGAGCCCGCTTTTTAAGCTGGCATCCAGAAAAAAAAA
GAATCCCAGCACCAAAATATTGTTTTCTTCACCAACCATCAGTTCATAGGTCCATTCTC
TTAGCGCAACTACAGAGAACAGGGGCACAAACAGGCAAAAAACGGGCACAACCTCAATG
GAGTGATGCAACCTGCCTGGAGTAAATGATGACACAAGGCAATTGACCCACGCATGTAT
CTATCTCATTTTCTTACACCTTCTATTACCTTCTGCTCTCTCTGATTTGGAAAAAGCTG
AAAAAAAAGGTTGAAACCAGTTCCCTGAAATTATTCCCCTACTTGACTAATAAGTATAT
AAAGACGGTAGGTATTGATTGTAATTCTGTAAATCTATTTCTTAAACTTCTTAAATTCT
ACTTTTATAGTTAGTCTTTTTTTTAGTTTTAAAACACCAAGAACTTAGTTTCGAATAAA
CACACATAAACAAACAAAATGGGAGGTCCGATGAGCGGTTTCCATTCGGGGGAGGCGCT
GCTCGGTGACCTCGCCACCGGTCAGCTGACCAGGCTGTGCGAGGTGGCGGGGCTGACCG
AGGCCGACACGGCGGCCTACACGGGGGTGCTGATCGAAAGTCTGGGGACGTCGGCCGGA
CGGCCGTTGTCCCTGCCACCCCCGTCGCGGACCTTTCTCTCCGACGACCACACCCCCGT
GGAGTTCTCCCTGGCCTTCCTGCCGGGACGCGCACCGCACCTGCGGGTCCTGGTGGAAC
CGGGCTGCTCCAGCGGCGACGACCTGGCGGAAAACGGCCGGGCCGGTCTGCGGGCGGTC
CACACCATGGCGGACCGCTGGGGATTCTCCACCGAGCAACTCGACCGGCTGGAGGACCT
GTTCTTCCCCTCCTCCCCCGAGGGCCCGCTGGCCCTGTGGTGCGCCCTGGAGCTCCGCT
CCGGTGGGGTGCCGGGGGTGAAGGTCTACCTCAACCCCGCGGCGAATGGCGCCGACCGG
GCCGCCGAGACGGTACGCGAGGCGCTGGCCAGGCTGGGCCACCTGCAGGCGTTCGACGC
GCTGCCCCGGGCGGACGGCTTCCCGTTCCTCGCCCTGGACCTCGGCGACTGGGACGCCC
CGCGGGTGAAGATCTACCTCAAACACCTCGGCATGTCCGCCGCCGACGCGGGCTCCCTC
CCCCGGATGTCGCCCGCACCGAGCCGGGAGCAGCTGGAGGAGTTCTTCCGCACCGCCGG
TGACCTCCCGGCCCCGGGAGACCCGGGGCCCACCGAGGACACCGGCCGGCTCGCCGGGC
GCCCCGCCCTCACCTGCCACTCCTTCACGGAGACGGCGACCGGGCGGCCCAGCGGCTAC
ACCCTCCACGTGCCGGTCCGCGACTACGTCCGGCACGACGGCGAGGCACGGGACCGGGC
GGTGGCCGTGCTGCGCGAACATGACATGGACAGTGCGGCACTGGACCGGGCGCTGGCCG
CCGTGAGCCCCCGCCCGCTGAGTGACGGGGTGGGCCTGATCGCCTATCTGGCACTGGTC
CACCAGCGCGGCCGGCCGACACGGGTGACCGTCTACGTCTCCTCCGAGGCGTACGAGGT
GCGGCCGCCCCGCGAGACGGTCCCCACCCGCGACCGGGCGCGGGCACGGCTGCATCATC
ATCATCATCATTGAATCGCGTGCATTCATCCGCTCTAACCGAAAAGGAAGGAGTTAGAC
AACCTGAAGTCTAGGTCCCTATTTATTTTTTTATAGTTATGTTAGTATTAAGAACGTTA
TTTATATTTCAAATTTTTCTTTTTTTTCTGTACAGACGCGTGTACGCATGTAACATTAT
ACTGAAAACCTTGCTTGAGAAGGTTTTGGGACGCTCGAAGATCGCGTCCCAATTCGCCC
TATAGTGAGTCGTATTACGCGCGCTCACTGGCCGTCGTTTTACAACGTCGTGACTGGGA
AAACCCTGGCGTTACCCCTGCAGGACTAGTGCTGAGGCATTAATACGACTCTCTCGAAA
TTTTTCTTAACGCGTCCTTGTACTGCGTCTAACGCTTTTGCCACTTGGATTTCTATTAT
AGGAAATAGTCTCACTTACTGGGCGACGAATTTTCGCGTTTTGATGAAGCACAGGAAGA
ATTTCTTTTTTTTTTGGCTTCTTCTGGTTCCGTTTTTTACGCGCACAAATCTAAAAAAA
GAAATAATTATAACCTAGTCTCGAAAATTTTCATCGATCCATTCGTTCCTTTTTTTCGA
TTTTTTCAGATCAAAATTCTTGTTTCTTTCTTTGTCTTAGTTTATATTAAAAGATATTT
TGATTTTACTCCTGAACTATTTATTCTTTCTAAGAAGGCCAGAACACTACAGCTGTTTT
AACCGACTACGAAGTTCTCCATTCTCGAACACTAGCCTTCATTTACCAAACAGGAACTA
GCGTATATCATTAGTCCTTATTCGAAAAGAGATTGGTAGATATTTATTGTAGTTTGTGA
GAAGGAGAAAATACTGTCATTGGACTGATAGTTAGAGGACATTAACCTCTCTTACGTTC
GCTCA
SEQ. ID NO: 52
CGAGATCTTTGTGTTCGGTTACCCGGCTCAGATCCTAACTTCTTCTTTTGGTATGTTTA
TTCGTATAAGTTACTGTTGTCCACAGGCAATACTCTGCAGAAAATTAAAACGGCATTAA
TGCTAGGACAACCAGAATTGTTACTACTGTATGTGCGATAGTTGATAACTGCAACATTA
TGCCCGGTATATTCTCAAAAAACCCTATTACTGCATACGAAGAAATCGCAAGAGAAATC
TTTCGGTTTGGAAAAGCTCACTGTGAGGTTCCTTGGAGCCAATAGTAATACAGCACAAT
CCAAGGAAAAATCTGGCCTATATGCAAGGAAGGAGAGATAGTCAAAAGCATTCTTTCCC
CTAGAAGTTGGTGCATATATGGCATCGTTAAAACATATTACCCCCAAAATTTCTTCTCT
AAACGATGTGCTTGGCCTTTGTTTTGGTTTTTGATGTCGGTCGTTTGAGGCCCCTTGCG
GAAAATCGAGATCGCCGAATGGCACGCGAGGGAAGGGAAATAAGGTTTAAAGGCACTGA
AACAATAGGCAAGAAGTAGGCGAGAGCCGACATACGAGACTAAATTAAGTCCTCAGCGA
GCTCGCATGGAATGCGTGCGATGAGCGACCTCATGCTATACCTGAGAAAGCAACCTGAC
CTACAGGAAAGAGTTACTCAAGAATAAGAATTTTCGTTTTAAAACCTAAGAGTCACTTT
AAAATTTGTATACACTTATTTTTTTTATAACTTATTTAATAATAAAAATCATAAATCAT
AAGAAATTCGCTTATTTAGAAGTGTCAACAACGTATCTACCAACGGAATGCGTGCGATT
CATTTGTCATCGTCATCCTTGTAGTCCATTGCCTTCAAATGTTCCTTATTAAACTTTCT
CAAATCGTAAACCAAAGAGTTCAAGATATCCAATTCAACATGTTCCATAACAACGATCT
TGTACCAGTTGTTAGTTGGGTTATGAACTTCTGGAACTAAAAAGTACTTTTCAGCGATT
TCCTTATTAACATATTGTTCTTCAATTGTAACAATATTCATTGAATCTTCTTTGTAATA
TTTAATTCTCATATCGTTCAATTGCTTACACAACCATTTAGTTCTATTTCTCAACTTAT
TAATCTTTTCCATCCAACCGTATGGACCATAAGAAGCTAAAACCATCCAAATTGCAACA
GCATTTGAACCAGATCTTGAACCAGACAATGTAACATCCAAATTTTCGATGTAAGTTGC
TTCCTTTGTCAAAGTGTTATGGATCAAATTCTTTCTTGAAACGAAGATACCAGTACCGT
ATGGAGCTTGCAACATCTTATGACCATCTAATGTGATTGAAGAAACGTTCTTATTAGAG
AAATCAGTTTTACATTCCTTATTATCAATTGGATATATAAAACCACCAAATGCACCATC
AACATGAATTTTGTATTCCAAATTGTACTTATCGAAGATGTTAGCGTACAAATCTGGAT
CATCAACTGAACCAAACATTGTAGTACCCATGTTAGAGATAACGATGAAGTACTTTTTA
CCAATTTCTTTAGCTTCTTTAACAATTGAATCCAATGTATTTTCTTGAATTTTTCTTGA
ATAAAAATCAACTGGAACCTTAATAATATCGATGTTCAACAAATCTGAACCTTTGTATG
CAGAGTAATGTGTATCTGCTGAAGTGATGATAGCGATTTCTTCATGCTTAGCCTTTCTT
TCTTTCTTGAAGTAGTTTCTGTAAACCCACATTGCTTGGATGTTAGCTTCTGTACCACC
TTGAGTAACGTAACCATCAAATTCTTCATCGTTACCGTTCAAAACATCGATTGCTAACA
ATTGGATTAATTCTCTTTCGATATCGAATGTACCACCGAACAAGATATCAGCCTTATCA
TAAGTATGACAACCGATATGGTTTGGGTTTTGAATAAAAGTTCTCAAGTATGGTGAATG
CTTAACGAAAGAATGATCATCATAGAAAACTGTATCATCCAACTTAGTACCTGGAATAC
CGATTGTCTTAGTGTTATCGTAGTTCAAAGTCTTTTCCAAAGATTCAGTAATCTTTTCA
TCCATTTCTTGTTGTGTGTACTTTCTCCAGAACTTCATTGTTTTATATTTGTTGTAAAA
AGTAGATAATTACTTCCTTGATGATCTGTAAAAAAGAGAAAAAGAAAGCATCTAAGAAC
TTGAAAAACTACGAATTAGAAAAGACCAAATATGTATTTCTTGCATTGACCAATTTATG
CAAGTTTATATATATGTAAATGTAAGTTTCACGAGGTTCTACTAAACTAAACCACCCCC
TTGGTTAGAAGAAAAGAGTGTGTGAGAACAGGCTGTTGTTGTCACACGATTCGGACAAT
TCTGTTTGAAAGAGAGAGAGTAACAGTACGATCGAACGAACTTTGCTCTGGAGATCACA
GTGGGCATCATAGCATGTGGTACTAAACCCTTTCCCGCCATTCCAGAACCTTCGATTGC
TTGTTACAAAACCTGTGAGCCGTCGCTAGGACCTTGTTGTGTGACGAAATTGGAAGCTG
CAATCAATAGGAAGACAGGAAGTCGAGCGTGTCTGGGTTTTTTCAGTTTTGTTCTTTTT
GCAAACAACAGTTTATTCCTGGCATCCACTAAATATAATGGAGCCCGCTTTTTAAGCTG
GCATCCAGAAAAAAAAAGAATCCCAGCACCAAAATATTGTTTTCTTCACCAACCATCAG
TTCATAGGTCCATTCTCTTAGCGCAACTACAGAGAACAGGGGCACAAACAGGCAAAAAA
CGGGCACAACCTCAATGGAGTGATGCAACCTGCCTGGAGTAAATGATGACACAAGGCAA
TTGACCCACGCATGTATCTATCTCATTTTCTTACACCTTCTATTACCTTCTGCTCTCTC
TGATTTGGAAAAAGCTGAAAAAAAAGGTTGAAACCAGTTCCCTGAAATTATTCCCCTAC
TTGACTAATAAGTATATAAAGACGGTAGGTATTGATTGTAATTCTGTAAATCTATTTCT
TAAACTTCTTAAATTCTACTTTTATAGTTAGTCTTTTTTTTAGTTTTAAAACACCAAGA
ACTTAGTTTCGAATAAACACACATAAACAAACAAAATGGCTTCTAGTTCTTCCGATGTC
TTCGTTTTGGGTCTAGGTGTTGTTTTGGCTGCCTTGTATATCTTCAGAGACCAATTATT
CGCTGCTTCTAAGCCAAAGGTGGCTCCAGTTTCCACTACGAAGCCTGCCAACGGTTCCG
CTAACCCAAGAGACTTCATCGCCAAGATGAAACAAGGTAAGAAGAGAATCGTAATCTTC
TACGGTTCTCAAACTGGTACCGCTGAAGAATATGCTATTCGTTTGGCTAAGGAAGCTAA
GCAAAAGTTCGGTCTAGCCTCCTTGGTTTGTGATCCAGAAGAATACGATTTTGAAAAGT
TGGACCAATTGCCAGAAGATTCTATTGCTTTCTTCGTCGTTGCTACCTATGGTGAAGGT
GAACCTACAGACAACGCTGTCCAATTGTTGCAAAACTTGCAAGATGAAAGCTTCGAATT
CTCCTCTGGTGAGAGAAAGTTGTCAGGTTTGAAGTACGTTGTTTTTGGTCTGGGTAACA
AGACCTACGAACATTACAACCTCATTGGGAGAACTGTTGACGCTCAATTGGCCAAGATG
GGTGCTATCAGAATCGGTGAAAGAGGTGAAGGTGATGATGACAAGTCCATGGAAGAAGA
CTACTTGGAATGGAAGGATGGTATGTGGGAAGCGTTTGCCACTGCTATGGGTGTTGAAG
AAGGTCAAGGTGGTGACTCCGCTGATTTCGTCGTTTCCGAATTGGAATCTCACCCACCA
GAAAAGGTTTACCAAGGTGAATTTTCTGCTAGAGCTTTAACCAAAACCAAGGGTATTCA
CGACGCTAAGAATCCTTTTGCTGCTCCAATTGCGGTTGCTAGAGAATTGTTCCAATCTG
TTGTCGATAGAAACTGTGTCCACGTCGAATTCAACATTGAAGGCTCTGGTATCACCTAT
CAACACGGTGACCACGTTGGTTTGTGGCCATTGAATCCAGATGTTGAAGTCGAACGGTT
GTTGTGTGTTTTAGGTTTAGCTGAAAAGAGAGATGCTGTCATCTCCATTGAATCCTTAG
ACCCGGCTTTGGCTAAGGTTCCATTCCCAGTCCCAACTACTTACGGTGCTGTGTTGAGA
CACTACATTGACATCTCTGCTGTCGCCGGTAGACAAATCTTGGGTACTTTGTCCAAATT
CGCTCCAACCCCAGAAGCTGAAGCTTTCTTGAGAAACTTGAACACTAACAAGGAAGAAT
ACCACAACGTCGTCGCTAACGGTTGTTTGAAATTGGGTGAAATTTTGCAAATCGCTACC
GGTAACGACATTACTGTCCCACCAACTACTGCCAACACCACCAAATGGCCAATTCCATT
CGACATCATTGTTTCTGCCATCCCAAGATTGCAACCAAGATACTACTCTATCTCTTCTT
CCCCAAAAATTCATCCAAACACCATCCACGCTACCGTTGTTGTGCTCAAATACGAAAAC
GTTCCAACCGAACCAATCCCAAGAAAGTGGGTTTACGGTGTCGGTAGTAACTTCTTGTT
GAATTTAAAGTACGCTGTTAACAAGGAACCAGTTCCATACATCACTCAAAATGGCGAAC
AAAGAGTCGGTGTCCCGGAATACTTGATTGCTGGTCCACGTGGTTCTTACAAGACTGAA
TCTTTCTACAAGGCTCCAATCCATGTTAGACGTTCTACTTTCCGTTTGCCAACCAACCC
AAAGTCTCCAGTCATCATGATTGGTCCAGGTACTGGTGTCGCCCCATTCAGAGGCTTCG
TTCAAGAAAGAGTTGCCTTGGCCAGAAGATCCATCGAAAAGAACGGTCCTGACTCTTTG
GCTGACTGGGGTCGTATTTCCTTGTTCTACGGTTGTAGAAGATCCGACGAAGACTTCTT
GTACAAGGACGAATGGCCACAATACGAAGCTGAGTTGAAGGGTAAGTTCAAGTTGCACT
GTGCTTTCTCCAGACAAAACTACAAGCCAGACGGTTCTAAGATTTACGTCCAAGATTTG
ATCTGGGAAGACAGAGAACACATTGCCGATGCCATCTTAAACGGTAAGGGTTACGTCTA
CATCTGCGGTGAAGCTAAGTCCATGTCTAAACAAGTTGAAGAAGTTCTAGCCAAGATCT
TGGGCGAAGCCAAAGGTGGTTCCGGTCCAGTTGAAGGTGTTGCTGAAGTCAAGTTACTG
AAGGAACGGTCCAGATTGATGTTGGATGTCTGGTCTGAACAAAAGTTAATTTCTGAAGA
AGATTTGGAATGAATCGCGTGCATTCATCCGCTCTAACCGAAAAGGAAGGAGTTAGACA
ACCTGAAGTCTAGGTCCCTATTTATTTTTTTATAGTTATGTTAGTATTAAGAACGTTAT
TTATATTTCAAATTTTTCTTTTTTTTCTGTACAGACGCGTGTACGCATGTAACATTATA
CTGAAAACCTTGCTTGAGAAGGTTTTGGGACGCTCGAAGATCGCGTCCCAATTCGCCCT
ATAGTGAGTCGTATTACGCGCGCTCACTGGCCGTCGTTTTACAACGTCGTGACTGGGAA
AACCCTGGCGTTACCCCTGCAGGACTAGTGCTGAGGCATTAATACCACTTTTCAATGAA
ACGGATATTGATATGCTAGTAAAAGGACGAGCTCAAGAGCGAAAATATAAGTAAAGAAT
TCGAGTGCACTTGTCTCCATGCAGCAAGATTTCATATGAGTCTTTTTTATCTTTTTACT
TTTTACATTACACGATATGCACTTTATGAAAATTTAACGAGGTTGGAAGCCGGATAATC
AACCAAAATCAGGCACGAAGGCACACTCGTATATGCATGTTGTTGAAACTCTGTTACGC
TGAACTAACAATCACACATGTAGAGGTCACCGGGAAAAGTTGCGACCCCATGGAAGGTC
GATCTCTTCGTTTGGCTTTGCTTGGCTGGCGGCATTGCGCTTCTTCGCTTATACCCGTC
TCTTGACGCTCGAGCTCGTTCATTGAGATACCTTTATTCTTGCACATTTTCTGGCTTTT
TTCGCTACTCGGGTACATGTAATCATGCACACAGAAGGTGCTGTAGGGTGAAAGTTCCT
TTGTGCTGTCGTTTGTTTTTAATGCCAAACTTTCCGGTGATCAATAACCACCTC
SEQ. ID NO: 53
CGGCATGCAAACATCTACACAATTAGCAAGGGCAATCCATATTTTGTCTTTTCGCGCCC
TGGAAAGGCCTAAGTAATGTCGTAAACGCATTCTATCTGTACTTCAACTCTCCTCTGTG
CATTGGTTTGTGCAAATCACATTTTACGATACTGCCAGATATATGCAAAAAGAGAAAAC
CAAGGGACCAGAACAAAGCAAAATTACGATATTCTTCGAATTCCTTCGTGCTTGACTAA
GACAAAGGGATGGACGTAGCGATTTTTAGCGGGCCAAGAACTGGTTCCGAAAAAGCACA
GGTACACCGAACCCTCAGCTAAGGAGGGACAGCACCGATGCGGAAGGACAAACTTTCTT
TTTGCCTATCACAGTATCTTATCGAGCTAACTATTTTCGACACACATGAAAAAGCAGAA
ATATTAACGAAAAAGAAAAGAAAGACCATGTCATGTACGGGCAATCAGAATCTGTAACA
AGCGCCATTTTTTTTTCTGTATCGGGCCCTCCTTACTGCTCTCCTTCCGTGTAACGCGT
TATGAAATTAAGTCCTCAGCGAGCTCGCATGGAATGCGTGCGATGAGCGACCTCATGCT
ATACCTGAGAAAGCAACCTGACCTACAGGAAAGAGTTACTCAAGAATAAGAATTTTCGT
TTTAAAACCTAAGAGTCACTTTAAAATTTGTATACACTTATTTTTTTTATAACTTATTT
AATAATAAAAATCATAAATCATAAGAAATTCGCTTATTTAGAAGTGTCAACAACGTATC
TACCAACGGAATGCGTGCGATTGTTTTATATTTGTTGTAAAAAGTAGATAATTACTTCC
TTGATGATCTGTAAAAAAGAGAAAAAGAAAGCATCTAAGAACTTGAAAAACTACGAATT
AGAAAAGACCAAATATGTATTTCTTGCATTGACCAATTTATGCAAGTTTATATATATGT
AAATGTAAGTTTCACGAGGTTCTACTAAACTAAACCACCCCCTTGGTTAGAAGAAAAGA
GTGTGTGAGAACAGGCTGTTGTTGTCACACGATTCGGACAATTCTGTTTGAAAGAGAGA
GAGTAACAGTACGATCGAACGAACTTTGCTCTGGAGATCACAGTGGGCATCATAGCATG
TGGTACTAAACCCTTTCCCGCCATTCCAGAACCTTCGATTGCTTGTTACAAAACCTGTG
AGCCGTCGCTAGGACCTTGTTGTGTGACGAAATTGGAAGCTGCAATCAATAGGAAGACA
GGAAGTCGAGCGTGTCTGGGTTTTTTCAGTTTTGTTCTTTTTGCAAACAACAGTTTATT
CCTGGCATCCACTAAATATAATGGAGCCCGCTTTTTAAGCTGGCATCCAGAAAAAAAAA
GAATCCCAGCACCAAAATATTGTTTTCTTCACCAACCATCAGTTCATAGGTCCATTCTC
TTAGCGCAACTACAGAGAACAGGGGCACAAACAGGCAAAAAACGGGCACAACCTCAATG
GAGTGATGCAACCTGCCTGGAGTAAATGATGACACAAGGCAATTGACCCACGCATGTAT
CTATCTCATTTTCTTACACCTTCTATTACCTTCTGCTCTCTCTGATTTGGAAAAAGCTG
AAAAAAAAGGTTGAAACCAGTTCCCTGAAATTATTCCCCTACTTGACTAATAAGTATAT
AAAGACGGTAGGTATTGATTGTAATTCTGTAAATCTATTTCTTAAACTTCTTAAATTCT
ACTTTTATAGTTAGTCTTTTTTTTAGTTTTAAAACACCAAGAACTTAGTTTCGAATAAA
CACACATAAACAAACAAAATGTCCATCGGTGCTGAAATTGACTCTTTGGTTCCAGCTCC
ACCAGGTTTGAACGGTACCGCTGCTGGTTACCCAGCCAAGACTCAAAAGGAATTGTCTA
ACGGCGATTTCGATGCTCACGATGGTCTGTCCTTGGCTCAATTGACTCCATACGATGTT
TTAACCGCTGCTTTGCCATTGCCAGCGCCAGCTTCTAGTACTGGTTTCTGGTGGAGAGA
AACTGGTCCAGTTATGTCTAAGCTCTTGGCTAAAGCCAACTACCCATTGTACACCCATT
ACAAGTATTTAATGTTGTACCACACTCACATTTTACCTTTGTTAGGTCCAAGACCACCT
TTGGAAAATTCTACCCACCCATCTCCATCAAATGCTCCTTGGAGATCCTTCTTGACCGA
TGACTTCACCCCATTAGAACCATCTTGGAACGTTAACGGTAACTCCGAAGCACAATCCA
CTATCAGATTGGGTATTGAACCAATTGGTTTCGAAGCCGGTGCTGCTGCCGACCCATTC
AACCAAGCTGCCGTCACCCAATTCATGCACTCCTACGAAGCTACTGAAGTTGGTGCCAC
TCTAACTTTGTTCGAACACTTCAGAAACGACATGTTCGTCGGTCCAGAGACTTACGCTG
CCTTGAGAGCTAAGATTCCTGAAGGTGAGCACACCACTCAATCTTTCTTGGCTTTCGAC
TTGGACGCCGGTCGTGTCACTACCAAGGCTTACTTCTTCCCAATCTTGATGTCTTTGAA
GACCGGTCAATCTACGACCAAAGTIGTTTCCGATTCTATCTTGCACCTAGCTTTGAAGT
CTGAAGTTTGGGGTGTCCAAACCATTGCCGCTATGTCGGTCATGGAAGCTTGGATCGGT
TCTTACGGTGGTGCTGCTAAGACCGAAATGATCTCCGTTGACTGTGTCAACGAAGCTGA
CTCCAGAATCAAGATCTACGTTAGAATGCCACACACTAGCTTGAGAAAGGTCAAAGAAG
CTTATTGTTTGGGTGGCCGTTTGACTGACGAAAACACCAAGGAAGGTTTGAAATTGTTG
GATGAATTGTGGAGAACTGTTTTCGGTATCGATGACGAAGATGCTGAATTACCACAAAA
CTCTCACAGAACTGCTGGTACTATTTTTAACTTTGAACTAAGACCAGGTAAGTGGTTCC
CAGAACCAAAGGTCTACTTGCCAGTCAGACACTACTGTGAATCCGACATGCAAATTGCC
TCCAGATTACAAACTTTCTTTGGTCGTTTGGGTTGGCACAACATGGAAAAGGACTACTG
CAAGCATTTGGAAGACTTATTCCCTCACCACCCATTGTCCTCCTCTACCGGTACCCACA
CTTTCTTGTCTTTTTCTTACAAGAAGCAAAAGGGTGTTTACATGACCATGTACTACAAC
TTGAGAGTTTATTCTACACACCACCATCATCATCATTGAATCGCGTGCATTCATCCGCT
CTAACCGAAAAGGAAGGAGTTAGACAACCTGAAGTCTAGGTCCCTATTTATTTTTTTAT
AGTTATGTTAGTATTAAGAACGTTATTTATATTTCAAATTTTTCTTTTTTTTCTGTACA
GACGCGTGTACGCATGTAACATTATACTGAAAACCTTGCTTGAGAAGGTTTTGGGACGC
TCGAAGATCGCGTCCCAATTCGCCCTATAGTGAGTCGTATTACGCGCGCTCACTGGCCG
TCGTTTTACAACGTCGTGACTGGGAAAACCCTGGCGTTACCCCTGCAGGACTAGTGCTG
AGGCATTAATACGACTCTCTCGAAATTTTTCTTAACGCGTCCTTGTACTGCGTCTAACG
CTTTTGCCACTTGGATTTCTATTATAGGAAATAGTCTCACTTACTGGGCGACGAATTTT
CGCGTTTTGATGAAGCACAGGAAGAATTTCTTTTTTTTTTGGCTTCTTCTGGTTCCGTT
TTTTACGCGCACAAATCTAAAAAAAGAAATAATTATAACCTAGTCTCGAAAATTTTCAT
CGATCCATTCGTTCCTTTTTTTCGATTTTTTCAGATCAAAATTCTTGTTTCTTTCTTTG
TCTTAGTTTATATTAAAAGATATTTTGATTTTACTCCTGAACTATTTATTCTTTCTAAG
AAGGCCAGAACACTACAGCTGTTTTAACCGACTACGAAGTTCTCCATTCTCGAACACTA
GCCTTCATTTACCAAACAGGAACTAGCGTATATCATTAGTCCTTATTCGAAAAGAGATT
GGTAGATATTTATTGTAGTTTGTGAGAAGGAGAAAATACTGTCATTGGACTGATAGTTA
GAGGACATTAACCTCTCTTACGTTCGCTCA
SEQ. ID NO: 54
ATCCGGATATAGTTCCTCCTTTCAGCAAAAAACCCCTCAAGACCCGTTTAGAGGCCCCA
AGGGGTTATGCTAGTTATTGCTCAGCGGTGGCAGCAGCCAACTCAGCTTCCTTTCGGGC
TTTGTTAGCAGCCGGATCTCAGTGGTGGTGGTGGTGGTGCTCGAGTGCGGCCGCAAGCT
TGTCGACGGAGCTCGAATTCGGATCCGAATTAATTCCGATATCCATGGCCATCGCCGGC
TGGGCAGCGAGGAGCAGCAGACCAGCAGCAGCGGTCGGCAGCAGGTATTTCATATGTAT
ATCTCCTTCTTAAAGTTAAACAAAATTATTTCTAGAGGGGAATTGTTATCCGCTCACAA
TTCCCCTATAGTGAGTCGTATTAATTTCGCGGGATCGAGATCTCGATCCTCTACGCCGG
ACGCATCGTGGCCGGCATCACCGGCGCCACAGGTGCGGTTGCTGGCGCCTATATCGCCG
ACATCACCGATGGGGAAGATCGGGCTCGCCACTTCGGGCTCATGAGCGCTTGTTTCGGC
GTGGGTATGGTGGCAGGCCCCGTGGCCGGGGGACTGTTGGGCGCCATCTCCTTGCATGC
ACCATTCCTTGCGGCGGCGGTGCTCAACGGCCTCAACCTACTACTGGGCTGCTTCCTAA
TGCAGGAGTCGCATAAGGGAGAGCGTCGAGATCCCGGACACCATCGAATGGCGCAAAAC
CTTTCGCGGTATGGCATGATAGCGCCCGGAAGAGAGTCAATTCAGGGTGGTGAATGTGA
AACCAGTAACGTTATACGATGTCGCAGAGTATGCCGGTGTCTCTTATCAGACCGTTTCC
CGCGTGGTGAACCAGGCCAGCCACGTTTCTGCGAAAACGCGGGAAAAAGTGGAAGCGGC
GATGGCGGAGCTGAATTACATTCCCAACCGCGTGGCACAACAACTGGCGGGCAAACAGT
CGTTGCTGATTGGCGTTGCCACCTCCAGTCTGGCCCTGCACGCGCCGTCGCAAATTGTC
GCGGCGATTAAATCTCGCGCCGATCAACTGGGTGCCAGCGTGGTGGTGTCGATGGTAGA
ACGAAGCGGCGTCGAAGCCTGTAAAGCGGCGGTGCACAATCTTCTCGCGCAACGCGTCA
GTGGGCTGATCATTAACTATCCGCTGGATGACCAGGATGCCATTGCTGTGGAAGCTGCC
TGCACTAATGTTCCGGCGTTATTTCTTGATGTCTCTGACCAGACACCCATCAACAGTAT
TATTTTCTCCCATGAAGACGGTACGCGACTGGGCGTGGAGCATCTGGTCGCATTGGGTC
ACCAGCAAATCGCGCTGTTAGCGGGCCCATTAAGTTCTGTCTCGGCGCGTCTGCGTCTG
GCTGGCTGGCATAAATATCTCACTCGCAATCAAATTCAGCCGATAGCGGAACGGGAAGG
CGACTGGAGTGCCATGTCCGGTTTTCAACAAACCATGCAAATGCTGAATGAGGGCATCG
TTCCCACTGCGATGCTGGTTGCCAACGATCAGATGGCGCTGGGCGCAATGCGCGCCATT
ACCGAGTCCGGGCTGCGCGTTGGTGCGGATATCTCGGTAGTGGGATACGACGATACCGA
AGACAGCTCATGTTATATCCCGCCGTTAACCACCATCAAACAGGATTTTCGCCTGCTGG
GGCAAACCAGCGTGGACCGCTTGCTGCAACTCTCTCAGGGCCAGGCGGTGAAGGGCAAT
CAGCTGTTGCCCGTCTCACTGGTGAAAAGAAAAACCACCCTGGCGCCCAATACGCAAAC
CGCCTCTCCCCGCGCGTTGGCCGATTCATTAATGCAGCTGGCACGACAGGTTTCCCGAC
TGGAAAGCGGGCAGTGAGCGCAACGCAATTAATGTAAGTTAGCTCACTCATTAGGCACC
GGGATCTCGACCGATGCCCTTGAGAGCCTTCAACCCAGTCAGCTCCTTCCGGTGGGCGC
GGGGCATGACTATCGTCGCCGCACTTATGACTGTCTTCTTTATCATGCAACTCGTAGGA
CAGGTGCCGGCAGCGCTCTGGGTCATTTTCGGCGAGGACCGCTTTCGCTGGAGCGCGAC
GATGATCGGCCTGTCGCTTGCGGTATTCGGAATCTTGCACGCCCTCGCTCAAGCCTTCG
TCACTGGTCCCGCCACCAAACGTTTCGGCGAGAAGCAGGCCATTATCGCCGGCATGGCG
GCCCCACGGGTGCGCATGATCGTGCTCCTGTCGTTGAGGACCCGGCTAGGCTGGCGGGG
TTGCCTTACTGGTTAGCAGAATGAATCACCGATACGCGAGCGAACGTGAAGCGACTGCT
GCTGCAAAACGTCTGCGACCTGAGCAACAACATGAATGGTCTTCGGTTTCCGTGTTTCG
TAAAGTCTGGAAACGCGGAAGTCAGCGCCCTGCACCATTATGTTCCGGATCTGCATCGC
AGGATGCTGCTGGCTACCCTGTGGAACACCTACATCTGTATTAACGAAGCGCTGGCATT
GACCCTGAGTGATTTTTCTCTGGTCCCGCCGCATCCATACCGCCAGTTGTTTACCCTCA
CAACGTTCCAGTAACCGGGCATGTTCATCATCAGTAACCCGTATCGTGAGCATCCTCTC
TCGTTTCATCGGTATCATTACCCCCATGAACAGAAATCCCCCTTACACGGAGGCATCAG
TGACCAAACAGGAAAAAACCGCCCTTAACATGGCCCGCTTTATCAGAAGCCAGACATTA
ACGCTTCTGGAGAAACTCAACGAGCTGGACGCGGATGAACAGGCAGACATCTGTGAATC
GCTTCACGACCACGCTGATGAGCTTTACCGCAGCTGCCTCGCGCGTTTCGGTGATGACG
GTGAAAACCTCTGACACATGCAGCTCCCGGAGACGGTCACAGCTTGTCTGTAAGCGGAT
GCCGGGAGCAGACAAGCCCGTCAGGGCGCGTCAGCGGGTGTTGGCGGGTGTCGGGGCGC
AGCCATGACCCAGTCACGTAGCGATAGCGGAGTGTATACTGGCTTAACTATGCGGCATC
AGAGCAGATTGTACTGAGAGTGCACCATATATGCGGTGTGAAATACCGCACAGATGCGT
AAGGAGAAAATACCGCATCAGGCGCTCTTCCGCTTCCTCGCTCACTGACTCGCTGCGCT
CGGTCGTTCGGCTGCGGCGAGCGGTATCAGCTCACTCAAAGGCGGTAATACGGTTATCC
ACAGAATCAGGGGATAACGCAGGAAAGAACATGTGAGCAAAAGGCCAGCAAAAGGCCAG
GAACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGG
ACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAACAATAAAACTGT
CTGCTTACATAAACAGTAATACAAGGGGTGTTATGAGCCATATTCAACGGGAAACGTCT
TGCTCTAGGCCGCGATTAAATTCCAACATGGATGCTGATTTATATGGGTATAAATGGGC
TCGCGATAATGTCGGGCAATCAGGTGCGACAATCTATCGATTGTATGGGAAGCCCGATG
CGCCAGAGTTGTTTCTGAAACATGGCAAAGGTAGCGTTGCCAATGATGTTACAGATGAG
ATGGTCAGACTAAACTGGCTGACGGAATTTATGCCTCTTCCGACCATCAAGCATTTTAT
CCGTACTCCTGATGATGCATGGTTACTCACCACTGCGATCCCCGGGAAAACAGCATTCC
AGGTATTAGAAGAATATCCTGATTCAGGTGAAAATATTGTTGATGCGCTGGCAGTGTTC
CTGCGCCGGTTGCATTCGATTCCTGTTTGTAATTGTCCTTTTAACAGCGATCGCGTATT
TCGTCTCGCTCAGGCGCAATCACGAATGAATAACGGTTTGGTTGATGCGAGTGATTTTG
ATGACGAGCGTAATGGCTGGCCTGTTGAACAAGTCTGGAAAGAAATGCATAAACTTTTG
CCATTCTCACCGGATTCAGTCGTCACTCATGGTGATTTCTCACTTGATAACCTTATTTT
TGACGAGGGGAAATTAATAGGTTGTATTGATGTTGGACGAGTCGGAATCGCAGACCGAT
ACCAGGATCTTGCCATCCTATGGAACTGCCTCGGTGAGTTTTCTCCTTCATTACAGAAA
CGGCTTTTTCAAAAATATGGTATTGATAATCCTGATATGAATAAATTGCAGTTTCATTT
GATGCTCGATGAGTTTTTCTAAGAATTAATTCATGAGCGGATACATATTTGAATGTATT
TAGAAAAATAAACAAATAGGGGTTCCGCGCACATTTCCCCGAAAAGTGCCACCTGAAAT
TGTAAACGTTAATATTTTGTTAAAATTCGCGTTAAATTTTTGTTAAATCAGCTCATTTT
TTAACCAATAGGCCGAAATCGGCAAAATCCCTTATAAATCAAAAGAATAGACCGAGATA
GGGTTGAGTGTTGTTCCAGTTTGGAACAAGAGTCCACTATTAAAGAACGTGGACTCCAA
CGTCAAAGGGCGAAAAACCGTCTATCAGGGCGATGGCCCACTACGTGAACCATCACCCT
AATCAAGTTTTTTGGGGTCGAGGTGCCGTAAAGCACTAAATCGGAACCCTAAAGGGAGC
CCCCGATTTAGAGCTTGACGGGGAAAGCCGGCGAACGTGGCGAGAAAGGAAGGGAAGAA
AGCGAAAGGAGCGGGCGCTAGGGCGCTGGCAAGTGTAGCGGTCACGCTGCGCGTAACCA
CCACACCCGCCGCGCTTAATGCGCCGCTACAGGGCGCGTCCCATTCGCCA

### EXAMPLES

### Example 1—Biosynthesis of a First Multi-Substituent Psilocybin Derivative

E. coli strain E1 was constructed as follows. For plasmid cloning, Top10 or XL1-blue strains were used depending on antibiotic markers. Standard LB media was used for culturing. For gene expression and feeding experiments, the parent host strain employed was BL21 (DE3). First, the plasmid pET28a(+)-PfTrpB-B0A9-HIS was created by inserting an in-frame, HIS tagged (SEQ. ID NO: 46) PfTrpB-BOA9 gene (SEQ. ID NO: 1) into the NdeI/XhoI site of pET28a(+) (SEQ. ID NO: 36). As a second step, from plasmid pCDM4 (SEQ. ID NO: 35), the plasmid pCDM4-BaTDC-HIS was created by inserting an in-frame, HIS-tagged (SEQ. ID NO: 46) BaTDC gene (SEQ. ID NO: 3) into the NdeI/XhoI site of pCDM4. Finally, from plasmid pET23a(+) (SEQ. ID NO: 37), the plasmid pET23a(+)-PsmF-HIS was created by inserting an in-frame, HIS-tagged (SEQ. ID NO: 46) PsmF gene (SEQ. ID NO: 9) into the NdeI/XhoI site of pET23a(+). The target plasmids pET28a(+)-PfTrpB-B0A9-HIS, pCDM4-BaTDC-HIS and pET23a(+)-PsmF-HIS were transformed into BL21 (DE3) cells as follows: pCDM4-BaTDC-HIS was transformed into BL21 (DE3) first, and transformants selected using streptomycin were transformed with pET28a(+)-PfTrpB-B0A9-HIS and pET23a(+)-PsmF-HIS together. The final E. coli strain (Ec-1) was selected with streptomycin, ampicillin, and kanamycin. Scaled-up culturing of engineered E. coli was conducted as follows: seed cultures were inoculated in AMM (Jones et al., 2015, Sci Rep. 5: 11301) medium overnight. The overnight culture was then divided into two flasks containing 500 mL each of AMM medium additionally containing 0.5% (w/v) serine, 1M IPTG, 50 ug/L streptomycin, ampicillin, and kanamycin, and 100 mg/L indole feedstock (6-fluoro-1H-indol-4-ylamine; www.bldpharm.com) for conversion by Ec-1. Cultures were grown for 24 h. Cultures were then centrifuged (10,000 g×5 minutes) to remove cellular content, and culture broth containing secreted derivative was combined and stored at −80° C. until further processing. To 1.0 L of broth, 10M NaOH solution was added until the pH reached ˜7. The culture was then extracted by ethyl acetate (4×600 ml). The organic layer was combined and dried over Na₂SO₄, followed by concentration under reduced pressure. The residue was purified by flash chromatography on silica gel (1→2% metanol in dichloromethane), to give the compound as a light yellow solid (7 mg). Following purification, high-resolution MS (HRMS), ¹H NMR, and selective ¹³C NMR were performed to assess purity, estimate total quantity, and confirm molecular structure. ¹H NMR (400 MHz, CD₃OD): δ=1.94 (s, 3H), 3.00 (m, 2H), 3.38 (m, 2H), 6.10 (dd, J=11.7, 2.2 Hz, 1H), 6.38 (dd, J=9.7, 2.2 Hz, 1H), 6.84 (s, 1H). ¹³C NMR (100 MHz, CD₃OD): 5=21.0, 29.2, 41.9, 87.1 (d, J_{C, F}=26.1 Hz), 92.6 (d, J_{C, F}=27.8 Hz), 111.6, 112.4, 120.7, 138.0 (d, J_{C, F}=15.0 Hz), 141.7 (d, J_{C, F}=13.2 Hz), 160.7 (d, J_{C, F}=232.5 Hz), 172.1. HRMS (ESI) m/z: calcd. for C₁₂H₁₄FN₃O [M+H]⁺ 236.1194, found 236.1189. Purity was determined as 95% w/w. It is noted that these data confirm a chemical structure corresponding with that of example compound (IX):

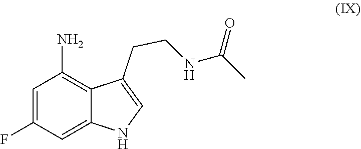 [SMILES: CC(=O)NCCc1c[nH]c2cc(F)cc(N)c12]

set forth herein.

### Assessment of Cell Viability Upon Treatment of a Psilocybin Derivative

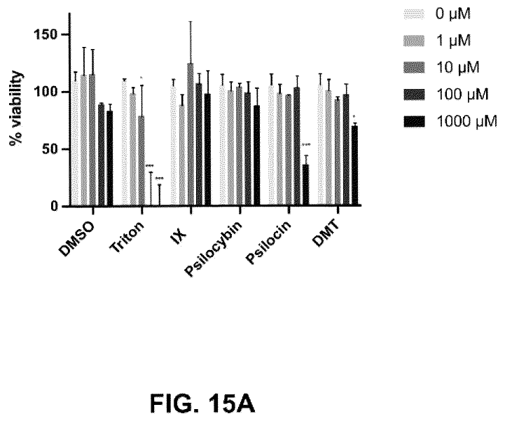

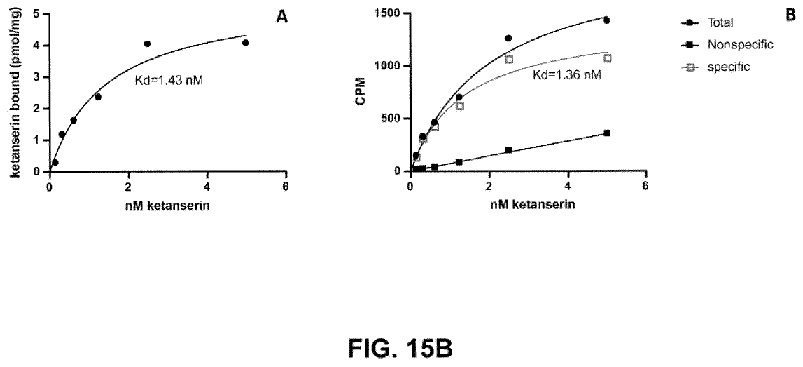

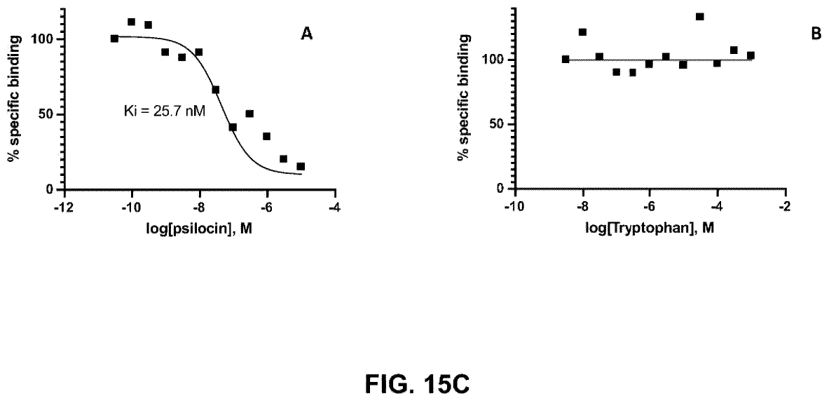

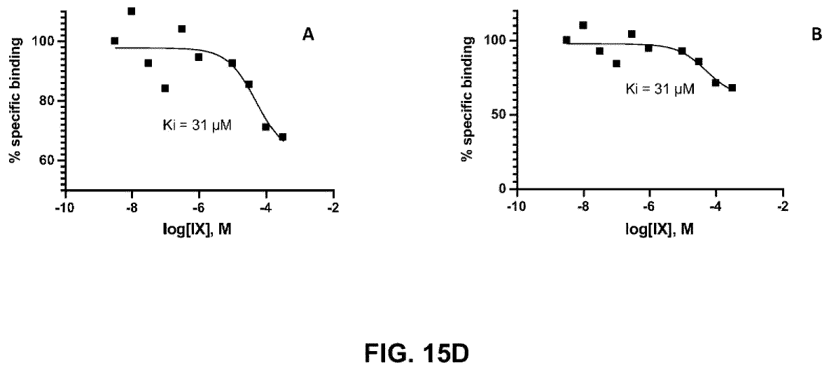

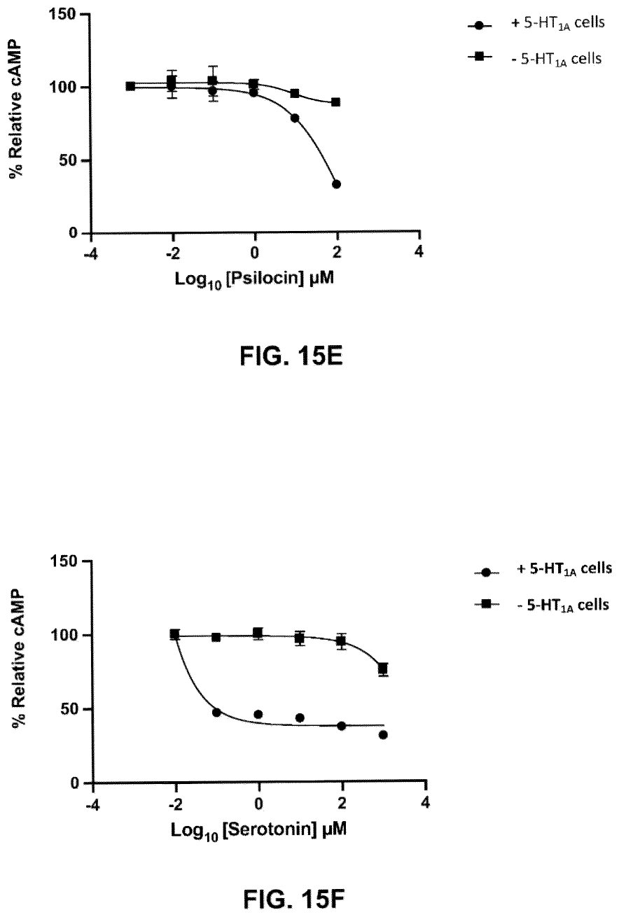

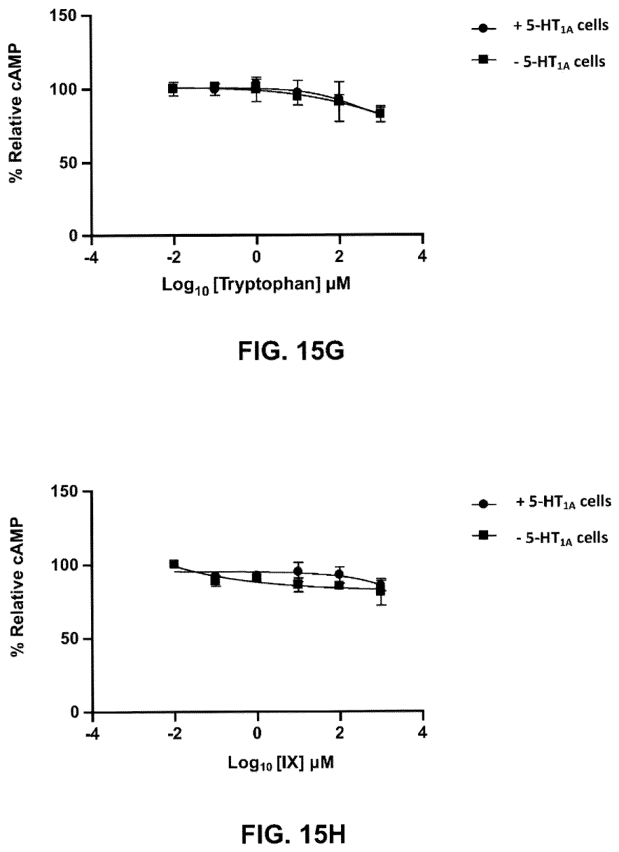

To establish suitable ligand concentrations for competitive binding assays, PrestoBlue assays were first performed. The PrestoBlue assay measures cell metabolic activity based on tetrazolium salt formation, and is a preferred method for routine cell viability assays (Terrasso et al., 2017, J Pharmacol Toxicol Methods 83: 72). Results of these assays were conducted using both control ligands (e.g., psilocybin, psilocin, DMT) and novel derivative, in part as a pre-screen for any remarkable toxic effects on cell cultures up to concentrations of 1 mM. A known cellular toxin (Triton X-100, Pyrgiotakis G. et al., 2009, Ann. Biomed. Eng. 37: 1464-1473) was included as a general marker of toxicity. Drug-induced changes in cell health within simple in vitro systems such as the HepG2 cell line are commonly adopted as first-line screening approaches in the pharmaceutical industry (Weaver et al., 2017, Expert Opin Drug Metab Toxicol 13: 767). HepG2 is a human hepatoma that is most commonly used in drug metabolism and hepatotoxicity studies (Donato et al., 2015, Methods Mol Biol 1250: 77). Herein, HepG2 cells were cultured using standard procedures using the manufacture's protocols (ATCC, HB-8065). Briefly, cells were cultured in Eagle's minimum essential medium supplemented with 10% fetal bovine serum and grown at 37° C. in the presence of 5% CO₂. To test the various compounds with the cell line, cells were seeded in a clear 96-well culture plate at 20,000 cells per well. After allowing cells to attach and grow for 24 hours, compounds were added at 1 μM, 10 μM, 100 μM, and 1 mM. Methanol was used as vehicle, at concentrations 0.001, 0.01, 0.1, and 1%. As a positive control for toxicity, TritonX concentrations used were 0.0001, 0.001, 0.01 and 0.1%. Cells were incubated with compounds for 48 hours before accessing cell viability with the PrestoBlue assay following the manufacture's protocol (ThermoFisher Scientific, P50200). PrestoBlue reagent was added to cells and allowed to incubate for 1 hour before reading. Absorbance readings were performed at 570 nm with the reference at 600 nm on a SpectraMax iD3 plate reader. Non-treated cells were assigned 100% viability. Bar graphs show the mean+/−SD, n=3. Significance was determined by 2-way ANOVA followed by Dunnett's multiple comparison test and is indicated by *** (P<0.0001), **(P<0.001), *(P<0.005). Data acquired for the derivative having chemical formula (IX) is displayed as “IX” on the x-axis of FIG. 15A.

### Radioligand Receptor Binding Assays.

Evaluation of drug binding is an essential step to characterization of all drug-target interactions (Fang 2012, Exp Opin Drug Discov 7:969). The binding affinity of a drug to a target is traditionally viewed as an acceptable surrogate of its in vivo efficacy (Núñez et al., 2012, Drug Disc Today 17: 10). Competition assays, also called displacement or modulation binding assays, are a common approach to measure activity of a ligand at a target receptor (Flanagan 2016, Methods Cell Biol 132: 191). In these assays, standard radioligands acting either as agonists or antagonists are ascribed to specific receptors. In the case of G protein-coupled receptor 5-HT_{2A}, [³H]ketanserin is a well-established antagonist used routinely in competition assays to evaluate competitive activity of novel drug candidates at the 5-HT_{2A} receptor (Maguire et al., 2012, Methods Mol Biol 897: 31). Thus, to evaluate activity of novel psilocybin derivatives at the 5-HT_{2A} receptor, competition assays using [³H]ketanserin were employed as follows. SPA beads (RPNQ0010), [³H] ketanserin (NET1233025UC), membranes containing 5-HT_{2A} (ES-313-M400UA), and isoplate-96 microplate (6005040) were all purchased from PerkinElmer. Radioactive binding assays were carried out using Scintillation Proximity Assay (SPA). For saturation binding assays, mixtures of 10 ug of membrane containing 5-HT_{2A} receptor was pre-coupled to 1 mg of SPA beads at room temperature in a tube rotator for 1 hour in binding buffer (50 mM Tris-HCl pH7.4, 4 mM CaCl₂, 1 mM ascorbic acid, 10 mM pargyline HCl). After pre-coupling, the beads and membrane were aliquoted in an isoplate-96 microplate with increasing amounts of [³H]ketanserin (0.1525 nM to 5 nM) and incubated for two hours at room temperature in the dark with shaking. After incubation, the samples were read on a MicroBeta 2 Microplate Counter (Perkin Elmer). Determination of non-specific binding was carried out in the presence of 20 mM of spiperone (S7395-250MG, Sigma). Equilibrium binding constants for ketanserin (K_{d}) were determined from saturation binding curves using the ‘one-site saturation binding analysis’ method of GraphPad PRISM software (Version 9.2.0). Competition binding assays were performed using fixed (1 nM) [³H]ketanserin and different concentrations of tryptophan (3 nM to 1 mM), psilocin (30 μM to 10 mM) or unlabeled test compound (3 nM to 1 mM) similar to the saturation binding assay. Kᵢ values were calculated from the competition displacement data using the competitive binding analysis from GraphPad PRISM software. Tryptophan was included as a negative control as it has no activity at the 5-HT_{2A} receptor. In contrast, psilocin was used as a positive control since it has established binding activity at the 5-HT_{2A} receptor (Kim et al., 2020, Cell 182: 1574). FIG. 15B depicts the saturation binding curves for [³H]ketanserin at the 5-HT_{2A} receptor. Panel A shows the specific saturation ligand binding of [³H]ketanserin (from 0.1525 nM to 5 nM) to membranes containing 5-HT_{2A} receptor, which was obtained after subtracting non-specific binding values (shown in Panel B). Specific binding in counts per minute (cpm) was calculated by subtracting non-specific binding from total binding. Specific binding (pmol/mg) was calculated from pmol of [³H]ketanserin bound per mg of protein in the assay. The K_{d} was calculated by fitting the data with the one-site binding model of PRISM software (version 9.2.0). FIG. 15C (Panel A) shows the competition binding curve for psilocin as a positive control (binding). (Panel B) shows the competition binding curve for tryptophan as a negative control (no binding). FIG. 15D shows competition binding curves for compound with formula (IX), designated “IX” in the figure. Notably, competition of compound (IX) for 5-HT_{2A} sites occupied by [³H]ketanserin does not appear complete, as suggested by only ˜50% specific binding (refer to Panel B, which replots data of Panel A with a reformatted y-axis for clarity). It is known that ketanserin binds both primary sites normally occupied by agonist (e.g., serotonin) in addition to other sites of 5-HT_{2A} (Sleight et al., 1996, Biochem Pharmacol 51: 71); thus, incomplete competition by compound (IX) implies this derivative competes for a particular subset (i.e., fraction) of the total sites bound by ketanserin.

### Cell Lines and Control Ligands Used to Assess Activity at 5-HT_{IA}.

CHO-K1/Gα₁₅ (GenScript, M00257) (−5-HT_{1A}) and CHO-K1/5-HT_{1A}/Gα₁₅ (GenScript, M00330) (+5-HT_{1A}) cells lines were used. Briefly, CHO-K1/Gα₁₅ is a control cell line that constitutively expresses Gα₁₅ which is a promiscuous G_{q} protein. This control cell line lacks any transgene encoding 5-HT_{1A} receptors, but still responds to forskolin; thus, cAMP response to forskolin should be the same regardless of whether or not 5-HT_{1A} agonists are present. Conversely, CHO-K1/5-HT_{1A}/Gα₁₅ cells stably express 5-HT_{1A} receptor in the CHO-K1 host background. Notably, Gα₁₅ is a promiscuous G protein known to induce calcium flux response, present in both control and 5-HT_{1A} cell lines. In +5-HT_{1A} cells, Gα₁₅ may be recruited in place of G_{αi/o}, which could theoretically dampen cAMP response (Rojas and Fiedler 2016, Front Cell Neurosci 10: 272). Thus, we included two known 5-HT_{1A} agonists, psilocin (Blair et al., 2000, J Med Chem 43: 4701) and serotonin (Rojas and Fiedler 2016, Front Cell Neurosci 10: 272) as positive controls to ensure sufficient cAMP response was observed, thereby indicating measurable recruitment of G_{αi/o} protein to activated 5-HT_{1A} receptors. In contrast, tryptophan is not known to activate, or modulate in any way, 5-HT_{1A} receptors, and was thus used as a negative control. Cells were maintained in complete growth media as recommended by supplier (GenScript) which is constituted as follows: Ham's F12 Nutrient mix (HAM's F12, GIBCO #11765-047) with 10% fetal bovine serum (FBS) (Thermo Scientific #12483020), 200 μg/ml zeocin (Thermo Scientific #R25005) and/or 100 μg/ml hygromycin (Thermo Scientific #10687010). The cells were cultured in a humidified incubator with 37° C. and 5% CO₂. Cells maintenance was carried out as recommended by the cell supplier. Briefly, vials with cells were removed from the liquid nitrogen and thawed quickly in 37° C. water bath. Just before the cells were completely thawed the vial's outside was decontaminated by 70% ethanol spray. The cell suspension was then retrieved from the vial and added to warm (37° C.) complete growth media, and centrifuged at 1,000 rpm for 5 minutes. The supernatant was discarded, and the cell pellet was then resuspended in another 10 ml of complete growth media, and added to the 10 cm cell culture dish (Greiner Bio-One #664160). The media was changed every third day until the cells were about 90% confluent. The ˜90% confluent cells were then split 10:1 for maintenance or used for experiment.

### Evaluation of 5-HT_{IA} Receptor Modulation

As 5-HT_{1A} activation inhibits cAMP formation, the ability of test molecules to modulate 5-HT_{1A} response was measured via changes in the levels of cAMP produced due to application of 4 μM forskolin. Changes (should any significant change occur) in intracellular cAMP levels due to the treatment of novel molecule was evaluated using cAMP-Glo Assay kit (Promega #V1501). Briefly, +5-HT_{1A} cells were seeded on 1-6 columns and base −5-HT_{1A} cells were seeded on columns 7-12 of the white walled clear bottom 96-well plate (Corning, #3903). Both cells were seeded at the density of 30,000 cells/well in 100 μl complete growth media and cultured 24 hrs in humidified incubator at 37° C. and 5% CO₂. On the experiment day, the media of cells was replaced with serum/antibiotic free culture media. Then the cells were treated for 20 minutes with test molecules dissolved in induction medium (serum/antibiotic free culture media containing 4 μM forskolin, 500 mM IBMX (isobutyl-1-methylxanthine, Sigma-Aldrich, Cat. #17018) and 100 mM (RO 20-1724, Sigma-Aldrich, Cat. #B8279)). Forskolin induced cAMP formation whereas IBMX and RO 20-1724 inhibited the degradation of cAMP. PKA was added to the lysate, mixed, and subsequently the substrate of the PKA was added. PKA was activated by cAMP, and the amount of ATP consumed due to PKA phosphorylation directly corresponded to cAMP levels in the lysate. Reduced ATP caused reduced conversion of luciferin to oxyluciferin, conferring diminished luminescence as the result 5-HT_{1A} activation. In summary: this signal cascade permits 5-HT_{1A} activation (positive modulation) by a test molecule to be measured in terms of decreasing % cAMP formation. Conversely, enhanced % cAMP is expected when 5-HT_{1A} receptor is negatively modulated by a test molecule. Finally, no significant change in % cAMP—beyond that observed for negative control experiments (e.g., with tryptophan)—indicates that a test molecule does not bind 5-HT_{1A} or that binding imparts a silent response. FIG. 15E shows decreased % cAMP in +5HT_{1A} compared to −5HT_{1A} cultures, in the presence of fixed (4 μM) forskolin, as dosages of psilocin increase, revealing 5-HT_{1A} activity of psilocin. FIG. 15F shows decreased % cAMP in +5HT_{1A} compared to −5HT_{1A} cultures, in the presence of fixed (4 μM) forskolin, as dosages of serotonin increase, revealing 5-HT_{1A} activity of serotonin. FIG. 15G shows no significant difference in % cAMP for +5HT_{1A} compared to −5HT_{1A} cultures, in the presence of fixed (4 μM) forskolin, as dosages of tryptophan increase, revealing no modulation of 5-HT_{1A} activity for tryptophan. FIG. 15H shows no significant difference in % cAMP for +5HT_{1A} compared to −5HT_{1A} cultures, in the presence of fixed (4 μM) forskolin, as dosages of compound (X) increase, revealing no modulation of 5-HT_{1A} activity for compound (X). Note that compound (X) is shown simply as “X” along the x-axis. FIG. 15H shows no significant difference in % cAMP for +5HT_{1A} compared to −5HT_{1A} cultures, in the presence of fixed (4 μM) forskolin, as dosages of compound (IX) increase, revealing no modulation of 5-HT_{1A} activity for compound (IX). Note that compound (IX) is shown simply as “IX” along the x-axis. For FIGS. 15E-15H, cAMP levels are reported relative (%) to values observed in ligand-free (0 mM) samples; the value “0” along the x-axis refers to a ligand concentration of 0.0001 mM; and when present, error bars represent results of three experiments (n=3).

### Example 2—Biosynthesis of a Second Multi-Substituent Psilocybin Derivative

Escherichia coli strain Ec-1 was used to biosynthesize psilocybin derivative with formula (X) from derivatized indole feedstock. The construction of Ec-1 is described in Example 1. Scaled-up culturing and material storage of engineered E. coli was conducted as described in Example 1, except that 4-fluoro-1H-indol-6-ylamine (Combi-Blocks, www.combi-blocks.com) was used in place of 6-fluoro-1H-indol-4-ylamine. Scaled-up culturing and processing of engineered E. coli was conducted as described in Example 1, except that a total of 2.0 L was cultured. Two litres of E. coli culture broth was extracted by ethyl acetate (4×1.2 L). The organic layer was combined and dried over Na₂SO₄, followed by concentration under reduced pressure. The residue was purified by flash chromatography on silica gel (1→3% metanol in dichloromethane), to give the compound as a yellow solid (5 mg). Following purification, high-resolution MS (HRMS), ¹H NMR, and selective ¹³C NMR were performed to assess purity, estimate total quantity, and confirm molecular structure. ¹H NMR (400 MHz, CD₃OD): δ=1.92 (s, 3H), 2.14 (s, 3H), 2.98 (t, J=7.2, 2H), 3.47 (t, J=7.2, 2H), 6.82 (dd, J=12.9, 1.6 Hz, 1H), 7.00 (s, 1H), 7.56 (d, J=1.6 Hz, 1H). ¹³C NMR (100 MHz, CD₃OD): b=21.1, 22.4, 25.9, 40.5 (d, J_{C, F}=2.0 Hz), 97.5 (d, J_{C, F}=24.0 Hz), 98.9 (d, J_{C,F}=3.4 Hz), 110.5 (d, J_{C, F}=2.8 Hz), 112.6 (d, J_{C, F}=20.0 Hz), 122.6, 133.1 (d, J_{C, F}=10.5 Hz), 139.2 (d, J_{C, F}=13.6 Hz), 156.2 (d, J_{C, F}=242.5 Hz), 170.0, 171.8. HRMS (ESI) m/z: calcd. for C₁₂H₁₆FN₃O₂ [M+H]⁺ 278.1299, found 278.1298. Purity was determined as 95% w/w. It is noted that these data confirm a chemical structure corresponding with that of example compound (X):

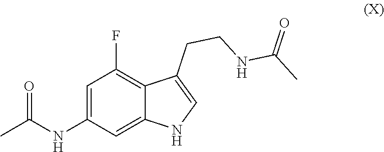 [SMILES: CC(=O)NCCc1c[nH]c2cc(NC(C)=O)cc(F)c12]

set forth herein.

### Assessment of Cell Viability Upon Treatment of Psilocybin Derivative

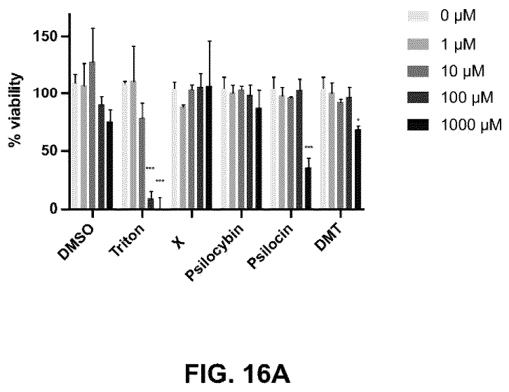

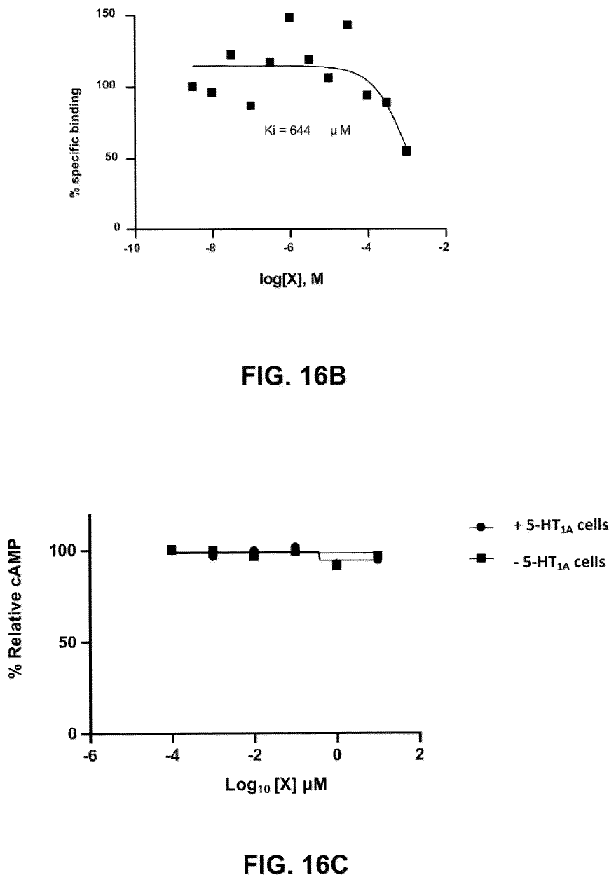

Cell viability was assessed as described for Example 1, except the compound with formula (X) was evaluated in place of the compound with formula (IX). FIG. 16A shows PrestoBlue assay results for compound with formula (X), depicted on the x-axis as “X”.

### Radioligand Receptor Binding Assays.

Activity at 5-HT_{2A} receptor was assessed as described for Example 1, except the compound with formula (X) was evaluated in place of the compound with formula (IX). FIG. 16B shows radioligand competition assay results for compound with formula (X), depicted on the x-axis simply as “X”. The relatively high Kᵢ value for the compound (X) compared with that of psilocin indicates comparatively ‘loose’ binding, or a mild degree of competition by compound (X) with ketanserin for 5-HT_{2A} interaction. Similarly high Kᵢ values for 5-HT_{2A} (e.g., micromolar range) signifying mild or ‘loose’ binding profiles are noted for drugs such as selegiline (Toll et al., NIDA Res. Monogr. 1998, 178: 440), an important treatment for major depressive disorder, MDMA (Simmler et al., Br. J. Pharmacol. 2013, 168: 458; Setola et al., Molec Pharmacol 2003, 63: 1223) and mescaline (Rickli et al., Neuropharmacology 2015, 99: 546) which are high-profile, potential depression treatments (Liechti et al., Curr Top Behav Neurosci 2021, doi: 10.1007/7854_2021_270).

### Cell Lines, Control Ligands, and Evaluation of 5-HT_{IA} Receptor Modulation

Cell lines and control ligands were as described in Example 1. Activity at 5-HT_{1A} receptor was assessed as described for Example 1, except the compound with formula (X) was evaluated in place of the compound with formula (IX). FIG. 16C shows 5-HT_{1A} assay results as measured in units of % relative cAMP, where compound with formula (X) is depicted on the x-axis simply as “X”. FIG. 16C shows no significant difference in % cAMP for +5HT_{1A} compared to −5HT_{1A} cultures, in the presence of fixed (4 μM) forskolin, as dosages of compound (X) increase, revealing no modulation of 5-HT_{1A} activity for compound (X).

### Example 3—Biosynthesis of a Third Multi-Substituent Psilocybin Derivative

Escherichia coli strain Ec-1 was used to biosynthesize psilocybin derivative with formula (XII) from derivatized indole feedstock. The construction of Ec-1 is described in Example 1. Scaled-up culturing and material storage of engineered E. coli was conducted as described in Example 1, except that 5-fluoro-7-nitro-1H-indole (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific), employing a modified version of a method described previously (Chang et al., 2015, Plant Physiol. 169: 1127-1140), with the exception that liquid chromatography was carried out using an UltiMate 3000 HPLC (Thermo Fisher Scientific) equipped with a Poroshell 120 SB-C18 column (Agilent Technologies) instead of an Accela HPLC system (Thermo Fisher Scientific) equipped with a Zorbax C18 column (Agilent Technologies). Briefly, 100 microliters of culture media were dried and resuspended in 100 microliters of DMSO. One tenth (10 microliters) of this suspension was injected at a flow rate of 0.5 mL/min and a gradient of solvent A (water with 0.1% of formic acid) and solvent B (ACN with 0.1% formic acid) as follows: 100% to 0% (v/v) solvent A over 5 min; isocratic at 0% (v/v) for 1 min; 0% to 100% (v/v) over 0.1 min; and isocratic at 100% (v/v) for 1.9 min. Total run time was 8 minutes. Heated ESI source and interface conditions were operated in positive ion mode as follows: vaporizer temperature, 400° C.; source voltage, 3 kV; sheath gas, 60 au, auxiliary gas, 20 au; capillary temperature, 380° C.; capillary voltage, 6 V; tube lens, 45 V. Instrumentation was performed as a single, HR scan event using Orbitrap detection of m/z in the range of 100-500 m/z. Ion injection time was 300 ms with scan time of 1 s. External and internal calibration procedures ensured <2 ppm error to facilitate elemental formulae predictions. Singly protonated product with exact m/z and expected elemental formula matching the singly protonated form of N-[2-(5-fluoro-7-nitro-1H-indol-3-yl)ethyl]acetamide, having chemical formula (XII):

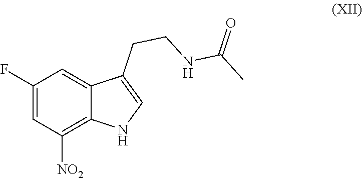 [SMILES: CC(=O)NCCc1c[nH]c2c([N+](=O)[O-])cc(F)cc12]

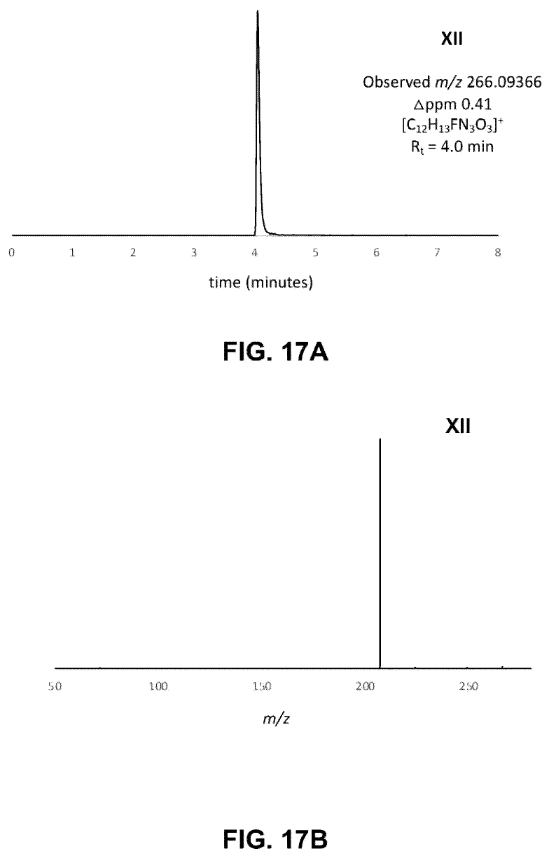

eluted at 4.0 minutes (EIC, see: FIG. 17A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XII) as follows (FIG. 17B, Table 1) (Servillo L. et al., 2013, J. Agric. Chem. 61: 5156-5162).

TABLE 1

| m/z | % Relative abundance | Ionic species | Δ ppm |
|---|---|---|---|
| 207.05636 | 100 | [M + H − C₂H₅NO]⁺ | 0.34 |
| 266.09362 | 1.1 | [M + H]⁺ | 0.26 |
| 224.08303 | 0.8 | | |
| 249.06697 | 0.7 | | |
| 71.70087 | 0.5 | | |
| 66.12294 | 0.4 | | |
| 161.06346 | 0.3 | | |
| 127.24116 | 0.2 | | |
| 98.30615 | 0.2 | | |
| 192.96957 | 0.2 | | |

### Example 4—Biosynthesis of a Fourth Multi-Substituent Psilocybin Derivative

Escherichia coli strain Ec-1 was used to biosynthesize psilocybin derivative with formula (XIV) from derivatized indole feedstock. The construction of Ec-1 is described in Example 1. Scaled-up culturing and material storage of engineered E. coli was conducted as described in Example 1, except that 5-fluoro-1H-indol-6-ylamine (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(6-acetylamino-5-fluoro-1H-indol-3-yl)ethyl]acetamide, having chemical formula (XIV):

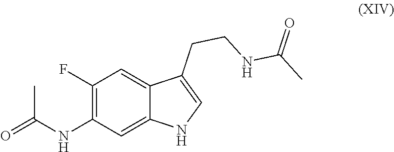 [SMILES: CC(=O)NCCc1c[nH]c2cc(NC(C)=O)c(F)cc12]

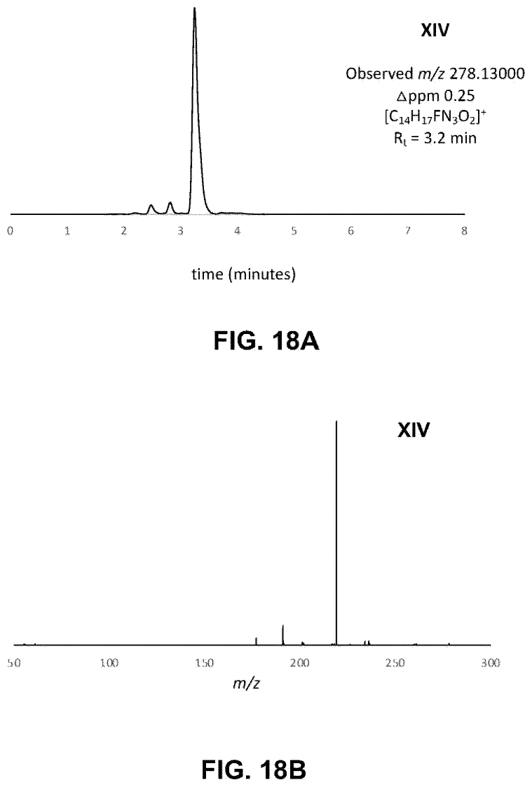

eluted at 3.2 minutes (EIC, see: FIG. 18A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XIV) as follows (FIG. 18B, Table 2) (Servillo L. et al., 2013, J. Agric. Chem. 61: 5156-5162).

TABLE 2

| m/z | % Relative abundance | Ionic species | Δ ppm |
|---|---|---|---|
| 219.09292 | 100 | [M + H − C₂H₅NO]⁺ | 0.46 |
| 191.09787 | 9.0 | | |
| 177.08215 | 4.2 | | |
| 236.11933 | 2.4 | | |
| 234.10378 | 2.0 | | |
| 261.10332 | 1.4 | | |
| 278.12980 | 1.2 | [M + H]⁺ | 0.47 |
| 226.11267 | 0.8 | | |
| 218.10916 | 0.8 | | |
| 260.11925 | 0.6 | | |

### Example 5—Biosynthesis of a Fourth Multi-Substituent Psilocybin Derivative

Escherichia coli strain Ec-1 was used to biosynthesize psilocybin derivative with formula (XV) from derivatized indole feedstock. The construction of Ec-1 is described in Example 1. Scaled-up culturing and material storage of engineered E. coli was conducted as described in Example 1, except that culturing was performed for 14 hours instead of 24 hours, and 5-fluoro-1H-indol-6-ylamine (www.bldpharm.com) was used in place of 6-fluoro-1H-indol-4-ylamine. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(6-acetylamino-5-fluoro-1H-indol-3-yl)ethyl]acetamide, having chemical formula (XV):

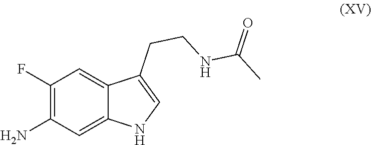 [SMILES: CC(=O)NCCc1c[nH]c2cc(N)c(F)cc12]

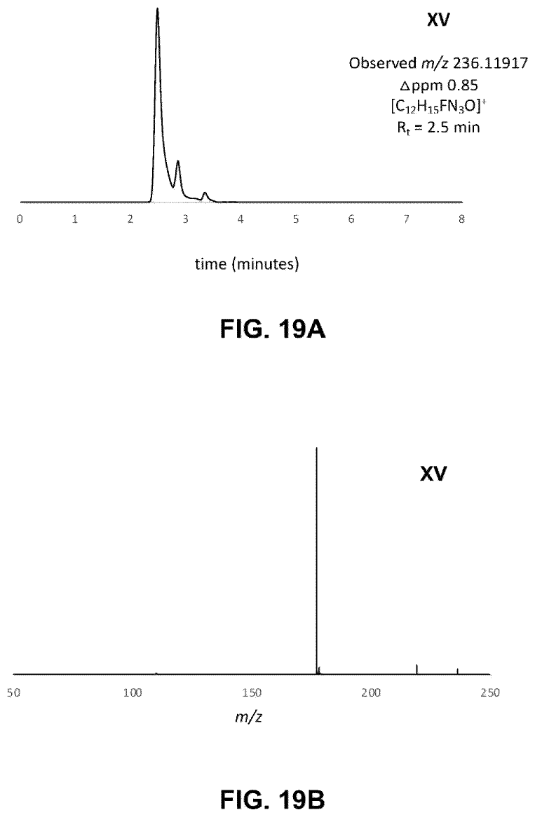

eluted at 2.5 minutes (EIC, see: FIG. 19A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XV) as follows (FIG. 19B, Table 3) (Servillo L. et al., 2013, J. Agric. Chem. 61: 5156-5162).

TABLE 3

| m/z | % Relative abundance | Ionic species | Δ ppm |
|---|---|---|---|
| 177.08158 | 100 | [M + H − C₂H₅NO]⁺ | 3.78 |
| 219.09224 | 4.1 | | |
| 236.11880 | 3.0 | [M + H]⁺ | 2.41 |
| 109.66972 | 0.6 | | |
| 157.07565 | 0.4 | | |
| 181.73305 | 0.2 | | |
| 199.48006 | 0.1 | | |
| 194.10827 | 0.1 | | |
| 220.09515 | 0.1 | | |
| 207.09179 | 0.1 | | |

### Example 6—Biosynthesis of a Sixth Multi-Substituent Psilocybin Derivative

Escherichia coli strain Ec-1 was used to biosynthesize psilocybin derivative with formula (XVII) from derivatized indole feedstock. The construction of Ec-1 is described in Example 1. Scaled-up culturing and material storage of engineered E. coli was conducted as described in Example 1, except that 4-fluoro-1H-indole-5-carbonitrile (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(5-cyano-4-fluoro-1H-indol-3-yl)ethyl]acetamide, having chemical formula (XVII):

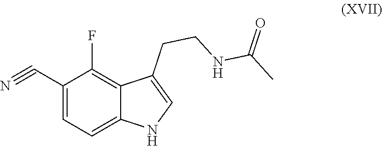 [SMILES: CC(=O)NCCc1c[nH]c2ccc(C#N)c(F)c12]

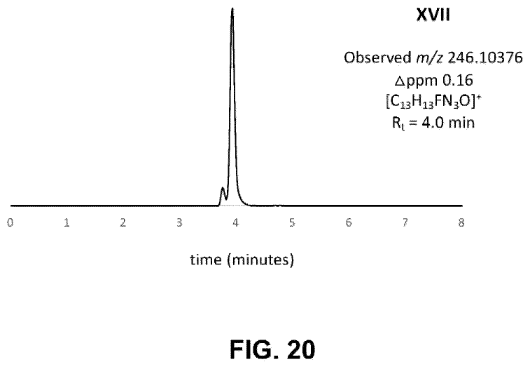

eluted at 4.0 minutes (EIC, see: FIG. 20).

### Example 7—Biosynthesis of a Seventh Multi-Substituent Psilocybin Derivative

Escherichia coli strain Ec-1 was used to biosynthesize psilocybin derivative with formula (XVIII) from derivatized indole feedstock. The construction of Ec-1 is described in Example 1. Scaled-up culturing and material storage of engineered E. coli was conducted as described in Example 1, except that 6-bromo-1H-indol-4-ol (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(6-bromo-4-hydroxy-1H-indol-3-yl)ethyl]acetamide, having chemical formula (XVIII):

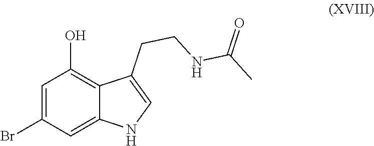 [SMILES: CC(=O)NCCc1c[nH]c2cc(Br)cc(O)c12]

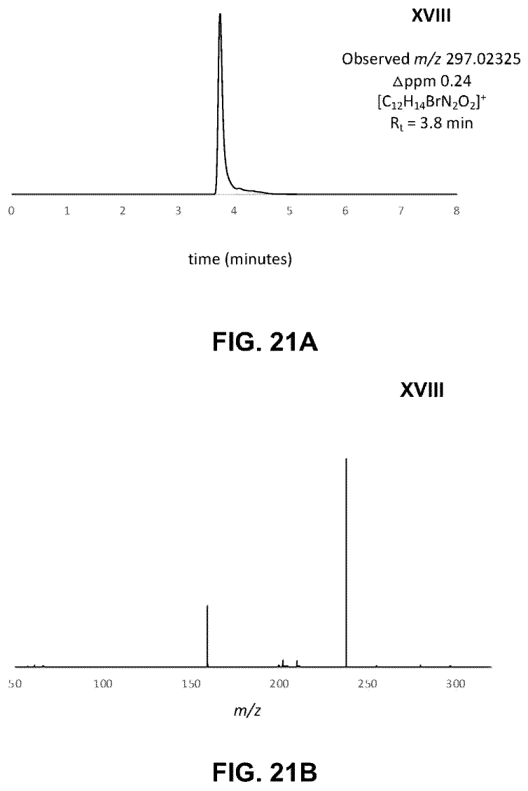

eluted at 3.8 minutes (EIC, see: FIG. 21A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XVIII) as follows (FIG. 21B, Table 4) (Servillo L. et al., 2013, J. Agric. Chem. 61: 5156-5162).

TABLE 4

| m/z | % Relative abundance | Ionic species | Δ ppm |
|---|---|---|---|
| 237.98574 | 100 | [M + H − C₂H₆NO]⁺ | 1.93 |
| 159.06737 | 29.5 | | |
| 209.99081 | 3.3 | | |
| 255.01230 | 1.2 | | |
| 279.99649 | 1.0 | | |
| 199.51968 | 1.0 | | |
| 61.09401 | 0.9 | | |
| 297.02267 | 0.7 | [M + H]⁺ | 2.19 |
| 203.89784 | 0.6 | | |
| 177.16207 | 0.6 | | |

### Example 8—Biosynthesis of an Eighth Multi-Substituent Psilocybin Derivative

Escherichia coli strain Ec-1 was used to biosynthesize psilocybin derivative with formula (XXI) from derivatized indole feedstock. The construction of Ec-1 is described in Example 1. Scaled-up culturing and material storage of engineered E. coli was conducted as described in Example 1. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(4-acetylamino-6-fluoro-1H-indol-3-yl)ethyl]acetamide, having chemical formula (XXI):

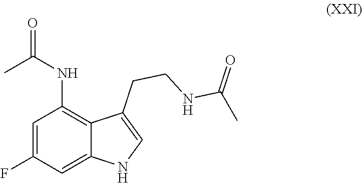 [SMILES: CC(=O)NCCc1c[nH]c2cc(F)cc(NC(C)=O)c12]

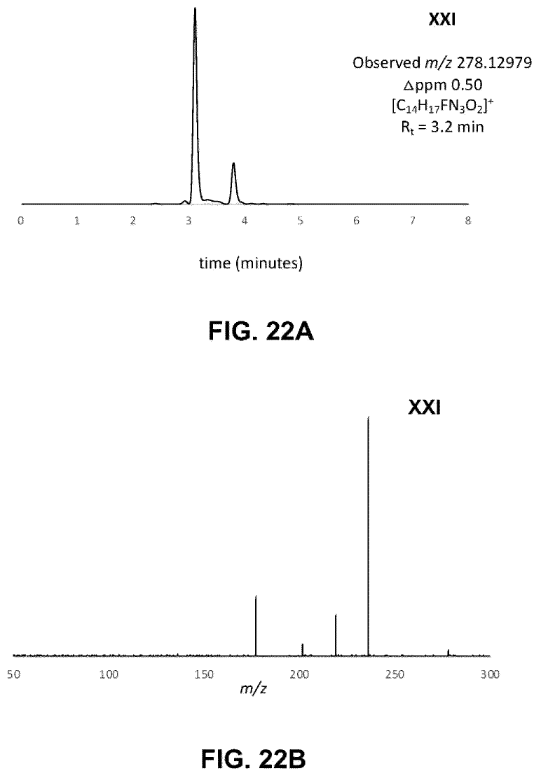

eluted at 3.2 minutes (EIC, see: FIG. 22A). Notably, while the same indole feedstock was provided in this example compared to the feedstock used Example 1, the product (XXI) is not the same as product (IX). In fact, both products (XXI) and (IX) are achieved by feeding 6-fluoro-1H-indol-4-ylamine to Ec-1. However, in Example 1, only product (IX) was purified. Conversely, in this Example, only product (XXI) was analyzed. As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXI) as follows (FIG. 22B, Table 5) (Servillo L. et al, 2013, J. Agric. Chem. 61: 5156-5162).

TABLE 5

| | % Relative | | |
|---|---|---|---|
| m/z | abundance | Ionic species | Δ ppm |
| 236.11928 | 100 | [M + H − C₂H₂O]⁺ | 0.38 |
| 177.08202 | 26.0 | [M + H − C₂H₆NO − C₂H₂O]⁺ | 1.30 |
| 219.09267 | 17.9 | [M + H − C₂H₆NO]⁺ | 0.68 |
| 278.12986 | 3.0 | [M + H]⁺ | 0.25 |
| 136.07571 | 1.1 | | |
| 56.17127 | 0.9 | | |
| 199.20892 | 0.8 | | |
| 88.82042 | 0.8 | | |
| 232.86285 | 0.8 | | |
| 158.02567 | 0.8 | | |

### Example 9—Biosynthesis of a Ninth Multi-Substituent Psilocybin Derivative

Escherichia coli strain Ec-1 was used to biosynthesize psilocybin derivative with formula (XXIII) from derivatized indole feedstock. The construction of Ec-1 is described in Example 1. Scaled-up culturing and material storage of engineered E. coli was conducted as described in Example 1, except that 4-fluoro-1H-indole-7-carbonitrile (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(7-cyano-4-fluoro-1H-indol-3-yl)ethyl]acetamide, having chemical formula (XXIII):

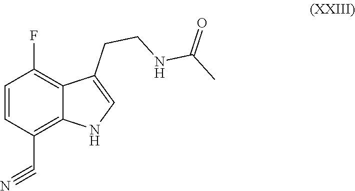 [SMILES: CC(=O)NCCc1c[nH]c2c(C#N)ccc(F)c12]

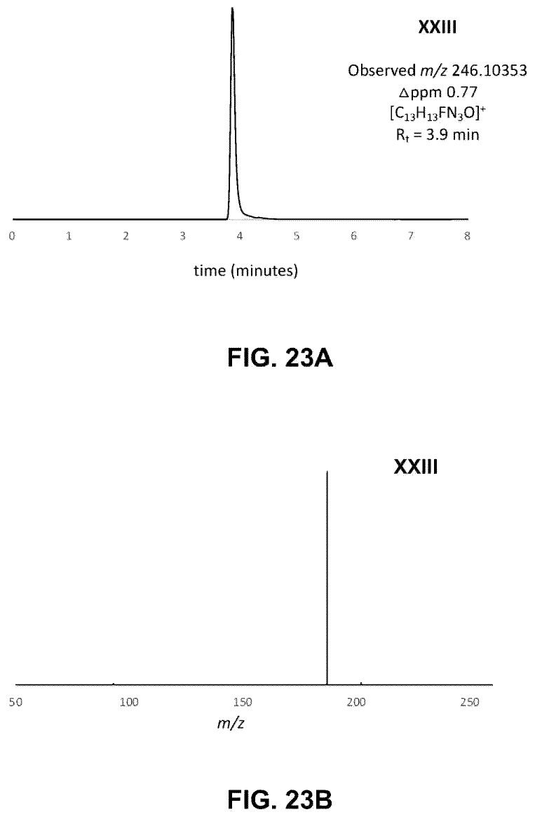

eluted at 3.9 minutes (EIC, see: FIG. 23A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXIII) as follows (FIG. 23B, Table 6) (Servillo L. et al., 2013, J. Agric. Chem. 61: 5156-5162).

TABLE 6

| m/z | % Relative abundance | Ionic species | Δ ppm |
|---|---|---|---|
| 187.06606 | 100 | [M + H − C₂H₃NO]⁺ | 2.89 |
| 93.13169 | 1.0 | | |
| 204.09270 | 0.6 | | |
| 229.07666 | 0.2 | | |
| 246.10356 | 0.2 | [M + H]⁺ | 0.61 |
| 199.66049 | 0.2 | | |
| 59.34151 | 0.1 | | |

### Example 10—Biosynthesis of a Tenth Multi-Substituent Psilocybin Derivative

Escherichia coli strain Ec-1 was used to biosynthesize psilocybin derivative with formula (XXV) from derivatized indole feedstock. The construction of Ec-1 is described in Example 1. Scaled-up culturing and material storage of engineered E. coli was conducted as described in Example 1, except that 4-fluoro-1H-indol-6-ylamine was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(6-amino-4-fluoro-1H-indol-3-yl)ethyl]acetamide, having chemical formula (XXV):

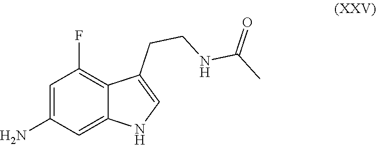 [SMILES: CC(=O)NCCc1c[nH]c2cc(N)cc(F)c12]

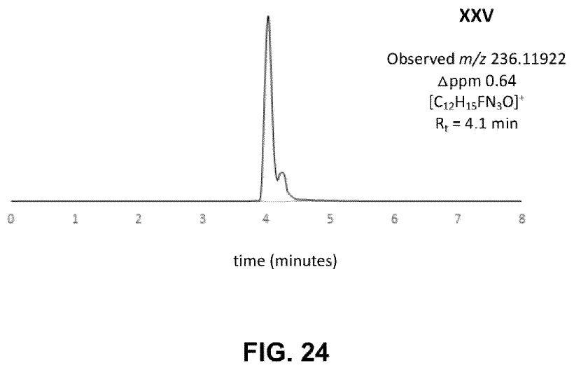

eluted at 4.1 minutes (EIC, see: FIG. 24). Notably, while the same indole feedstock was provided in this example compared to the feedstock used Example 2, the product (XXV) is not the same as product (X). In fact, both products (XXV) and (X) are achieved by feeding 4-fluoro-1H-indol-6-ylamine to Ec-1. However, in Example 2, only product (X) was purified. Conversely, in this Example, only product (XXV) was analyzed.

### Example 11—Biosynthesis of an Eleventh Multi-Substituent Psilocybin Derivative

Escherichia coli strain Ec-1 was used to biosynthesize psilocybin derivative with formula (XXVIII) from derivatized indole feedstock. The construction of Ec-1 is described in Example 1. Scaled-up culturing and material storage of engineered E. coli was conducted as described in Example 1, except that 4-amino-1H-indole-6-carbonitrile (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(4-amino-6-cyano-1H-indol-3-yl)ethyl]acetamide, having chemical formula (XXVIII):

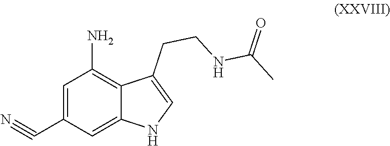 [SMILES: CC(=O)NCCc1c[nH]c2cc(C#N)cc(N)c12]

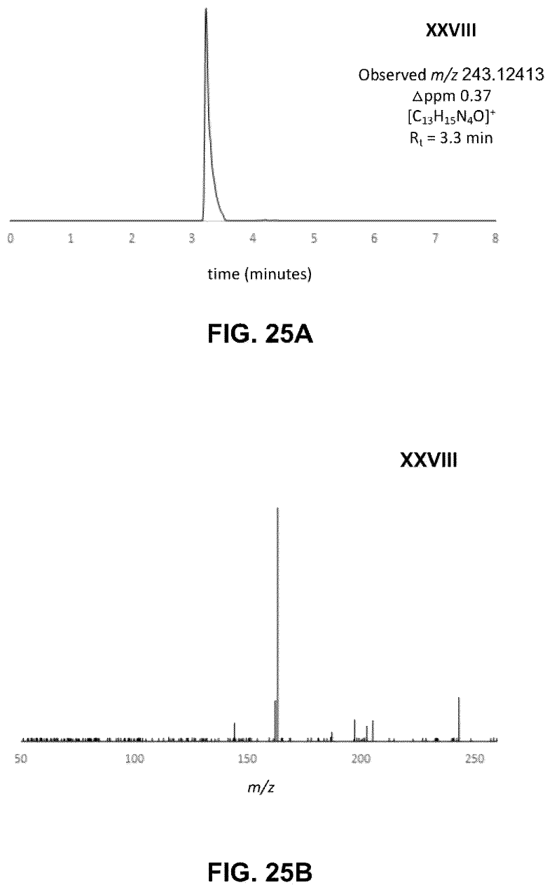

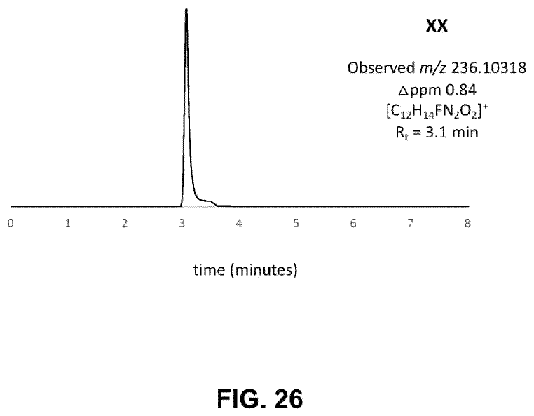

eluted at 3.3 minutes (EIC, see: FIG. 25A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXVIII) as follows (FIG. 25B, Table 7) (Servillo L. et al., 2013, J. Agric. Chem. 61: 5156-5162).

TABLE 7

| | % Relative | | |
|---|---|---|---|
| m/z | abundance | Ionic species | Empirical Formula |
| 163.1076 | 100 | | |
| 243.1340 | 19.2 | [M + H]⁺ | C₁₄H₁₅N₄O |
| 162.0760 | 17.3 | | |
| 197.1284 | 9.2 | | |
| 205.1182 | 9.1 | | |
| 144.0653 | 7.8 | | |
| 202.4956 | 6.9 | | |
| 187.1077 | 4.2 | | |
| 115.0388 | 2.0 | | |
| 186.4933 | 1.9 | | |

### Example 12—Biosynthesis of a Twelfth Multi-Substituent Psilocybin Derivative

Escherichia coli strain Ec-1 was used to biosynthesize psilocybin derivative with formula (XX) from derivatized indole feedstock. The construction of Ec-1 is described in Example 1. Scaled-up culturing and material storage of engineered E. coli was conducted as described in Example 1, except that 4-fluoro-1H-indol-5-ol (www.bldpharm.com) was used in place of 6-fluoro-1H-indol-4-ylamine. Scaled-up culturing and processing of engineered E. coli was conducted as described in Example 1. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-[2-(4-fluoro-5-hydroxy-1H-indol-3-yl)ethyl]acetamide, having chemical formula (XX):

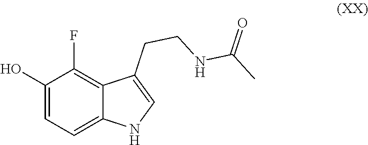 [SMILES: CC(=O)NCCc1c[nH]c2ccc(O)c(F)c12]

eluted at 3.1 minutes (EIC, see: FIG. 2₆).

### Example 13—Biosynthesis of a Thirteenth Multi-Substituent Psilocybin Derivative

Escherichia coli strain Ec-2 was used to biosynthesize psilocybin derivative with formula (XIII) from derivatized indole feedstock. E. coli strain Ec-2 was constructed as follows. For plasmid cloning, Top10 or XL1-blue strains were used depending on antibiotic markers. Standard LB media was used for culturing. For gene expression and feeding experiments, the parent host strain employed was BL21 (DE3). Plasmids pET28a(+)-PfTrpB-BOA9-HIS and pCDM4-BaTDC-HIS were created as described in Example 1. The target plasmids pET28a(+)-PfTrpB-BOA9-HIS and pCDM4-BaTDC-HIS were sequentially transformed into BL21 (DE3) cells as follows: pCDM4-BaTDC-HIS was transformed into BL21 (DE3) first. Transformants selected using streptomycin were next transformed with pET28a(+)-PfTrpB-B0A9-HIS and selected with both streptomycin and kanamycin. Scaled-up culturing and material storage of engineered E. coli was conducted as described in Example 1, except that (1) only streptomycin and kanamycin were used for selection purposes; and (2) 5-fluoro-7-nitro-1H-indole (www.bldpharm.com) was used in place of 6-fluoro-1H-indol-4-ylamine. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-(5-fluoro-7-nitro-1H-indol-3-yl)ethylamine, having chemical formula (XIII):

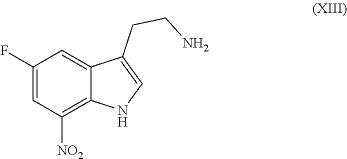 [SMILES: NCCc1c[nH]c2c([N+](=O)[O-])cc(F)cc12]

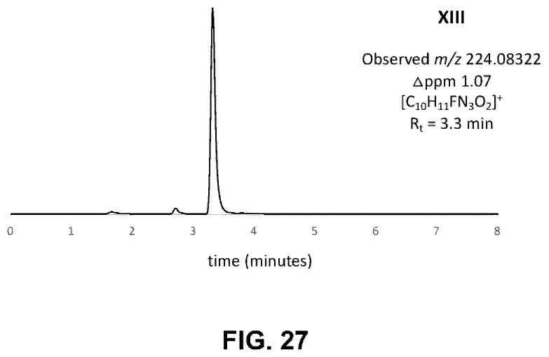

eluted at 3.3 minutes (EIC, see: FIG. 27).

### Example 14—Biosynthesis of a Fourteenth Multi-Substituent Psilocybin Derivative

Escherichia coli strain Ec-2 was used to biosynthesize psilocybin derivative with formula (XVI) from derivatized indole feedstock. The construction of Ec-2 is described in Example 13. Scaled-up culturing and material storage of engineered E. coli was conducted as described in Example 13, except that 5-fluoro-1H-indol-6-ylamine (www.bldpharm.com) was used in place of 5-fluoro-7-nitro-1H-indole. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-5-fluoro-1H-indol-6-amine, having chemical formula (XVI):

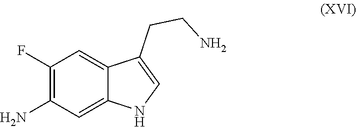 [SMILES: NCCc1c[nH]c2cc(N)c(F)cc12]

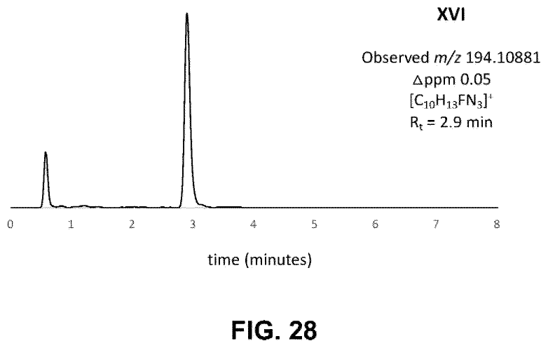

eluted at 2.9 minutes (EIC, see: FIG. 28).

### Example 15—Biosynthesis of a Fifteenth Multi-Substituent Psilocybin Derivative

Escherichia coli strain Ec-2 was used to biosynthesize psilocybin derivative with formula (XIX) from derivatized indole feedstock. The construction of Ec-2 is described in Example 13. Scaled-up culturing and material storage of engineered E. coli was conducted as described in Example 13, except that 4-fluoro-1H-indole-5-carbonitrile (www.bldpharm.com) was used in place of 5-fluoro-7-nitro-1H-indole. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-4-fluoro-1H-indole-5-carbonitrile, having chemical formula (XIX):

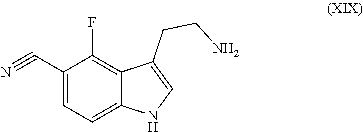 [SMILES: N#Cc1ccc2[nH]cc(CCN)c2c1F]

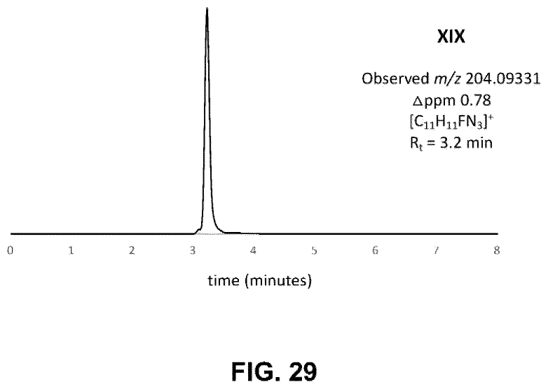

eluted at 3.2 minutes (EIC, see: FIG. 29).

### Example 16—Biosynthesis of a Sixteenth Multi-Substituent Psilocybin Derivative

Escherichia coli strain Ec-2 was used to biosynthesize psilocybin derivative with formula (XXII) from derivatized indole feedstock. The construction of Ec-2 is described in Example 13. Scaled-up culturing and material storage of engineered E. coli was conducted as described in Example 13, except that 6-fluoro-1H-indol-4-ylamine (www.bldpharm.com) was used in place of 5-fluoro-7-nitro-1H-indole. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-6-fluoro-1H-indol-4-amine, having chemical formula (XXII):

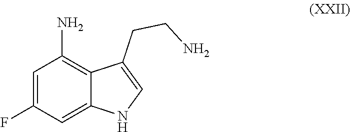 [SMILES: NCCc1c[nH]c2cc(F)cc(N)c12]

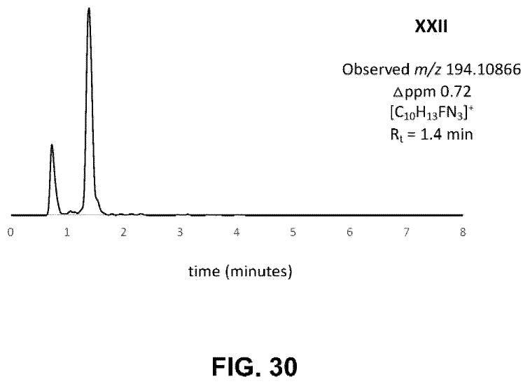

eluted at 1.4 minutes (EIC, see: FIG. 30).

### Example 17—Biosynthesis of a Seventeenth Multi-Substituent Psilocybin Derivative

Escherichia coli strain Ec-2 was used to biosynthesize psilocybin derivative with formula (XXVI) from derivatized indole feedstock. The construction of Ec-2 is described in Example 13. Scaled-up culturing and material storage of engineered E. coli was conducted as described in Example 13, except that 3-(2-aminoethyl)-4-fluoro-1H-indol-6-amine (www.bldpharm.com) was used in place of 5-fluoro-7-nitro-1H-indole. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-4-fluoro-1H-indol-6-amine, having chemical formula (XXVI):

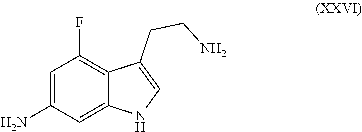 [SMILES: NCCc1c[nH]c2cc(N)cc(F)c12]

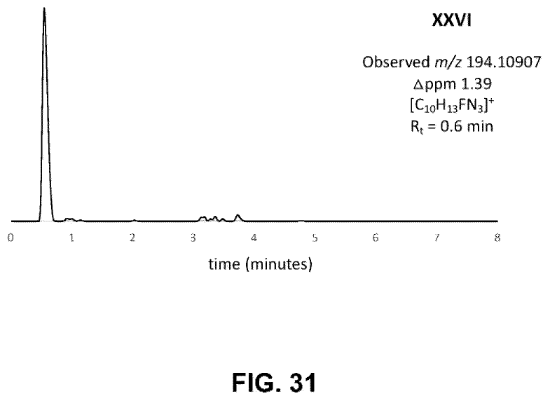

eluted at 0.6 minutes (EIC, see: FIG. 31).

### Example 18—Biosynthesis of an Eighteenth Multi-Substituent Psilocybin Derivative

Escherichia coli strain Ec-2 was used to biosynthesize psilocybin derivative with formula (XXIX) from derivatized indole feedstock. The construction of Ec-2 is described in Example 13. Scaled-up culturing and material storage of engineered E. coli was conducted as described in Example 13, except that 4-amino-1H-indole-6-carbonitrile (www.bldpharm.com) was used in place of 5-fluoro-7-nitro-1H-indole. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 4-amino-3-(2-aminoethyl)-1H-indole-6-carbonitrile, having chemical formula (XXIX):

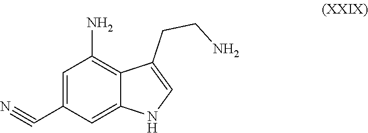 [SMILES: N#Cc1cc(N)c2c(CCN)c[nH]c2c1]

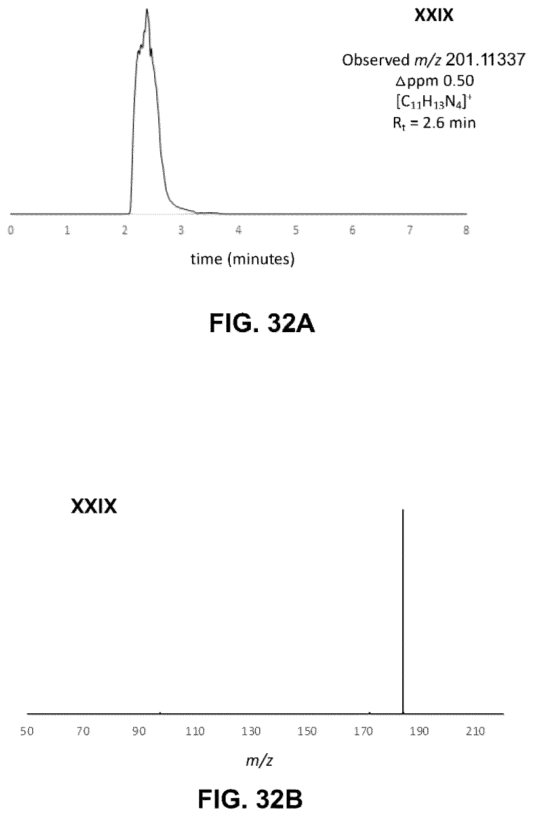

eluted at 2.6 minutes (EIC, see: FIG. 32A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXIX) as follows (FIG. 32B, Table 8) (Servillo L. et al., 2013, J. Agric. Chem. 61: 5156-5162).

TABLE 8

| m/z | % Relative abundance | Ionic species | Δ ppm |
|---|---|---|---|
| 184.08655 | 100 | [M + H − NH₃]⁺ | 2.01 |
| 97.51806 | 1.0 | | |
| 172.08669 | 0.7 | | |
| 201.11313 | 0.6 | [M + H]⁺ | 1.69 |
| 199.55735 | 0.1 | | |
| 61.09273 | 0.1 | | |
| 143.08142 | 0.1 | | |
| 60.25552 | 0.1 | | |
| 209.32732 | 0.1 | | |
| 202.04140 | 0.1 | | |

### Example 19—Biosynthesis of a Nineteenth Multi-Substituent Psilocybin Derivative

Escherichia coli strain Ec-3 was used to biosynthesize psilocybin derivative with formula (XXVII) from derivatized indole feedstock. E. coli strain Ec-2 was constructed as follows. For plasmid cloning, Top10 or XL1-blue strains were used depending on antibiotic markers. Standard LB media was used for culturing. For gene expression and feeding experiments, the parent host strain employed was BL21 (DE3). First, the plasmid pET28a(+)-EsNMT-HIS was created by inserting an in-frame, HIS tagged (SEQ. ID NO: 46) EsNMT gene (SEQ. ID NO: 11) into the NdeI/XhoI site of pET28a(+) (SEQ. ID NO: 36). As a second step, from plasmid pCDM4 (SEQ. ID NO: 35), the plasmid pCDM4-PsiD-HIS was created by inserting an in-frame, HIS-tagged (SEQ. ID NO: 46) PsiD gene (SEQ. ID NO: 7) into the NdeI/XhoI site of pCDM4. These target plasmids were sequentially transformed into BL21 (DE3) cells as follows: pCDM4-PsiD-HIS was transformed into BL21 (DE3) first. Transformants selected using streptomycin were next transformed with pET28a(+)-EsNMT-HIS and selected with both streptomycin and kanamycin. Scaled-up culturing and material storage of engineered E. coli was conducted as described in Example 1, except that (1) only streptomycin and kanamycin were used for selection purposes; and (2) 4-amino-1H-indole-6-carbonitrile (www.bldpharm.com) was used in place of 6-fluoro-1H-indol-4-ylamine. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 4-amino-3-[2-(methylamino)ethyl]-1H-indole-6-carbonitrile, having chemical formula (XXVII):

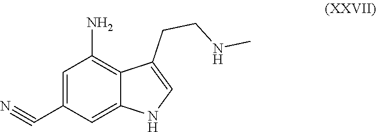 [SMILES: CNCCc1c[nH]c2cc(C#N)cc(N)c12]

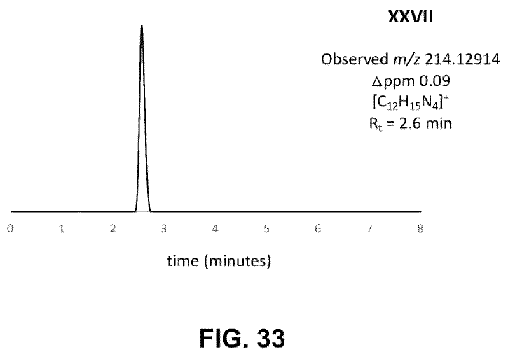

eluted at 2.6 minutes (EIC, see: FIG. 33).

### Example 20—Biosynthesis of a Twentieth Multi-Substituent Psilocybin Derivative

Yeast (Saccharomyces cerevisiae) strain Sc-1 was created through genetic engineering of a parent yeast strain, to enable bioconversion of commercially obtained, derivatized indole, tryptophan, or tryptamine feedstock to generate final product. The parent yeast (Saccharomyces cerevisiae) strain was CEN.PK with genotype Matα; ura3-52; trp1-289; leu2-3,112; his3Δ1; MAL2-8C; SUC2. The parent strain was engineered to include 7DMATS (SEQ. ID NO: 16), ClostSporTDC (SEQ. ID NO: 6), and PsmF (SEQ. ID NO: 10) which catalyzed three enzymatic steps. Engineering also included CPR (SEQ. ID NO: 26) although this enzyme was not used in the bioconversion process. 7DMATS, ClostSporTDC, and CPR were included in the strain through chromosomal homologous recombination of integration cassettes as described previously (Dastmalchi et al., 2019, Nat. Chem Biol. 15: 384-390; Chen et al., 2018, Nat. Chem Biol. 14: 738-743). Conversely, PsmF was built into a protein expression plasmid and transformed to the genomically integrated strain already harboring 7DMATS, ClostSporTDC, and CPR. 7DMATS, ClostSporTDC, and CPR were encoded by SEQ. ID NO: 15, SEQ. ID NO: 5 and SEQ. ID NO: 25, respectively, with addition of in-frame, C-terminal HIS (SEQ. ID NO: 46, SEQ. ID NO: 47), FLAG (SEQ. ID NO: 42, SEQ. ID NO: 43), and c-MYC (SEQ. ID NO: 40, SEQ. ID NO: 41) epitope tags, respectively. Integration cassettes were built using yeast promoter sequences amplified from S. cerevisiae genomic DNA as described (Dastmalchi et al., 2019; Chen et al., 2018) enabling constitutive gene expression. Amplified promoters included PGK1 (SEQ. ID NO: 30), TDH3 (SEQ. ID NO: 31), CLN1 (SEQ. ID NO: 32), and UGA1 (SEQ. ID NO: 33). Two integration cassettes were assembled: the first, (X-3)::TADH1-ClostSporTDC-Flag-PPGK1-PTDH3-CPR-c-myc-TCYC1 (SEQ. ID NO: 52), harboured tagged ClostSporTDC and CPR. The second (Xii-2):PTDH3-7DMATS-His-TCYC1 (SEQ. ID NO: 53), harboured only tagged 7DMATS. Successive genomic integration of these cassettes was performed as described previously (Chen et al., 2018). Following stable integration of these two cassettes, the strain was further manipulated by transformation with a yeast episomal vector encoding a promiscuous N-acetyltransferase, PsmF (pMM1-pTDH3-PsmF-His-tCYC1). For construction of pMM1-pTDH3-PsmF-His-tCYC1, the gene PsmF (SEQ. ID NO: 9) fused in-frame with a HIS epitope tag (SEQ. ID NO: 46) was ligated to empty plasmid pMM1 (SEQ. ID NO: 34) using BamHI/SacII restriction sites. For this Example, heterologous expression of a non-native or engineered TrpB gene was not necessary, as endogenous tryptophan synthase activity proved sufficient. The final engineered strain was called Sc-1. For scaled-up production of derivative product, culturing was performed as follows. Seed cultures were inoculated in SD-drop-out medium overnight. The overnight culture was then divided into two flasks containing 500 ml each of SD-drop-out medium containing 2% (w/v) glucose, 0.3% (w/v) KH₂PO₄, 0.05% (w/v) MgSO₄·7H₂O, 0.5% (w/v) (NH₄)₂SO₄ plus 500 μM 4-chloro-1H-indole (www.combi-blocks.com) for conversion by Sc-1. Yeast cultures were grown for 48 h. Cultures were then centrifuged (10,000 g×5 minutes) to remove cellular content, and culture broth containing secreted derivative product was stored at −80° C. until further processing. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-(2-[4-chloro-7-(3-methyl-2-butenyl)-1H-indol-3-yl]ethyl)acetamide, having chemical formula (XXXIV):

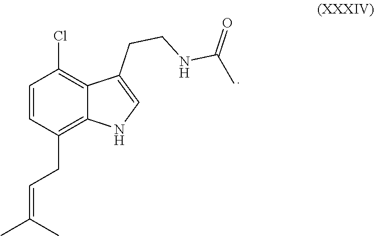 [SMILES: CC(=O)NCCc1c[nH]c2c(CC=C(C)C)ccc(Cl)c12]

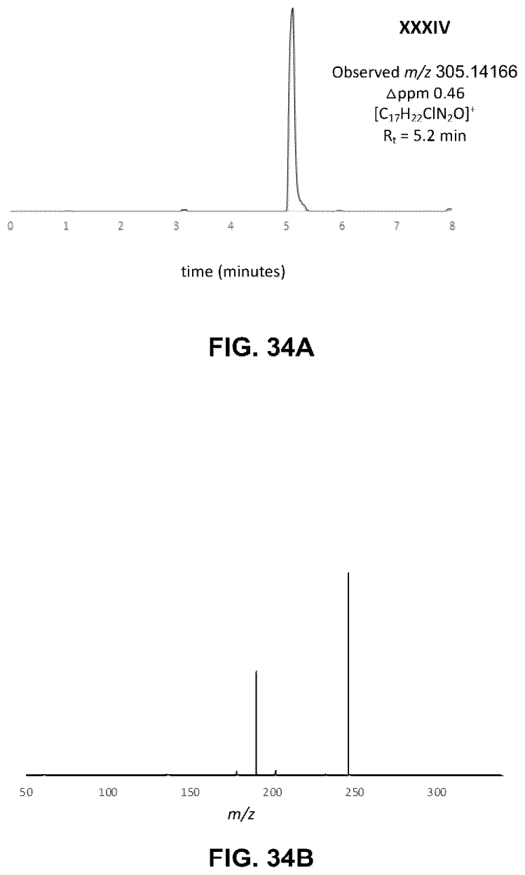

eluted at 5.2 minutes (EIC, see: FIG. 34A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXXIV) as follows (FIG. 34B, Table 9) (Servillo L. et al., 2013, J. Agric. Chem. 61: 5156-5162).

TABLE 9

| m/z | % Relative abundance | Ionic species | Δ ppm |
|---|---|---|---|
| 246.10397 | 100 | [M + H − NH₃]⁺ | 1.74 |
| 190.04136 | 53.1 | | |
| 178.04141 | 2.5 | | |
| 232.05183 | 0.8 | | |
| 135.46806 | 0.7 | | |
| 61.09248 | 0.6 | | |
| 151.39211 | 0.6 | | |
| 134.24040 | 0.6 | | |
| 199.43795 | 0.6 | | |
| 118.45281 | 0.5 | | |

### Example 21—Biosynthesis of a Twenty-First Multi-Substituent Psilocybin Derivative

The same yeast strain (Sc-1) and procedures described in Example 20 were used to biosynthesize a psilocybin derivative with chemical formula (XXXIII), with the following exception: in place of 4-chloro-1H-indole, 500 μM 5-fluoro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-[5-fluoro-7-(3-methyl-2-butenyl)-1H-indol-3-yl]ethylamine having chemical formula (XXXII):

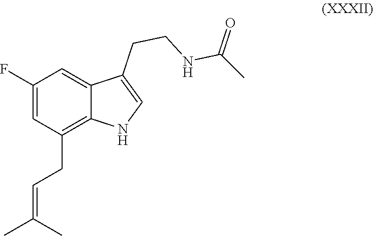 [SMILES: CC(=O)NCCc1c[nH]c2c(CC=C(C)C)cc(F)cc12]

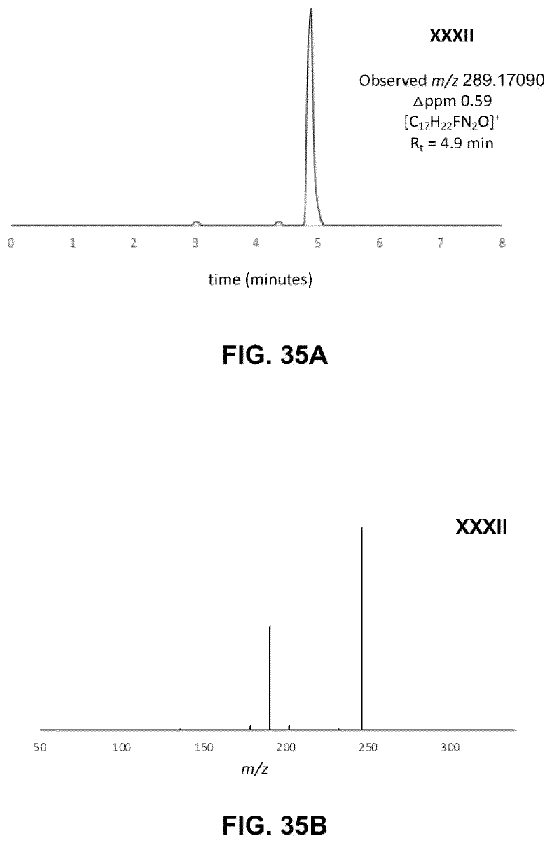

eluted at 4.9 minutes (EIC, see: FIG. 35A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXXII) as follows (FIG. 35B, Table 10) (Servillo L. et al., 2013, J. Agric. Chem. 61: 5156-5162).

TABLE 10

| m/z | % Relative abundance | Ionic species | Δ ppm |
|---|---|---|---|
| 230.13354 | 100 | [M + H − C₂H₆NO]⁺ | 1.78 |
| 174.07090 | 11.0 | | |
| 272.14384 | 1.9 | | |
| 233.10802 | 1.6 | | |
| 199.41144 | 0.8 | | |
| 151.39218 | 0.8 | | |
| 162.07105 | 0.8 | | |
| 216.08147 | 0.7 | | |
| 289.17040 | 0.7 | [M + H]⁺ | 2.32 |
| 143.09824 | 0.7 | | |

### Example 22—Biosynthesis of a Twenty-Second Multi-Substituent Psilocybin Derivative

The same yeast strain (Sc-1) and procedures described in Example 20 were used to biosynthesize a psilocybin derivative with chemical formula (XXXIII), with the following exception: in place of 4-chloro-1H-indole, 500 μM 5-fluoro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-(2-[5-fluoro-7-(3-methyl-2-butenyl)-1H-indol-3-yl]ethyl)acetamide having chemical formula (XXXIII):

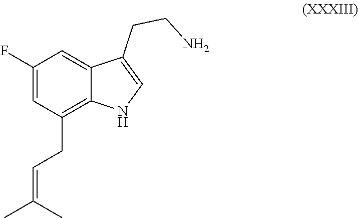 [SMILES: CC(C)=CCc1cc(F)cc2c(CCN)c[nH]c12]

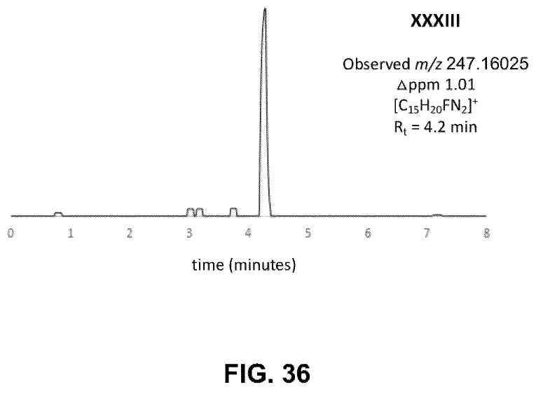

eluted at 4.2 minutes (EIC, see: FIG. 36). Notably, while the same indole feedstock was provided in this example compared to the feedstock used Example 21, the product (XXXII) is not the same as product (XXXIII). In fact, both products (XXXII) and (XXXIII) are achieved by feeding 5-fluoro-1H-indole to Sc-1. However, in Example 21, only product (XXXII) was analyzed. Conversely, in this Example, only product (XXXIII) was analyzed.

### Example 23—Biosynthesis of a Twenty-Third Multi-Substituent Psilocybin Derivative

The same yeast strain (Sc-1) and procedures described in Example 20 were used to biosynthesize a psilocybin derivative with chemical formula (XLIX), with the following exception: in place of 4-chloro-1H-indole, 500 μM 1H-indol-4-ol (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[4-hydroxy-7-(3-methyl-2-butenyl)-1H-indol-3-yl]propionic acid having chemical formula (XLIX):

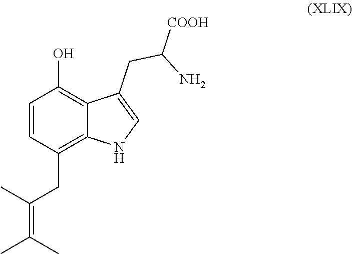 [SMILES: CC(C)=C(C)Cc1ccc(O)c2c(CC(N)C(=O)O)c[nH]c12]

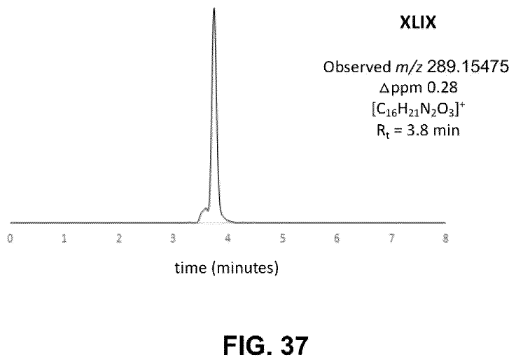

eluted at 3.8 minutes (EIC, see: FIG. 37). Notably, compound (XLIX) retains a carboxylic acid group despite the presence of ClostSporTDC enzyme in Sc-1. The decarboxylated version of compound (XLIX) was not detectable in the culture media, possibly owing to an inherent inability of ClostSporTDC enzyme to accept hydroxylated substrates.

### Example 24—Biosynthesis of a Twenty-Fourth Multi-Substituent Psilocybin Derivative

Yeast (Saccharomyces cerevisiae) strain Sc-2 was created through plasmid transformation of a parent yeast strain, to enable bioconversion of commercially obtained, derivatized indole, tryptophan, or tryptamine feedstock to generate final product. The parent yeast (Saccharomyces cerevisiae) strain was CEN.PK with genotype Mata; ura3-52; trp1-289; leu2-3,112; his3Δ1; MAL2-8C; SUC2. The parent strain was transformed with a yeast episomal vector (pMM1-pTDH3-PriB-His-tCYC1) encoding a HIS-tagged (SEQ. ID NO: 46, SEQ. ID NO: 47), promiscuous 6-prenyltransferase enzyme, PriB (SEQ. ID NO: 18). For construction of pMM1-pTDH3-PriB-His-tCYC1, the gene PriB (SEQ. ID NO: 17) fused in-frame with a HIS epitope tag (SEQ. ID NO: 46) was ligated to empty plasmid pMM1 (SEQ. ID NO: 34) using BamHI/SacII restriction sites. For this Example, heterologous expression of a non-native or engineered TrpB gene was not necessary, as endogenous tryptophan synthase activity proved sufficient. The final engineered strain was called Sc-2. For scaled-up production of derivative product, culturing was performed as described in Example 20, with the following exception: in place of 4-chloro-1H-indole, 500 μM 1H-indol-4-ol (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[4-hydroxy-6-(3-methyl-2-butenyl)-1H-indol-3-yl]propionic acid having chemical formula (L):

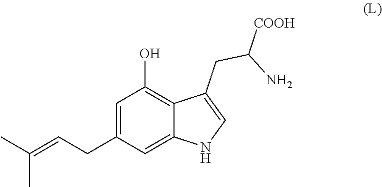 [SMILES: CC(C)=CCc1cc(O)c2c(CC(N)C(=O)O)c[nH]c2c1]

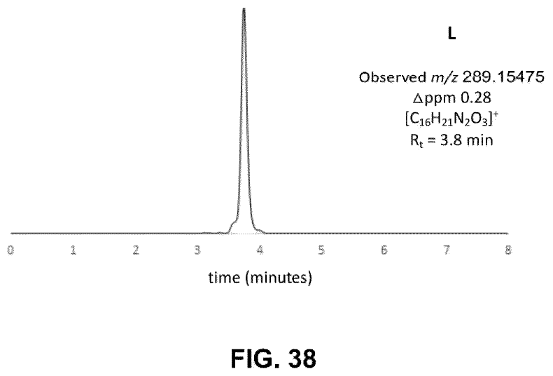

eluted at 3.8 minutes (EIC, see: FIG. 38).

### Example 25—Biosynthesis of a Twenty-Fifth Multi-Substituent Psilocybin Derivative

The same yeast strain (Sc-2) and procedures described in Example 24 were used to biosynthesize a psilocybin derivative with chemical formula (XLVIII), with the following exception: in place of 1H-indol-4-ol, 5-bromo-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[5-bromo-6-(3-methyl-2-butenyl)-1H-indol-3-yl]propionic acid having chemical formula (XLVIII):

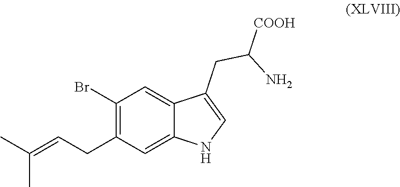 [SMILES: CC(C)=CCc1cc2[nH]cc(CC(N)C(=O)O)c2cc1Br]

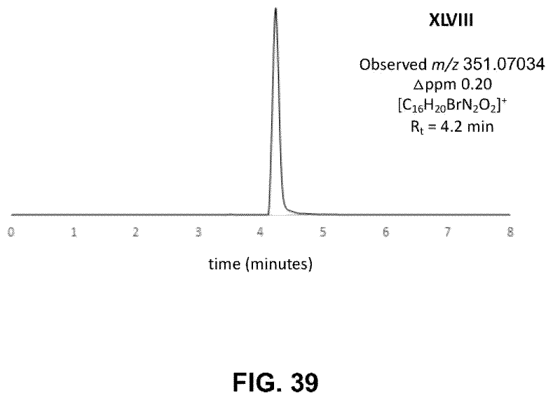

eluted at 4.2 minutes (EIC, see: FIG. 39).

### Example 26—Biosynthesis of a Twenty-Sixth Multi-Substituent Psilocybin Derivative

Yeast (Saccharomyces cerevisiae) strain Sc-3 was created through plasmid transformation of a parent yeast strain, to enable bioconversion of commercially obtained, derivatized indole, tryptophan, or tryptamine feedstock to generate final product. The parent yeast (Saccharomyces cerevisiae) strain was CEN.PK with genotype Mata; ura3-52; trp1-289; leu2-3,112; his3Δ1; MAL2-8C; SUC2. The parent strain was transformed with a yeast episomal vector (pMM1-pTDH3-SCO7467-tCYC1) encoding a 5-prenyltransferase enzyme, SCO7467 (SEQ. ID NO: 20). For construction of pMM1-pTDH3-SCO7467-tCYC1, the gene SCO7467 (SEQ. ID NO: 19) was ligated to empty plasmid pMM1 (SEQ. ID NO: 34) using BamHI/SacII restriction sites. For this Example, heterologous expression of a non-native or engineered TrpB gene was not necessary, as endogenous tryptophan synthase activity proved sufficient. The final engineered strain was called Sc-3. For scaled-up production of derivative product, culturing was performed as described in Example 20, with the following exception: in place of 4-chloro-1H-indole, 500 μM 1H-Indol-4-ol (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[4-hydroxy-5-(3-methyl-2-butenyl)-1H-indol-3-yl]propionic acid, having chemical formula (LI):

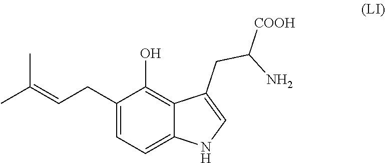 [SMILES: CC(C)=CCc1ccc2[nH]cc(CC(N)C(=O)O)c2c1O]

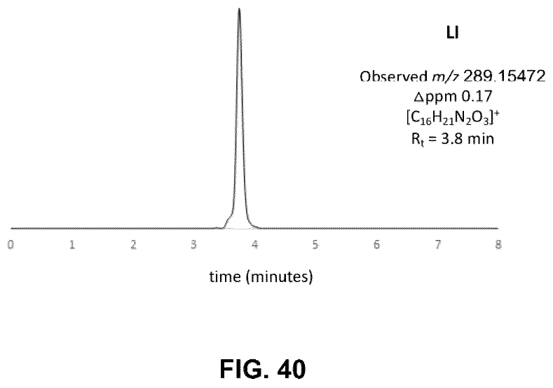

eluted at 3.8 minutes (EIC, see: FIG. 40).

### Example 27—Biosynthesis of a Twenty-Seventh Multi-Substituent Psilocybin Derivative

Yeast (Saccharomyces cerevisiae) strain Sc-4 was created through genetic engineering of a parent yeast strain, to enable bioconversion of commercially obtained, derivatized indole, tryptophan, or tryptamine feedstock to generate final product. The parent yeast (Saccharomyces cerevisiae) strain was CEN.PK with genotype Mata; ura3-52; trp1-289; leu2-3,112; his3Δ1; MAL2-8C; SUC2. The parent strain was engineered to include PriB (SEQ. ID NO: 18), BaTDC (SEQ. ID NO: 4), and PsmF (SEQ. ID NO: 10) which catalyzed three enzymatic steps. Engineering also included CPR (SEQ. ID NO: 26) although this enzyme was not used in the bioconversion process. PriB, BaTDC, and CPR were included in the strain through chromosomal homologous recombination of integration cassettes as described previously (Dastmalchi et al., 2019, Nat. Chem Biol. 15: 384-390; Chen et al., 2018, Nat. Chem Biol. 14: 738-743). Conversely, PsmF was built into a protein expression plasmid and transformed to the genomically integrated strain already harboring PriB, BaTDC, and CPR. PriB, BaTDC, and CPR were encoded by SEQ. ID NO: 17, SEQ. ID NO: 3 and SEQ. ID NO: 25, respectively, with addition of in-frame, C-terminal HIS (SEQ. ID NO: 46, SEQ. ID NO: 47), FLAG (SEQ. ID NO: 42, SEQ. ID NO: 43), and c-MYC (SEQ. ID NO: 40, SEQ. ID NO: 41) epitope tags, respectively. Integration cassettes were built using yeast promoter sequences amplified from S. cerevisiae genomic DNA as described (Dastmalchi et al., 2019; Chen et al., 2018) enabling constitutive gene expression. Amplified promoters included PGK1 (SEQ. ID NO: 30), TDH3 (SEQ. ID NO: 31), CLN1 (SEQ. ID NO: 32), and UGA1 (SEQ. ID NO: 33). Two integration cassettes were assembled: the first, (X-3)::TADH1-BaTDC-Flag-PPGK1-PTDH3-CPR-c-myc-TCYC1 (SEQ. ID NO: 50), harboured tagged BaTDC and CPR, The second (Xii-2)::PTDH3-PriB-His-TCYC1 (SEQ. ID NO: 51), harboured only tagged PriB. Successive genomic integration of these cassettes was performed as described previously (Chen et al., 2018). Following stable integration of these two cassettes, the strain was further manipulated by transformation with a yeast episomal vector encoding a promiscuous N-acetyltransferase, PsmF (pMM1-pTDH3-PsmF-His-tCYC1). For construction of pMM1-pTDH3-PsmF-His-tCYC1, the gene PsmF (SEQ. ID NO: 9) fused in-frame with a HIS epitope tag (SEQ. ID NO: 46) was ligated to empty plasmid pMM1 (SEQ. ID NO: 34) using BamHI/SacII restriction sites. For this Example, heterologous expression of a non-native or engineered TrpB gene was not necessary, as endogenous tryptophan synthase activity proved sufficient. The final engineered strain was called Sc-4. For scaled-up production of derivative product, culturing was performed as described in Example 20, with the following exception: in place of 4-chloro-1H-indole, 500 μM 5-chloro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-(2-[5-chloro-6-(3-methyl-2-butenyl)-1H-indol-3-yl]ethyl)acetamide, having chemical formula (XXXVII):

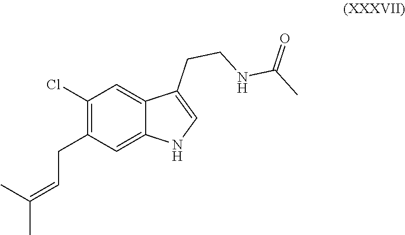 [SMILES: CC(=O)NCCc1c[nH]c2cc(CC=C(C)C)c(Cl)cc12]

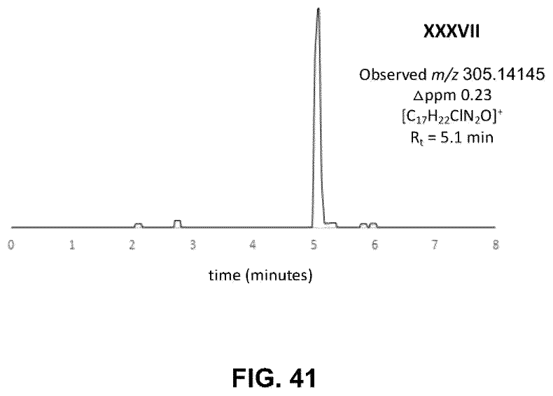

eluted at 5.1 minutes (EIC, see: FIG. 41).

### Example 28—Biosynthesis of a Twenty-Eighth Multi-Substituent Psilocybin Derivative

The same yeast strain (Sc-4) and procedures described in Example 27 were used to biosynthesize a psilocybin derivative with chemical formula (XXXV), with the following exception: in place of 5-chloro-1H-indole, 500 μM 5-fluoro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N-(2-[5-fluoro-6-(3-methyl-2-butenyl)-1H-indol-3-yl]ethyl)acetamide, having chemical formula (XXXV):

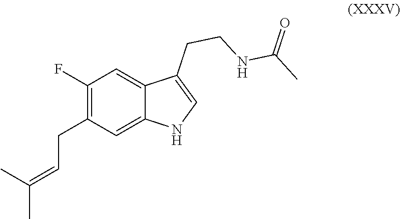 [SMILES: CC(=O)NCCc1c[nH]c2cc(CC=C(C)C)c(F)cc12]

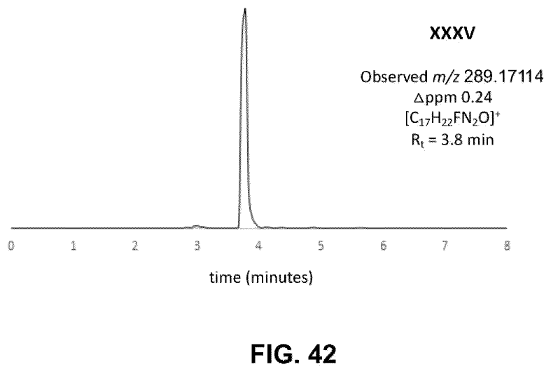

eluted at 3.8 minutes (EIC, see: FIG. 42).

### Example 29—Biosynthesis of a Twenty-Ninth Multi-Substituent Psilocybin Derivative

Yeast (Saccharomyces cerevisiae) strain Sc-5 was obtained as an intermediate in the process of assembling Sc-4. The strain Sc-5 is essentially identical to Sc-4, with the exception that Sc-5 does not harbour an additional episomal vector (pMM1-pTDH3-PsmF-His-tCYC1) encoding the promiscuous N-acetyltransferase, PsmF (SEQ. ID NO: 10). Thus, Sc-5 hosts only two enzymes through chromosomal integration—BaTDC (SEQ. ID NO: 4) and PriB (SEQ. ID NO: 18)—which participate in derivative formation. A third enzyme, CPR (SEQ. ID NO: 26) is similarly integrated but does not contribute to derivative production. Heterologous expression of a non-native or engineered TrpB gene was not necessary, as endogenous tryptophan synthase activity proved sufficient. For scaled-up production of derivative product, culturing was performed as described in Example 20, with the following exception: in place of 4-chloro-1H-indole, 500 μM 5-fluoro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-[5-fluoro-6-(3-methyl-2-butenyl)-1H-indol-3-yl]ethylamine, having chemical formula (XXXVI):

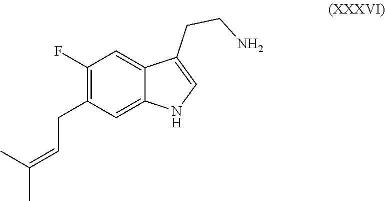 [SMILES: CC(C)=CCc1cc2[nH]cc(CCN)c2cc1F]

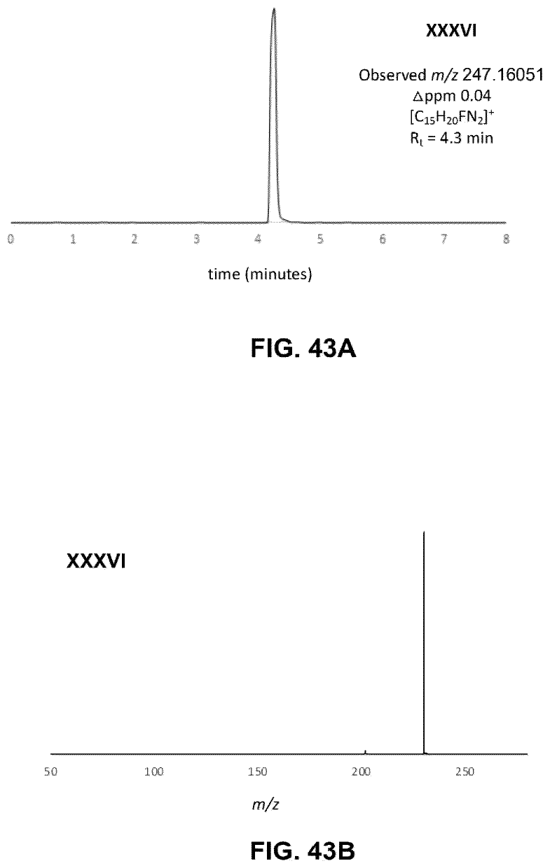

eluted at 4.3 minutes (EIC, see: FIG. 43). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XXXVI) as follows (FIG. 43B, Table 11) (Servillo L. et al, 2013, J. Agric. Chem. 61: 5156-5162).

TABLE 11

| m/z | % Relative abundance | Ionic species | Δ ppm |
|---|---|---|---|
| 230.13358 | 100 | [M + H − NH₃]⁺ | 1.61 |
| 198.47229 | 0.6 | | |
| 89.48803 | 0.5 | | |
| 233.50280 | 0.5 | | |
| 154.92723 | 0.5 | | |
| 115.92102 | 0.5 | | |
| 132.59683 | 0.5 | | |
| 92.18462 | 0.5 | | |
| 199.41139 | 0.4 | | |
| 102.89690 | 0.4 | | |

### Example 30—Biosynthesis of a Thirtieth Multi-Substituent Psilocybin Derivative

The same yeast strain (Sc-5) and procedures described in Example 29 were used to biosynthesize a psilocybin derivative with chemical formula (XXXVIII), with the following exception: in place of 5-fluoro-1H-indole, 500 μM 5-chloro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-[5-chloro-6-(3-methyl-2-butenyl)-1H-indol-3-yl]ethylamine, having chemical formula (XXXVIII):

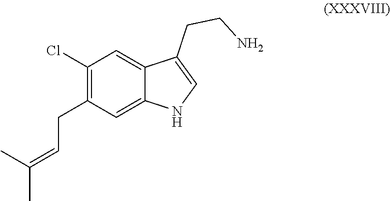 [SMILES: CC(C)=CCc1cc2[nH]cc(CCN)c2cc1Cl]

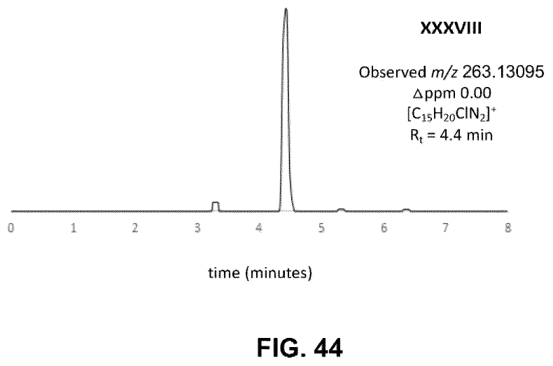

eluted at 4.4 minutes (EIC, see: FIG. 44).

### Example 31—Biosynthesis of a Thirty-First Multi-Substituent Psilocybin Derivative

Yeast (Saccharomyces cerevisiae) strain Sc-6 was created through genetic engineering of a parent yeast strain, to enable bioconversion of commercially obtained, derivatized indole, tryptophan, or tryptamine feedstock to generate final product. The parent yeast (Saccharomyces cerevisiae) strain was CEN PK with genotype Mata; ura3-52; trp1-289; leu2-3,112; his3Δ1; MAL2-8C; SUC2. The parent strain was engineered to include PsiH (SEQ. ID NO: 24), CPR (SEQ. ID NO: 26), ClostSporTDC (SEQ. ID NO: 6) and PsiM (SEQ. ID NO: 14) which catalyzed or supported several enzymatic steps. Engineering also included PsiK (SEQ. ID NO: 49) although this enzyme did not appear capable of contributing to the bioconversion process. PsiH, CPR, PsiM and PsiK were included in the strain through chromosomal homologous recombination of integration cassettes as described previously (Dastmalchi et al., 2019, Nat. Chem Biol. 15: 384-390; Chen et al., 2018, Nat. Chem Biol. 14: 738-743). Conversely, ClostSporTDC was built into a protein expression plasmid and transformed to the genomically integrated strain already harboring PsiH, CPR, PsiM and PsiK. PsiH, CPR, ClostSporTDC and PsiM were encoded by SEQ. ID NO: 23, SEQ. ID NO: 25, SEQ. ID NO: 5, and SEQ. ID NO: 13, respectively, with addition of in-frame, C-terminal HA (SEQ. ID NO: 38, SEQ. ID NO: 39), c-MYC (SEQ. ID NO: 40, SEQ. ID NO: 41), HIS (SEQ. ID NO: 46, SEQ. ID NO: 47) and FLAG (SEQ. ID NO: 42, SEQ. ID NO: 43) epitope tags, respectively. Integration cassettes were built using yeast promoter sequences amplified from S. cerevisiae genomic DNA as described (Dastmalchi et al., 2019; Chen et al., 2018) enabling constitutive gene expression. Amplified promoters included PGK1 (SEQ. ID NO: 30), TDH3 (SEQ. ID NO: 31), CLN1 (SEQ. ID NO: 32), and UGA1 (SEQ. ID NO: 33). Two integration cassettes were assembled: the first, XII-4::TADH1-PsiH-HA-PPGK1-PTDH3-CPR-c-myc-TCYC1 (SEQ. ID NO: 27), harboured tagged PsiH and CPR. The second, XII-5::TADH1-PsiK-V5-PPGK1-PTDH3-PsiM-FLAG-TCYC1 (SEQ. ID NO: 28), harboured tagged PsiK and PsiM. Successive genomic integration of these cassettes was performed as described previously (Chen et al., 2018). Following stable integration of these two cassettes, the strain was further manipulated by transformation with a yeast episomal vector encoding ClostSporTDC (pMM1-pTDH3-ClostSpor-His-tCYC1). For construction of pMM1-pTDH3-ClostSpor-His-tCYC1, the gene ClostSporTDC (SEQ. ID NO: 5) fused in-frame with a HIS epitope tag (SEQ. ID NO: 46) was ligated to empty plasmid pMM1 (SEQ. ID NO: 34) using BamHI/SacII restriction sites. For this Example, heterologous expression of a non-native or engineered TrpB gene was not necessary, as endogenous tryptophan synthase activity proved sufficient. The final engineered strain was called Sc-6. Employing Sc-6, the same procedures described in Example 20 were used to biosynthesize a psilocybin derivative with chemical formula (XL), with the following exception: in place of 4-chloro-1H-indole, 500 μM 6-chloro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 6-chloro-3-[2-(dimethylamino)ethyl]-1H-indol-4-ol, having chemical formula (XL):

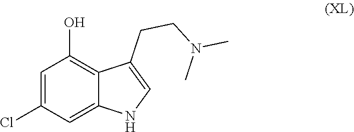 [SMILES: CN(C)CCc1c[nH]c2cc(Cl)cc(O)c12]

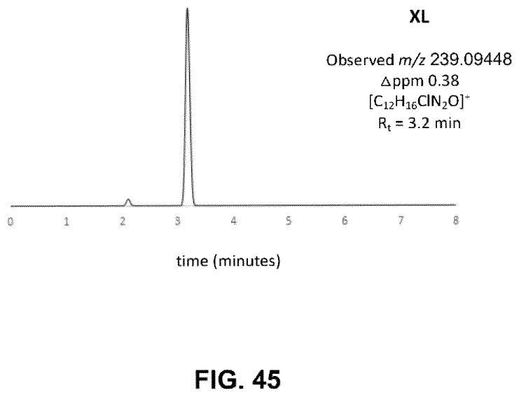

eluted at 0.38 minutes (EIC, see: FIG. 45).

### Example 32—Biosynthesis of a Thirty-Second Multi-Substituent Psilocybin Derivative

The same yeast strain (Sc-6) and procedures described in Example 31 were used to biosynthesize a psilocybin derivative with chemical formula (XXXIX), with the following exception: in place of 4-chloro-1H-indole, 500 μM 7-chloro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 7-chloro-3-[2-(dimethylamino)ethyl]-1H-indol-4-ol, having chemical formula (XXXIX):

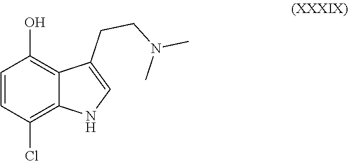 [SMILES: CN(C)CCc1c[nH]c2c(Cl)ccc(O)c12]

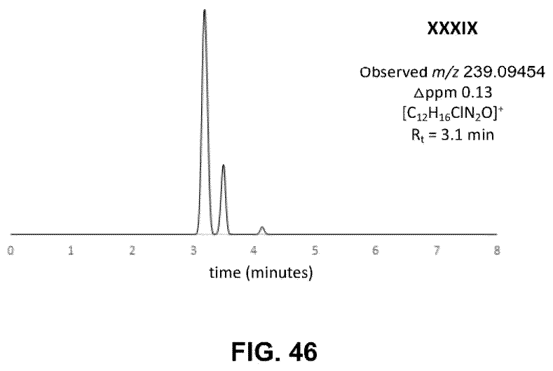

eluted at 3.1 minutes (EIC, see: FIG. 46).

### Example 33—Biosynthesis of a Thirty-Third Multi-Substituent Psilocybin Derivative

The same yeast strain (Sc-6) and procedures described in Example 31 were used to biosynthesize a psilocybin derivative with chemical formula (LII), with the following exception: in place of 4-chloro-1H-indole, 500 μM 6-fluoro-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-6-fluoro-1H-indol-4-ol, having chemical formula (LII):

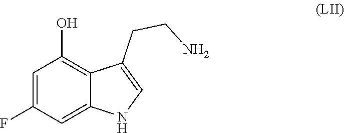 [SMILES: NCCc1c[nH]c2cc(F)cc(O)c12]

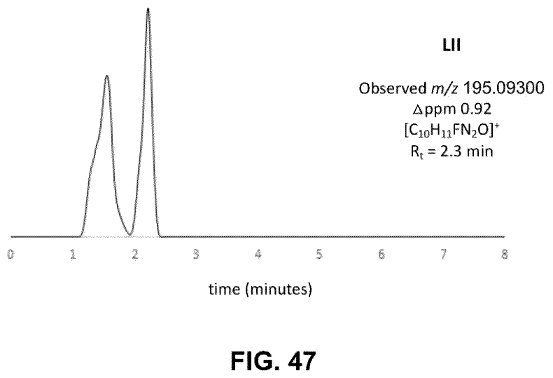

eluted at 2.3 minutes (EIC, see: FIG. 47). Notably, the product (LII) did not bear an N,N-dimethyl function despite the presence of methyltransferase enzyme PsiM, implying that PsiM was incapable of accepting this product (LII) as a substrate.

### Example 34—Biosynthesis of a Thirty-Fourth Multi-Substituent Psilocybin Derivative

The same yeast strain (Sc-6) and procedures described in Example 31 were used to biosynthesize a psilocybin derivative with chemical formula (LIII), with the following exception: in place of 4-chloro-1H-indole, 500 μM 6-bromo-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 6-bromo-3-[2-(dimethylamino)ethyl]-1H-indol-4-ol, having chemical formula (LIII):

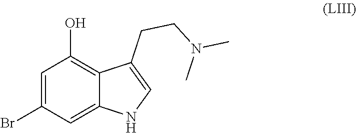 [SMILES: CN(C)CCc1c[nH]c2cc(Br)cc(O)c12]

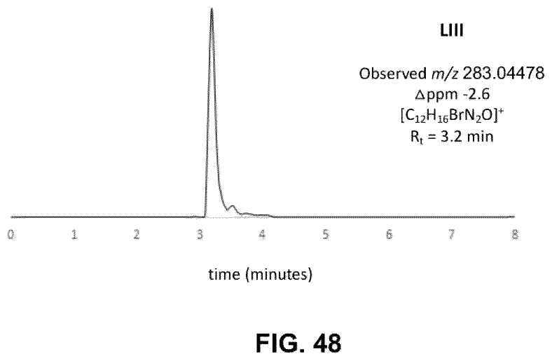

eluted at 3.2 minutes (EIC, see: FIG. 48).

### Example 35—Biosynthesis of a Thirty-Fifth Multi-Substituent Psilocybin Derivative

The same yeast strain (Sc-6) and procedures described in Example 31 were used to biosynthesize a psilocybin derivative with chemical formula (LIV), with the following exception: in place of 4-chloro-1H-indole, 500 μM 6-bromo-1H-indole (Combi-Blocks, www.combi-blocks.com) was supplied as feedstock for bioconversion. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-aminoethyl)-6-bromo-1H-indol-4-ol, having chemical formula (LIV):

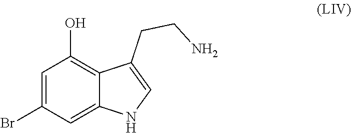 [SMILES: NCCc1c[nH]c2cc(Br)cc(O)c12]

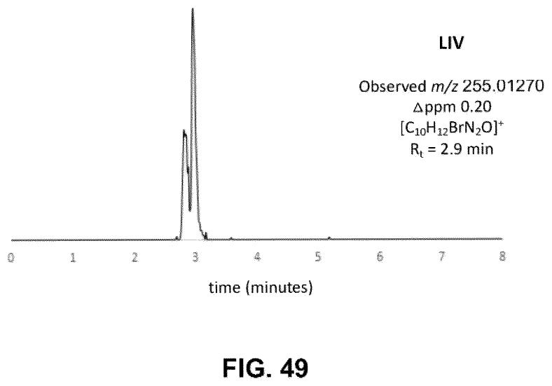

eluted at 2.9 minutes (EIC, see: FIG. 49). Notably, while the same indole feedstock was provided in this example compared to the feedstock used in Example 34, the product (LIII) is not the same as product (LIV). In fact, both products (LIII) and (LIV) are achieved by feeding 6-bromo-1H-indole to Sc-6. However, in Example 34, only product (LIII) was analyzed. Conversely, in this Example, only product (LIV) was analyzed.

### Example 36—Biosynthesis of a Thirty-Sixth Multi-Substituent Psilocybin Derivative

Synthesis of a psilocybin derivative was accomplished using FgaPT2 enzyme and an in vitro procedure. cDNA encoding FgaPT2 (SEQ. ID NO: 21) was synthesized and subcloned at GenScript (www.genscript.com) using Nde1 and Xho1 sites to pET26b(+) plasmid (SEQ. ID NO: 54). The final plasmid pET26b(+)-FgaPT2 encoded an in-frame, C-terminal HIS tag fusion of FgaPT2. Purified, recombinant FgaPT2 enzyme (SEQ. ID NO: 22) was raised in E. coli and isolated as follows. The plasmid pET26b(+)-FgaPT2 was transformed into Rosetta (DE3) competent E. coli cells. Transformed Rosetta (DE3) E. coli cells were grown in LB media at 30° C. for overnight and then transferred into TB (terrific broth) media to grow at 37° C. until optical density (OD₆₀₀) reached 0.6-1.5. The cell culture was then transferred to a 16° C. incubator with the addition of IPTG at 0.2 mM to initiate recombinant protein expression. After 20 hours the cells were harvested by centrifugation at 5,000×g for 6 minutes and the cell pellet was stored in −80° C. before protein extraction. For extraction and purification of FgaPT2 recombinant protein, E. coli cells were resuspended in a buffer containing 50 mM sodium phosphate (pH 7.0) and 300 mM NaCl and then sonicated for 5-10 minutes to break the cells. The cell lysate was centrifuged at 12,000 g for 30 minutes to collect the supernatant containing soluble crude protein. The supernatant was applied to cobalt resin (TALON Superflow™, Cytiva) to isolate HIS-tagged target protein. Purified protein was stored at −80° C. in a buffer containing 50 mM Tris-HCl (pH 7.0), 100 mM NaCl, and 10% glycerol. The tryptophan derivative 2-amino-3-(5-bromo-1H-indol-3-yl)propionic acid (www.sigmaaldrich.com) and DMAPP (www.sigmaaldrich.com) were used as co-substrates in the reaction. Briefly, reactions were set up as follows: 50 mM Tris-HCl (pH 8.0), 180 μM DMAPP, 0.5 mM tryptophan derivative, and 300 μg/mL of FgaPT2 were added together and the reaction proceeded at 37° C. for 2 hours. Equal volume of MeOH was added to quench the reaction and precipitate the protein. The sample was then centrifuged at 13,000 g for 20 minutes, allowing removal of the supernatant which contained the desired product. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[5-bromo-4-(3-methyl-2-butenyl)-1H-indol-3-yl]propionic acid, having chemical formula (XXX):

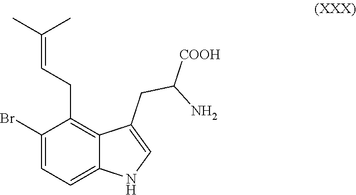 [SMILES: CC(C)=CCc1c(Br)ccc2[nH]cc(CC(N)C(=O)O)c12]

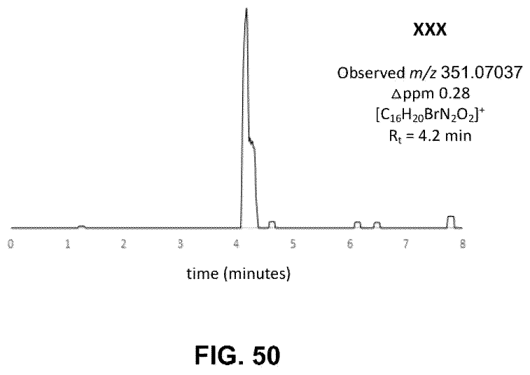

eluted at 4.2 minutes (EIC, see: FIG. 50).

### Example 37—Biosynthesis of a Thirty-Seventh Multi-Substituent Psilocybin Derivative

Synthesis of a psilocybin derivative was accomplished using FgaPT2 enzyme and the in vitro procedure described in Example 36, with the exception that 2-amino-3-(6-fluoro-1H-indol-3-yl)propionic acid (www.sigmaaldrich.com) was used in place of 2-amino-3-(5-bromo-1H-indol-3-yl)propionic acid substrate. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 2-amino-3-[6-fluoro-4-(3-methyl-2-butenyl)-1H-indol-3-yl]propionic acid, having chemical formula (XXXI):

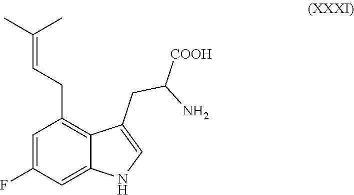 [SMILES: CC(C)=CCc1cc(F)cc2[nH]cc(CC(N)C(=O)O)c12]

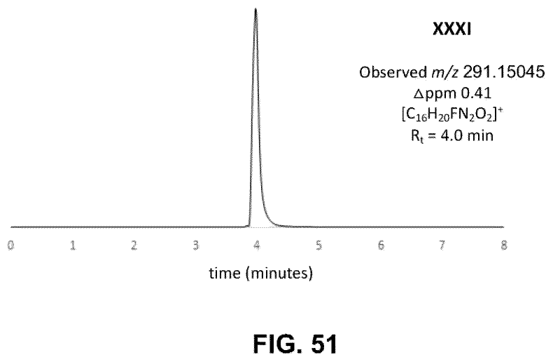

eluted at 4.0 minutes (EIC, see: FIG. 51).

### Example 38—Biosynthesis of a Thirty-Eighth Multi-Substituent Psilocybin Derivative

Synthesis of a psilocybin derivative was accomplished using PriB enzyme and an in vitro procedure. cDNA encoding PriB (SEQ. ID NO: 17) was synthesized and subcloned at GenScript (www.genscript.com) using Nde1 and Xho1 sites to pET26b(+) plasmid (SEQ. ID NO: 54). The final plasmid pET26b(+)-PriB encoded an in-frame, C-terminal HIS tag fusion of PriB. Purified, recombinant PriB enzyme (SEQ. ID NO: 18) was raised in E. coli and isolated as follows. The plasmid pET26b(+)-PriB was transformed into Rosetta (DE3) competent E. coli cells. Transformed Rosetta (DE3) E. coli cells were grown in LB media at 30° C. for overnight and then transferred into TB (terrific broth) media to grow at 37° C. until optical density (OD₆₀₀) reached 0.6-1.5. The cell culture was then transferred to a 16° C. incubator with the addition of IPTG at 0.5 mM to initiate recombinant protein expression. After 20 hours the cells were harvested by centrifugation at 5,000×g for 6 minutes and the cell pellet was stored in −80° C. before protein extraction. For extraction and purification of PriB recombinant protein, E. coli cells were resuspended in a buffer containing 50 mM sodium phosphate (pH 7.0) and 300 mM NaCl and then sonicated for 5-10 minutes to break the cells. The cell lysate was centrifuged at 12,000 g for 30 minutes to collect the supernatant containing soluble crude protein. The supernatant was applied to cobalt resin (TALON Superflow™, Cytiva) to isolate HIS-tagged target protein. Purified protein was stored at −80° C. in a buffer containing 50 mM Tris-HCl (pH 7.0), 100 mM NaCl, and 10% glycerol. The co-substrates 3-[2-(dimethylamino)ethyl]-1H-indol-4-yl propionate (Indole Shop; www.theindoleshop.com) and DMAPP (www.sigmaaldrich.com) were used in the reaction. Briefly, reactions were set up as follows: 50 mM Tris-HCl (pH 8.0), 180 μM DMAPP, 0.5 mM 3-[2-(dimethylamino)ethyl]-1H-indol-4-yl propionate and 392 μg/mL of PriB were added together and the reaction proceeded at 37° C. for 2 hours. Equal volume of MeOH was added to quench the reaction and precipitate the protein. The sample was then centrifuged at 13,000 g for 20 minutes, allowing removal of the supernatant which contained the desired product. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-[2-(dimethylamino)ethyl]-6-(3-methyl-2-butenyl)-1H-indol-4-yl propionate, having chemical formula (XLI):

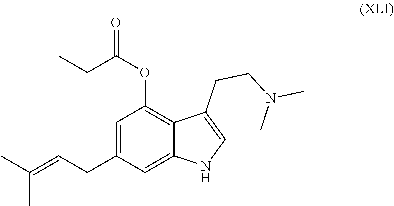 [SMILES: CCC(=O)Oc1cc(CC=C(C)C)cc2[nH]cc(CCN(C)C)c12]

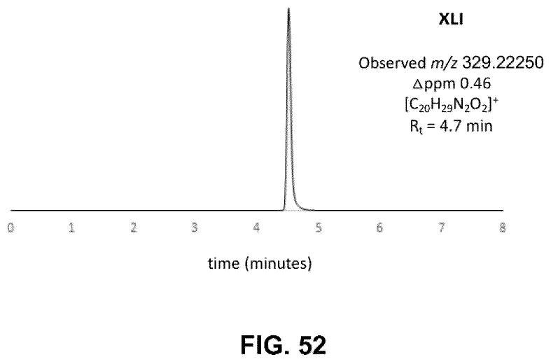

eluted at 4.7 minutes (EIC, see: FIG. 52).

### Example 39—Biosynthesis of a Thirty-Ninth Multi-Substituent Psilocybin Derivative

Synthesis of a psilocybin derivative was accomplished using PriB enzyme and the in vitro procedure described in Example 38, with the exception that 3-[2-(dimethylamino)ethyl]-1H-indol-4-yl acetate (Indole Shop; www.theindoleshop.com) was used in place of 3-[2-(dimethylamino)ethyl]-1H-indol-4-yl propionate substrate. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-[2-(dimethylamino)ethyl]-6-(3-methyl-2-butenyl)-1H-indol-4-yl acetate, having chemical formula (XLII):

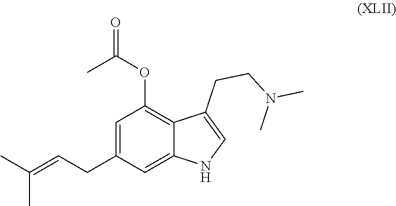 [SMILES: CC(=O)Oc1cc(CC=C(C)C)cc2[nH]cc(CCN(C)C)c12]

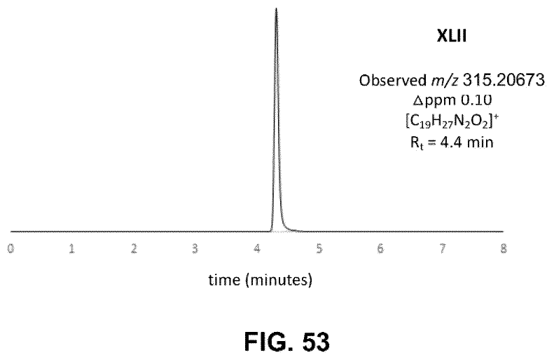

eluted at 4.4 minutes (EIC, see: FIG. 53).

### Example 40—Biosynthesis of a Fortieth Multi-Substituent Psilocybin Derivative

Synthesis of a psilocybin derivative was accomplished using PriB enzyme and the in vitro procedure described in Example 38, with the exception that 3-[2-(diethylamino)ethyl]-1H-indol-4-yl acetate (Indole Shop; www.theindoleshop.com) was used in place of 3-[2-(dimethylamino)ethyl]-1H-indol-4-yl propionate substrate. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-[2-(diethylamino)ethyl]-6-(3-methyl-2-butenyl)-1H-indol-4-yl acetate, having chemical formula (XLV):

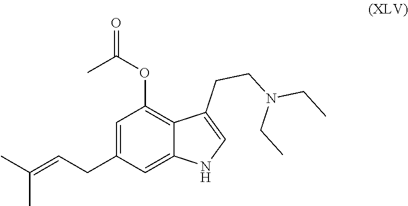 [SMILES: CCN(CC)CCc1c[nH]c2cc(CC=C(C)C)cc(OC(C)=O)c12]

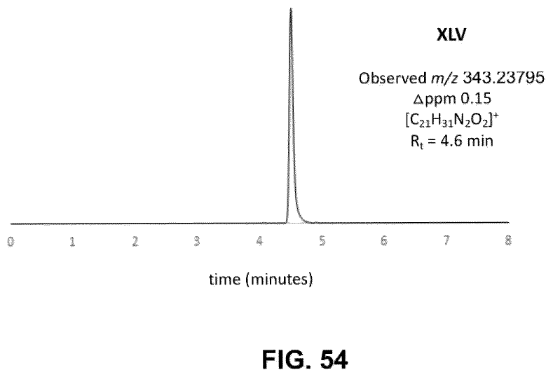

eluted at 4.6 minutes (EIC, see: FIG. 54).

### Example 41—Synthesis of a Forty-First Multi-Substituent Psilocybin Derivative

Synthesis of a psilocybin derivative was accomplished using PriB enzyme and the in vitro procedure described in Example 38, with the exception that N,N-dimethyl[2-(5-chloro-1H-indol-3-yl)ethyl]amine (Indole Shop; www.theindoleshop.com) was used in place of 3-[2-(dimethylamino)ethyl]-1H-indol-4-yl propionate substrate. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of N,N-dimethyl(2-[5-chloro-6-(3-methyl-2-butenyl)-1H-indol-3-yl]ethyl)amine, having chemical formula (XLIV):

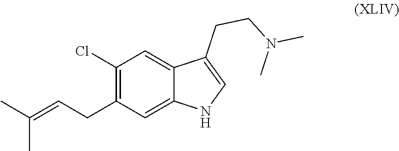 [SMILES: CC(C)=CCc1cc2[nH]cc(CCN(C)C)c2cc1Cl]

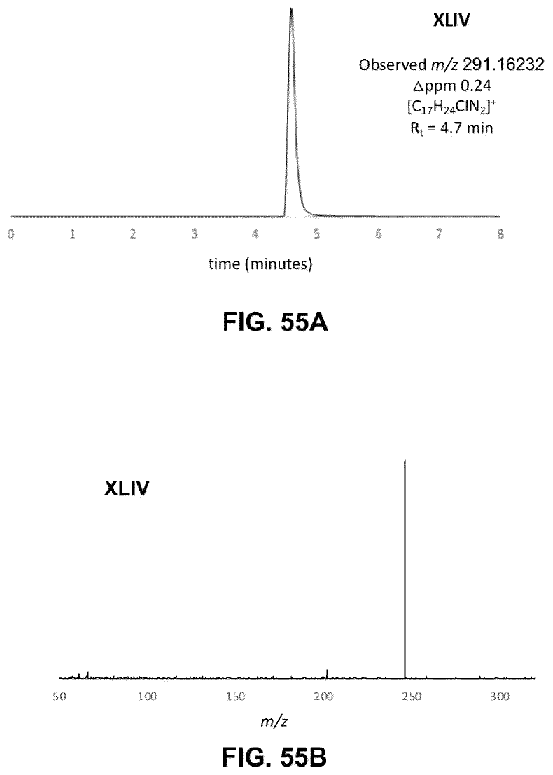

eluted at 4.7 minutes (EIC, see: FIG. 55A). As per standard procedures (Menéndez-Perdomo et al., 2021, Mass Spectrom 56: 34683) further analysis using high energy collisions (HCD) was achieved in a dedicated, post-LTQ, nitrogen collision cell. Orbitrap-based, HR fragment detection was employed (normalized collision energy, NCE 35), enabling opportunity to assign elemental formulae to subsequent diagnostic ion species characteristic of the targeted psilocybin derivative with formula (XLIV) as follows (FIG. 55B, Table 12) (Servillo L. et al., 2013, J. Agric. Chem. 61: 5156-5162).

TABLE 12

| m/z | % Relative abundance | Ionic species | Δ ppm |
|---|---|---|---|
| 246.10398 | 100 | [M + H − C₆H₉N]⁺ | 1.71 |
| 61.09263 | 2.0 | | |
| 199.41002 | 1.5 | | |
| 181.73308 | 1.4 | | |
| 65.66295 | 1.2 | | |
| 116.30919 | 1.2 | | |
| 56.98338 | 1.1 | | |
| 80.77037 | 1.1 | | |
| 164.10424 | 0.9 | | |
| 151.38086 | 0.9 | | |

### Example 42—Biosynthesis of a Forty-Second Multi-Substituent Psilocybin Derivative

Synthesis of a psilocybin derivative was accomplished using (1) chemical synthesis, followed by (2) an in vitro enzymatic conversion by PriB enzyme. Chemical synthesis was conducted using the synthesis procedure shown in FIG. 13D.

To a solution of 4-Benzyloxyindole 13D-1 (1.00 mmol, 1.00 eq) in anhydrous diethyl ether (10 mL) under argon sparging at 0° C., was added oxalyl chloride (2.05 mmol, 2.05 eq) dropwise over the course of 30 minutes, and the reaction was continued at 0-5° C. for 3 hours. A solution of N-methylisopropylamine (5.00 mmol, 5.00 eq) in anhydrous diethyl ether (5 mL) was added dropwise over the course of 1 hour. The solution was concentrated in vacuo, and the residue was redissolved in dichloromethane (30 mL). The organic solution was washed with water (4×10 mL) and brine (1×10 mL), then dried over anhydrous Na₂SO₄ and concentrated under reduced pressure to yield compound 13D-2, which was used in the following step without further purification. A solution of lithium aluminum hydride in a mixture of anhydrous THF (1 M, 5.20 eq) and 1,4-dioxane (2.0 mL) was brought to 60° C. under argon. A solution of compound 13D-2 in a mixture of anhydrous THF (2.0 mL) and 1,4-dioxane (3.5 mL) was added dropwise over 30 minutes, and the reaction was brought to 70° C. for 2 hours. The reaction was further refluxed at 95° C. for 20 hours. After cooling to 0° C., excess lithium aluminum hydride was quenched through a dropwise addition of a mixture of water (0.4 mL)-THF (2.0 mL). Diethyl ether (10 mL) was added, and the reaction mixture was allowed to stir at room temperature for 30 minutes. The precipitate was removed via vacuum filtration, the filtrate was dried over anhydrous Na₂SO₄, and concentrated under vacuo to yield compound 3 which was used without further purification. The crude compound 13D-3 was dissolved in 95% EtOH (10 mL), and 10% palladium on activated charcoal (0.110 eq) was added. The reaction flask was evacuated then backfilled with hydrogen. After stirring at room temperature for 2 hours, the catalyst was removed by a filtration and solvent was removed under reduced pressure to yield compound 13D-4, which was purified by a reverse-phase column chromatography on C18 silica gel using a water−acetonitrile+0.1% formic acid as the eluent. Compound 13D-4 was then used as a substrate for bioconversion by PriB enzyme, the latter which was generated and purified using the procedure described in Example 38. The in vitro conversion by PriB was conducted using the same procedure described in Example 38, with the exception that compound 13D-4 was used in place of 3-[2-(dimethylamino)ethyl]-1H-indol-4-yl propionate substrate. Analysis was carried out using high-resolution LC-HESI-LTQ-Orbitrap-XL MS (Thermo Fisher Scientific) as described in Example 3. Singly protonated product with exact m/z and expected elemental formula matching the singly protonated form of 3-(2-[(isopropyl)-N-methylamino]ethyl)-6-(3-methyl-2-butenyl)-1H-indol-4-ol, having chemical formula (LXXVI):

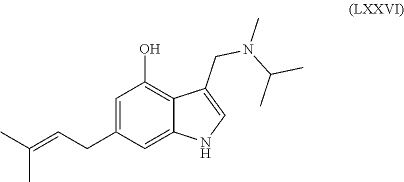 [SMILES: CC(C)=CCc1cc(O)c2c(CN(C)C(C)C)c[nH]c2c1]

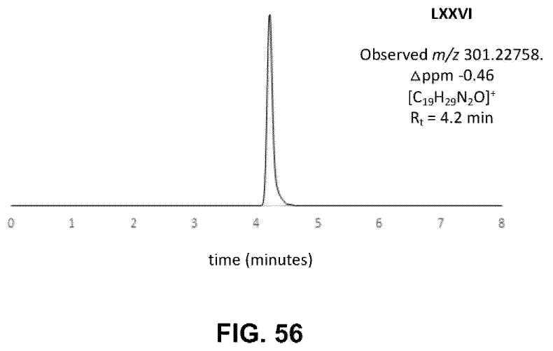

eluted at 4.2 minutes (EIC, see: FIG. 56).

1. A chemical compound or a salt thereof having a formula (I): 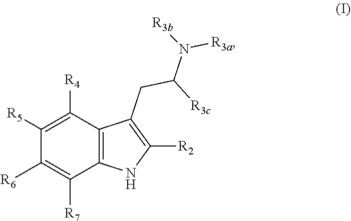
wherein, at least two of R₂, R₄, R₅, R₆, or R₇ are substituents independently selected from at least one of each of (i) a halogen atom, and (ii) a prenyl group, and wherein each non-substituted R₂, R₅, R₆, or R₇ is a hydrogen atom, and when R₄ is not substituted with any of the foregoing substituents, R₄ is a hydrogen atom, an O-alkyl group, an O-acyl group, or a phosphate group, and wherein R₃ₐ and R_{3b} are each independently a hydrogen atom, an alkyl group, a cycloalkyl group, an acyl group, or an aryl group, and R_{3c} is a hydrogen atom.

2. A chemical compound according to claim 1, wherein two of R₂, R₄, R₅, R₆, or R₇ are substituted.

3. A chemical compound according to claim 1, wherein at least two of R₅, R₆, and R₇ are substituted, and wherein R₂ and R₄ are hydrogen atoms.

4. A chemical compound according to claim 1, wherein two of R₅, R₆, and R₇ are substituted, and wherein R₂ and R₄ are hydrogen atoms.

5. A chemical compound according to claim 1, wherein R₅ and R₆ are substituted, and wherein R₂, R₄, and R₇ are hydrogen atoms.

6. A chemical compound according to claim 5, wherein R₅ is a halogen atom, and R₆ is a prenyl group.

7. A chemical compound according to claim 1, wherein the halogen atom is a fluorine atom.

8. A chemical compound according to claim 4, wherein, the halogen atom is a fluorine atom.

9. A chemical compound according to claim 5, wherein, the halogen atom is a fluorine atom.

10. A chemical compound according to claim 6, wherein, the halogen atom is a fluorine atom.

11. A chemical compound according to claim 5, wherein R₃ₐ is a hydrogen atom and R_{3b} is a (C₁-C₃)-alkyl group.

12. A chemical compound according to claim 5, wherein R₃ₐ is a hydrogen atom and R_{3b} is a methyl group (—CH₃).

13. A chemical compound according to claim 5, wherein R₃ₐ and R_{3b} are a (C₁-C₃)-alkyl group.

14. A chemical compound according to claim 5, wherein R₃ₐ and R_{3b} are a methyl group (—CH₃).

15. A chemical compound according to claim 5, wherein R₃ₐ and R_{3b} are a hydrogen atom.

16. A chemical compound according to claim 6, wherein R₃ₐ is a hydrogen atom and R_{3b} is a (C₁-C₃)-alkyl group.

17. A chemical compound according to claim 6, wherein R₃ₐ is a hydrogen atom and R_{3b} is a methyl group (—CH₃).

18. A chemical compound according to claim 6, wherein R₃ₐ and R_{3b} are a (C₁-C₃)-alkyl group.

19. A chemical compound according to claim 6, wherein R₃ₐ and R_{3b} are a methyl group (—CH₃).

20. A chemical compound according to claim 6, wherein R₃ₐ and R_{3b} are a hydrogen atom.

21. A chemical compound according to claim 10, wherein R₃ₐ is a hydrogen atom and R_{3b} is a (C₁-C₃)-alkyl group.

22. A chemical compound according to claim 10, wherein R₃ₐ is a hydrogen atom and R_{3b} is a methyl group (—CH₃).

23. A chemical compound according to claim 10, wherein R₃ₐ and R_{3b} are a (C₁-C₃)-alkyl group.

24. A chemical compound according to claim 9, wherein R₃ₐ and R_{3b} are a methyl group (—CH₃).

25. A chemical compound according to claim 10, wherein R₃ₐ and R_{3b} are a hydrogen atom.

26. A chemical compound according to claim 1, wherein chemical compound (I) is a compound having a chemical formula (A): 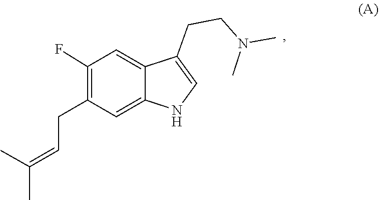 [SMILES: CC(C)=CCc1cc2[nH]cc(CCN(C)C)c2cc1F]
or a salt thereof.

27. A pharmaceutical or recreational drug formulation comprising an effective amount of a chemical compound according to claim 1, together with a pharmaceutically acceptable excipient, diluent, or carrier.

28. A method for treating a psychiatric disorder, the method comprising administering to a subject in need thereof a pharmaceutical formulation comprising a chemical compound according to claim 1, wherein the pharmaceutical formulation is administered in an effective amount to treat the psychiatric disorder in the subject.

29. A pharmaceutical or recreational drug formulation comprising an effective amount of a chemical compound according to claim 26, together with a pharmaceutically acceptable excipient, diluent, or carrier.

30. A method for treating a psychiatric disorder, the method comprising administering to a subject in need thereof a pharmaceutical formulation comprising a chemical compound according to claim 26, wherein the pharmaceutical formulation is administered in an effective amount to treat the psychiatric disorder in the subject.


SEQUENCE LISTING (54 sequences)

SEQ ID NO: 1 - DNA, 1167 bp, Pyrococcus furiosus
  source 1..1167: mol_type = genomic DNA
atgtggttcggtgagtttggtggacaatatgtgccagagactttagtgggtcctcttaaggaattggaaaaggcatataaaaggttcaaggacgatgaggagttcaacaggcaactaaactattatttgaagacatgggccggtagaccaacgcccttgtattatgctaagaggttaactgaaaagattggcggcgcgaaagtgtatctgaaaagagaagacctagttcatggtggagcacacaagacaaataatgccattggacaagcactattggcaaagctaatgggtaaaactagattgatagctgagacaggagcgggtcaacatggggtcgcgacagcgatggctggtgcactactggggatgaaggtagatatttacatgggtgctgaggacgttgagcgtcagaaactaaatgtcttcaggatgaagctattaggtgccaatgttatacctgtaaattctggctcaagaacactaaaggacgccttcgacgaggctcttagagactgggttgccactttcgagtatactcattacttgatcggttcagtggttggaccacatccatacccaaccatcgttagggactttcagagcgtgattggtagagaggctaaggcacagatcttagaagcagagggacagctacctgacgtcatagttgcctgcgtcggcggtggctctaacgcaatgggtatattctatccattcgttaatgacaagaaggttaaattagtaggagtcgaagctggcggaaaggggttagagtcgggtaaacactcagcaagcttaaatgcaggacaggtaggggtgtcccacggcatgttgtcgtatttcttgcaagacgaggaaggtcagataaagccaagtcattcaattgctccaggccttgaccaccccggtgttggtccagagcacgcttacttaaagaagattcaaagggccgagtacgtcgctgtaacagacgaagaggcattgaaagctttccatgagctatccagaactgaggggattatacccgcccttgagtctgcccatgctgtggcgtacgccatgaagttagctaaagagatgtcccgtgacgaaatcatcattgtaaatctatcagggagaggagacaaggatttggacattgtattgaaggcaagcggaaatgtttga

SEQ ID NO: 2 - protein, 388 aa, Pyrococcus furiosus
MWFGEFGGQYVPETLVGPLKELEKAYKRFKDDEEFNRQLNYYLKTWAGRPTPLYYAKRLTEKIGGAKVYLKREDLVHGGAHKTNNAIGQALLAKLMGKTRLIAETGAGQHGVATAMAGALLGMKVDIYMGAEDVERQKLNVFRMKLLGANVIPVNSGSRTLKDAFDEALRDWVATFEYTHYLIGSVVGPHPYPTIVRDFQSVIGREAKAQILEAEGQLPDVIVACVGGGSNAMGIFYPFVNDKKVKLVGVEAGGKGLESGKHSASLNAGQVGVSHGMLSYFLQDEEGQIKPSHSIAPGLDHPGVGPEHAYLKKIQRAEYVAVTDEEALKAFHELSRTEGIIPALESAHAVAYAMKLAKEMSRDEIIIVNLSGRGDKDLDIVLKASGNV

SEQ ID NO: 3 - DNA, 1446 bp, Bacillus atrophaeaus
  source 1..1446: mol_type = genomic DNA
atgatgtctgaaaatttgcaattgtcagctgaagaaatgagacaattgggttaccaagcagttgatttgatcatcgatcacatgaaccatttgaagtctaagccagtttcagaaacaatcgattctgatatcttgagaaataagttgactgaatctatcccagaaaatggttcagatccaaaggaattgttgcatttcttgaacagaaacgtttttaatcaaattacacatgttgatcatccacatttcttggcttttgttccaggtccaaataattacgttggtgttgttgcagatttcttggcttctggttttaatgtttttccaactgcatggattgctggtgcaggtgctgaacaaatcgaattgactacaattaattggttgaaatctatgttgggttttccagattcagctgaaggtttatttgtttctggtggttcaatggcaaatttgacagctttgactgttgcaagacaggctaagttgaacaacgatatcgaaaatgctgttgtttacttctctgatcaaacacatttctcagttgatagagcattgaaggttttaggttttaaacatcatcaaatctgtagaatcgaaacagatgaacatttgagaatctctgtttcagctttgaagaaacaaattaaagaagatagaactaagggtaaaaagccattctgtgttattgcaaatgctggtactacaaattgtggtgctgttgattctttgaacgaattagcagatttgtgtaacgatgaagatgtttggttgcatgctgatggttcttatggtgctccagctatcttgtctgaaaagggttcagctatgttgcaaggtattcatagagcagattctttgactttagatccacataagtggttgttccaaccatacgatgttggttgtgttttgatcagaaactctcaatatttgtcaaagacttttagaatgatgccagaatacatcaaggattcagaaactaacgttgaaggtgaaattaatttcggtgaatgtggtatcgaattgtcaagaagattcagagctttgaaggtttggttgtcttttaaagttttcggtgttgctgcttttagacaagcaatcgatcatggtatcatgttagcagaacaagttgaagcatttttgggtaaagcaaaagattgggaagttgttacaccagctcaattgggtatcgttacttttagatacattccatctgaattggcatcaacagatactattaatgaaattaataagaaattggttaaggaaatcacacatagaggtttcgctatgttatctactacagaattgaaggaaaaggttgttattagattgtgttcaattaatccaagaactacaactgaagaaatgttgcaaatcatgatgaagattaaagcattggctgaagaagtttctatttcatacccatgtgttgctgaataa

SEQ ID NO: 4 - protein, 481 aa, Bacillus atrophaeaus
MMSENLQLSAEEMRQLGYQAVDLIIDHMNHLKSKPVSETIDSDILRNKLTESIPENGSDPKELLHFLNRNVFNQITHVDHPHFLAFVPGPNNYVGVVADFLASGFNVFPTAWIAGAGAEQIELTTINWLKSMLGFPDSAEGLFVSGGSMANLTALTVARQAKLNNDIENAVVYFSDQTHFSVDRALKVLGFKHHQICRIETDEHLRISVSALKKQIKEDRTKGKKPFCVIANAGTTNCGAVDSLNELADLCNDEDVWLHADGSYGAPAILSEKGSAMLQGIHRADSLTLDPHKWLFQPYDVGCVLIRNSQYLSKTFRMMPEYIKDSETNVEGEINFGECGIELSRRFRALKVWLSFKVFGVAAFRQAIDHGIMLAEQVEAFLGKAKDWEVVTPAQLGIVTFRYIPSELASTDTINEINKKLVKEITHRGFAMLSTTELKEKVVIRLCSINPRTTTEEMLQIMMKIKALAEEVSISYPCVAE

SEQ ID NO: 5 - DNA, 1254 bp, Clostridium sporidium
  source 1..1254: mol_type = genomic DNA
atgaagttctggagaaagtacacacaacaagaaatggatgaaaagattactgaatctttggaaaagactttgaactacgataacactaagacaatcggtattccaggtactaagttggatgatacagttttctatgatgatcattctttcgttaagcattcaccatacttgagaacttttattcaaaacccaaaccatatcggttgtcatacttatgataaggctgatatcttgttcggtggtacattcgatatcgaaagagaattaatccaattgttagcaatcgatgttttgaacggtaacgatgaagaatttgatggttacgttactcaaggtggtacagaagctaacatccaagcaatgtgggtttacagaaactacttcaagaaagaaagaaaggctaagcatgaagaaatcgctatcatcacttcagcagatacacattactctgcatacaaaggttcagatttgttgaacatcgatattattaaggttccagttgatttttattcaagaaaaattcaagaaaatacattggattcaattgttaaagaagctaaagaaattggtaaaaagtacttcatcgttatctctaacatgggtactacaatgtttggttcagttgatgatccagatttgtacgctaacatcttcgataagtacaatttggaatacaaaattcatgttgatggtgcatttggtggttttatatatccaattgataataaggaatgtaaaactgatttctctaataagaacgtttcttcaatcacattagatggtcataagatgttgcaagctccatacggtactggtatcttcgtttcaagaaagaatttgatccataacactttgacaaaggaagcaacttacatcgaaaatttggatgttacattgtctggttcaagatctggttcaaatgctgttgcaatttggatggttttagcttcttatggtccatacggttggatggaaaagattaataagttgagaaatagaactaaatggttgtgtaagcaattgaacgatatgagaattaaatattacaaagaagattcaatgaatattgttacaattgaagaacaatatgttaataaggaaatcgctgaaaagtactttttagttccagaagttcataacccaactaacaactggtacaagatcgttgttatggaacatgttgaattggatatcttgaactctttggtttacgatttgagaaagtttaataaggaacatttgaaggcaatgtaa

SEQ ID NO: 6 - protein, 417 aa, Clostridium sporidium
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 7 - DNA, 1320 bp, Psilocybe cubensis
  source 1..1320: mol_type = genomic DNA
atgcaggtgatacccgcgtgcaactcggcagcaataagatcactatgtcctactcccgagtcttttagaaacatgggatggctctctgtcagcgatgcggtctacagcgagttcataggagagttggctacccgcgcttccaatcgaaattactccaacgagttcggcctcatgcaacctatccaggaattcaaggctttcattgaaagcgacccggtggtgcaccaagaatttattgacatgttcgagggcattcaggactctccaaggaattatcaggaactatgtaatatgttcaacgatatctttcgcaaagctcccgtctacggagaccttggccctcccgtttatatgattatggccaaattaatgaacacccgagcgggcttctctgcattcacgagacaaaggttgaaccttcacttcaaaaaacttttcgatacctggggattgttcctgtcttcgaaagattctcgaaatgttcttgtggccgaccagttcgacgacagacattgcggctggttgaacgagcgggccttgtctgctatggttaaacattacaatggacgcgcatttgatgaagtcttcctctgcgataaaaatgccccatactacggcttcaactcttacgacgacttctttaatcgcagatttcgaaaccgagatatcgaccgacctgtagtcggtggagttaacaacaccaccctcatttctgctgcttgcgaatcactttcctacaacgtctcttatgacgtccagtctctcgacactttagttttcaaaggagagacttattcgcttaagcatttgctgaataatgaccctttcaccccacaattcgagcatgggagtattctacaaggattcttgaacgtcaccgcttaccaccgatggcacgcacccgtcaatgggacaatcgtcaaaatcatcaacgttccaggtacctactttgcgcaagccccgagcacgattggcgaccctatcccggataacgattacgacccacctccttaccttaagtctcttgtctacttctctaatattgccgcaaggcaaattatgtttattgaagccgacaacaaggaaattggcctcattttccttgtgttcatcggcatgaccgaaatctcgacatgtgaagccacggtgtccgaaggtcaacacgtcaatcgtggcgatgacttgggaatgttccatttcggtggttcttcgttcgcgcttggtctgaggaaggattgcagggcagagatcgttgaaaagttcaccgaacccggaacagtgatcagaatcaacgaagtcgtcgctgctctaaaggcttag

SEQ ID NO: 8 - protein, 439 aa, Psilocybe cubensis
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 9 - DNA, 546 bp, Streptomyces griseofuscus
  source 1..546: mol_type = genomic DNA
atgaacaccttcagaacagccactgccagagacatacctgatgtagcagcaactcttacggaagccttcgcaactgatccacccacgcagtgggtgttccccgacggtactgccgccgtcagcaggttctttacacatgttgcagatagggttcacacggccggtggtattgttgagctactaccagacagagccgccatgattgcattgccaccacacgtgaggctgccaggagaagctgccgacggaaggcaggcggaaattcagagaaggctggcagacaggcacccgctgacacctcactactacctgctgttttacggagttagaacggcacaccagggttcgggattgggcggaagaatgctggccagattaactagcagagctgatagggacagggtgggtacatatactgaggcatccacctggcgtggcgctagactgatgctgagacatggattccatgctacaaggccactaagattgccagatggacccagcatgtttccactttggagagatccaatccatgatcattctgat

SEQ ID NO: 10 - protein, 182 aa, Streptomyces griseofuscus
MNTFRTATARDIPDVAATLTEAFATDPPTQWVFPDGTAAVSRFFTHVADRVHTAGGIVELLPDRAAMIALPPHVRLPGEAADGRQAEIQRRLADRHPLTPHYYLLFYGVRTAHQGSGLGGRMLARLTSRADRDRVGTYTEASTWRGARLMLRHGFHATRPLRLPDGPSMFPLWRDPIHDHSD

SEQ ID NO: 11 - DNA, 1092 bp, Ephedra sinica
  source 1..1092: mol_type = genomic DNA
atgggatccatggaagaagcaaaaatggcgaccctgggcggtgcgtcctatgcgatgattgtgaaaacgatgatgcgctctctggaagcaaacctgattccggattttgtgctgcgtcgcctgacgcgtatcctgctggctagtcgcctgaaactgggttataagcagaccgctgaactgcaactggcggatctgatgtcattcgttgcgtcgctgaaaacgatgccgattgccctgtgcaccgaagaagcaaagggtcagcattacgaactgccgaccagctttttcaaactggtcctgggcaaacatctgaagtatagctctgcctacttttctgaacacacccgtacgctggatgaagcggaagaagccatgctggcactgtattgcgaacgcgccaaaattgaagatggtcagaagattctggacatcggctgtggttggggcagtttttccctgtatgtggcagaacgttacccgaaatgcgaaattacgggcctgtgtaacagttccacccaaaaagccttcatcgaacagcaatgcagcgaacgtcgcctgtgtaatgttaccatttatgcagatgacatcagcacctttgatacggaatctacctacgaccgcattatcagcatcgaaatgttcgaacacatgaagaactacagtacgctgctgaagaaaattagcaagtggatgaatcaggaatgcctgctgtttgtccattatttctgtcacaaaacctttgcgtaccacttcgaagatgtggacgaagatgactggatggctcgttatttctttaccggcggcaccatgccggcgtcatcgctgctgctgtactttcaggatgacgtctcagtggttgatcattggctgattaacggtaaacactatgctcaaacctcggaagaatggctgaagcgtatggaccacaatctgagctctattctgccgatctttaacgaaacgtatggcgaaaatgcggccaaaaagtggctggcatactggcgcacctttttcatcgcagttgctgaactgttcaaatacaacgatggcgaagaatggatggtgtcccacttcctgttcaaaaagaaataa

SEQ ID NO: 12 - protein, 363 aa, Ephedra sinica
MGSMEEAKMATLGGASYAMIVKTMMRSLEANLIPDFVLRRLTRILLASRLKLGYKQTAELQLADLMSFVASLKTMPIALCTEEAKGQHYELPTSFFKLVLGKHLKYSSAYFSEHTRTLDEAEEAMLALYCERAKIEDGQKILDIGCGWGSFSLYVAERYPKCEITGLCNSSTQKAFIEQQCSERRLCNVTIYADDISTFDTESTYDRIISIEMFEHMKNYSTLLKKISKWMNQECLLFVHYFCHKTFAYHFEDVDEDDWMARYFFTGGTMPASSLLLYFQDDVSVVDHWLINGKHYAQTSEEWLKRMDHNLSSILPIFNETYGENAAKKWLAYWRTFFIAVAELFKYNDGEEWMVSHFLFKKK

SEQ ID NO: 13 - DNA, 930 bp, Psilocybe cubensis
  source 1..930: mol_type = genomic DNA
atgcatatcagaaatccttaccgtacaccaattgactatcaagcactttcagaggccttccctcccctcaagccatttgtgtctgtcaatgcagatggtaccagttctgttgacctcactatcccagaagcccagagggcgttcacggccgctcttcttcatcgtgacttcgggctcaccatgaccataccagaagaccgtctgtgcccaacagtccccaataggttgaactacgttctgtggattgaagatattttcaactacacgaacaaaaccctcggcctgtcggatgaccgtcctattaaaggcgttgatattggtacaggagcctccgcaatttatcctatgcttgcctgtgctcggttcaaggcatggtctatggttggaacagaggtcgagaggaagtgcattgacacggcccgcctcaatgtcgtcgcgaacaatctccaagaccgtctctcgatattagagacatccattgatggtcctattctcgtccccattttcgaggcgactgaagaatacgaatacgagtttactatgtgtaaccctccattctacgacggtgctgccgatatgcagacttcggatgctgccaaaggatttggatttggcgtgggcgctccccattctggaacagtcatcgaaatgtcgactgagggaggtgaatcggctttcgtcgctcagatggtccgtgagagcttgaagcttcgaacacgatgcagatggtacacgagtaacttgggaaagctgaaatccttgaaagaaatagtggggctgctgaaagaacttgagataagcaactatgccattaacgaatacgttcaggggtccacacgtcgttatgccgttgcgtggtctttcactgatattcaactgcctgaggagctttctcgtccctctaaccccgagctcagctctcttttctag

SEQ ID NO: 14 - protein, 309 aa, Psilocybe cubensis
MHIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 15 - DNA, 1419 bp, Aspergillus fumigatus
  source 1..1419: mol_type = genomic DNA
atgtccatcggagccgagatcgattcgctggttcctgctccaccgggcctcaacggcaccgctgcgggctatccagccaagacgcagaaggagttaagcaacggagactttgacgcgcacgatggtctttctcttgcacaactgacaccgtacgatgtcttgacggctgcacttccgctgccggctccggcttcgagcacagggttctggtggcgggagacgggccctgttatgagcaagcttttggccaaggcgaactaccctctttacactcattacaagtaccttatgttataccatacccatattctcccattgttgggacctcgaccgccgctcgagaactcgacgcacccgtcgccgagtaacgcgccgtggaggtccttcctgacagacgacttcactccgctcgagccgagctggaacgtgaacgggaactcggaagcacagagcacaatccgtcttggtattgaacctataggctttgaagccggggctgcagcggacccattcaaccaagctgccgtgacgcagttcatgcactcatacgaggcaaccgaagtcggtgccacgctgacgctgttcgagcacttccgcaacgacatgtttgttggcccagaaacgtacgctgcgttaagagcgaagataccagaaggcgagcataccacacagagtttcctggcgttcgacctggacgcgggtcgtgtcaccacaaaggcgtactttttcccgattctcatgtcgttgaaaactggacagagcacaacaaaggtggtctctgattccattctgcatctagcgctgaagagtgaggtgtggggtgtgcagaccatcgccgcgatgtcggtcatggaggcgtggataggtagctacggtggcgcggcaaagacggagatgatcagcgtcgattgcgtgaacgaggcagactctcggatcaagatatacgtgcggatgccacatacatccttgcggaaggtaaaagaggcgtactgcttaggtgggcggttgacagacgagaacacaaaggagggcctgaagctgctggacgagctgtggaggacggtcttcggcatcgacgacgaggacgcggagctgccacagaatagccatcgcaccgcaggcacaatattcaatttcgagctgaggccagggaaatggttccccgagcccaaggtatacctgcccgtccgacactactgtgaaagtgatatgcagattgctagtcggctacaaacgttctttggaaggctcggatggcacaacatggagaaagattattgcaagcatctggaagatttgtttccccatcatccactgtcctcgtcaacgggcacacacacctttctctcattttcgtataagaagcagaagggggtctatatgaccatgtattataatctccgggtgtacagcacctaa

SEQ ID NO: 16 - protein, 472 aa, Aspergillus fumigatus
MSIGAEIDSLVPAPPGLNGTAAGYPAKTQKELSNGDFDAHDGLSLAQLTPYDVLTAALPLPAPASSTGFWWRETGPVMSKLLAKANYPLYTHYKYLMLYHTHILPLLGPRPPLENSTHPSPSNAPWRSFLTDDFTPLEPSWNVNGNSEAQSTIRLGIEPIGFEAGAAADPFNQAAVTQFMHSYEATEVGATLTLFEHFRNDMFVGPETYAALRAKIPEGEHTTQSFLAFDLDAGRVTTKAYFFPILMSLKTGQSTTKVVSDSILHLALKSEVWGVQTIAAMSVMEAWIGSYGGAAKTEMISVDCVNEADSRIKIYVRMPHTSLRKVKEAYCLGGRLTDENTKEGLKLLDELWRTVFGIDDEDAELPQNSHRTAGTIFNFELRPGKWFPEPKVYLPVRHYCESDMQIASRLQTFFGRLGWHNMEKDYCKHLEDLFPHHPLSSSTGTHTFLSFSYKKQKGVYMTMYYNLRVYST

SEQ ID NO: 17 - DNA, 1158 bp, unidentified
  source 1..1158: mol_type = other DNA; note = Streptomyces sp. RM-5-8
atgggaggtccgatgagcggtttccattcgggggaggcgctgctcggtgacctcgccaccggtcagctgaccaggctgtgcgaggtggcggggctgaccgaggccgacacggcggcctacacgggggtgctgatcgaaagtctggggacgtcggccggacggccgttgtccctgccacccccgtcgcggacctttctctccgacgaccacacccccgtggagttctccctggccttcctgccgggacgcgcaccgcacctgcgggtcctggtggaaccgggctgctccagcggcgacgacctggcggaaaacggccgggccggtctgcgggcggtccacaccatggcggaccgctggggattctccaccgagcaactcgaccggctggaggacctgttcttcccctcctcccccgagggcccgctggccctgtggtgcgccctggagctccgctccggtggggtgccgggggtgaaggtctacctcaaccccgcggcgaatggcgccgaccgggccgccgagacggtacgcgaggcgctggccaggctgggccacctgcaggcgttcgacgcgctgccccgggcggacggcttcccgttcctcgccctggacctcggcgactgggacgccccgcgggtgaagatctacctcaaacacctcggcatgtccgccgccgacgcgggctccctcccccggatgtcgcccgcaccgagccgggagcagctggaggagttcttccgcaccgccggtgacctcccggccccgggagacccggggcccaccgaggacaccggccggctcgccgggcgccccgccctcacctgccactccttcacggagacggcgaccgggcggcccagcggctacaccctccacgtgccggtccgcgactacgtccggcacgacggcgaggcacgggaccgggcggtggccgtgctgcgcgaacatgacatggacagtgcggcactggaccgggcgctggccgccgtgagcccccgcccgctgagtgacggggtgggcctgatcgcctatctggcactggtccaccagcgcggccggccgacacgggtgaccgtctacgtctcctccgaggcgtacgaggtgcggccgccccgcgagacggtccccacccgcgaccgggcgcgggcacggctgtga

SEQ ID NO: 18 - protein, 385 aa, unidentified
  source 1..385: note = Streptomyces sp. RM-5-8
MGGPMSGFHSGEALLGDLATGQLTRLCEVAGLTEADTAAYTGVLIESLGTSAGRPLSLPPPSRTFLSDDHTPVEFSLAFLPGRAPHLRVLVEPGCSSGDDLAENGRAGLRAVHTMADRWGFSTEQLDRLEDLFFPSSPEGPLALWCALELRSGGVPGVKVYLNPAANGADRAAETVREALARLGHLQAFDALPRADGFPFLALDLGDWDAPRVKIYLKHLGMSAADAGSLPRMSPAPSREQLEEFFRTAGDLPAPGDPGPTEDTGRLAGRPALTCHSFTETATGRPSGYTLHVPVRDYVRHDGEARDRAVAVLREHDMDSAALDRALAAVSPRPLSDGVGLIAYLALVHQRGRPTRVTVYVSSEAYEVRPPRETVPTRDRARARL

SEQ ID NO: 19 - DNA, 1128 bp, Streptomyces coelicolor
  source 1..1128: mol_type = genomic DNA
atgagggccgcgtcgacgggcgcggacccgcaggacgcatccacgctcggctctttcaccggcggccagttgcgaagactcggctcggtcgccggtctgtcccgcgccgacgtcgagacctacgcacaggtcctgaccgacgcattgggcccggtggcccagcggccgctgagcctggcgccgcccacccgcaccttcctgtcggacgaccacacccccgtggagttctccctctccttccggcccggggcggcgcccgccatgcgggtcctcgtggaaccgggctgcggtgcgaccagcctggccgacaacggccgtgccggtcttgaggcggtccgcacgatggcgcggcgctggcacttcaccaccgacgccctcgacgaactcctggacctgttcctgccgcccgctccgcagggccccctcgccctgtggtgcgccctggaactcaggcccgggggtgtaccgggcgtcaaggtctatctgaaccctgcggtgggcggggaggaacgttccgccgcgacggtgcgcgaggccctgcgccggctcgggcaccaccaggccttcgacagcctcccccagggcagtggatacccgttcctcgccctggacctcgggaactggacggagccccgggcgaaggtctacctgcgccacgacaacctcacggccggtcgggccgcacggctgtcccggacggactcgggcctcgtgccgaccgcggtcgagggtttcttccgcaccgccgcgggtcccggctccgacgcgggtgggctcgacgggcggcctgctcagtcctgccactccttcaccgaccccggcgcggagcggccgagcggcttcaccctgtacatcccggttcgtgactacgtccggcatgacggggaggccctggcgcgggcgtccaccgtgctgcaccaccacggcatggacgcctccgtgctccaccgcgccctggccgccctcaccgagcggcggcccgaggacggggtgggcctgatcgcctacctggccctcgccggccaacgggaccagccgccgcgggtgacggcctacctctcctcggaggcctacacggtccggccgccggtcgtggagaccgtccgccaaccgctgtcggtcggctga

SEQ ID NO: 20 - protein, 375 aa, Streptomyces coelicolor
MRAASTGADPQDASTLGSFTGGQLRRLGSVAGLSRADVETYAQVLTDALGPVAQRPLSLAPPTRTFLSDDHTPVEFSLSFRPGAAPAMRVLVEPGCGATSLADNGRAGLEAVRTMARRWHFTTDALDELLDLFLPPAPQGPLALWCALELRPGGVPGVKVYLNPAVGGEERSAATVREALRRLGHHQAFDSLPQGSGYPFLALDLGNWTEPRAKVYLRHDNLTAGRAARLSRTDSGLVPTAVEGFFRTAAGPGSDAGGLDGRPAQSCHSFTDPGAERPSGFTLYIPVRDYVRHDGEALARASTVLHHHGMDASVLHRALAALTERRPEDGVGLIAYLALAGQRDQPPRVTAYLSSEAYTVRPPVVETVRQPLSVG

SEQ ID NO: 21 - DNA, 1380 bp, Aspergillus fumigatus
  source 1..1380: mol_type = genomic DNA
atgaaggcagccaatgcctccagtgcggaggcctatcgagttcttagtcgcgcctttagattcgataatgaagatcagaagctgtggtggcacagcactgccccgatgtttgcaaaaatgctggaaactgccaactacaccacaccttgtcagtatcaatacctcatcacctataaggagtgcgtaattcccagtctcggatgctatccgaccaacagcgccccccgctggttgagcatcctcactcgatacggcactccgttcgaattgagcctaaattgctctaattcaatagtgagatacacattcgagccgatcaatcaacataccggaacagataaagacccattcaatacgcacgccatctgggagagcctgcagcacctgcttccactggagaagagcattgatctggagtggttccgccacttcaagcacgatctcaccctcaacagtgaagaatctgcttttctggctcataatgatcgcctcgtgggcggcactatcaggacgcagaacaagctcgcgctcgatctgaaggatggccgctttgcacttaagacgtacatatacccggctctcaaagctgtcgtcaccggcaagacaattcatgagttggtctttggctcagtccgccggctggcagtgagggagccccgaatcttgcccccactcaacatgctggaggaatacatccgatcacgcggttccaagagcactgccagtccccgcctagtgtcctgtgatctgaccagtcctgccaagtcgagaatcaagatctacctgctggagcagatggtttcactagaagccatggaggacctgtggactctgggcggacggcgccgagacgcttccactttagaggggctctctctggtgcgtgagctttgggatctgatccaactgtcgccgggattgaagtcctatccggcgccgtatctgcctctcggggttatcccagacgagaggctgccgcttatggccaatttcaccctgcaccagaatgacccggtcccagagccgcaagtatatttcacaaccttcggcatgaacgacatggcggtggcggatgccctgacgacgttcttcgagcgccggggttggagtgaaatggcccgcacctacgaaactactttgaagtcgtactacccccatgcggatcatgacaaacttaactacctccacgcctacatatccttctcctacagggaccgtaccccttatctgagtgtctatcttcaatccttcgagacaggggactgggcagttgcaaacttatccgaatcaaaggtcaagtgtcaggatgcggcctgtcaacccacagctttacctccagatctgtcaaagacaggggtatattattccggtctccactga

SEQ ID NO: 22 - protein, 459 aa, Aspergillus fumigatus
MKAANASSAEAYRVLSRAFRFDNEDQKLWWHSTAPMFAKMLETANYTTPCQYQYLITYKECVIPSLGCYPTNSAPRWLSILTRYGTPFELSLNCSNSIVRYTFEPINQHTGTDKDPFNTHAIWESLQHLLPLEKSIDLEWFRHFKHDLTLNSEESAFLAHNDRLVGGTIRTQNKLALDLKDGRFALKTYIYPALKAVVTGKTIHELVFGSVRRLAVREPRILPPLNMLEEYIRSRGSKSTASPRLVSCDLTSPAKSRIKIYLLEQMVSLEAMEDLWTLGGRRRDASTLEGLSLVRELWDLIQLSPGLKSYPAPYLPLGVIPDERLPLMANFTLHQNDPVPEPQVYFTTFGMNDMAVADALTTFFERRGWSEMARTYETTLKSYYPHADHDKLNYLHAYISFSYRDRTPYLSVYLQSFETGDWAVANLSESKVKCQDAACQPTALPPDLSKTGVYYSGLH

SEQ ID NO: 23 - DNA, 2155 bp, Psilocybe cubensis
  source 1..2155: mol_type = genomic DNA
atgatcgctgtactattctccttcgtcattgcaggatgcatatactacatcgtttctcgtagagtgaggcggtcgcgcttgccaccagggccgcctggcattcctattcccttcattgggaacatgtttgatatgcctgaagaatctccatggttaacatttctacaatggggacgggattacagtctgtcttgccgcgttgacttctaatatatgaacagctaatatattgtcagacaccgatattctctacgtggatgctggagggacagaaatggttattcttaacacgttggagaccattaccgatctattagaaaagcgagggtccatttattctggccggtgagctgatgttgagttttttgcaattgaatttgtggtcacacgtttccagacttgagagtacaatggtcaacgaacttatggggtgggagtttgacttagggttcatcacatacggcgacaggtggcgcgaagaaaggcgcatgttcgccaaggagttcagtgagaagggcatcaagcaatttcgccatgctcaagtgaaagctgcccatcagcttgtccaacagcttaccaaaacgccagaccgctgggcacaacatattcgccagtaagtactacttgaggaaaatagcgtacgcttcgctgaccggtccgtacatcaaagtcagatagcggcaatgtcactggatattggttatggaattgatcttgcagaagacgacccttggctggaagcgacccatttggctaatgaaggcctcgccatagcatcagtgccgggcaaattttgggtcgattcgttcccttctcgtgagcatccttcttctatgtaggaagggaaggagtctaacaagtgttagtaaaataccttcctgcttggttcccaggtgctgtcttcaagcgcaaagcgaaggtctggcgagaagccgccgaccatatggttgacatgccttatgaaactatgaggaaattagcagttagtcaaatgcgttctccccgtattttttcaatactctaacttcagctcacagcctcaaggattgactcgtccgtcgtatgcttcagctcgtctgcaagccatggatctcaacggtgaccttgagcatcaagaacacgtaatcaagaacacagccgcagaggttaatgtcggtaagtcaaaagcgtccgtcggcaattcaaaattcaggcgctaaagtgggtcttctcaccaaggtggaggcgatactgtaaggatttctcaatcgttagagtataagtgttctaatgcagtacatactccaccaaccagactgtctctgctatgtctgcgttcatcttggccatggtgaagtaccctgaggtccagcgaaaggttcaagcggagcttgatgctctgaccaataacggccaaattcctgactatgacgaagaagatgactccttgccatacctcaccgcatgtatcaaggagcttttccggtggaatcaaatcgcacccctcgctataccgcacaaattaatgaaggacgacgtgtaccgcgggtatctgattcccaagaacactctagtcttcgcaaacacctggtgaggctgtccattcattcctagtacatccgttgccccactaatagcatcttgataacagggcagtattaaacgatccagaagtctatccagatccctctgtgttccgcccagaaagatatcttggtcctgacgggaagcctgataacactgtacgcgacccacgtaaagcggcatttggctatggacgacgaaattggtaagtgcgctttcagaacccccccttccgttgactagtgccatgcgcgcatacaatatcgctattgatctgatataacttccctgcggcatttattttggcattcctttagtcccggaattcatctagcgcagtcgacggtttggattgcaggggcaaccctcttatcagcgttcaatatcgagcgacctgtcgatcagaatgggaagcccattgacataccggctgattttactacaggattcttcaggtagctaatttccgtctttgtgtgcataatacccctaacgacgcacgtttacctttttgtaaagacacccagtgcctttccagtgcaggtttgttcctcgaacagagcaagtctcacagtcggtatccggaccctga

SEQ ID NO: 24 - protein, 508 aa, Psilocybe cubensis
MIAVLFSFVIAGCIYYIVSRRVRRSRLPPGPPGIPIPFIGNMFDMPEESPWLTFLQWGRDYNTDILYVDAGGTEMVILNTLETITDLLEKRGSIYSGRLESTMVNELMGWEFDLGFITYGDRWREERRMFAKEFSEKGIKQFRHAQVKAAHQLVQQLTKTPDRWAQHIRHQIAAMSLDIGYGIDLAEDDPWLEATHLANEGLAIASVPGKFWVDSFPSLKYLPAWFPGAVFKRKAKVWREAADHMVDMPYETMRKLAPQGLTRPSYASARLQAMDLNGDLEHQEHVIKNTAAEVNVGGGDTTVSAMSAFILAMVKYPEVQRKVQAELDALTNNGQIPDYDEEDDSLPYLTACIKELFRWNQIAPLAIPHKLMKDDVYRGYLIPKNTLVFANTWAVLNDPEVYPDPSVFRPERYLGPDGKPDNTVRDPRKAAFGYGRRNCPGIHLAQSTVWIAGATLLSAFNIERPVDQNGKPIDIPADFTTGFFRHPVPFQCRFVPRTEQVSQSVSGP

SEQ ID NO: 25 - DNA, 2187 bp, Psilocybe cubensis
  source 1..2187: mol_type = genomic DNA
atggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtctagg

SEQ ID NO: 26 - protein, 728 aa, Psilocybe cubensis
MASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDESFEFSSGERKLSGLKYVVFGLGNKTYEHYNLIGRTVDAQLAKMGAIRIGERGEGDDDKSMEEDYLEWKDGMWEAFATAMGVEEGQGGDSADFVVSELESHPPEKVYQGEFSARALTKTKGIHDAKNPFAAPIAVARELFQSVVDRNCVHVEFNIEGSGITYQHGDHVGLWPLNPDVEVERLLCVLGLAEKRDAVISIESLDPALAKVPFPVPTTYGAVLRHYIDISAVAGRQILGTLSKFAPTPEAEAFLRNLNTNKEEYHNVVANGCLKLGEILQIATGNDITVPPTTANTTKWPIPFDIIVSAIPRLQPRYYSISSSPKIHPNTIHATVVVLKYENVPTEPIPRKWVYGVGSNFLLNLKYAVNKEPVPYITQNGEQRVGVPEYLIAGPRGSYKTESFYKAPIHVRRSTFRLPTNPKSPVIMIGPGTGVAPFRGFVQERVALARRSIEKNGPDSLADWGRISLFYGCRRSDEDFLYKDEWPQYEAELKGKFKLHCAFSRQNYKPDGSKIYVQDLIWEDREHIADAILNGKGYVYICGEAKSMSKQVEEVLAKILGEAKGGSGPVEGVAEVKLLKERSRLMLDVWS

SEQ ID NO: 27 - DNA, 6347 bp, synthetic construct
  misc_feature 1..6347: note = Synthetic construct
  source 1..6347: mol_type = other DNA
gtatccggctgttccttcatagccctttcaatgaacgttgcagccctttgaagattggccattttgtcaggactcgagcctgacagttggaccaacgcaactttaattttttgtgaaagaatcttcgaagcactcatactggcgatcttcacgccctcctgctattacaaaagctgtgtttttacaagaatcaaattaagttagcaagatattatacaacattattgataatttcaatatcgtgttcgtacctgatgacgtatctgtgcattgataaggcccgcatggtttcagaaagcagagcggaacgattccaaattagtggccttgtgctttgcatgtcaattgtgttaccttcagctcgtggatttgttttatcaatacacagtctacagtcaagaattttttttatcaaattttgcgttcgagcgtataaaatagccgctgtagctacttaagttcctgttcagcgatagtttttttccatcacacgtactatggcaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcaggcgtagtctggaacgtcgtatgggtatggaccagagacggattgagaaacttgttcggttcttgggacgaatctacattggaatggaactgggtgcctgaagaaaccagtggtgaaatcagctgggatgtcaattggcttaccgttttggtcaactggtctttcaatgttgaaagcagacaacaaagtagcaccggcaatccaaacagtagattgagctaggtgaatacctgggcagtttcttctaccgtaaccgaaagcagcctttcttgggtctctaacagtgttgtctggtttaccatcaggacccaagtatctttctggacggaaaacggatggatctggatagacttctgggtcattcaaaactgcccaggtgttagcaaaaaccaatgtgttctttggaatcaaataacctctgtaaacatcatccttcatcaatttatgagggatggctaatggagcaatttggttccatctgaataattccttgatacaagcggtcaaatatgggagtgagtcgtcttcctcatcgtagtcagggatttgaccgttgttggtcaaagcatccaattcagcttgaacctttctttgcacttctgggtatttaaccatggccaatatgaaagcggacatagcagagacggtagtgtcgccaccaccaacgttaacttcagcagcagtgttcttgataacatgttcttggtgttctaaatcaccgttcaagtccatagcttgtaatctagcagaagcgtaagatggtctggttagaccttgtggagccaactttctcatagtttcgtatggcatgtcaaccatgtggtcagcggcttctctccagaccttggccttacgcttgaagacggcacctgggaaccaagctggcaagtacttcaaagatgggaaggagtcaacccagaacttacctggaacactagcaatggccaaaccttcgttggctaagtgggtagcttctaaccatgggtcatcttcggctagatcgataccgtaaccaatgtccaaagacatagcagcgatttgatgtctgatgtgttgagcccaccggtccggagtcttggtcaattgttgaaccaattggtgagcagccttgacttgagcgtgtctgaattgcttgatacccttttcagagaattccttagcaaacattcttctttcttctctccaacggtcaccgtaagtgataaaacccagatcaaattcccaacccatcaattcattgaccatagtggattccaatctaccggagtagatggaaccacgtttttccaagagatccgtgatagtttccaaggtgtttaaaatgaccatttcggtaccaccagcatctacgtacaagatatcagtgttgtagtcacgaccccattgcaagaaagtcaaccatggagattcttctggcatgtcgaacatgttaccaataaatgggattggaattcctggtggaccaggtggcaatctggatctacgaactcttctggagacgatgtagtaaatacaaccagcaatgacgaaagagaacaagacagcaatcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtctgaacaaaagttaatttctgaagaagatttggaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaatagtttactcaattcttgaagccaatttgtacaattccccattagagtcaaataaaaggatgcctcacggaggtatgttacccgcgctatttcacatggctcattgaattagaggtggaatttggtgtaccctcccctcctcatctgatgaagtagtgatccgacaattcttaaaagttgtagacattacttttaccaccaactaagttgtatttatattgctacccttatccttttatatctaactagcgctcataaggttggggcaatactaaaactgtgttcttattcaactcattaaatacgtggcagtacgtaccctattagaaacaataggaaacagcagagtcggaagaagccaaatgccagatttgaagtccaaaaccttgtcaagccaatctttgggagcggctattcctccagaaattgtgtaccaaatacttacataccagtttagggatttgttaagaaatgaccatccaggtacggcagaaa

SEQ ID NO: 28 - DNA, 4792 bp, synthetic construct
  misc_feature 1..4792: note = Synthetic construct
  source 1..4792: mol_type = other DNA
caatctggcggcttgagttctcaacatgttttattttttacttatattgctggtagggtaaaaaaatataactcctaggaataggttgtctatatgtttttgtcttgcttctataattgtaacaaacaaggaaagggaaaatactgggtgtaaaagccattgagtcaagttaggtcatcccttttatacaaaatttttcaattttttttccaagattcttgtacgattaattattttttttttgcgtcctacagcgtgatgaaaatttcgcctgctgcaagatgagcgggaacgggcgaaatgtgcacgcgcacaacttacgaaacgcggatgagtcactgacagccaccgcagaggttctgactcctactgagctctattggaggtggcagaaccggtaccggaggaggccgctataaccggtttgaatttattgtcacagtgtcacatcagcattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcaggtggagtccaaacccaacaaaggatttggaattggcttaccggcagtggaggattcctttagcaacgtggaagtgatttcaccgttgtcgttgttacctctagcatcgtggaaagcagcaacacccttcttaacaaagttgattctttcttcttcagaaccccattgcatgaagtcagtccacataacaatgtgagcggcgataccagcggtaaccttggcgtagttgatggaatgcttggaagtacgggcgtaagattgcaagtaagcttgtctcatggttgtaccaacttgttcgtcttggaatctgctaatcaagtaacagtcacccaagaagtaacccaaatccaatgaagctggaccgtacttacacaattcccagtctaagatgtagatcttttgcaacttagatgggttaccttcttcaagttgcaacaagatgttcccagaccacaagtcagccatgaccaaagtttcttcggagtgcataacatcgtcaactagatccttgacaacagttggcaacaatggatcatcgacgccgtatttagcggcgtttgggataatagtttggtacaattggtcagaggtggttctaccgacaatgttaccagagaagaacttgaattctgggtcgtctcttctttctctacctatgttgtgcaatctggcgacgaaaccaccaatctcggtaccaaccaatctagcaatatcggtagccaaaggtggcttagcagtaacgtagtctaataaggtcttcattttaccgacatcttgcataatcaaagcattgttttccaagtcatagttgagaccttctggaacagagacaataccatcaacaccacccaaaacttctctgttagccatcatcaacttgatagcttggtattcgtagacagaacgttcaacaccgattttgaaatcttcatcagtagacatgtgtggttgagcgtgcttcaaaatgatagaagtgtgaccttggtatggagcgttcaatttaatacgccaggtgacgttaacgaaaccaccggataatctcttgacaccagaagtgtcaacatctaaagacaaatgcttggtcaaataggtgattaaaccgtcttcagtcttcaagtcaaaggccattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgcatatcagaaacccatatagaactccaattgactaccaagctttgtctgaagctttcccaccattgaagccatttgtttccgttaacgctgatggtacctcctcagttgacttgaccattccagaagcccaaagagcttttaccgctgcccttttgcacagagacttcggcttgactatgactatcccagaagatcgtttgtgtccaaccgttccaaacagattgaactacgttttgtggattgaagacattttcaactacaccaacaagactttgggtttatctgacgaccgtccaatcaagggtgttgatatcggtaccggtgcttctgccatttacccaatgttggcttgcgccagattcaaggcttggtccatggttggtactgaagttgaaagaaagtgtatcgacactgctagattaaacgttgttgctaacaacttgcaagatcgtctatccatcttggaaacctctattgacggtccaattttagtcccaattttcgaagctaccgaagaatacgaatacgaattcaccatgtgtaacccacctttctacgatggtgccgctgacatgcaaactagcgatgctgcaaagggttttggtttcggtgtcggtgctccacactctggtacagtcatcgaaatgtctactgaaggtggtgaatccgctttcgtggctcaaatggttagagaatctcttaagttgagaaccagatgtagatggtacacttctaacttaggtaagttgaaatctttgaaggaaattgtcggtttgttgaaggaattagaaatctctaattacgccatcaacgaatatgtccaaggttccactagaagatacgctgtcgcttggagtttcactgatatccaattgccagaagaattgtccagaccatctaatcctgaattgtcctctttgttcgactacaaggatgacgatgacaaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtacccaattcgccctatagtgagtcgtattacgcgcgctcactggccgtcgttttacaacgtcgtgactgggaaaaccctggcgttacccctgcaggactagtgctgaggcattaatgcaactcagaagtttgacagcaagcaagttcatcattcgaactagccttattgttttagttcagtgacagcgaactgccgtactcgatgctttatttctcacggtagagcggaagaacagataggggcagcgtgagaagagttagaaagtaaatttttatcacgtctgaagtattcttattcataggaaattttgcaaggttttttagctcaataacgggctaagttatataaggtgttcacgcgattttcttgttatgtatacctcttctctgaggaatggtactactgtcctgatgtaggctccttaaattggtgggcaagaataacttatcgatattttgtatattggtcttggagttcaccacgtaatgcctgtttaagaccatcagttaactctagtattatttggtcttggctactggccgtttgctattattcaagtcttttgtgccttcccgtcgggtaagggagttatttagggatacagaatctaacgaaaactaaatctcaatgattaactctatttaatccttttttgaaaggcaaaagaggtcccttgttcacttacaacgttcttagccaaattcgcttatcacttactacttcacgatatacagaagtaaaaacatataaaaagatgtctgtttgtttagccatcacaaaaggtatcgcag

SEQ ID NO: 29 - DNA, 9145 bp, synthetic construct
  misc_feature 1..9145: note = Synthetic construct
  source 1..9145: mol_type = other DNA
tgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaggatccatgatgaagttctggagaaagtacacacaacaagaaatggatgaaaagattactgaatctttggaaaagactttgaactacgataacactaagacaatcggtattccaggtactaagttggatgatacagttttctatgatgatcattctttcgttaagcattcaccatacttgagaacttttattcaaaacccaaaccatatcggttgtcatacttatgataaggctgatatcttgttcggtggtacattcgatatcgaaagagaattaatccaattgttagcaatcgatgttttgaacggtaacgatgaagaatttgatggttacgttactcaaggtggtacagaagctaacatccaagcaatgtgggtttacagaaactacttcaagaaagaaagaaaggctaagcatgaagaaatcgctatcatcacttcagcagatacacattactctgcatacaaaggttcagatttgttgaacatcgatattattaaggttccagttgatttttattcaagaaaaattcaagaaaatacattggattcaattgttaaagaagctaaagaaattggtaaaaagtacttcatcgttatctctaacatgggtactacaatgtttggttcagttgatgatccagatttgtacgctaacatcttcgataagtacaatttggaatacaaaattcatgttgatggtgcatttggtggttttatatatccaattgataataaggaatgtaaaactgatttctctaataagaacgtttcttcaatcacattagatggtcataagatgttgcaagctccatacggtactggtatcttcgtttcaagaaagaatttgatccataacactttgacaaaggaagcaacttacatcgaaaatttggatgttacattgtctggttcaagatctggttcaaatgctgttgcaatttggatggttttagcttcttatggtccatacggttggatggaaaagattaataagttgagaaatagaactaaatggttgtgtaagcaattgaacgatatgagaattaaatattacaaagaagattcaatgaatattgttacaattgaagaacaatatgttaataaggaaatcgctgaaaagtactttttagttccagaagttcataacccaactaacaactggtacaagatcgttgttatggaacatgttgaattggatatcttgaactctttggtttacgatttgagaaagtttaataaggaacatttgaaggcaatgcatcatcatcatcatcattaaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatttcacaccgcatagatccgtcgagttcaagagaaaaaaaaagaaaaagcaaaaagaaaaaaggaaagcgcgcctcgttcagaatgacacgtatagaatgatgcattaccttgtcatcttcagtatcatactgttcgtatacatacttactgacattcataggtatacatatatacacatgtatatatatcgtatgctgcagctttaaataatcggtgtcactacataagaacacctttggtggagggaacatcgttggtaccattgggcgaggtggcttctcttatggcaaccgcaagagccttgaacgcactctcactacggtgatgatcattcttgcctcgcagacaatcaacgtggagggtaattctgctagcctctgcaaagctttcaagaaaatgcgggatcatctcgcaagagagatctcctactttctccctttgcaaaccaagttcgacaactgcgtacggcctgttcgaaagatctaccaccgctctggaaagtgcctcatccaaaggcgcaaatcctgatccaaacctttttactccacgcacggcccctagggcctctttaaaagcttgaccgagagcaatcccgcagtcttcagtggtgtgatggtcgtctatgtgtaagtcaccaatgcactcaacgattagcgaccagccggaatgcttggccagagcatgtatcatatggtccagaaaccctatacctgtgtggacgttaatcacttgcgattgtgtggcctgttctgctactgcttctgcctctttttctgggaagatcgagtgctctatcgctaggggaccaccctttaaagagatcgcaatctgaatcttggtttcatttgtaatacgctttactagggctttctgctctgtcatctttgccttcgtttatcttgcctgctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgacttccctgactaatgccgtgttcaaacgatacctggcagtgactcctagcgctcaccaagctcttaaaacgggaatttatggtgcactctcagtacacgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttttgcggccgc

SEQ ID NO: 30 - DNA, 499 bp, synthetic construct
  misc_feature 1..499: note = Synthetic construct
  source 1..499: mol_type = other DNA
gtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 31 - DNA, 500 bp, synthetic construct
  misc_feature 1..500: note = Synthetic construct
  source 1..500: mol_type = other DNA
acagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 32 - DNA, 500 bp, synthetic construct
  misc_feature 1..500: note = Synthetic construct
  source 1..500: mol_type = other DNA
gctggattgagctgaatggtgccaggtcgaggctgggagggagactaactcgaaagtgacgaagactcgaaaattaaaaaaaaagatactgcagaaggcaagattgagaatggagtaaaggcagcgtgggtcccctgtggaaaccgcagttttcctgcgccaagtggtaccggtgcgagtgcagcaattaatctctcgatattttcttagtatctctttttatataagaatatattttggaattggtaatgcttatcttcaatagtttcttagttgaatgcacacttaagagcaaattggccaaggagttcttcgttcgctttaatttatttcctggttattgtcaatttattcatcccatctccccaggatagaagaaattagtgtaattttgctgacaatacattttaacgacgataacaataatagcaattaaataaaatagcactaccaccactccactgctcgttagctatttctgtaaaataaataaaaagatc

SEQ ID NO: 33 - DNA, 501 bp, synthetic construct
  misc_feature 1..501: note = Synthetic construct
  source 1..501: mol_type = other DNA
attcgcgctatctcgatttctacctatatagttaatctctgtacaaaaacaatctttccaactatccattaatcatagtatattatcagcgtcggcgattttaccacgcttgacaaaagccgcgggcgggattcctgtgggtagtggcaccggcagttaatctaatcaaaggcgcttgaaggaagagatagataatagaacaaagcaatcgccgctttggacggcaaatatgtttatccattggtgcggtgattggatatgatttgtctccagtagtataagcaagcgccagatctgtttactgtaaaattaagtgagtaatctcgcgggatgtaatgatttaagggaatctggttcaggttttcacatatatttgtatataaggccatttgtaatttcaatagttttaggatttttccttctcccaaaatactcacttactgtgttacattacagaaagaacagacaagaaaccgtcaataagaaatataactaagaaca

SEQ ID NO: 34 - DNA, 7882 bp, synthetic construct
  misc_feature 1..7882: note = Synthetic construct
  source 1..7882: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggatcttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatcttagctagccgcggtaccaagctggtggatcctttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacagcggccgcaaaagatctgagctcttaattaacaattcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctccaattcagctggcgtaatagcgaagaggcccgcaccgatcgcccttcccaacagttgcgcagcctgaatggcgaattcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctggcgcgtgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 35 - DNA, 3810 bp, synthetic construct
  misc_feature 1..3810: note = Synthetic contruct
  source 1..3810: mol_type = other DNA
gctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagcctaggatcgagatcgatctcgatcccgcgaaattaatacgactcactataggggaattgtgagcggataacaattcccctctagaaataattttgtttaactttaagaaggagatatacatatggcagatctcaattggatatcggccggccacgcgatcgctgacgtcggtaccctcgagtctggtaaagaaaccgctgctgcgaaatttgaacgccagcacatggactcgtctactagtcgcagcttaattaacctaaactgctgccaccgctgagcaataactagcataaccccttggggcctctaaacgggtcttgaggggttttttgctagcgaaaggaggagtcgacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagccagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggccc

SEQ ID NO: 36 - DNA, 5369 bp, synthetic construct
  misc_feature 1..5369: note = Synthetic construct
  source 1..5369: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 37 - DNA, 3592 bp, synthetic construct
  misc_feature 1..3592: note = Synthetic construct
  source 1..3592: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatcctagagggaaaccgttgtggtctccctatagtgagtcgtattaatttcgcgggatcgagatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 38 - DNA, 27 bp, synthetic construct
  misc_feature 1..27: note = Synthetic construct
  source 1..27: mol_type = other DNA
tacccatacgacgttccagactacgcc

SEQ ID NO: 39 - protein, 9 aa, synthetic construct
  REGION 1..9: note = Synthetic construct
YPYDVPDYA

SEQ ID NO: 40 - DNA, 33 bp, synthetic construct
  misc_feature 1..33: note = Synthetic construct
  source 1..33: mol_type = other DNA
gaacaaaagttaatttctgaagaagatttggaa

SEQ ID NO: 41 - protein, 10 aa, synthetic construct
  REGION 1..10: note = Synthetic construct
EQKLISEEDL

SEQ ID NO: 42 - DNA, 24 bp, synthetic construct
  misc_feature 1..24: note = Synthetic construct
  source 1..24: mol_type = other DNA
gactacaaggatgacgatgacaaa

SEQ ID NO: 43 - protein, 8 aa, synthetic construct
  REGION 1..8: note = Synthetic construct
DYKDDDDK

SEQ ID NO: 44 - DNA, 42 bp, synthetic construct
  misc_feature 1..42: note = Synthetic construct
  source 1..42: mol_type = other DNA
ggtaagccaattccaaatcctttgttgggtttggactccacc

SEQ ID NO: 45 - protein, 14 aa, synthetic construct
  REGION 1..14: note = Synthetic construct
GKPIPNPLLGLDST

SEQ ID NO: 46 - DNA, 18 bp, synthetic construct
  misc_feature 1..18: note = Synthetic construct
  source 1..18: mol_type = other DNA
catcatcatcatcatcat

SEQ ID NO: 47 - protein, 6 aa, synthetic construct
  REGION 1..6: note = Synthetic construct
HHHHHH

SEQ ID NO: 48 - DNA, 1089 bp, Psilocybe cubensis
  source 1..1089: mol_type = genomic DNA
atggcgttcgatctcaagactgaagacggcctcatcacatatctcactaaacatctttctttggacgtcgacacgagcggagtgaagcgccttagcggaggctttgtcaatgtaacctggcgcattaagctcaatgctccttatcaaggtcatacgagcatcatcctgaagcatgctcagccgcacatgtctacggatgaggattttaagataggtgtagaacgttcggtttacgaataccaggctatcaagctcatgatggccaatcgggaggttctgggaggcgtggatggcatagtttctgtgccagaaggcctgaactacgacttagagaataatgcattgatcatgcaagatgtcgggaagatgaagacccttttagattatgtcaccgccaaaccgccacttgcgacggatatagcccgccttgttgggacagaaattggggggttcgttgccagactccataacataggccgcgagaggcgagacgatcctgagttcaaattcttctctggaaatattgtcggaaggacgacttcagaccagctgtatcaaaccatcatacccaacgcagcgaaatatggcgtcgatgaccccttgctgcctactgtggttaaggaccttgtggacgatgtcatgcacagcgaagagacccttgtcatggcggacctgtggagtggaaatattcttctccagttggaggagggaaacccatcgaagctgcagaagatatatatcctggattgggaactttgcaagtacggcccagcgtcgttggacctgggctatttcttgggtgactgctatttgatatcccgctttcaagacgagcaggtcggtacgacgatgcggcaagcctacttgcaaagctatgcgcgtacgagcaagcattcgatcaactacgccaaagtcactgcaggtattgctgctcatattgtgatgtggaccgactttatgcagtgggggagcgaggaagaaaggataaattttgtgaaaaagggggtagctgcctttcacgacgccaggggcaacaacgacaatggggaaattacgtctaccttactgaaggaatcatccactgcgtaa

SEQ ID NO: 49 - protein, 362 aa, Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 50 - DNA, 6379 bp, synthetic construct
  misc_feature 1..6379: note = Synthetic construct
  source 1..6379: mol_type = other DNA
cgagatctttgtgttcggttacccggctcagatcctaacttcttcttttggtatgtttattcgtataagttactgttgtccacaggcaatactctgcagaaaattaaaacggcattaatgctaggacaaccagaattgttactactgtatgtgcgatagttgataactgcaacattatgcccggtatattctcaaaaaaccctattactgcatacgaagaaatcgcaagagaaatctttcggtttggaaaagctcactgtgaggttccttggagccaatagtaatacagcacaatccaaggaaaaatctggcctatatgcaaggaaggagagatagtcaaaagcattctttcccctagaagttggtgcatatatggcatcgttaaaacatattacccccaaaatttcttctctaaacgatgtgcttggcctttgttttggtttttgatgtcggtcgtttgaggccccttgcggaaaatcgagatcgccgaatggcacgcgagggaagggaaataaggtttaaaggcactgaaacaataggcaagaagtaggcgagagccgacatacgagactaaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcatttgtcatcgtcatccttgtagtcttcagcaacacatgggtatgaaatagaaacttcttcagccaatgctttaatcttcatcatgatttgcaacatttcttcagttgtagttcttggattaattgaacacaatctaataacaaccttttccttcaattctgtagtagataacatagcgaaacctctatgtgtgatttccttaaccaatttcttattaatttcattaatagtatctgttgatgccaattcagatggaatgtatctaaaagtaacgatacccaattgagctggtgtaacaacttcccaatcttttgctttacccaaaaatgcttcaacttgttctgctaacatgataccatgatcgattgcttgtctaaaagcagcaacaccgaaaactttaaaagacaaccaaaccttcaaagctctgaatcttcttgacaattcgataccacattcaccgaaattaatttcaccttcaacgttagtttctgaatccttgatgtattctggcatcattctaaaagtctttgacaaatattgagagtttctgatcaaaacacaaccaacatcgtatggttggaacaaccacttatgtggatctaaagtcaaagaatctgctctatgaataccttgcaacatagctgaacccttttcagacaagatagctggagcaccataagaaccatcagcatgcaaccaaacatcttcatcgttacacaaatctgctaattcgttcaaagaatcaacagcaccacaatttgtagtaccagcatttgcaataacacagaatggctttttacccttagttctatcttctttaatttgtttcttcaaagctgaaacagagattctcaaatgttcatctgtttcgattctacagatttgatgatgtttaaaacctaaaaccttcaatgctctatcaactgagaaatgtgtttgatcagagaagtaaacaacagcattttcgatatcgttgttcaacttagcctgtcttgcaacagtcaaagctgtcaaatttgccattgaaccaccagaaacaaataaaccttcagctgaatctggaaaacccaacatagatttcaaccaattaattgtagtcaattcgatttgttcagcacctgcaccagcaatccatgcagttggaaaaacattaaaaccagaagccaagaaatctgcaacaacaccaacgtaattatttggacctggaacaaaagccaagaaatgtggatgatcaacatgtgtaatttgattaaaaacgtttctgttcaagaaatgcaacaattcctttggatctgaaccattttctgggatagattcagtcaacttatttctcaagatatcagaatcgattgtttctgaaactggcttagacttcaaatggttcatgtgatcgatgatcaaatcaactgcttggtaacccaattgtctcatttcttcagctgacaattgcaaattttcagacattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtctgaacaaaagttaatttctgaagaagatttggaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaataccacttttcaatgaaacggatattgatatgctagtaaaaggacgagctcaagagcgaaaatataagtaaagaattcgagtgcacttgtctccatgcagcaagatttcatatgagtcttttttatctttttactttttacattacacgatatgcactttatgaaaatttaacgaggttggaagccggataatcaaccaaaatcaggcacgaaggcacactcgtatatgcatgttgttgaaactctgttacgctgaactaacaatcacacatgtagaggtcaccgggaaaagttgcgaccccatggaaggtcgatctcttcgtttggctttgcttggctggcggcattgcgcttcttcgcttatacccgtctcttgacgctcgagctcgttcattgagatacctttattcttgcacattttctggcttttttcgctactcgggtacatgtaatcatgcacacagaaggtgctgtagggtgaaagttcctttgtgctgtcgtttgtttttaatgccaaactttccggtgatcaataaccacctc

SEQ ID NO: 51 - DNA, 3840 bp, synthetic construct
  misc_feature 1..3840: note = Synthetic construct
  source 1..3840: mol_type = other DNA
cggcatgcaaacatctacacaattagcaagggcaatccatattttgtcttttcgcgccctggaaaggcctaagtaatgtcgtaaacgcattctatctgtacttcaactctcctctgtgcattggtttgtgcaaatcacattttacgatactgccagatatatgcaaaaagagaaaaccaagggaccagaacaaagcaaaattacgatattcttcgaattccttcgtgcttgactaagacaaagggatggacgtagcgatttttagcgggccaagaactggttccgaaaaagcacaggtacaccgaaccctcagctaaggagggacagcaccgatgcggaaggacaaactttctttttgcctatcacagtatcttatcgagctaactattttcgacacacatgaaaaagcagaaatattaacgaaaaagaaaagaaagaccatgtcatgtacgggcaatcagaatctgtaacaagcgccatttttttttctgtatcgggccctccttactgctctccttccgtgtaacgcgttatgaaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgggaggtccgatgagcggtttccattcgggggaggcgctgctcggtgacctcgccaccggtcagctgaccaggctgtgcgaggtggcggggctgaccgaggccgacacggcggcctacacgggggtgctgatcgaaagtctggggacgtcggccggacggccgttgtccctgccacccccgtcgcggacctttctctccgacgaccacacccccgtggagttctccctggccttcctgccgggacgcgcaccgcacctgcgggtcctggtggaaccgggctgctccagcggcgacgacctggcggaaaacggccgggccggtctgcgggcggtccacaccatggcggaccgctggggattctccaccgagcaactcgaccggctggaggacctgttcttcccctcctcccccgagggcccgctggccctgtggtgcgccctggagctccgctccggtggggtgccgggggtgaaggtctacctcaaccccgcggcgaatggcgccgaccgggccgccgagacggtacgcgaggcgctggccaggctgggccacctgcaggcgttcgacgcgctgccccgggcggacggcttcccgttcctcgccctggacctcggcgactgggacgccccgcgggtgaagatctacctcaaacacctcggcatgtccgccgccgacgcgggctccctcccccggatgtcgcccgcaccgagccgggagcagctggaggagttcttccgcaccgccggtgacctcccggccccgggagacccggggcccaccgaggacaccggccggctcgccgggcgccccgccctcacctgccactccttcacggagacggcgaccgggcggcccagcggctacaccctccacgtgccggtccgcgactacgtccggcacgacggcgaggcacgggaccgggcggtggccgtgctgcgcgaacatgacatggacagtgcggcactggaccgggcgctggccgccgtgagcccccgcccgctgagtgacggggtgggcctgatcgcctatctggcactggtccaccagcgcggccggccgacacgggtgaccgtctacgtctcctccgaggcgtacgaggtgcggccgccccgcgagacggtccccacccgcgaccgggcgcgggcacggctgcatcatcatcatcatcattgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaatacgactctctcgaaatttttcttaacgcgtccttgtactgcgtctaacgcttttgccacttggatttctattataggaaatagtctcacttactgggcgacgaattttcgcgttttgatgaagcacaggaagaatttcttttttttttggcttcttctggttccgttttttacgcgcacaaatctaaaaaaagaaataattataacctagtctcgaaaattttcatcgatccattcgttcctttttttcgattttttcagatcaaaattcttgtttctttctttgtcttagtttatattaaaagatattttgattttactcctgaactatttattctttctaagaaggccagaacactacagctgttttaaccgactacgaagttctccattctcgaacactagccttcatttaccaaacaggaactagcgtatatcattagtccttattcgaaaagagattggtagatatttattgtagtttgtgagaaggagaaaatactgtcattggactgatagttagaggacattaacctctcttacgttcgctca

SEQ ID NO: 52 - DNA, 6190 bp, synthetic construct
  misc_feature 1..6190: note = Synthetic construct
  source 1..6190: mol_type = other DNA
cgagatctttgtgttcggttacccggctcagatcctaacttcttcttttggtatgtttattcgtataagttactgttgtccacaggcaatactctgcagaaaattaaaacggcattaatgctaggacaaccagaattgttactactgtatgtgcgatagttgataactgcaacattatgcccggtatattctcaaaaaaccctattactgcatacgaagaaatcgcaagagaaatctttcggtttggaaaagctcactgtgaggttccttggagccaatagtaatacagcacaatccaaggaaaaatctggcctatatgcaaggaaggagagatagtcaaaagcattctttcccctagaagttggtgcatatatggcatcgttaaaacatattacccccaaaatttcttctctaaacgatgtgcttggcctttgttttggtttttgatgtcggtcgtttgaggccccttgcggaaaatcgagatcgccgaatggcacgcgagggaagggaaataaggtttaaaggcactgaaacaataggcaagaagtaggcgagagccgacatacgagactaaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattcatttgtcatcgtcatccttgtagtccattgccttcaaatgttccttattaaactttctcaaatcgtaaaccaaagagttcaagatatccaattcaacatgttccataacaacgatcttgtaccagttgttagttgggttatgaacttctggaactaaaaagtacttttcagcgatttccttattaacatattgttcttcaattgtaacaatattcattgaatcttctttgtaatatttaattctcatatcgttcaattgcttacacaaccatttagttctatttctcaacttattaatcttttccatccaaccgtatggaccataagaagctaaaaccatccaaattgcaacagcatttgaaccagatcttgaaccagacaatgtaacatccaaattttcgatgtaagttgcttcctttgtcaaagtgttatggatcaaattctttcttgaaacgaagataccagtaccgtatggagcttgcaacatcttatgaccatctaatgtgattgaagaaacgttcttattagagaaatcagttttacattccttattatcaattggatatataaaaccaccaaatgcaccatcaacatgaattttgtattccaaattgtacttatcgaagatgttagcgtacaaatctggatcatcaactgaaccaaacattgtagtacccatgttagagataacgatgaagtactttttaccaatttctttagcttctttaacaattgaatccaatgtattttcttgaatttttcttgaataaaaatcaactggaaccttaataatatcgatgttcaacaaatctgaacctttgtatgcagagtaatgtgtatctgctgaagtgatgatagcgatttcttcatgcttagcctttctttctttcttgaagtagtttctgtaaacccacattgcttggatgttagcttctgtaccaccttgagtaacgtaaccatcaaattcttcatcgttaccgttcaaaacatcgattgctaacaattggattaattctctttcgatatcgaatgtaccaccgaacaagatatcagccttatcataagtatgacaaccgatatggtttgggttttgaataaaagttctcaagtatggtgaatgcttaacgaaagaatgatcatcatagaaaactgtatcatccaacttagtacctggaataccgattgtcttagtgttatcgtagttcaaagtcttttccaaagattcagtaatcttttcatccatttcttgttgtgtgtactttctccagaacttcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggcttctagttcttccgatgtcttcgttttgggtctaggtgttgttttggctgccttgtatatcttcagagaccaattattcgctgcttctaagccaaaggtggctccagtttccactacgaagcctgccaacggttccgctaacccaagagacttcatcgccaagatgaaacaaggtaagaagagaatcgtaatcttctacggttctcaaactggtaccgctgaagaatatgctattcgtttggctaaggaagctaagcaaaagttcggtctagcctccttggtttgtgatccagaagaatacgattttgaaaagttggaccaattgccagaagattctattgctttcttcgtcgttgctacctatggtgaaggtgaacctacagacaacgctgtccaattgttgcaaaacttgcaagatgaaagcttcgaattctcctctggtgagagaaagttgtcaggtttgaagtacgttgtttttggtctgggtaacaagacctacgaacattacaacctcattgggagaactgttgacgctcaattggccaagatgggtgctatcagaatcggtgaaagaggtgaaggtgatgatgacaagtccatggaagaagactacttggaatggaaggatggtatgtgggaagcgtttgccactgctatgggtgttgaagaaggtcaaggtggtgactccgctgatttcgtcgtttccgaattggaatctcacccaccagaaaaggtttaccaaggtgaattttctgctagagctttaaccaaaaccaagggtattcacgacgctaagaatccttttgctgctccaattgcggttgctagagaattgttccaatctgttgtcgatagaaactgtgtccacgtcgaattcaacattgaaggctctggtatcacctatcaacacggtgaccacgttggtttgtggccattgaatccagatgttgaagtcgaacggttgttgtgtgttttaggtttagctgaaaagagagatgctgtcatctccattgaatccttagacccggctttggctaaggttccattcccagtcccaactacttacggtgctgtgttgagacactacattgacatctctgctgtcgccggtagacaaatcttgggtactttgtccaaattcgctccaaccccagaagctgaagctttcttgagaaacttgaacactaacaaggaagaataccacaacgtcgtcgctaacggttgtttgaaattgggtgaaattttgcaaatcgctaccggtaacgacattactgtcccaccaactactgccaacaccaccaaatggccaattccattcgacatcattgtttctgccatcccaagattgcaaccaagatactactctatctcttcttccccaaaaattcatccaaacaccatccacgctaccgttgttgtgctcaaatacgaaaacgttccaaccgaaccaatcccaagaaagtgggtttacggtgtcggtagtaacttcttgttgaatttaaagtacgctgttaacaaggaaccagttccatacatcactcaaaatggcgaacaaagagtcggtgtcccggaatacttgattgctggtccacgtggttcttacaagactgaatctttctacaaggctccaatccatgttagacgttctactttccgtttgccaaccaacccaaagtctccagtcatcatgattggtccaggtactggtgtcgccccattcagaggcttcgttcaagaaagagttgccttggccagaagatccatcgaaaagaacggtcctgactctttggctgactggggtcgtatttccttgttctacggttgtagaagatccgacgaagacttcttgtacaaggacgaatggccacaatacgaagctgagttgaagggtaagttcaagttgcactgtgctttctccagacaaaactacaagccagacggttctaagatttacgtccaagatttgatctgggaagacagagaacacattgccgatgccatcttaaacggtaagggttacgtctacatctgcggtgaagctaagtccatgtctaaacaagttgaagaagttctagccaagatcttgggcgaagccaaaggtggttccggtccagttgaaggtgttgctgaagtcaagttactgaaggaacggtccagattgatgttggatgtctggtctgaacaaaagttaatttctgaagaagatttggaatgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaataccacttttcaatgaaacggatattgatatgctagtaaaaggacgagctcaagagcgaaaatataagtaaagaattcgagtgcacttgtctccatgcagcaagatttcatatgagtcttttttatctttttactttttacattacacgatatgcactttatgaaaatttaacgaggttggaagccggataatcaaccaaaatcaggcacgaaggcacactcgtatatgcatgttgttgaaactctgttacgctgaactaacaatcacacatgtagaggtcaccgggaaaagttgcgaccccatggaaggtcgatctcttcgtttggctttgcttggctggcggcattgcgcttcttcgcttatacccgtctcttgacgctcgagctcgttcattgagatacctttattcttgcacattttctggcttttttcgctactcgggtacatgtaatcatgcacacagaaggtgctgtagggtgaaagttcctttgtgctgtcgtttgtttttaatgccaaactttccggtgatcaataaccacctc

SEQ ID NO: 53 - DNA, 4101 bp, synthetic construct
  misc_feature 1..4101: note = Synthetic construct
  source 1..4101: mol_type = other DNA
cggcatgcaaacatctacacaattagcaagggcaatccatattttgtcttttcgcgccctggaaaggcctaagtaatgtcgtaaacgcattctatctgtacttcaactctcctctgtgcattggtttgtgcaaatcacattttacgatactgccagatatatgcaaaaagagaaaaccaagggaccagaacaaagcaaaattacgatattcttcgaattccttcgtgcttgactaagacaaagggatggacgtagcgatttttagcgggccaagaactggttccgaaaaagcacaggtacaccgaaccctcagctaaggagggacagcaccgatgcggaaggacaaactttctttttgcctatcacagtatcttatcgagctaactattttcgacacacatgaaaaagcagaaatattaacgaaaaagaaaagaaagaccatgtcatgtacgggcaatcagaatctgtaacaagcgccatttttttttctgtatcgggccctccttactgctctccttccgtgtaacgcgttatgaaattaagtcctcagcgagctcgcatggaatgcgtgcgatgagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacggaatgcgtgcgattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtccatcggtgctgaaattgactctttggttccagctccaccaggtttgaacggtaccgctgctggttacccagccaagactcaaaaggaattgtctaacggcgatttcgatgctcacgatggtctgtccttggctcaattgactccatacgatgttttaaccgctgctttgccattgccagcgccagcttctagtactggtttctggtggagagaaactggtccagttatgtctaagctcttggctaaagccaactacccattgtacacccattacaagtatttaatgttgtaccacactcacattttacctttgttaggtccaagaccacctttggaaaattctacccacccatctccatcaaatgctccttggagatccttcttgaccgatgacttcaccccattagaaccatcttggaacgttaacggtaactccgaagcacaatccactatcagattgggtattgaaccaattggtttcgaagccggtgctgctgccgacccattcaaccaagctgccgtcacccaattcatgcactcctacgaagctactgaagttggtgccactctaactttgttcgaacacttcagaaacgacatgttcgtcggtccagagacttacgctgccttgagagctaagattcctgaaggtgagcacaccactcaatctttcttggctttcgacttggacgccggtcgtgtcactaccaaggcttacttcttcccaatcttgatgtctttgaagaccggtcaatctacgaccaaagttgtttccgattctatcttgcacctagctttgaagtctgaagtttggggtgtccaaaccattgccgctatgtcggtcatggaagcttggatcggttcttacggtggtgctgctaagaccgaaatgatctccgttgactgtgtcaacgaagctgactccagaatcaagatctacgttagaatgccacacactagcttgagaaaggtcaaagaagcttattgtttgggtggccgtttgactgacgaaaacaccaaggaaggtttgaaattgttggatgaattgtggagaactgttttcggtatcgatgacgaagatgctgaattaccacaaaactctcacagaactgctggtactatttttaactttgaactaagaccaggtaagtggttcccagaaccaaaggtctacttgccagtcagacactactgtgaatccgacatgcaaattgcctccagattacaaactttctttggtcgtttgggttggcacaacatggaaaaggactactgcaagcatttggaagacttattccctcaccacccattgtcctcctctaccggtacccacactttcttgtctttttcttacaagaagcaaaagggtgtttacatgaccatgtactacaacttgagagtttattctacacaccaccatcatcatcattgaatcgcgtgcattcatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatcgcgtcccaattcgccctatagtgagtcgtattacgcgcgctcactggccgtcgttttacaacgtcgtgactgggaaaaccctggcgttacccctgcaggactagtgctgaggcattaatacgactctctcgaaatttttcttaacgcgtccttgtactgcgtctaacgcttttgccacttggatttctattataggaaatagtctcacttactgggcgacgaattttcgcgttttgatgaagcacaggaagaatttcttttttttttggcttcttctggttccgttttttacgcgcacaaatctaaaaaaagaaataattataacctagtctcgaaaattttcatcgatccattcgttcctttttttcgattttttcagatcaaaattcttgtttctttctttgtcttagtttatattaaaagatattttgattttactcctgaactatttattctttctaagaaggccagaacactacagctgttttaaccgactacgaagttctccattctcgaacactagccttcatttaccaaacaggaactagcgtatatcattagtccttattcgaaaagagattggtagatatttattgtagtttgtgagaaggagaaaatactgtcattggactgatagttagaggacattaacctctcttacgttcgctca

SEQ ID NO: 54 - DNA, 5360 bp, synthetic construct
  misc_feature 1..5360: note = Synthetic construct
  source 1..5360: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgaattaattccgatatccatggccatcgccggctgggcagcgaggagcagcagaccagcagcagcggtcggcagcaggtatttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca